SEQUENCE LISTING (229 sequences)

SEQ ID NO: 1 - DNA, 4206 bp, Homo sapiens
atgaaatcctgcggagtgtcgctcgctaccgccgccgctgccgccgccgctttcggtgatgaggaaaagaaaatggcggcgggaaaagcgagcggcgagagcgaggaggcgtcccccagcctgacagccgaggagagggaggcgctcggcggactggacagccgcctctttgggttcgtgagatttcatgaagatggcgccaggacgaaggccctactgggcaaggctgttcgctgctatgaatctctaatcttaaaagctgaaggaaaagtggagtctgatttcttttgtcaattaggtcacttcaacctcttattggaagattatccaaaagcattatctgcataccagaggtactacagtttacagtctgactactggaagaatgctgcctttttatatggtcttggtttggtctacttccattataatgcatttcagtgggcaattaaagcatttcaggaggtgctttatgttgatcccagcttttgtcgagccaaggaaattcatttacgacttgggcttatgttcaaagtgaacacagactatgagtctagtttaaagcattttcagttagctttggttgactgtaatccctgcactttgtccaatgctgaaattcaatttcacattgcccacttatatgaaacccagaggaaatatcattctgcaaaagaagcttatgaacaacttttgcagacagagaatctttctgcacaagtaaaagcaactgtcttacaacagttaggttggatgcatcacactgtagatctcctgggagataaagccaccaaggaaagctatgctattcagtatctccaaaagtccttggaagcagatcctaattctggccagtcctggtatttcctcggaaggtgctattcaagtattgggaaagttcaggatgcctttatatcttacaggcagtctattgataaatcagaagcaagtgcagatacatggtgttcaataggtgtgctatatcagcagcaaaatcagcccatggatgctttacaggcctatatttgtgctgtacaattggaccatggccatgctgcagcctggatggacctaggcactctctatgaatcctgcaaccagcctcaggatgccattaaatgctacttaaatgcaactagaagcaaaagttgtagtaatacctctgcacttgcagcacgaattaagtatttacaggctcagttgtgtaaccttccacaaggtagtctacagaataaaactaaattacttcctagtattgaggaggcgtggagcctaccaattcccgcagagcttacctccaggcagggtgccatgaacacagcacagcagaatacttctgacaattggagtggtggacatgctgtgtcacatcctccagtacagcaacaagctcattcatggtgtttgacaccacagaaattacagcatttggaacagctccgcgcaaatagaaataatttaaatccagcacagaaactgatgctggaacagctggaaagtcagtttgtcttaatgcaacaacaccaaatgagaccaacaggagttgcacaggtacgatctactggaattcctaatgggccaacagctgactcatcactgcctacaaactcagtctctggccagcagccacagcttgctctgaccagagtgcctagcgtctctcagcctggagtccgtcctgcctgccctgggcagcctttggccaatggacccttttctgcaggccatgttccctgtagcacatcaagaacgctgggaagtacagacactattttgataggcaataatcatataacaggaagtggaagtaatggaaacgtgccttacctgcagcgaaacgcactcactctacctcataaccgcacaaacctgaccagcagcgcagaggagccgtggaaaaaccaactatctaactccactcaggggcttcacaaaggtcagagttcacattcggcaggtcctaatggtgaacgacctctctcttccactgggccttcccagcatctccaggcagctggctctggtattcagaatcagaacggacatcccaccctgcctagcaattcagtaacacagggggctgctctcaatcacctctcctctcacactgctacctcaggtggacaacaaggcattaccttaaccaaagagagcaagccttcaggaaacatattgacggtgcctgaaacaagcaggcacactggagagacacctaacagcactgccagtgtcgagggacttcctaatcatgtccatcagatgacggcagatgctgtttgcagtcctagccatggagattctaagtcaccaggtttactaagttcagacaatcctcagctctctgccttgttgatgggaaaagccaataacaatgtgggtactggaacctgtgacaaagtcaataacatccacccagctgttcatacaaagactgataactctgttgcctcttcaccatcttcagccatttcaacagcaacaccttctccaaaatccactgagcagacaaccacaaacagtgttaccagccttaacagccctcacagtgggctacacacaattaatggagaagggatggaagaatctcagagccccatgaaaacagatctgcttctggttaaccacaaacctagtccacagatcataccatcaatgtctgtgtccatataccccagctcagcagaagttctgaaggcatgcaggaatctaggtaaaaatggcttatctaacagtagcattttgttggataaatgtccacctccaagaccaccatcttcaccataccctcccttgccaaaggacaagttgaatccacctacacctagtatttacttggaaaataaacgtgatgctttctttcctccattacatcaattttgtacaaatccgaacaaccctgttacagtaatacgtggccttgctggagctcttaagttagacctgggacttttctctactaaaactttggtggaagctaacaatgaacatatggtagaagtgaggacacagttgttgcagccagcagatgaaaactgggatcccactggaacaaagaaaatctggcattgtgaaagtaatagatctcatactacaattgctaaatatgcacagtaccaggcctcctcattccaggaatcattgagagaagaaaatgaaaaaagaagtcatcataaagaccactcagatagtgaatctacatcgtcagataattctgggaggaggaggaaaggaccctttaaaaccataaagtttgggaccaatattgacctatctgatgacaaaaagtggaagttgcagctacatgagctgactaaacttcctgcttttgtgcgtgtcgtatcagcaggaaatcttctaagccatgttggtcataccatattgggcatgaacacagttcaactatacatgaaagttccagggagcagaacaccaggtcatcaggaaaataacaacttctgttcagttaacataaatattggcccaggtgactgtgaatggtttgttgttcctgaaggttactggggtgttctgaatgacttctgtgaaaaaaataatttgaatttcctaatgggttcttggtggcccaatcttgaagatctttatgaagcaaatgttccagtgtataggtttattcagcgacctggagatttggtctggataaatgcaggcactgttcattgggttcaggctattggctggtgcaacaacattgcttggaatgttggtccacttacagcctgccagtataaattggcagtggaacggtacgaatggaacaaattgcaaagtgtgaagtcaatagtacccatggttcatctttcctggaatatggcacgaaatatcaaggtctcagatccaaagctttttgaaatgattaagtattgtcttctaagaactctgaagcaatgtcagacattgagggaagctctcattgctgcaggaaaagagattatatggcatgggcggacaaaagaagaaccagctcattactgtagcatttgtgaagtggaggtttttgatctgctttttgtcactaatgagagtaattcacgaaagacctacatagtacattgccaagattgtgcacgaaaaacaagcggaaacttggaaaactttgtggtgctagaacagtacaaaatggaggacctgatgcaagtctatgaccaatttacattagctcctccattaccatccgcctcatcttga

SEQ ID NO: 2 - protein, 1401 aa, Homo sapiens
MKSCGVSLATAAAAAAAFGDEEKKMAAGKASGESEEASPSLTAEEREALGGLDSRLFGFVRFHEDGARTKALLGKAVRCYESLILKAEGKVESDFFCQLGHFNLLLEDYPKALSAYQRYYSLQSDYWKNAAFLYGLGLVYFHYNAFQWAIKAFQEVLYVDPSFCRAKEIHLRLGLMFKVNTDYESSLKHFQLALVDCNPCTLSNAEIQFHIAHLYETQRKYHSAKEAYEQLLQTENLSAQVKATVLQQLGWMHHTVDLLGDKATKESYAIQYLQKSLEADPNSGQSWYFLGRCYSSIGKVQDAFISYRQSIDKSEASADTWCSIGVLYQQQNQPMDALQAYICAVQLDHGHAAAWMDLGTLYESCNQPQDAIKCYLNATRSKSCSNTSALAARIKYLQAQLCNLPQGSLQNKTKLLPSIEEAWSLPIPAELTSRQGAMNTAQQNTSDNWSGGHAVSHPPVQQQAHSWCLTPQKLQHLEQLRANRNNLNPAQKLMLEQLESQFVLMQQHQMRPTGVAQVRSTGIPNGPTADSSLPTNSVSGQQPQLALTRVPSVSQPGVRPACPGQPLANGPFSAGHVPCSTSRTLGSTDTILIGNNHITGSGSNGNVPYLQRNALTLPHNRTNLTSSAEEPWKNQLSNSTQGLHKGQSSHSAGPNGERPLSSTGPSQHLQAAGSGIQNQNGHPTLPSNSVTQGAALNHLSSHTATSGGQQGITLTKESKPSGNILTVPETSRHTGETPNSTASVEGLPNHVHQMTADAVCSPSHGDSKSPGLLSSDNPQLSALLMGKANNNVGTGTCDKVNNIHPAVHTKTDNSVASSPSSAISTATPSPKSTEQTTTNSVTSLNSPHSGLHTINGEGMEESQSPMKTDLLLVNHKPSPQIIPSMSVSIYPSSAEVLKACRNLGKNGLSNSSILLDKCPPPRPPSSPYPPLPKDKLNPPTPSIYLENKRDAFFPPLHQFCTNPNNPVTVIRGLAGALKLDLGLFSTKTLVEANNEHMVEVRTQLLQPADENWDPTGTKKIWHCESNRSHTTIAKYAQYQASSFQESLREENEKRSHHKDHSDSESTSSDNSGRRRKGPFKTIKFGTNIDLSDDKKWKLQLHELTKLPAFVRVVSAGNLLSHVGHTILGMNTVQLYMKVPGSRTPGHQENNNFCSVNINIGPGDCEWFVVPEGYWGVLNDFCEKNNLNFLMGSWWPNLEDLYEANVPVYRFIQRPGDLVWINAGTVHWVQAIGWCNNIAWNVGPLTACQYKLAVERYEWNKLQSVKSIVPMVHLSWNMARNIKVSDPKLFEMIKYCLLRTLKQCQTLREALIAAGKEIIWHGRTKEEPAHYCSICEVEVFDLLFVTNESNSRKTYIVHCQDCARKTSGNLENFVVLEQYKMEDLMQVYDQFTLAPPLPSASS

SEQ ID NO: 3 - DNA, 4275 bp, Mus musculus
atgaaatcctgcggagtgtcgctcgctaccgccgccgccgccgccgccgccgccgctttcggtgatgaggaaaagaaaatggcggcgggaaaagcgagcggcgagagcgaggaggcgtcccccagcctgacagcggaggagagggaggcgctcggcggactggacagccgccttttcgggttcgtgaggtttcatgaagatggcgccaggatgaaggccctgctgggcaaggctgttcgctgctacgaatctctaatcttaaaagctgaagggaaagtggagtctgatttcttttgtcaattaggtcacttcaacctcttattggaagattatccaaaagcattatctgcataccagaggtactacagtttacagtctgattactggaagaatgctgcctttttatatggtcttggtttggtctacttccattacaatgcatttcagtgggctattaaagcatttcaggaggtgctttatgtcgatcccagcttttgtcgagccaaggaaattcatttacgacttgggcttatgttcaaagtgaacacagactatgagtctagtttaaagcattttcagttagctttggttgactgtaatccctgcactttgtccaatgctgaaattcagtttcacattgcccacttatatgaaacccagaggaagtatcattctgcaaaagaagcttatgagcaacttttgcagacagaaaacctttctgcacaagtaaaagcaactattttacaacaattaggttggatgcatcacactgtggatctcctgggagataaggccaccaaggaaagttatgctattcagtatctccagaagtccttggaagcagatccaaattctggccagtcctggtatttccttggaaggtgctattcaagtattgggaaagttcaggatgcctttatatcttacaggcaatctattgataaatcagaagcaagtgcagatacatggtgttcaataggtgtgctctatcaacagcaaaatcagcctatggatgctttgcaagcttatatttgtgctgtacaattggaccacggtcatgctgcagcctggatggatctaggcactctctatgaatcctgcaaccaacctcaggatgctattaaatgctatttaaatgcaactagaagcaaaaattgtagtaatacctctggacttgcagcacgaattaagtatttacaggctcagttgtgtaaccttccacaaggtagtctacagaataaaactaaattacttcctagtattgaggaggcatggagcctaccaatccccgcagagcttacctccaggcagggtgccatgaacacagcacagcagaatacttctgataattggagtggtggcaatgcaccacctccagtagaacaacaaactcattcatggtgtttgacaccacagaaattacagcacttggaacagctccgagcaaacagaaataatttaaatccagcacagaaactaatgctggaacagctggaaagtcagtttgtcttaatgcagcaacaccaaatgagacaaacaggagttgcacaggtacggcctactggaattcttaatgggccaacagttgactcatcactgcctacaaactcagtttctggccagcagccacagcttcctctgaccagaatgcctagtgtctctcagcctggagtccacactgcctgtcctaggcagactttggccaatggacccttttctgcaggccatgttccctgtagcacatcaagaacactgggaagtacagacactgttttgataggcaataatcatgtaacaggaagtggaagtaatggaaacgtgccttacctgcagcgaaacgcacccactctacctcataaccgcacaaacctgaccagcagcacagaggagccgtggaaaaaccaactatctaactccactcaggggcttcacaaaggtccgagttcacatttggcaggtcctaatggtgaacgacctctatcttccactgggccttcccagcatctccaggcagctggctctggtattcagaatcagaatggacatcccaccctgcctagcaattcagtaacacagggggctgctctcaatcacctctcctctcacactgctacctcaggtggacaacaaggcattaccttaaccaaagagagcaagccttcaggaaacacattgacggtgcctgaaacaagcaggcaaactggagagacacctaacagcactgccagtgttgagggacttcctaatcatgtccatcaggtgatggcagatgctgtttgcagtcctagccatggagattctaagtcaccaggtttactaagttcagacaatcctcagctctctgccttgttgatgggaaaagctaataacaatgtgggtcctggaacctgtgacaaagtcaataacatccacccaactgtccatacaaagactgataattctgttgcctcttcaccatcttcagccatttccacagcaacaccttctcctaagtccactgaacagacaaccacaaacagtgttaccagccttaacagccctcacagtgggctgcacacaattaatggagaaggaatggaagaatctcagagccccattaaaacagatctgcttctagttagccacagacctagtcctcagatcataccatcaatgtctgtgtccatatatcccagctcagcagaagttctgaaagcttgcaggaatctaggtaaaaacggcctgtctaatagtagcattctgttggataaatgtccgcctccaagaccaccatcctcaccataccctcccttgccaaaggacaagttgaatccacctacacctagtatttatttggaaaataaacgtgatgctttctttcctccattacatcaattttgtacaaacccaaacaaccctgttacagtaatacgtggccttgctggagctcttaaattagacttgggacttttctctactaaaactttggtggaagctaacaatgaacatatggtagaagtgaggacacagttgttacaaccagcagatgaaaattgggaccctactggaaccaagaaaatctggcactgtgaaagtaatagatctcatactacaattgctaaatatgctcagtaccaggcctcctcattccaagaatcattgagagaagaaaatgagaaaagaagtcaccataaagaccactcagacagtgaatctacatcatcagataattctgggaaaagaagaaaaggaccctttaaaaccattaagtttgggaccaacattgacctgtctgatgacaaaaagtggaagttacagctacatgagctgactaaacttcctgccttcgtgagagttgtatctgcaggaaatcttttaagccacgttggtcatactatactgggcatgaacacagttcaactatacatgaaagttccaggaagcagaacaccaggtcatcaagaaaataacaacttctgttcagttaatataaatattggcccaggtgactgtgaatggtttgttgttcctgaaggctactggggtgttttgaatgacttctgtgaaaaaaataatttgaatttcttaatgggttcttggtggcccaaccttgaagatctatatgaagcaaatgttccagtgtataggtttattcagcgacctggagatctggtctggataaatgctggcactgttcattgggttcaagctattggctggtgcaacaatattgcttggaatgttggtccacttacagcctgtcagtataagttagcagtggaacgttatgaatggaacaagttgcaaaatgtaaagtcaatagtacccatggttcatctttcctggaatatggcacgaaatatcaaggtttcagatccaaagctttttgaaatgattaagtattgtcttctgagaacgctgaagcaatgtcagacattgagggaagctctaattgctgcaggaaaagagatcatatggcacgggcggacaaaagaagaaccagctcattattgtagtatttgtgaggtggaggtttttgatctgctttttgtcactaatgagagtaattctcgaaaaacctacatagtacattgccaagattgtgcacgaaaaacaagtgggaatctggaaaattttgtggtgctagaacagtacaaaatggaggatctgatgcaagtctatgaccaatttacattagtaagtgaaatcaacatgctcctccattaccatccgcctcatcttgatattgttccatggacattaaacatgagaccttttctgctattcagaaagtaa

SEQ ID NO: 4 - protein, 1424 aa, Mus musculus
MKSCGVSLATAAAAAAAAAFGDEEKKMAAGKASGESEEASPSLTAEEREALGGLDSRLFGFVRFHEDGARMKALLGKAVRCYESLILKAEGKVESDFFCQLGHFNLLLEDYPKALSAYQRYYSLQSDYWKNAAFLYGLGLVYFHYNAFQWAIKAFQEVLYVDPSFCRAKEIHLRLGLMFKVNTDYESSLKHFQLALVDCNPCTLSNAEIQFHIAHLYETQRKYHSAKEAYEQLLQTENLSAQVKATILQQLGWMHHTVDLLGDKATKESYAIQYLQKSLEADPNSGQSWYFLGRCYSSIGKVQDAFISYRQSIDKSEASADTWCSIGVLYQQQNQPMDALQAYICAVQLDHGHAAAWMDLGTLYESCNQPQDAIKCYLNATRSKNCSNTSGLAARIKYLQAQLCNLPQGSLQNKTKLLPSIEEAWSLPIPAELTSRQGAMNTAQQNTSDNWSGGNAPPPVEQQTHSWCLTPQKLQHLEQLRANRNNLNPAQKLMLEQLESQFVLMQQHQMRQTGVAQVRPTGILNGPTVDSSLPTNSVSGQQPQLPLTRMPSVSQPGVHTACPRQTLANGPFSAGHVPCSTSRTLGSTDTVLIGNNHVTGSGSNGNVPYLQRNAPTLPHNRTNLTSSTEEPWKNQLSNSTQGLHKGPSSHLAGPNGERPLSSTGPSQHLQAAGSGIQNQNGHPTLPSNSVTQGAALNHLSSHTATSGGQQGITLTKESKPSGNTLTVPETSRQTGETPNSTASVEGLPNHVHQVMADAVCSPSHGDSKSPGLLSSDNPQLSALLMGKANNNVGPGTCDKVNNIHPTVHTKTDNSVASSPSSAISTATPSPKSTEQTTTNSVTSLNSPHSGLHTINGEGMEESQSPIKTDLLLVSHRPSPQIIPSMSVSIYPSSAEVLKACRNLGKNGLSNSSILLDKCPPPRPPSSPYPPLPKDKLNPPTPSIYLENKRDAFFPPLHQFCTNPNNPVTVIRGLAGALKLDLGLFSTKTLVEANNEHMVEVRTQLLQPADENWDPTGTKKIWHCESNRSHTTIAKYAQYQASSFQESLREENEKRSHHKDHSDSESTSSDNSGKRRKGPFKTIKFGTNIDLSDDKKWKLQLHELTKLPAFVRVVSAGNLLSHVGHTILGMNTVQLYMKVPGSRTPGHQENNNFCSVNINIGPGDCEWFVVPEGYWGVLNDFCEKNNLNFLMGSWWPNLEDLYEANVPVYRFIQRPGDLVWINAGTVHWVQAIGWCNNIAWNVGPLTACQYKLAVERYEWNKLQNVKSIVPMVHLSWNMARNIKVSDPKLFEMIKYCLLRTLKQCQTLREALIAAGKEIIWHGRTKEEPAHYCSICEVEVFDLLFVTNESNSRKTYIVHCQDCARKTSGNLENFVVLEQYKMEDLMQVYDQFTLVSEINMLLHYHPPHLDIVPWTLNMRPFLLFRK

SEQ ID NO: 5 - DNA, 5049 bp, Homo sapiens
atgcatcgggcagtggaccctccaggggcccgcgctgcacgggaagcctttgcccttgggggcctgagctgtgctggggcctggagctcctgcccgcctcatccccctcctcgtagcgcatggctgcctggaggcagatgctcagccagcattgggcagcccccgcttcctgctcccctacccccttcacatggcagtagttctgggcaccccagcaaaccatattatgctccaggggcgcccactccaagacccctccatgggaagctggaatccctgcatggctgtgtgcaggcattgctccgggagccagcccagccagggctttgggaacagcttgggcaactgtacgagtcagagcacgatagtgaggaggccacacgctgctaccacagcgcccttcgatacggaggaagcttcgctgagctggggccccgcattggccgactgcagcaggcccagctctggaactttcatactggctcctgccagcaccgagccaaggtcctgcccccactggagcaagtgtggaacttgctacaccttgagcacaaacggaactatggagccaagcggggaggtcccccggtgaagcgagctgctgaacccccagtggtgcagcctgtgcctcctgcagcactctcaggcccctcaggggaggagggcctcagccctggaggcaagcgaaggagaggctgcaactctgaacagactggccttcccccagggctgccactgcctccaccaccattaccaccaccaccaccaccaccaccaccaccaccaccacccctgcctggcctggctaccagccccccatttcagctaaccaagccagggctgtggagtaccctgcatggagatgcctggggcccagagcgcaagggttcagcacccccagagcgccaggagcagcggcactcgctgcctcacccatatccatacccagctccagcgtacaccgcgcacccccctggccaccggctggtcccggctgctcccccaggcccaggcccccgccccccaggagcagagagccatggctgcctgcctgccacccgtccccccggaagtgaccttagagagagcagagttcagaggtcgcggatggactccagcgtttcaccagcagcaaccaccgcctgcgtgccttacgccccttcccggccccctggcctccccggcaccaccaccagcagcagcagtagcagcagcagcaacactggtctccggggcgtggagccgaacccaggcattcccggcgctgaccattaccaaactcccgcgctggaggtctctcaccatggccgcctggggccctcggcacacagcagtcggaaaccgttcttgggggctcccgctgccactccccacctatccctgccacctggaccttcctcaccccctccacccccctgtccccgcctcttacgccccccaccaccccctgcctggttgaagggtccggcctgccgggcagcccgagaggatggagagatcttagaagagctcttctttgggactgagggacccccccgccctgccccaccacccctcccccatcgcgagggcttcttggggcctccggcctcccgcttttctgtgggcactcaggattctcacacccctcccactcccccaaccccaaccaccagcagtagcaacagcaacagtggcagccacagcagcagccctgctgggcctgtgtcctttcccccaccaccctatctggccagaagtatagacccccttccccggcctcccagcccagcacagaacccccaggacccacctcttgtacccctgactcttgccctgcctccagcccctccttcctcctgccaccaaaatacctcaggaagcttcaggcgcccggagagcccccggcccagggtctccttcccaaagacccccgaggtggggccggggccacccccaggccccctgagtaaagccccccagcctgtgccgcccggggttggggagctgcctgcccgaggccctcgactctttgattttccccccactccgctggaggaccagtttgaggagccagccgaattcaagatcctacctgatgggctggccaacatcatgaagatgctggacgaatccattcgcaaggaagaggaacagcaacaacacgaagcaggcgtggccccccaacccccgctgaaggagccctttgcatctctgcagtctcctttccccaccgacacagcccccaccactactgctcctgctgtcgccgtcaccaccaccaccaccaccaccaccaccaccacggccacccaggaagaggagaagaagccaccaccagccctaccaccaccaccgcctctagccaagttccctccaccctctcagccacagccaccaccacccccaccccccagcccggccagcctgctcaaatccttggcctccgtgctggagggacaaaagtactgttatcgggggactggagcagctgtttccacccggcctgggcccttgcccaccactcagtattcccctggccccccatcaggtgctaccgccctgccgcccacctcagcggcccctagcgcccagggctccccacagccctctgcttcctcgtcatctcagttctctacctcaggcgggccctgggcccgggagcgcagggcgggcgaagagccagtcccgggccccatgacccccacccaaccgcccccacccctatctctgccccctgctcgctctgagtctgaggtgctagaagagatcagccgggcttgcgagacccttgtggagcgggtgggccggagtgccactgacccagccgacccagtggacacagcagagccagcggacagtgggactgagcgactgctgccccccgcacaggccaaggaggaggctggcggggtggcggcagtgtcaggcagctgtaagcggcgacagaaggagcatcagaaggagcatcggcggcacaggcgggcctgtaaggacagtgtgggtcgtcggccccgtgagggcagggcaaaggccaaggccaaggtccccaaagaaaagagccgccgggtgctggggaacctggacctgcagagcgaggagatccagggtcgtgagaagtcccggcccgatcttggcggggcctccaaggccaagccacccacagctccagcccctccatcagctcctgcaccttctgcccagcccacacccccgtcagcctctgtccctggaaagaaggctcgggaggaagccccagggccaccgggtgtcagccgggccgacatgctgaagctgcgctcacttagtgaggggccccccaaggagctgaagatccggctcatcaaggtagagagtggtgacaaggagacctttatcgcctctgaggtggaagagcggcggctgcgcatggcagacctcaccatcagccactgtgctgctgacgtcgtgcgcgccagcaggaatgccaaggtgaaagggaagtttcgagagtcctacctttcccctgcccagtctgtgaaaccgaagatcaacactgaggagaagctgccccgggaaaaactcaacccccctacacccagcatctatctggagagcaaacgggatgccttctcacctgtcctgctgcagttctgtacagaccctcgaaatcccatcacagtgatccggggcctggcgggctccctgcggctcaacttgggcctcttctccaccaagaccctggtggaagcgagtggcgaacacaccgtggaagttcgcacccaggtgcagcagccctcagatgagaactgggatctgacaggcactcggcagatctggccttgtgagagctcccgttcccacaccaccattgccaagtacgcacagtaccaggcctcatccttccaggagtctctgcaggaggagaaggagagtgaggatgaggagtcagaggagccagacagcaccactggaacccctcctagcagcgcaccagacccgaagaaccatcacatcatcaagtttggcaccaacatcgacttgtctgatgctaagcggtggaagccccagctgcaggagctgctgaagctgcccgccttcatgcgggtaacatccacgggcaacatgctgagccacgtgggccacaccatcctgggcatgaacacggtgcagctgtacatgaaggtgcccggcagccgaacgccaggccaccaggagaataacaacttctgctccgtcaacatcaacattggcccaggcgactgcgagtggttcgcggtgcacgagcactactgggagaccatcagcgctttctgtgatcggcacggcgtggactacttgacgggttcctggtggccaatcctggatgatctctatgcatccaatattcctgtgtaccgcttcgtgcagcgacccggagacctcgtgtggattaatgcggggactgtgcactgggtgcaggccaccggctggtgcaacaacattgcctggaacgtggggcccctcaccgcctatcagtaccagctggccctggaacgatacgagtggaatgaggtgaagaacgtcaaatccatcgtgcccatgattcacgtgtcatggaacgtggctcgcacggtcaaaatcagcgaccccgacttgttcaagatgatcaagttctgcctgctgcagtccatgaagcactgccaggtgcaacgcgagagcctggtgcgggcagggaagaaaatcgcttaccagggccgtgtcaaggacgagccagcctactactgcaacgagtgcgatgtggaggtgtttaacatcctgttcgtgacaagtgagaatggcagccgcaacacgtacctggtacactgcgagggctgtgcccggcgccgcagcgcaggcctgcagggcgtggtggtgctggagcagtaccgcactgaggagctggctcaggcctacgacgccttcacgctggtgagggcccggcgggcgcgcgggcagcggaggagggcactggggcaggctgcagggacgggcttcgggagcccggccgcgcctttccctgagcccccgccggctttctccccccaggccccagccagcacgtcgcgatga

SEQ ID NO: 6 - protein, 1682 aa, Homo sapiens
MHRAVDPPGARAAREAFALGGLSCAGAWSSCPPHPPPRSAWLPGGRCSASIGQPPLPAPLPPSHGSSSGHPSKPYYAPGAPTPRPLHGKLESLHGCVQALLREPAQPGLWEQLGQLYESEHDSEEATRCYHSALRYGGSFAELGPRIGRLQQAQLWNFHTGSCQHRAKVLPPLEQVWNLLHLEHKRNYGAKRGGPPVKRAAEPPVVQPVPPAALSGPSGEEGLSPGGKRRRGCNSEQTGLPPGLPLPPPPLPPPPPPPPPPPPPLPGLATSPPFQLTKPGLWSTLHGDAWGPERKGSAPPERQEQRHSLPHPYPYPAPAYTAHPPGHRLVPAAPPGPGPRPPGAESHGCLPATRPPGSDLRESRVQRSRMDSSVSPAATTACVPYAPSRPPGLPGTTTSSSSSSSSNTGLRGVEPNPGIPGADHYQTPALEVSHHGRLGPSAHSSRKPFLGAPAATPHLSLPPGPSSPPPPPCPRLLRPPPPPAWLKGPACRAAREDGEILEELFFGTEGPPRPAPPPLPHREGFLGPPASRFSVGTQDSHTPPTPPTPTTSSSNSNSGSHSSSPAGPVSFPPPPYLARSIDPLPRPPSPAQNPQDPPLVPLTLALPPAPPSSCHQNTSGSFRRPESPRPRVSFPKTPEVGPGPPPGPLSKAPQPVPPGVGELPARGPRLFDFPPTPLEDQFEEPAEFKILPDGLANIMKMLDESIRKEEEQQQHEAGVAPQPPLKEPFASLQSPFPTDTAPTTTAPAVAVTTTTTTTTTTTATQEEEKKPPPALPPPPPLAKFPPPSQPQPPPPPPPSPASLLKSLASVLEGQKYCYRGTGAAVSTRPGPLPTTQYSPGPPSGATALPPTSAAPSAQGSPQPSASSSSQFSTSGGPWARERRAGEEPVPGPMTPTQPPPPLSLPPARSESEVLEEISRACETLVERVGRSATDPADPVDTAEPADSGTERLLPPAQAKEEAGGVAAVSGSCKRRQKEHQKEHRRHRRACKDSVGRRPREGRAKAKAKVPKEKSRRVLGNLDLQSEEIQGREKSRPDLGGASKAKPPTAPAPPSAPAPSAQPTPPSASVPGKKAREEAPGPPGVSRADMLKLRSLSEGPPKELKIRLIKVESGDKETFIASEVEERRLRMADLTISHCAADVVRASRNAKVKGKFRESYLSPAQSVKPKINTEEKLPREKLNPPTPSIYLESKRDAFSPVLLQFCTDPRNPITVIRGLAGSLRLNLGLFSTKTLVEASGEHTVEVRTQVQQPSDENWDLTGTRQIWPCESSRSHTTIAKYAQYQASSFQESLQEEKESEDEESEEPDSTTGTPPSSAPDPKNHHIIKFGTNIDLSDAKRWKPQLQELLKLPAFMRVTSTGNMLSHVGHTILGMNTVQLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNVGPLTAYQYQLALERYEWNEVKNVKSIVPMIHVSWNVARTVKISDPDLFKMIKFCLLQSMKHCQVQRESLVRAGKKIAYQGRVKDEPAYYCNECDVEVFNILFVTSENGSRNTYLVHCEGCARRRSAGLQGVVVLEQYRTEELAQAYDAFTLVRARRARGQRRRALGQAAGTGFGSPAAPFPEPPPAFSPQAPASTSR

SEQ ID NO: 7 - DNA, 4926 bp, Mus musculus
atgcatcgggcagtggaccctccaggggcccgctctgcacgggaagcctttgcccttgggggcttgagctgtgctggggcttggagctcctgcccaccccatcctcctccccgaagctcatggctgcccggaggcagatgctctgccagcgttgggcagcccccactctcagctcctttacccccatctcatggcagtagctccgggcaccctaacaaaccctattatgctcctgggacacccaccccaagaccccttcacgggaagttggaatccctacatggctgtgtccaggcattgctccgggagccagcgcagccagggttgtgggaacagcttggacagttgtatgaatcagagcacgacagtgaggaagccgtatgctgctaccatagggcccttcgctatggaggaagcttcgccgagctgggaccccggattggccgcttgcagcaggcccagctctggaactttcatgccggttcctgtcagcacagagccaaggtcctgcctcccctggagcaagtctggaatttgctgcaccttgagcacaaacggaactatggggctaagcgagggggccctccagtgaagagatctgctgaaccccccgtggtccagcctatgcctcctgcagccctctcaggcccctcaggagaggagggccttagccctggaggcaagcgcaggagaggctgcagctctgaacaggctggccttcccccaggtctgccactccctccaccacccccacccccaccgcctccaccaccaccaccaccccctccaccaccaccgctgcctggcctggctattagccccccatttcagctgactaagccagggctgtggaataccctgcatggagatgcttggggccccgagcgcaagggttcagcgccgccagagcgccaggagcagcggcactcgatgcctcattcatatccatacccagctcccgcctactccgctcatccgcccagccatcggctggtccccaacacaccccttggtccaggtccccgacccccaggagcagagagccatggctgcctgcctgccacccgtccccccggaagtgaccttagagagagcagagttcagaggtcgcggatggactccagcgtttcaccagcagcatctaccgcctgcgtgccttacgccccttcccggccccctggcctccccggcaccagcagcagcagcagcagcagcagtagcagtaacaacactggtcttcggggtgtggagccaagcccaggcattcctggcgctgaccattaccaaaaccctgcgctggagatatcccctcaccaggcccgcctgggtccctccgcacacagcagtcggaaaccatttttgacggcccctgctgccacgccccacttatccctaccccctgggaccccatcatcccctccacccccatgtcctcgcctcttgcgccctccaccgccccctgcttggatgaagggctcagcctgccgtgcagcccgagaggatggagagatcttaggggagctcttctttggtgctgagggacctccccgtcctcctcccccaccccttccccaccgtgatggcttcttggggcctccaaacccccgcttttctgtgggcactcaggattcgcataaccctcccattcccccaaccaccaccagcagcagcagcagcagcaacagccacagcagtagtcctactgggccggtgccctttccaccaccctcctatctggccagaagtatagaccccctccccaggccatccagcccaaccttgagcccccaggacccacctcttccaccactgactcttgccctgcctccagcccctccctcctcctgccaccaaaatacctcaggaagcttcaggcgctcggagagcccccggcccagggtctccttcccaaagacccccgaggtggggcaggggccacccccaggccctgtgagtaaagccccccagcctgtgccacctggggttggagagctgcctgcccgaggcccgaggctctttgatttcccacccactccgctggaggaccagtttgaagagccagccgaattcaagatcctacctgatgggctggcaaacatcatgaagatgctggatgaatccattcggaaggaggaggagcagcagcagcagcaggaggcaggcgtggctcccccacccccactcaaagagccctttgcatctctacagcctccatttcccagtgacacagccccagccaccaccactgctgcccccaccaccgccaccaccaccacaaccaccaccaccaccaccacccaagaagaggagaagaagccaccaccagccctaccaccaccaccgcctctagccaagtttcctccacctccccagccacagcccccaccacctccaccagccagcccagccagcctgctcaaatcgttggcctctgttcttgagggacaaaagtactgttaccgggggactggagcagccgtctcaaccaggcccgggtccgtgcccgccactcagtattcccctagtcctgcatcaggtgctaccgccccaccacccacttcagtggcccctagtgcccagggctcccccaagccctcggtttcctcgtcatctcagttctctacctcaggcgggccttgggcccgggagcacagggcgggtgaagagccagcaccaggccccgtgacccctgcccagttgcccccacctctgccgctgccccctgctcgttctgagtctgaggtgctagaagaaatcagtcgggcttgtgagacccttgtagagcgggtgggccggagtgccatcaacccagtggacacggcagacccagtggacagtgggactgagccacagccgccgcctgcgcaggccaaggaggagagtgggggggtggcggtagcagcagcaggtccaggtagtggcaagcgtcgtcaaaaggagcatcggcggcacaggcgggcctgtagggacagtgtgggtcgacgaccccgcgaggggagggccaaggccaaggccaaggctcccaaagaaaaaagccgaagggtgctggggaacctcgacttgcagagtgaggagatccagggccgggagaaggcccggcccgatgtcggtggggtttccaaagtcaagacacccacagctccagcacccccgcctgctcctgcacccgctgctcagccaacacccccatcagctcctgtccctgggaagaagactcgtgaggaggctccggggcctccaggtgtgagccgggcagatatgctgaagctccggtcacttagtgaggggcctcccaaggagctgaagatcaggctcatcaaggtggaaagtggggacaaggagacctttatcgcctctgaggtggaagagcggcggctgcgcatggcagacctcaccatcagccactgtgccgccgatgtcatgcgtgccagcaagaatgccaaggtgaaagggaaattccgagagtcctacctgtcccctgcccagtctgtgaaacccaagatcaacactgaggagaagctgccccgggaaaaactcaacccccctacccccagcatctatttggagagcaaacgagatgccttctcgccggtcctgctacagttctgtacagacccccggaaccccatcaccgtcatcaggggcctggctggttcacttcggctcaacttaggccttttctccaccaagactctggtggaggcgagcggtgaacatacggtggaggtccgtacccaagtacagcagccctcagacgagaactgggacctgacaggtaccagacaaatctggccctgtgagagctcccgttcccacaccaccatcgctaaatacgcacagtaccaggcctcgtccttccaggagtcactgcaggaggagagggagagtgaggatgaggaatccgaggaaccagacagcactacaggaacctctcccagcagtgcaccggaccccaagaaccatcacatcatcaagtttggcactaacatcgacctgtctgatgccaagaggtggaagccacagctacaggagctgctgaaactgcccgccttcatgcgggtaacatccacaggcaacatgctcagccacgtgggccacaccatcctgggcatgaacaccgtgcagctatacatgaaggtccctggcagccgaacgccaggccaccaagagaataacaatttctgctcagtcaacatcaacattggccctggggactgcgagtggttcgcggtacatgagcactattgggagaccatcagcgccttctgcgaccggcatggtgtggactacttgactggttcctggtggccaatcttggatgacctctatgcgtccaatattcctgtttaccgcttcgtgcagcgccctggagaccttgtgtggattaatgcagggactgtacattgggtgcaggctaccggctggtgcaacaacattgcctggaacgtggggcccctcaccgcctatcagtaccagctggccctggagcgatatgagtggaacgaggtgaagaacgtcaagtccattgtgcccatgattcatgtgtcctggaacgtcgctcgaacggtcaagatcagcgatcctgacttgttcaagatgatcaagttctgcctcctgcagtcaatgaagcactgtcaggtacagcgggagagcctggtgcgggcagggaagaagatcgcttaccaaggccgtgtcaaagacgagcctgcctactactgcaacgaatgcgacgtggaggtgttcaacatcctgttcgttacaagtgagaatggcagccgaaacacgtacctggtgcactgcgagggctgtgcgcgccgtcgcagcgcgggcctacagggcgtggtggtgctagagcagtaccgcacggaggagctggcgcaggcctacgatgccttcacactggctcccgccagcacgtctcgatga

SEQ ID NO: 8 - protein, 1641 aa, Mus musculus
MHRAVDPPGARSAREAFALGGLSCAGAWSSCPPHPPPRSSWLPGGRCSASVGQPPLSAPLPPSHGSSSGHPNKPYYAPGTPTPRPLHGKLESLHGCVQALLREPAQPGLWEQLGQLYESEHDSEEAVCCYHRALRYGGSFAELGPRIGRLQQAQLWNFHAGSCQHRAKVLPPLEQVWNLLHLEHKRNYGAKRGGPPVKRSAEPPVVQPMPPAALSGPSGEEGLSPGGKRRRGCSSEQAGLPPGLPLPPPPPPPPPPPPPPPPPPPPLPGLAISPPFQLTKPGLWNTLHGDAWGPERKGSAPPERQEQRHSMPHSYPYPAPAYSAHPPSHRLVPNTPLGPGPRPPGAESHGCLPATRPPGSDLRESRVQRSRMDSSVSPAASTACVPYAPSRPPGLPGTSSSSSSSSSSNNTGLRGVEPSPGIPGADHYQNPALEISPHQARLGPSAHSSRKPFLTAPAATPHLSLPPGTPSSPPPPCPRLLRPPPPPAWMKGSACRAAREDGEILGELFFGAEGPPRPPPPPLPHRDGFLGPPNPRFSVGTQDSHNPPIPPTTTSSSSSSNSHSSSPTGPVPFPPPSYLARSIDPLPRPSSPTLSPQDPPLPPLTLALPPAPPSSCHQNTSGSFRRSESPRPRVSFPKTPEVGQGPPPGPVSKAPQPVPPGVGELPARGPRLFDFPPTPLEDQFEEPAEFKILPDGLANIMKMLDESIRKEEEQQQQQEAGVAPPPPLKEPFASLQPPFPSDTAPATTTAAPTTATTTTTTTTTTTQEEEKKPPPALPPPPPLAKFPPPPQPQPPPPPPASPASLLKSLASVLEGQKYCYRGTGAAVSTRPGSVPATQYSPSPASGATAPPPTSVAPSAQGSPKPSVSSSSQFSTSGGPWAREHRAGEEPAPGPVTPAQLPPPLPLPPARSESEVLEEISRACETLVERVGRSAINPVDTADPVDSGTEPQPPPAQAKEESGGVAVAAAGPGSGKRRQKEHRRHRRACRDSVGRRPREGRAKAKAKAPKEKSRRVLGNLDLQSEEIQGREKARPDVGGVSKVKTPTAPAPPPAPAPAAQPTPPSAPVPGKKTREEAPGPPGVSRADMLKLRSLSEGPPKELKIRLIKVESGDKETFIASEVEERRLRMADLTISHCAADVMRASKNAKVKGKFRESYLSPAQSVKPKINTEEKLPREKLNPPTPSIYLESKRDAFSPVLLQFCTDPRNPITVIRGLAGSLRLNLGLFSTKTLVEASGEHTVEVRTQVQQPSDENWDLTGTRQIWPCESSRSHTTIAKYAQYQASSFQESLQEERESEDEESEEPDSTTGTSPSSAPDPKNHHIIKFGTNIDLSDAKRWKPQLQELLKLPAFMRVTSTGNMLSHVGHTILGMNTVQLYMKVPGSRTPGHQENNNFCSVNINIGPGDCEWFAVHEHYWETISAFCDRHGVDYLTGSWWPILDDLYASNIPVYRFVQRPGDLVWINAGTVHWVQATGWCNNIAWNVGPLTAYQYQLALERYEWNEVKNVKSIVPMIHVSWNVARTVKISDPDLFKMIKFCLLQSMKHCQVQRESLVRAGKKIAYQGRVKDEPAYYCNECDVEVFNILFVTSENGSRNTYLVHCEGCARRRSAGLQGVVVLEQYRTEELAQAYDAFTLAPASTSR

SEQ ID NO: 9 - DNA, 2256 bp, Homo sapiens
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 10 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 11 - DNA, 2124 bp, Homo sapiens
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggaggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 12 - DNA, 2241 bp, Homo sapiens
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 13 - DNA, 2214 bp, Homo sapiens
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttcttgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 14 - DNA, 2088 bp, Homo sapiens
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttcttgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaagggtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 15 - DNA, 2241 bp, Mus musculus
atgggccagactgggaagaaatctgagaagggaccggtttgttggcggaagcgtgtaaaatcagagtacatgagactgagacagctcaagaggttcagaagagctgatgaagtaaagactatgtttagttccaatcgtcagaaaattttggaaagaactgaaaccttaaaccaagagtggaagcagcggaggatacagcctgtgcacatcatgacttctgtgagctcattgcgcgggactagggagtgttcagtcaccagtgacttggattttccagcacaagtcatcccgttaaagaccctgaatgcagtcgcctcggtgcctataatgtactcttggtcgcccttacaacagaattttatggtggaagacgaaactgttttacataacattccttatatgggggatgaagttctggatcaggatggcactttcattgaagaactaataaaaaattatgatggaaaagtgcatggtgacagagaatgtggatttataaatgatgaaatttttgtggagttggtaaatgctcttggtcaatataatgatgatgatgatgacgatgatggagatgatccagatgaaagagaagaaaaacagaaagatctagaggataatcgagatgataaagaaacttgcccacctcggaaatttcctgctgataaaatatttgaagccatttcctcaatgtttccagataagggcaccgcagaagaactgaaagaaaaatataaagaactcacggagcagcagctcccaggtgctctgcctcctgaatgtactccaaacatcgatggaccaaatgccaaatctgttcagagggagcaaagcttgcattcatttcatacgctcttctgtcgacgatgttttaagtatgactgcttcctacatcccttccatgcaacacccaacacatataagaggaagaacacagaaacagctttggacaacaagccttgtggaccacagtgttaccagcatctggagggagctaaggagtttgctgctgctcttactgctgagcgtataaagacaccacctaaacgcccagggggccgcagaagaggaagacttccgaataacagtagcagacccagcacccccaccatcagtgtgctggagtcaaaggatacagacagtgacagagaagcagggactgaaactgggggagagaacaatgataaagaagaagaagagaaaaaagatgagacgtccagctcctctgaagcaaattctcggtgtcaaacaccaataaagatgaagccaaatattgaacctcctgagaatgtggagtggagtggtgctgaagcctccatgtttagagtcctcattggtacttactacgataacttttgtgccattgctaggctaattgggaccaaaacatgtagacaggtgtatgagtttagagtcaaggagtccagtatcatagcacctgttcccactgaggatgtagacactcctccaagaaagaagaaaaggaaacatcggttgtgggctgcacactgcagaaagatacaactgaaaaaggacggctcctctaaccatgtttacaactatcaaccctgtgaccatccacggcagccttgtgacagttcgtgcccttgtgtgatagcacaaaatttttgtgaaaagttttgtcaatgtagttcagagtgtcaaaaccgctttcctggatgtcggtgcaaagcacaatgcaacaccaaacagtgtccatgctacctggctgtccgagagtgtgaccctgacctctgtctcacgtgtggagctgctgaccattgggacagtaaaaatgtatcctgtaagaactgtagcattcagcggggctctaaaaagcacttactgctggcaccgtctgatgtggcaggctggggcatctttatcaaagatcctgtacagaaaaatgaattcatctcagaatactgtggggagattatttctcaggatgaagcagacagaagaggaaaagtgtatgacaaatacatgtgcagctttctgttcaacttgaacaatgattttgtggtggatgcaacccgaaagggcaacaaaattcgttttgctaatcattcagtaaatccaaactgctatgcaaaagttatgatggttaatggtgaccacaggataggcatctttgctaagagggctatccagactggtgaagagttgttttttgattacagatacagccaggctgatgccctgaagtatgtgggcatcgaacgagaaatggaaatcccttga

SEQ ID NO: 16 - protein, 746 aa, Mus musculus
MGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 17 - DNA, 2229 bp, Mus musculus
atgggccagactgggaagaaatctgagaagggaccggtttgttggcggaagcgtgtaaaatcagagtacatgagactgagacagctcaagaggttcagaagagctgatgaagtaaagactatgtttagttccaatcgtcagaaaattttggaaagaactgaaaccttaaaccaagagtggaagcagcggaggatacagcctgtgcacatcatgacttcttgttcagtcaccagtgacttggattttccagcacaagtcatcccgttaaagaccctgaatgcagtcgcctcggtgcctataatgtactcttggtcgcccttacaacagaattttatggtggaagacgaaactgttttacataacattccttatatgggggatgaagttctggatcaggatggcactttcattgaagaactaataaaaaattatgatggaaaagtgcatggtgacagagaatgtggatttataaatgatgaaatttttgtggagttggtaaatgctcttggtcaatataatgatgatgatgatgacgatgatggagatgatccagatgaaagagaagaaaaacagaaagatctagaggataatcgagatgataaagaaacttgcccacctcggaaatttcctgctgataaaatatttgaagccatttcctcaatgtttccagataagggcaccgcagaagaactgaaagaaaaatataaagaactcacggagcagcagctcccaggtgctctgcctcctgaatgtactccaaacatcgatggaccaaatgccaaatctgttcagagggagcaaagcttgcattcatttcatacgctcttctgtcgacgatgttttaagtatgactgcttcctacatcgtaagtgcagttattccttccatgcaacacccaacacatataagaggaagaacacagaaacagctttggacaacaagccttgtggaccacagtgttaccagcatctggagggagctaaggagtttgctgctgctcttactgctgagcgtataaagacaccacctaaacgcccagggggccgcagaagaggaagacttccgaataacagtagcagacccagcacccccaccatcagtgtgctggagtcaaaggatacagacagtgacagagaagcagggactgaaactgggggagagaacaatgataaagaagaagaagagaaaaaagatgagacgtccagctcctctgaagcaaattctcggtgtcaaacaccaataaagatgaagccaaatattgaacctcctgagaatgtggagtggagtggtgctgaagcctccatgtttagagtcctcattggtacttactacgataacttttgtgccattgctaggctaattgggaccaaaacatgtagacaggtgtatgagtttagagtcaaggagtccagtatcatagcacctgttcccactgaggatgtagacactcctccaagaaagaagaaaaggaaacatcggttgtgggctgcacactgcagaaagatacaactgaaaaaggacggctcctctaaccatgtttacaactatcaaccctgtgaccatccacggcagccttgtgacagttcgtgcccttgtgtgatagcacaaaatttttgtgaaaagttttgtcaatgtagttcagagtgtcaaaaccgctttcctggatgtcggtgcaaagcacaatgcaacaccaaacagtgtccatgctacctggctgtccgagagtgtgaccctgacctctgtctcacgtgtggagctgctgaccattgggacagtaaaaatgtatcctgtaagaactgtagcattcagcggggctctaaaaagcacttactgctggcaccgtctgatgtggcaggctggggcatctttatcaaagatcctgtacagaaaaatgaattcatctcagaatactgtggggagattatttctcaggatgaagcagacagaagaggaaaagtgtatgacaaatacatgtgcagctttctgttcaacttgaacaatgattttgtggtggatgcaacccgaaagggcaacaaaattcgttttgctaatcattcagtaaatccaaactgctatgcaaaagttatgatggttaatggtgaccacaggataggcatctttgctaagagggctatccagactggtgaagagttgttttttgattacagatacagccaggctgatgccctgaagtatgtgggcatcgaacgagaaatggaaatcccttga

SEQ ID NO: 18 - protein, 742 aa, Mus musculus
MGQTGKKSEKGPVCWRKRVKSEYMRLRQLKRFRRADEVKTMFSSNRQKILERTETLNQEWKQRRIQPVHIMTSCSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHRKCSYSFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 19 - DNA, 303 bp, Homo sapiens
atgcccaagaggaaggtcagctccgccgaaggcgccgccaaggaagagcccaagaggagatcggcgcggttgtcagctaaacctcctgcaaaagtggaagcgaagccgaaaaaggcagcagcgaaggataaatcttcagacaaaaaagtgcaaacaaaagggaaaaggggagcaaagggaaaacaggccgaagtggctaaccaagaaactaaagaagacttacctgcggaaaacggggaaacgaagactgaggagagtccagcctctgatgaagcaggagagaaagaagccaagtctgattaa

SEQ ID NO: 20 - protein, 100 aa, Homo sapiens
MPKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSD

SEQ ID NO: 21 - DNA, 303 bp, Macaca mulatta
atgcccaagaggaaggtcagctccgccgaaggggccgccaaggaagagcccaaaaggagatcggcgcggttgtcagctaaacctcctgccaaagtggaagcgaagccgaaaaaggcagcagcgaaggataaatcttcagacaaaaaagtgcaaacaaaagggaaaaggggagcaaagggaaaacaggccgaagtggctaaccaagaaactaaagaagatttacctgcagaaaacggggaaacgaaaactgaggagagtccagcctctgatgaagcaggagagaaagaagccaagtctgattaa

SEQ ID NO: 22 - protein, 100 aa, Macaca mulatta; identical to SEQ ID NO: 20

SEQ ID NO: 23 - protein, 43 aa, Homo sapiens
DGSSNHVYNYQPCDHPRQPCDSSCPCVIAQNFCEKFCQCSSEC

SEQ ID NO: 24 - protein, 114 aa, Homo sapiens
HLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDY

SEQ ID NO: 25 - protein, 60 aa, Homo sapiens
MARTKQTARKSTGGKAPRKQLATKAARKSAPATGGVKKPHRYRPGTVALREIRRYQKSTE

SEQ ID NO: 26 - protein, 60 aa, Homo sapiens; identical to SEQ ID NO: 25

SEQ ID NO: 27 - protein, 60 aa, Homo sapiens
MARTKQTARKSTGGKAPRKQLATKAARKSAPSTGGVKKPHRYRPGTVALREIRRYQKSTE

SEQ ID NO: 28 - DNA, 20 bp, Artificial Sequence
  /note=“Description of Artificial Sequence Synthetic primer”
ccagctcccatagctcagtc

SEQ ID NO: 29 - DNA, 20 bp, Artificial Sequence
  /note=“Description of Artificial Sequence Synthetic primer”
cttcatgcaattgtcggtca

SEQ ID NO: 30 - DNA, 20 bp, Artificial Sequence
  /note=“Description of Artificial Sequence Synthetic primer”
gctgcctcacaaacttcaca

SEQ ID NO: 31 - DNA, 20 bp, Artificial Sequence
  /note=“Description of Artificial Sequence Synthetic primer”
tgagtttgccaagcagtcac

SEQ ID NO: 32 - DNA, 24 bp, Artificial Sequence
  /note=“Description of Artificial Sequence Synthetic primer”
aatgccctggctcacaaataccac

SEQ ID NO: 33 - DNA, 24 bp, Artificial Sequence
  /note=“Description of Artificial Sequence Synthetic primer”
tgtccttccgagtgagagacacaa

SEQ ID NO: 34 - DNA, 24 bp, Artificial Sequence
  /note=“Description of Artificial Sequence Synthetic primer”
tggccaggctgaaaggataggatt

SEQ ID NO: 35 - DNA, 24 bp, Artificial Sequence
  /note=“Description of Artificial Sequence Synthetic primer”
agaatccaggtccagggctgattt

SEQ ID NO: 36 - DNA, 21 bp, Artificial Sequence
  /note=“Description of Artificial Sequence Synthetic primer”
tttggcaaagaattcagatcc

SEQ ID NO: 37 - DNA, 23 bp, Artificial Sequence
  /note=“Description of Artificial Sequence Synthetic primer”
caaatgtggtatggctgattatg

SEQ ID NO: 38 - DNA, 24 bp, Artificial Sequence
  /note=“Description of Artificial Sequence Synthetic primer”
aagtagaagacccacgaggcaaca

SEQ ID NO: 39 - DNA, 24 bp, Artificial Sequence
  /note=“Description of Artificial Sequence Synthetic primer”
tgtggcggatcttgaagttcacct

SEQ ID NO: 40 - DNA, 41 bp, Artificial Sequence
  /note=“Description of Artificial Sequence Synthetic primer”
aatggactatcatatgcttaccgtaacttgaaagtatttcg

SEQ ID NO: 41 - DNA, 45 bp, Artificial Sequence
  /note=“Description of Artificial Sequence Synthetic primer”
ctttagtttgtatgtctgttgctattatgtctactattctttccc

SEQ ID NO: 42 - DNA, 74 bp, Artificial Sequence
  /note=“Description of Artificial Sequence Synthetic primer”
aatgatacggcgaccaccgaccgtaacttgaaagtatttcgatttcttggctttatatatcnnnnnnaaaggac

SEQ ID NO: 43 - DNA, 60 bp, Artificial Sequence
  /note=“Description of Artificial Sequence Synthetic primer”
caagcagaagacggcatacgagctcttccgatcttgtggatgaatactgccatttgtctc

SEQ ID NO: 44 - DNA, 41 bp, Artificial Sequence
  /note=“Description of Artificial Sequence Synthetic primer”
ccgtaacttgaaagtntttcgatttcttggctttntntatc

SEQ ID NO: 45 - DNA, 21 bp, Artificial Sequence
  /note=“Description of Artificial Sequence shRNA target sequence”
cgacatgaagtggcgatacaa

SEQ ID NO: 46 - DNA, 21 bp, Artificial Sequence
  /note=“Description of Artificial Sequence shRNA target sequence”
tgtggtcatggcagtccattt

SEQ ID NO: 47 - DNA, 21 bp, Artificial Sequence
  /note=“Description of Artificial Sequence shRNA target sequence”
actcggaagagagtctattta

SEQ ID NO: 48 - DNA, 21 bp, Artificial Sequence
  /note=“Description of Artificial Sequence shRNA target sequence”
ttctatctggtcccgtgtttc

SEQ ID NO: 49 - DNA, 21 bp, Artificial Sequence
  /note=“Description of Artificial Sequence shRNA target sequence”
tgtggctttcaacatttcaaa

SEQ ID NO: 50 - DNA, 21 bp, Artificial Sequence
  /note=“Description of Artificial Sequence shRNA target sequence”
gcgtttgttatgaactcggga

SEQ ID NO: 51 - DNA, 21 bp, Artificial Sequence
  /note=“Description of Artificial Sequence shRNA target sequence”
ccattgattattagccatgaa

SEQ ID NO: 52 - DNA, 21 bp, Artificial Sequence
  /note=“Description of Artificial Sequence shRNA target sequence”
gaaagaagctaagtccgacta

SEQ ID NO: 53 - DNA, 21 bp, Artificial Sequence
  /note=“Description of Artificial Sequence shRNA target sequence”
acggacgtgtaggcgtaaata

SEQ ID NO: 54 - DNA, 21 bp, Artificial Sequence
  /note=“Description of Artificial Sequence shRNA target sequence”
cgaaagtttcttcaacgaaat

SEQ ID NO: 55 - DNA, 21 bp, Artificial Sequence
  /note=“Description of Artificial Sequence shRNA target sequence”
tctacaacatccgaagcattg

SEQ ID NO: 56 - DNA, 21 bp, Artificial Sequence
  /note=“Description of Artificial Sequence shRNA target sequence”
ctcagtaatcgagaaggcgtt

SEQ ID NO: 57 - DNA, 21 bp, Artificial Sequence
  /note=“Description of Artificial Sequence shRNA target sequence”
gacttcaaggagctggacata

SEQ ID NO: 58 - DNA, 21 bp, Artificial Sequence
  /note=“Description of Artificial Sequence shRNA target sequence”
caggagataatgatgggcaga

SEQ ID NO: 59 - DNA, 21 bp, Artificial Sequence
  /note=“Description of Artificial Sequence shRNA target sequence”
cgttagcgggagagatgaatg

SEQ ID NO: 60 - DNA, 21 bp, Artificial Sequence
  /note=“Description of Artificial Sequence shRNA target sequence”
atcgacaacccgcagagcatt

SEQ ID NO: 61 - DNA, 21 bp, Artificial Sequence
  /note=“Description of Artificial Sequence shRNA target sequence”
atggagctatggacgttaatt

SEQ ID NO: 62 - DNA, 21 bp, Artificial Sequence
  /note=“Description of Artificial Sequence shRNA target sequence”
gcctttattcatggtgactta

SEQ ID NO: 63 - DNA, 21 bp, Artificial Sequence
  /note=“Description of Artificial Sequence shRNA target sequence”
ccggagctaccaccacggcta

SEQ ID NO: 64 - DNA, 21 bp, Artificial Sequence
  /note=“Description of Artificial Sequence shRNA target sequence”
cgaacagtaatcctaccattt

SEQ ID NO: 65 - DNA, 21 bp, Artificial Sequence
  /note=“Description of Artificial Sequence shRNA target sequence”
agccaatctcacggtcttaaa

SEQ ID NO: 66 - DNA, 21 bp, Artificial Sequence
  /note=“Description of Artificial Sequence shRNA target sequence”
tgcatcacaaagctaacaaat

SEQ ID NO: 67 - DNA, 21 bp, Artificial Sequence
  /note=“Description of Artificial Sequence shRNA target sequence”
gcgggaaaggataaagcatca

SEQ ID NO: 68 - DNA, 21 bp, Artificial Sequence
  /note=“Description of Artificial Sequence shRNA target sequence”
gcgctgagatagtacaaatat

SEQ ID NO: 69 - DNA, 21 bp, Artificial Sequence
  /note=“Description of Artificial Sequence shRNA target sequence”
cccataacaaacaccaagtat

SEQ ID NO: 70 - DNA, 21 bp, Artificial Sequence
  /note=“Description of Artificial Sequence shRNA target sequence”
ccctatgaccaatcaggacat

SEQ ID NO: 71 - DNA, 21 bp, Artificial Sequence
  /note=“Description of Artificial Sequence shRNA target sequence”
cacttcccaaattcttcatta

SEQ ID NO: 72 - DNA, 21 bp, Artificial Sequence
  /note=“Description of Artificial Sequence shRNA target sequence”
gctgactacttgaagttcaaa

SEQ ID NO: 73 - DNA, 21 bp, Artificial Sequence
  /note=“Description of Artificial Sequence shRNA target sequence”
aggcaaggtcttggatgtgat

SEQ ID NO: 74 - DNA, 21 bp, Artificial Sequence
  /note=“Description of Artificial Sequence shRNA target sequence”
gccgcaggaaatgggcttaat

SEQ ID NO: 75 - DNA, 21 bp, Artificial Sequence
  /note=“Description of Artificial Sequence shRNA target sequence”
acattgctgtcgtcaactatg

SEQ ID NO: 76 - DNA, 21 bp, Artificial Sequence
  /note=“Description of Artificial Sequence shRNA target sequence”
cgattacagatacagaagcta

SEQ ID NO: 77 - DNA, 21 bp, Artificial Sequence
  /note=“Description of Artificial Sequence shRNA target sequence”
gcattcctgtcagcgtttgtt

SEQ ID NO: 78 - DNA, 21 bp, Artificial Sequence
  /note=“Description of Artificial Sequence shRNA target sequence”
gctgtgtataaacatcctaaa

SEQ ID NO: 79 - DNA, 21 bp, Artificial Sequence
  /note=“Description of Artificial Sequence shRNA target sequence”
cccagtggcatgaagtttgaa

SEQ ID NO: 80 - DNA, 21 bp, Artificial Sequence
  /note=“Description of Artificial Sequence shRNA target sequence”
gaacagccgaacattgtgcat

SEQ ID NO: 81 - DNA, 21 bp, Artificial Sequence
  /note=“Description of Artificial Sequence shRNA target sequence”
cagacagcttacgtgatgaaa

SEQ ID NO: 82 - DNA, 21 bp, Artificial Sequence
  /note=“Description of Artificial Sequence shRNA target sequence”
cggacaggattctgctcaatg

SEQ ID NO: 83 - DNA, 21 bp, Artificial Sequence
  /note=“Description of Artificial Sequence shRNA target sequence”
cctgagttcaattctcagcaa

SEQ ID NO: 84 - DNA, 21 bp, Artificial Sequence
  /note=“Description of Artificial Sequence shRNA target sequence”
ctgaagtattccctcctacca

SEQ ID NO: 85 - DNA, 21 bp, Artificial Sequence
  /note=“Description of Artificial Sequence shRNA target sequence”
ctacgagatcatcttcaagat

SEQ ID NO: 86 - DNA, 21 bp, Artificial Sequence
  /note=“Description of Artificial Sequence shRNA target sequence”
cctcccgagattgagatcaaa

SEQ ID NO: 87 - DNA, 21 bp, Artificial Sequence
  /note=“Description of Artificial Sequence shRNA target sequence”
cgacagcatgaagtcgttctt

SEQ ID NO: 88 - DNA, 21 bp, Artificial Sequence
  /note=“Description of Artificial Sequence shRNA target sequence”
gcactgagttactgcctataa

SEQ ID NO: 89 - DNA, 21 bp, Artificial Sequence
  /note=“Description of Artificial Sequence shRNA target sequence”
cgtctgatgtaggtctggatt

SEQ ID NO: 90 - DNA, 21 bp, Artificial Sequence
  /note=“Description of Artificial Sequence shRNA target sequence”
ctaacgtcttggaaggcgatt

SEQ ID NO: 91 - DNA, 21 bp, Artificial Sequence
  /note=“Description of Artificial Sequence shRNA target sequence”
ttcgacagctcctccggaaat

SEQ ID NO: 92 - DNA, 21 bp, Artificial Sequence
  /note=“Description of Artificial Sequence shRNA target sequence”
tcaccggagctgatggttaat

SEQ ID NO: 93 - DNA, 21 bp, Artificial Sequence
  /note=“Description of Artificial Sequence shRNA target sequence”
gcgcaaagactactctgcttt

SEQ ID NO: 94 - DNA, 21 bp, Artificial Sequence
  /note=“Description of Artificial Sequence shRNA target sequence”
gcatcgccttcaaggtcaatg

SEQ ID NO: 95 - DNA, 21 bp, Artificial Sequence
  /note=“Description of Artificial Sequence shRNA target sequence”
gctctctgattgagtctgtaa

SEQ ID NO: 96 - DNA, 21 bp, Artificial Sequence
  /note=“Description of Artificial Sequence shRNA target sequence”
gcactggagaactcgaaagaa

SEQ ID NO: 97 - DNA, 21 bp, Artificial Sequence
  /note=“Description of Artificial Sequence shRNA target sequence”
cgagtatgttgtccgaaataa

SEQ ID NO: 98 - DNA, 21 bp, Artificial Sequence
  /note=“Description of Artificial Sequence shRNA target sequence”
caaactgaaggctcaagcaaa

SEQ ID NO: 99 - DNA, 21 bp, Artificial Sequence
  /note=“Description of Artificial Sequence shRNA target sequence”
ccagggagtctctttgacaat

SEQ ID NO: 100 - DNA, 21 bp, Artificial Sequence
  /note=“Description of Artificial Sequence shRNA target sequence”
gcacaccaaacagaccttctt

SEQ ID NO: 101 - DNA, 21 bp, Artificial Sequence
  /note=“Description of Artificial Sequence shRNA target sequence”
ctggtgtggtctgattctttc

SEQ ID NO: 102 - DNA, 21 bp, Artificial Sequence
  /note=“Description of Artificial Sequence shRNA target sequence”
ccagggtttgtgaattacaaa

SEQ ID NO: 103 - DNA, 21 bp, Artificial Sequence
  /note=“Description of Artificial Sequence shRNA target sequence”
gtgatgtttaccgacctaaat

SEQ ID NO: 104 - DNA, 21 bp, Artificial Sequence
  /note=“Description of Artificial Sequence shRNA target sequence”
actcggattcaaccttattat

SEQ ID NO: 105 - DNA, 21 bp, Artificial Sequence
  /note=“Description of Artificial Sequence shRNA target sequence”
gaaatgtgactttagtgtaat

SEQ ID NO: 106 - DNA, 21 bp, Artificial Sequence
  /note=“Description of Artificial Sequence shRNA target sequence”
cagtgattagtgaccatatat

SEQ ID NO: 107 - DNA, 21 bp, Artificial Sequence
  /note=“Description of Artificial Sequence shRNA target sequence”
aggcgttgattcagtcaactt

SEQ ID NO: 108 - DNA, 21 bp, Artificial Sequence
  /note=“Description of Artificial Sequence shRNA target sequence”
ccttgacctgaagctcatatt

SEQ ID NO: 109 - DNA, 21 bp, Artificial Sequence
  /note=“Description of Artificial Sequence shRNA target sequence”
cgatacaagctcaccctattt

SEQ ID NO: 110 - DNA, 21 bp, Artificial Sequence
  /note=“Description of Artificial Sequence shRNA target sequence”
cgccttcatggtaggatgtat

SEQ ID NO: 111 - DNA, 21 bp, Artificial Sequence
  /note=“Description of Artificial Sequence shRNA target sequence”
caacaccgtcaatgtcaatta

SEQ ID NO: 112 - DNA, 21 bp, Artificial Sequence
  /note=“Description of Artificial Sequence shRNA target sequence”
gctgtcaatgttgtacgcttt

SEQ ID NO: 113 - DNA, 21 bp, Artificial Sequence
  /note=“Description of Artificial Sequence shRNA target sequence”
gcgctgatgatctcgctcatt

SEQ ID NO: 114 - DNA, 21 bp, Artificial Sequence
  /note=“Description of Artificial Sequence shRNA target sequence”
gtcaggagataatgatgggca

SEQ ID NO: 115 - DNA, 21 bp, Artificial Sequence
  /note=“Description of Artificial Sequence shRNA target sequence”
gctgatgagcaaagcatcgtt

SEQ ID NO: 116 - DNA, 21 bp, Artificial Sequence
  /note=“Description of Artificial Sequence shRNA target sequence”
agtatactaaatggcaatttg

SEQ ID NO: 117 - DNA, 21 bp, Artificial Sequence
  /note=“Description of Artificial Sequence shRNA target sequence”
ccacagagattcagaatattc

SEQ ID NO: 118 - DNA, 21 bp, Artificial Sequence
  /note=“Description of Artificial Sequence shRNA target sequence”
gcctacattgaacgtgatgtt

SEQ ID NO: 119 - DNA, 21 bp, Artificial Sequence
  /note=“Description of Artificial Sequence shRNA target sequence”
cctgtggtttctctctccaaa

SEQ ID NO: 120 - DNA, 21 bp, Artificial Sequence
  /note=“Description of Artificial Sequence shRNA target sequence”
agcgagatcagcagtacttaa

SEQ ID NO: 121 - DNA, 21 bp, Artificial Sequence
  /note=“Description of Artificial Sequence shRNA target sequence”
gcggatctataatacccagaa

SEQ ID NO: 122 - DNA, 21 bp, Artificial Sequence
  /note=“Description of Artificial Sequence shRNA target sequence”
ccactattggaagatcaaata

SEQ ID NO: 123 - DNA, 21 bp, Artificial Sequence
  /note=“Description of Artificial Sequence shRNA target sequence”
gaccaatgatggatgtgggaa

SEQ ID NO: 124 - DNA, 21 bp, Artificial Sequence
  /note=“Description of Artificial Sequence shRNA target sequence”
cgagttaaactaccactaaat

SEQ ID NO: 125 - DNA, 21 bp, Artificial Sequence
  /note=“Description of Artificial Sequence shRNA target sequence”
acaggctgaatgactacgtgt

SEQ ID NO: 126 - DNA, 21 bp, Artificial Sequence
  /note=“Description of Artificial Sequence shRNA target sequence”
ccagcctttatgctctgataa

SEQ ID NO: 127 - DNA, 21 bp, Artificial Sequence
  /note=“Description of Artificial Sequence shRNA target sequence”
gttgctttctacatactacaa

SEQ ID NO: 128 - DNA, 21 bp, Artificial Sequence
  /note=“Description of Artificial Sequence shRNA target sequence”
cctgcaatactccgaggataa

SEQ ID NO: 129 - DNA, 21 bp, Artificial Sequence
  /note=“Description of Artificial Sequence shRNA target sequence”
ccaacacccttcgacattcaa

SEQ ID NO: 130 - DNA, 21 bp, Artificial Sequence
  /note=“Description of Artificial Sequence shRNA target sequence”
cggagtgcaatcaagattgtt

SEQ ID NO: 131 - DNA, 21 bp, Artificial Sequence
  /note=“Description of Artificial Sequence shRNA target sequence”
agtgttgctttctacatacta

SEQ ID NO: 132 - DNA, 21 bp, Artificial Sequence
  /note=“Description of Artificial Sequence shRNA target sequence”
cagaagaaagtccaagaagaa

SEQ ID NO: 133 - DNA, 21 bp, Artificial Sequence
  /note=“Description of Artificial Sequence shRNA target sequence”
gctcaagcaaaggatgccata

SEQ ID NO: 134 - DNA, 21 bp, Artificial Sequence
  /note=“Description of Artificial Sequence shRNA target sequence”
agagtaaagacacggatattt

SEQ ID NO: 135 - DNA, 21 bp, Artificial Sequence
  /note=“Description of Artificial Sequence shRNA target sequence”
cctaaagatcagacagtacat

SEQ ID NO: 136 - DNA, 21 bp, Artificial Sequence
  /note=“Description of Artificial Sequence shRNA target sequence”
cctccttatcacagttgtaat

SEQ ID NO: 137 - DNA, 21 bp, Artificial Sequence
  /note=“Description of Artificial Sequence shRNA target sequence”
gaatttgatatcgacatggat

SEQ ID NO: 138 - DNA, 21 bp, Artificial Sequence
  /note=“Description of Artificial Sequence shRNA target sequence”
ccagagatcctgagttcaatt

SEQ ID NO: 139 - DNA, 21 bp, Artificial Sequence
  /note=“Description of Artificial Sequence shRNA target sequence”
ccgtttcaaagcgtcaagaat

SEQ ID NO: 140 - DNA, 21 bp, Artificial Sequence
  /note=“Description of Artificial Sequence shRNA target sequence”
gaccggtgctaacaaaggaat

SEQ ID NO: 141 - DNA, 21 bp, Artificial Sequence
  /note=“Description of Artificial Sequence shRNA target sequence”
cattgataaagagccgttata

SEQ ID NO: 142 - DNA, 21 bp, Artificial Sequence
  /note=“Description of Artificial Sequence shRNA target sequence”
cggttctgtatctgtgctaca

SEQ ID NO: 143 - DNA, 21 bp, Artificial Sequence
  /note=“Description of Artificial Sequence shRNA target sequence”
cctagaaacctacgagatcat

SEQ ID NO: 144 - DNA, 21 bp, Artificial Sequence
  /note=“Description of Artificial Sequence shRNA target sequence”
cgtcattctgttgtggaagat

SEQ ID NO: 145 - DNA, 21 bp, Artificial Sequence
  /note=“Description of Artificial Sequence shRNA target sequence”
gcagcatatttatatgggata

SEQ ID NO: 146 - DNA, 21 bp, Artificial Sequence
  /note=“Description of Artificial Sequence shRNA target sequence”
gctgtggttcaagttgccata

SEQ ID NO: 147 - DNA, 21 bp, Artificial Sequence
  /note=“Description of Artificial Sequence shRNA target sequence”
cgtcaacatgatggacgagtt

SEQ ID NO: 148 - DNA, 21 bp, Artificial Sequence
  /note=“Description of Artificial Sequence shRNA target sequence”
gcttgccaaattctccgatta

SEQ ID NO: 149 - DNA, 21 bp, Artificial Sequence
  /note=“Description of Artificial Sequence shRNA target sequence”
cgcttcacctatgccagttac

SEQ ID NO: 150 - DNA, 21 bp, Artificial Sequence
  /note=“Description of Artificial Sequence shRNA target sequence”
gatcactcatacttcagcatt

SEQ ID NO: 151 - DNA, 21 bp, Artificial Sequence
  /note=“Description of Artificial Sequence shRNA target sequence”
cactccttattctgagacatt

SEQ ID NO: 152 - DNA, 21 bp, Artificial Sequence
  /note=“Description of Artificial Sequence shRNA target sequence”
cccattagtgaagtaaacaaa

SEQ ID NO: 153 - DNA, 21 bp, Artificial Sequence
  /note=“Description of Artificial Sequence shRNA target sequence”
cagaaagtggatgagagagaa

SEQ ID NO: 154 - DNA, 21 bp, Artificial Sequence
  /note=“Description of Artificial Sequence shRNA target sequence”
ccgatgacgttgataaggctt

SEQ ID NO: 155 - DNA, 21 bp, Artificial Sequence
  /note=“Description of Artificial Sequence shRNA target sequence”
tgggtggactgtgggtaattt

SEQ ID NO: 156 - DNA, 21 bp, Artificial Sequence
  /note=“Description of Artificial Sequence shRNA target sequence”
gcgaactgtatatgcacgata

SEQ ID NO: 157 - DNA, 21 bp, Artificial Sequence
  /note=“Description of Artificial Sequence shRNA target sequence”
cctggagtaaggagatcgaaa

SEQ ID NO: 158 - DNA, 21 bp, Artificial Sequence
  /note=“Description of Artificial Sequence shRNA target sequence”
acaccgtcagatgattaattt

SEQ ID NO: 159 - DNA, 21 bp, Artificial Sequence
  /note=“Description of Artificial Sequence shRNA target sequence”
cattcatacgatcacagataa

SEQ ID NO: 160 - DNA, 21 bp, Artificial Sequence
  /note=“Description of Artificial Sequence shRNA target sequence”
cggcgtgtttgtcaacattca

SEQ ID NO: 161 - DNA, 21 bp, Artificial Sequence
  /note=“Description of Artificial Sequence shRNA target sequence”
agactgtcattaatgtcttat

SEQ ID NO: 162 - DNA, 21 bp, Artificial Sequence
  /note=“Description of Artificial Sequence shRNA target sequence”
cctgccatacatgggctccta

SEQ ID NO: 163 - DNA, 21 bp, Artificial Sequence
  /note=“Description of Artificial Sequence shRNA target sequence”
cacgggaagttccttcagatt

SEQ ID NO: 164 - DNA, 21 bp, Artificial Sequence
  /note=“Description of Artificial Sequence shRNA target sequence”
atctagagcagatgatcaaag

SEQ ID NO: 165 - DNA, 21 bp, Artificial Sequence
  /note=“Description of Artificial Sequence shRNA target sequence”
ccgcagaaggtacagattctt

SEQ ID NO: 166 - DNA, 21 bp, Artificial Sequence
  /note=“Description of Artificial Sequence shRNA target sequence”
cggaagaaggttggcctaata

SEQ ID NO: 167 - DNA, 21 bp, Artificial Sequence
  /note=“Description of Artificial Sequence shRNA target sequence”
ccaggtgctatgagattagaa

SEQ ID NO: 168 - DNA, 21 bp, Artificial Sequence
  /note=“Description of Artificial Sequence shRNA target sequence”
gctggtttactgtatggttta

SEQ ID NO: 169 - DNA, 21 bp, Artificial Sequence
  /note=“Description of Artificial Sequence shRNA target sequence”
gtcactatttgagtgtgccta

SEQ ID NO: 170 - DNA, 21 bp, Artificial Sequence
  /note=“Description of Artificial Sequence shRNA target sequence”
gcatcctgtcagactttactt

SEQ ID NO: 171 - DNA, 21 bp, Artificial Sequence
  /note=“Description of Artificial Sequence shRNA target sequence”
gcatctattgtgggccttgat

SEQ ID NO: 172 - DNA, 21 bp, Artificial Sequence
  /note=“Description of Artificial Sequence shRNA target sequence”
gaaggcttctacgtggtcttt

SEQ ID NO: 173 - DNA, 21 bp, Artificial Sequence
  /note=“Description of Artificial Sequence shRNA target sequence”
ccaagcaaagattccagacat

SEQ ID NO: 174 - DNA, 21 bp, Artificial Sequence
  /note=“Description of Artificial Sequence shRNA target sequence”
tgctaacaaaggaatcggatt

SEQ ID NO: 175 - DNA, 21 bp, Artificial Sequence
  /note=“Description of Artificial Sequence shRNA target sequence”
cgaagtcatctccgatacaaa

SEQ ID NO: 176 - DNA, 21 bp, Artificial Sequence
  /note=“Description of Artificial Sequence shRNA target sequence”
gtggctgatttcagtcaattt

SEQ ID NO: 177 - DNA, 21 bp, Artificial Sequence
  /note=“Description of Artificial Sequence shRNA target sequence”
gccgacattcttctctcacat

SEQ ID NO: 178 - DNA, 21 bp, Artificial Sequence
  /note=“Description of Artificial Sequence shRNA target sequence”
gagagctacagatcagaattt

SEQ ID NO: 179 - DNA, 21 bp, Artificial Sequence
  /note=“Description of Artificial Sequence shRNA target sequence”
gcccaagtgtagcaatccaaa

SEQ ID NO: 180 - DNA, 21 bp, Artificial Sequence
  /note=“Description of Artificial Sequence shRNA target sequence”
cgttcccgaatcttggttaaa

SEQ ID NO: 181 - DNA, 21 bp, Artificial Sequence
  /note=“Description of Artificial Sequence shRNA target sequence”
gccaggaactccactttcttt

SEQ ID NO: 182 - DNA, 21 bp, Artificial Sequence
  /note=“Description of Artificial Sequence shRNA target sequence”
gcggtctttctttcttcgaat

SEQ ID NO: 183 - DNA, 21 bp, Artificial Sequence
  /note=“Description of Artificial Sequence shRNA target sequence”
cctcggagtaaccattgacaa

SEQ ID NO: 184 - DNA, 21 bp, Artificial Sequence
  /note=“Description of Artificial Sequence shRNA target sequence”
cataatgcaagaacgtttaat

SEQ ID NO: 185 - DNA, 21 bp, Artificial Sequence
  /note=“Description of Artificial Sequence shRNA target sequence”
acgaatgacgtggtgcagaat

SEQ ID NO: 186 - DNA, 21 bp, Artificial Sequence
  /note=“Description of Artificial Sequence shRNA target sequence”
ccaaacttgaagacatcagat

SEQ ID NO: 187 - DNA, 21 bp, Artificial Sequence
  /note=“Description of Artificial Sequence shRNA target sequence”
gcacagaaagaggacagtgaa

SEQ ID NO: 188 - DNA, 21 bp, Artificial Sequence
  /note=“Description of Artificial Sequence shRNA target sequence”
gcgcccaatatcttgctgtat

SEQ ID NO: 189 - DNA, 21 bp, Artificial Sequence
  /note=“Description of Artificial Sequence shRNA target sequence”
ccagagtgacatcaattccat

SEQ ID NO: 190 - DNA, 21 bp, Artificial Sequence
  /note=“Description of Artificial Sequence shRNA target sequence”
agtgatgagccaagccttaaa

SEQ ID NO: 191 - DNA, 21 bp, Artificial Sequence
  /note=“Description of Artificial Sequence shRNA target sequence”
gctcccaagaaacacttacaa

SEQ ID NO: 192 - DNA, 21 bp, Artificial Sequence
  /note=“Description of Artificial Sequence shRNA target sequence”
ccaagagaagaaggaagaaga

SEQ ID NO: 193 - DNA, 21 bp, Artificial Sequence
  /note=“Description of Artificial Sequence shRNA target sequence”
tgcttgttactgacaacgatt

SEQ ID NO: 194 - DNA, 21 bp, Artificial Sequence
  /note=“Description of Artificial Sequence shRNA target sequence”
gacgtggcaaacctagagaat

SEQ ID NO: 195 - DNA, 21 bp, Artificial Sequence
  /note=“Description of Artificial Sequence shRNA target sequence”
gcttctcatgtggactcttaa

SEQ ID NO: 196 - DNA, 21 bp, Artificial Sequence
  /note=“Description of Artificial Sequence shRNA target sequence”
gcagaagttcaacaagtggtt

SEQ ID NO: 197 - DNA, 21 bp, Artificial Sequence
  /note=“Description of Artificial Sequence shRNA target sequence”
ccaagtttgtataaaggagat

SEQ ID NO: 198 - DNA, 21 bp, Artificial Sequence
  /note=“Description of Artificial Sequence shRNA target sequence”
acatctatacgaatcgaatac

SEQ ID NO: 199 - DNA, 21 bp, Artificial Sequence
  /note=“Description of Artificial Sequence shRNA target sequence”
cgatggcacttggagcttaaa

SEQ ID NO: 200 - DNA, 21 bp, Artificial Sequence
  /note=“Description of Artificial Sequence shRNA target sequence”
gaagaatttgatatcgacatg

SEQ ID NO: 201 - DNA, 21 bp, Artificial Sequence
  /note=“Description of Artificial Sequence shRNA target sequence”
gagcaaggcaaaccagttatt

SEQ ID NO: 202 - DNA, 21 bp, Artificial Sequence
  /note=“Description of Artificial Sequence shRNA target sequence”
cgcttatatttgcttgcgatt

SEQ ID NO: 203 - DNA, 21 bp, Artificial Sequence
  /note=“Description of Artificial Sequence shRNA target sequence”
ttgaggatgaagtgctaatag

SEQ ID NO: 204 - DNA, 21 bp, Artificial Sequence
  /note=“Description of Artificial Sequence shRNA target sequence”
acagcttccggatccagttat

SEQ ID NO: 205 - DNA, 21 bp, Artificial Sequence
  /note=“Description of Artificial Sequence shRNA target sequence”
gccaagttgatcaagtccaaa

SEQ ID NO: 206 - DNA, 21 bp, Artificial Sequence
  /note=“Description of Artificial Sequence shRNA target sequence”
cccgaagctacgcaaagaatt

SEQ ID NO: 207 - DNA, 21 bp, Artificial Sequence
  /note=“Description of Artificial Sequence shRNA target sequence”
cctacggcgtgcagtgcttca

SEQ ID NO: 208 - DNA, 21 bp, Artificial Sequence
  /note=“Description of Artificial Sequence shRNA target sequence”
tgcccgacaaccactacctga

SEQ ID NO: 209 - DNA, 21 bp, Artificial Sequence
  /note=“Description of Artificial Sequence shRNA target sequence”
ccgtaatgcagaagaagacca

SEQ ID NO: 210 - DNA, 21 bp, Artificial Sequence
  /note=“Description of Artificial Sequence shRNA target sequence”
agaatcgtcgtatgcagtgaa

SEQ ID NO: 211 - DNA, 21 bp, Artificial Sequence
  /note=“Description of Artificial Sequence shRNA target sequence”
ccgtcatagcgataacgagtt

SEQ ID NO: 212 - DNA, 21 bp, Artificial Sequence
  /note=“Description of Artificial Sequence shRNA target sequence”
ccaacgtgacctatcccatta

SEQ ID NO: 213 - DNA, 21 bp, Artificial Sequence
  /note=“Description of Artificial Sequence shRNA target sequence”
tgaccctgaagttcatctgca

SEQ ID NO: 214 - DNA, 21 bp, Artificial Sequence
  /note=“Description of Artificial Sequence shRNA target sequence”
acaacagccacaacgtctata

SEQ ID NO: 215 - DNA, 21 bp, Artificial Sequence
  /note=“Description of Artificial Sequence shRNA target sequence”
gtcggcttacggcggtgattt

SEQ ID NO: 216 - DNA, 21 bp, Artificial Sequence
  /note=“Description of Artificial Sequence shRNA target sequence”
acttacgctgagtacttcgaa

SEQ ID NO: 217 - DNA, 21 bp, Artificial Sequence
  /note=“Description of Artificial Sequence shRNA target sequence”
ctcagttccagtacggctcca

SEQ ID NO: 218 - DNA, 21 bp, Artificial Sequence
  /note=“Description of Artificial Sequence shRNA target sequence”
ccacatgaagcagcacgactt

SEQ ID NO: 219 - DNA, 21 bp, Artificial Sequence
  /note=“Description of Artificial Sequence shRNA target sequence”
gcttcaagtgggagcgcgtga

SEQ ID NO: 220 - DNA, 21 bp, Artificial Sequence
  /note=“Description of Artificial Sequence shRNA target sequence”
gcgtccacactatggcaaatt

SEQ ID NO: 221 - DNA, 21 bp, Artificial Sequence
  /note=“Description of Artificial Sequence shRNA target sequence”
gcagacactaaatgctatcaa

SEQ ID NO: 222 - DNA, 21 bp, Artificial Sequence
  /note=“Description of Artificial Sequence shRNA target sequence”
gccatacccaaagtctcgttt

SEQ ID NO: 223 - DNA, 21 bp, Artificial Sequence
  /note=“Description of Artificial Sequence shRNA target sequence”
gcagcgattcattaagattga

SEQ ID NO: 224 - DNA, 21 bp, Artificial Sequence
  /note=“Description of Artificial Sequence shRNA target sequence”
gctgctgttccaagcatcaaa

SEQ ID NO: 225 - DNA, 21 bp, Artificial Sequence
  /note=“Description of Artificial Sequence shRNA target sequence”
ccctgactggagatgaagtaa

SEQ ID NO: 226 - DNA, 21 bp, Artificial Sequence
  /note=“Description of Artificial Sequence shRNA target sequence”
gtggagctgaagaaagtattt

SEQ ID NO: 227 - DNA, 21 bp, Artificial Sequence
  /note=“Description of Artificial Sequence shRNA target sequence”
ccgaaccaagttgcagaacaa

SEQ ID NO: 228 - DNA, 21 bp, Artificial Sequence
  /note=“Description of Artificial Sequence shRNA target sequence”
cgaactacaactggcaataaa

SEQ ID NO: 229 - DNA, 21 bp, Artificial Sequence
  /note=“Description of Artificial Sequence shRNA target sequence”
cgctacctaatggaggaagat

(19) United States
(12) Patent Application Publication
(10) Pub. No.: US 2016/0194718 A1
(43) Pub. Date: Jul. 7, 2016

(54) Compositions and Methods for Identification, Assessment, Prevention, and Treatment of Cancer Using Histone H3K27ME3 Biomarkers and Modulators

(71) Applicant: DANA-FARBER CANCER INSTITUTE, INC., Boston, MA (US)

(72) Inventors: Andrew Lane, Jamaica Plain, MA (US); David Weinstock, Jamaica Plain, MA (US)

(21) Appl. No.: 14/890,720

(22) PCT Filed: May 21, 2014
(86) PCT No.: PCT/US14/38938

§ 371 Date: Nov. 12, 2015

Related U.S. Application Data
(60) Provisional application No. 61/981,317, filed on Apr. 18, 2014.
(60) Provisional application No. 61/825,710, filed on May 21, 2013.

(51) Int. Cl. C12Q 1/68; A61K 31/496; G01N 33/574; A61K 31/55

(52) U.S. Cl. CPC C12Q 1/6886; A61K 31/55; A61K 31/496; G01N 33/57496; G01N 33/57492; G01N 33/57488; C12Q 2600/158; C12Q 2600/118; C12Q 2600/106

(57) ABSTRACT

The present invention relates to methods for identifying, assessing, preventing, and treating cancer (e.g., lymphoid and/or myeloid malignancies such as B-ALL in humans). A variety of histone H3K27rne3 biomarkers are provided, wherein alterations in the copy number of one or more of the biomarkers and/or alterations in the amount, structure, and/or activity of one or more of the biomarkers is associated with cancer status and indicates amenability to treatment or prevention by modulating H3K27me3 levels. The present invention further relates to methods of increasing the number of lymphoid progenitor cells (e.g., increase self-renewal and cell proliferation) by contacting the lymphoid progenitor cells (e.g., wild type and/or genomically altered cells) with an agent that inhibits polycomb repressor complex 2 (PRC2) activity or reduces H3K27roe3 levels.

### CROSS-REFERENCE TO RELATED APPLICATIONS

This application claims the benefit of U.S. Provisional Application No. 61/825,710, filed on 21 May 2013 and U.S. Provisional Application No. 61/981,317, filed on 18 Apr. 2014; the entire contents of said applications are incorporated herein in their entirety by this reference.

### STATEMENT OF RIGHTS

This invention was made with government support under Grant NIH RO1 CA15198-01 and Grant NIH RO1 CA172387-A01 awarded by the National Institutes of Health. The U.S. government has certain rights in the invention. This statement is included solely to comply with 37 C.F.R. §401.14(a)(f)(4) and should not be taken as an assertion or admission that the application discloses and/or claims only one invention.

### BACKGROUND OF THE INVENTION

Up to 3% of children with Down syndrome (DS) will develop B cell acute lymphoblastic leukemia (B-ALL) (Rabin and Whitlock, Oncologist 14:164-173) and polysomy 21 (i.e., extra copies of chromosome 21) is the most frequent somatic aneuploidy in B-ALL (Heerema et al. (2007) Genes Chrom. Cancer 46:684-693; Pui et al. N. Engl. J. Med. 350:1535-1548). Additional B-ALLs harbor an intrachromosomal amplification of chr.21q22 (iAmp21) (Moorman et al. Lancet Oncol. 11:429-438; Rand et al. Blood 117:6848-6855) that overlaps with the putative “Down Syndrome Critical Region (DSCR)” on chromosome 21q22.

The mechanistic links between loci in these regions (e.g., polysomy, gene copy modulation, gene expression modulation, and the like) and precursor B cell transformation remain undefined. A series of studies across four decades have attempted to define phenotypes within cells from patients with DS that could underlie the association with B-ALL and other lymphoid and/or myeloid malignancies. However, comparisons between patients with DS and controls may be confounded by genetic or environmental differences distinct from trisomy 21 itself. Accordingly, there is a great need to identify the genetic, molecular, and biochemical underpinnings of such lymphoid and/or myeloid malignancies in such subjects, including the generation of diagnostic, prognostic, and therapeutic agents to effectively control such disorders in subjects.

### SUMMARY OF THE INVENTION

Children with Down syndrome (DS) have a 20-fold increased risk of developing B cell acute lymphoblastic leukemia (B-ALL) (Rabin and Whitlock (2009) Oncologist 14:164-173), yet the mechanisms underlying this association are undefined. The present invention is based in part on the discovery that polysomy (e.g., triplication) of only 31 gene orthologous to the putative DS Critical Region (DSCR) on human chromosome 21q22 is sufficient to confer and promote B cell autonomous self-renewal in vitro, B cell maturation defects in vivo, and B-ALL in concert with either BCR-ABL or CRLF2 with activated JAK. Chr.21q22 triplication suppresses H3K27me3 in murine progenitor B cells and B-ALLs, and “bivalent” genes with both H3K27me3 and H3K4me3 at their promoters in wild-type progenitor B cells are preferentially overexpressed in triplicated cells. Human B-ALLs with polysomy 21 are distinguished by their overexpression of genes known to be marked with H3K27me3 in multiple cell types. B cells with amplified DSCR (e.g., copy number gains, enhanced expression, and the like) relative to wild type harbor a transcriptional signature characterized by de-repression of polycomb repressor complex 2 (PRC2) components and/or targets that is highly enriched among B-ALLs in children with DS. Inhibition of PRC2 function and/or modulation of H3K27me3 levels (e.g., by pharmacological inhibition of H3K27 methyltransferases) is sufficient to promote self-renewal in wild-type B cells while enhancement of H3K27me3 levels (e.g., by inhibiting demethylases that remove H3K27me3) completely block self-renewal induced by DSCR triplication. It has further been discovered that self-renewal in B cells with DSCR triplication requires overexpression of the DSCR locus encoding HMGN1, a nucleosome remodeling protein encoded on chr.21q22 (Catez et al (2002) EMBO Rep. 3:760-766; Lim et al. (200) EMBO J. 24:3038-3048 Rattner et al. (2009) Mol. Cell 34:620-626), suppresses H3K27me3 levels. Overexpression of HMGN1 suppresses H3K27me3 and promotes both B cell proliferation in vitro and B-ALL in vivo. HMGN1 overexpression and loss of H3K27me3 are implicated in progenitor B cell transformation and provide strategies to therapeutically target leukemias with polysomy 21.

In one aspect, a method of determining whether a subject afflicted with a cancer or at risk for developing a cancer would benefit from modulating histone H3K27me3 levels is provided, wherein the method comprises: a) obtaining a biological sample from the subject; b) determining the copy number, level of expression, or level of activity of one or more biomarkers listed in Tables 1-5 or a fragment thereof in a subject sample; c) determining the copy number, level of expression, or level of activity of the one or more biomarkers in a control; and d) comparing the copy number, level of expression, or level of activity of said one or more biomarkers detected in steps b) and c); wherein a significant modulation in the copy number, level of expression, or level of activity of the one or more biomarkers in the subject sample relative to the control copy number, level of expression, or level of activity of the one or more biomarkers indicates that the subject afflicted with the cancer or at risk for developing the cancer would benefit from modulating histone H3K27me3 levels. In one embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

In another aspect, a method for monitoring the progression of a cancer in a subject is provided, wherein the method comprises: a) detecting in a subject sample at a first point in time the copy number, level of expression, or level of activity of one or more biomarkers listed in Tables 1-5 or a fragment thereof; b) repeating step a) at a subsequent point in time; and c) comparing the copy number, level of expression, or level of activity of said one or more biomarkers detected in steps a) and b) to monitor the progression of the cancer. In one method, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, c) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof. In another embodiment, an at least twenty percent increase or an at least twenty percent decrease between the copy number, level of expression, or level of activity of the one or more biomarkers in the subject sample at a first point in time relative to the copy number, level of expression, or level of activity of the one or more biomarkers in the subject sample at a subsequent point in time indicates progression of the cancer; or wherein less than a twenty percent increase or less than a twenty percent decrease between the copy number, level of expression, or level of activity of the one or more biomarkers in the subject sample at a first point in time relative to the copy number, level of expression, or level of activity of the one or more biomarkers in the subject sample at a subsequent point in time indicates a lack of significant progression of the cancer. In still another embodiment, the subject has undergone treatment to modulate histone H3K27me3 levels between the first point in time and the subsequent point in time.

In still another aspect, a method for stratifying subjects afflicted with a cancer according to predicted clinical outcome of treatment with one or more modulators of histone H3K27me3 levels is provided, wherein the method comprises: a) determining the copy number, level of expression, or level of activity of one or more biomarkers listed in Tables 1-5 or a fragment thereof in a subject sample; b) determining the copy number, level of expression, or level of activity of the one or more biomarkers in a control sample; and c) comparing the copy number, level of expression, or level of activity of said one or more biomarkers detected in steps a) and b); wherein a significant modulation in the copy number, level of expression, or level of activity of the one or more biomarkers in the subject sample relative to the normal copy number, level of expression, or level of activity of the one or more biomarkers in the control sample predicts the clinical outcome of the patient to treatment with one or more modulators of histone 1H3K27me3 levels. In one embodiment, the predicted clinical outcome is (a) cellular growth, (b) cellular proliferation, or (c) survival time resulting from treatment with one or more modulators of histone H3K27me3 levels. In another embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, c) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof. In still another embodiment, an at least twenty percent increase or an at least twenty percent decrease between the copy number, level of expression, or level of activity of the one or more biomarkers in the subject sample compared to the control sample predicts that the subject has a poor clinical outcome; or wherein less than a twenty percent increase or less than a twenty percent decrease between the copy number, level of expression, or level of activity of the one or more biomarkers in the subject sample compared to the control sample predicts that the subject has a favorable clinical outcome. In yet another embodiment, the method further comprises treating the subject with a therapeutic agent that specifically modulates the copy number, level of expression, or level of activity of the one or more biomarkers. In another embodiment, the method further comprises treating the subject with one or more modulators of histone H3K27me3 levels.

In yet another aspect, a method of determining the efficacy of a test compound for inhibiting a cancer in a subject is provided, wherein the method comprises: a) determining the copy number, level of expression, or level of activity of one or more biomarkers listed in Tables 1-5 or a fragment thereof in a first sample obtained from the subject and exposed to the test compound; b) determining the copy number, level of expression, or level of activity of the one or more biomarkers in a second sample obtained from the subject, wherein the second sample is not exposed to the test compound, and c) comparing the copy number, level of expression, or level of activity of the one or more biomarkers in the first and second samples, wherein a significantly modulated copy number, level of expression, or level of activity of the biomarker, relative to the second sample, is an indication that the test compound is efficacious for inhibiting the cancer in the subject. In one embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof. In another embodiment, the first and second samples are portions of a single sample obtained from the subject or portions of pooled samples obtained from the subject.

In another aspect, a method of determining the efficacy of a therapy for inhibiting a cancer in a subject is provided, wherein the method comprises: a) determining the copy number, level of expression, or level of activity of one or more biomarkers listed in Tables 1-5 or a fragment thereof in a first sample obtained from the subject prior to providing at least a portion of the therapy to the subject; b) determining the copy number, level of expression, or level of activity of the one or more biomarkers in a second sample obtained from the subject following provision of the portion of the therapy; and c) comparing the copy number, level of expression, or level of activity of the one or more biomarkers in the first and second samples, wherein a significantly modulated copy number, level of expression, or level of activity of the one or more biomarkers in the second sample, relative to the first sample, is an indication that the therapy is efficacious for inhibiting the cancer in the subject. In one embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d). “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof: or wherein said therapy further comprises standard of care therapy for treating the cancer.

In still another aspect, a method for identifying a compound which inhibits a cancer is provided, wherein the method comprises: a) contacting one or more biomarkers listed in Tables 1-5 or a fragment thereof with a test compound; and b) determining the effect of the test compound on the copy number, level of expression, or level of activity of the one or more biomarkers to thereby identify a compound which inhibits the cancer. In one embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, c) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof. In another embodiment, the one or more biomarkers is expressed on or in a cell (e.g., cells isolated from an animal model of a cancer or cells from a subject afflicted with a cancer).

In yet another aspect, a method for inhibiting a cancer is provided, wherein the method comprises contacting a cell with an agent that modulates the copy number, level of expression, or level of activity of one or more biomarkers listed in Tables 1-5 or a fragment thereof to thereby inhibit the cancer. In one embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof. In another embodiment, the copy number, level of expression, or level of activity of the one or more biomarkers is downmodulated or upmodulated. In still another embodiment, the step of contacting occurs in vivo, ex vim or in vitro. In yet another embodiment, the method further comprises contacting the cell with an additional agent that inhibits the cancer.

In another aspect, a method for treating a subject afflicted with a cancer is provided, wherein the method comprises administering an agent that modulates the copy number, level of expression, or level of activity of one or more biomarkers listed in Tables 1-5 or a fragment thereof such that the cancer is treated. In one embodiment, the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof. In another embodiment, the agent downmodulates or upmodulates the copy number, level of expression, or level of activity of the one or more biomarkers. In still another embodiment, the method further comprises administering one or more additional agents that treats the cancer. In yet another embodiment, the agent is one or more modulators of histone H3K27me3 levels.

In still another aspect, a pharmaceutical composition comprising a polynucleotide encoding one or more biomarkers listed in Tables 1-5 or a fragment thereof useful for treating cancer in a pharmaceutically acceptable carrier. In one embodiment, the polynucleotide encoding the one or more biomarkers listed in Tables 1-5 or a fragment thereof further comprises an expression vector. In another embodiment, the pharmaceutical composition is used in a method for treating a cancer.

In yet another aspect, a kit is provided comprising an agent which selectively binds to one or more biomarkers listed in Tables 1-5 or a fragment thereof and instructions for use.

In another aspect, a kit is provided comprising an agent which selectively hybridizes to a polynucleotide encoding one or more biomarkers listed in Tables 1-5 or fragment thereof and instructions for use.

In still another aspect, a biochip is provided comprising a solid substrate, said substrate comprising a plurality of probes capable of detecting one or more biomarkers listed in Tables 1-5 or a fragment thereof wherein each probe is attached to the substrate at a spatially defined address. In one embodiment, the probes are complementary to a genomic or transcribed polynucleotide associated with the one or more biomarkers.

In yet another aspect, a method of increasing the number of lymphoid progenitor cells from an initial population of lymphoid progenitor cells is provided, wherein the method comprises contacting the lymphoid progenitor cells with an agent that inhibits polycomb repressor complex 2 (PRC2) activity or reduces H3K27me3 levels to thereby increase the number of lymphoid progenitor cells. In one embodiment, the agent inhibits the activity of the EZH2 histone H3K27 methyltransferase subunit of PRC2. In another embodiment, the agent is an inhibitor selected from the group consisting of a small molecule, antisense nucleic acid, interfering RNA, shRNA, siRNA, miRNA, aptamer, ribozyme, and dominant-negative protein binding partner. In still another embodiment, the lymphoid progenitor cells are comprised within bone marrow with marker selection or without marker selection. In yet another embodiment, the lymphoid progenitor cells comprise pre-pro B cells, pro B cells, large pre-B cells, small pre-B cells, immature B cells, or any combination thereof. In another embodiment, contacting the lymphoid progenitor cells with the agent is performed in vivo, ex vivo, or in vitro.

It is to be understood that any embodiments of the present invention can be combined and/or adapted for use in any of the compositions, methods, kits, biochips, and the like described herein. For example, pharmaceutical compositions, kits, or biochips described above can use one or more biomarkers selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

Regarding methods of the present invention, in one embodiment, the control is determined from a non-cancerous sample from the subject or member of the same species to which the subject belongs. In another embodiment, the sample comprises cells, cell lines, histological slides, paraffin embedded tissue, fresh frozen tissue, fresh tissue, biopsies, blood, plasma, serum, buccal scrape, saliva, cerebrospinal fluid, urine, stool, mucus, or bone marrow, obtained from the subject. In still another embodiment, the copy number is assessed by microarray, quantitative PCR (qPCR), high-throughput sequencing, comparative genomic hybridization (CGH), or fluorescent in situ hybridization (FISH). In yet another embodiment, the expression level of the one or more biomarkers is assessed by detecting the presence in the samples of a polynucleotide molecule encoding the biomarker or a portion of said polynucleotide molecule. In another embodiment, the polynucleotide molecule is a mRNA, cDNA, or functional variants or fragments thereof. In still another embodiment, the step of detecting further comprises amplifying the polynucleotide molecule. In yet another embodiment, the expression level of the one or more biomarkers is assessed by annealing a nucleic acid probe with the sample of the polynucleotide encoding the one or more biomarkers or a portion of said polynucleotide molecule under stringent hybridization conditions. In another embodiment, the expression level of the biomarker is assessed by detecting the presence in the samples of a protein of the biomarker, a polypeptide, or protein fragment thereof comprising said protein. In still another embodiment, the presence of said protein, polypeptide or protein fragment thereof is detected using a reagent which specifically binds with said protein, polypeptide or protein fragment thereof (e.g., a reagent selected from the group consisting of an antibody, an antibody derivative, and an antibody fragment). In yet another embodiment, the activity level of the biomarker is assessed by determining the magnitude of modulation of the activity or expression level of downstream targets of the one or more biomarkers. In another embodiment, the agent or test compound modulates histone H3K27me3 levels. In still another embodiment, the agent or test compound inhibits the expression and/or activity of Jumonji D3 family of histone HeK27 demethylases. In yet another embodiment, the agent or test compound is a small molecule inhibitor of KMD6A (UTX) and/or KDM6B (JMJD3). In another embodiment, the agent or test compound inhibits the expression and/or activity of HMGN1. In still another embodiment, the agent or test compound is an inhibitor selected from the group consisting of a small molecule, antisense nucleic acid, interfering RNA, shRNA, siRNA, aptamer, ribozyme, and dominant-negative protein binding partner. In yet another embodiment, the cancer is a leukemia (e.g., B-cell acute lymphoblastic leukemia). In another embodiment, the subject has an increased copy number of a) human chromosome 21 or the human DSCR region thereof, b) mouse chromosome 16 or the mouse iAmp, Ts65Dn, Ts1Rhr, Dp(16)1Yu, or Runx1 locus thereof, or c) orthologs of a) or b), relative to a wild type control. In still another embodiment, the subject is a human.

### BRIEF DESCRIPTION OF FIGURES

FIGS. 1A-1G show that segmental trisomy orthologous to human chr.21q22 promotes progenitor B cell transformation. FIG. 1A shows regions orthologous to human chromosome 21 that are triplicated in Ts1Rhr and Ts65Dn mice or amplified in iAMP21 B-ALL. FIG. 1B shows progenitor B cells (B220+CD43+) and Hardy subfractions as percentages of bone marrow (BM) cells (n=6/group in 2 independent experiments). FIG. 1C shows subfractions from mixed populations in recipient BM 16 weeks after competitive transplantation (n=5/group). FIG. 1D shows B cell colonies across 6 passages (n=3 biological replicates/genotype representative of 3 independent experiments, mean values shown, *P<0.05, **P<0.01), and bright field microscopy of 3 Ts1Rhr and 3 WT passage 2 cultures. FIG. 1E shows myeloid colonies across 4 passages (n=3 mice per genotype; NS, not significant). FIG. 1F shows leukemia-free survival of recipient mice after transplantation of Eμ-CRLF2 (C2)/Eμ-JAK2 R683G (J2)/Pax5^{+/-} (P5), with or without Ts1Rhr (Ts1) BM transduced with vector or dominant negative Ikaros (Ik6) (n=10 mice/group). FIG. 1G shows leukemia-free survival of recipient mice after transplantation of BM transduced with BCR-ABL (n=10 mice/group).

FIGS. 2A-2F show the results of abnormal differentiation in vivo and colony growth in vitro of B cells with triplication of chr.21 orthologs. FIG. 2A shows B220 and CD43 staining of bone marrow from Ts1Rhr and wild-type mice, highlighting the more immature B220+CD43+ and more mature B220+CD43− B cell populations (top panel) and CD24 and BP1 staining of the B220+CD43+ subpopulation demonstrates the early Hardy fractions: A (CD24− BP1−), B (CD24+BP1−), and C (CD24+BP1+). FIG. 2B shows Hardy subfractions of the B220+CD43+ population as absolute percentages of bone marrow mononuclear cells by flow cytometry from Ts65Dn (blue) or C57BL/6 Ts1Rhr (orange) animals compared to wild-type littermate (black) mice (n=4 mice per genotype) (bottom panel). FIG. 2C shows a schematic for the competitive bone marrow transplantation assay. FIG. 2D shows representative Hardy fraction staining in bone marrow gated on CD45.2 negative (left) competitor cells or CD45.2 positive (right) test cells. The top rows are wild-type test cells, and the bottom rows are Ts1Rhr test cells. There are fewer Ts1Rhr Hardy B/C cells and greater numbers of Ts1Rhr Hardy A cells in recipients of wild-type: Ts1Rhr competitive transplants (bottom right). FIG. 2E shows a schematic of the methylcellulose replating assay. Whole BM from Ts1Rhr or wild-type mice was plated in semi-solid medium containing cytokines favoring B cell or myeloid colony growth. 50,000 cells were collected from pooled colonies every seven days and replated in fresh media. FIG. 2F shows that the cell surface phenotype of passage 1 B cell colonies from Ts1Rhr and wild-type animals is similar. Representative flow cytometry plots of Hardy fraction cell surface phenotype of passage 1 Ts1Rhr and wild-type B cell colonies are shown. All cells are also B220+CD43+.

FIG. 3 shows that cell surface phenotype of passage 1 B cell colonies from wild-type and Ts1Rhr animals are similar. A representative flow cytometry plots of Hardy fraction cell surface phenotype of passage 1 wild-type and Ts1Rhr B cell colonies is shown. All cells are also B220+D43+.

FIG. 4 shows that passage 6 Ts1Rhr B cell colonies can form serially transplantable B-ALL in vivo. Passage 6 Ts1Rhr B cells were transplanted into immunodeficient Nod.Scid.IL2Rγ^{-/-} (NSG) primary recipients (left). Primary recipient mice (n=3) died within 150 days with progenitor B cell proliferations similar in disease phenotype to those seen with BCR-ABL transduction and transplantation. When splenocytes from a moribund mouse were transplanted into secondary sublethally-irradiated syngeneic (FVB×C57BL/6 F1) immunocompetent animals (n=5), all mice succumbed to rapidly progressive fatal B-ALL within two weeks (right).

FIGS. 5A-5G show characterization of the B-ALL that arises in Ts1Rhr bone marrow. FIG. SA shows a representative phenotype of C2/J2/P5/Ts1+Ik6 B-ALL demonstrating expression of human CRLF2 in the leukemic B cells that also co-express dominant negative Ikaros (Ik6). FIG. 5B shows leukemia-free survival for wild-type mice after transplantation with bone marrow of the genotypes listed transduced with dominant negative Ikaros (Ik6) (n=6-8 mice/group, **P<0.01 for C2/J2/P5+Ik6 versus any other genotype by log-rank test). FIG. 5C shows transduced Ts1Rhr and wild-type bone marrow using flow cytometry for B220 and GFP (BCR-ABL) demonstrating approximately equal proportions of GFP+ cells at the time of transplantation. FIG. 5D shows that Ts1Rhr and wild-type BCR-ABL B-ALLs demonstrate similar splenomegaly at the time of death with leukemia. Red dotted line represents upper limit of normal spleen weight. FIG. 5E shows bone marrow and spleen histology by hematoxylin and eosin staining demonstrating similar infiltration with B-ALL cells in Ts1Rhr and wild-type B-ALLs (scale bar=50 μm). FIG. 5F shows survival curves for recipients of Ts1Rhr or wild-type bone marrow cells (on a C57BL/6 background) transduced with BCR-ABL (n=9 mice per group, curves compared by log-rank test). FIG. 5G shows an increase in B-ALL from Ts1Rhr bone marrow is progenitor B cell autonomous. Hardy B cells were sorted from Ts1Rhr or wild-type bone marrow, transduced with BCR-ABL, and equal numbers of cells were transplanted into wild-type recipients (n=5 mice per group, curves compared by log-rank test).

FIGS. 6A-6D show that triplication of the DSCR cooperates with BCR-ABL to promote B-ALL in vivo. FIG. 6A shows Kaplan-Meier survival curves showing the probability of B-ALL-free survival among wild-type recipients of 10⁶, 10⁵, or 10⁴ wild-type or Ts1Rhr bone marrow cells transduced with BCR-ABL (n=20 per genotype at 10⁶, n=10 per genotype at 10⁵ and 10⁴; curves compared by the log-rank test). FIG. 6B shows limiting dilution analysis of recipient survival at 80 days after transplantation using a Poisson distribution calculation (Wang et al. (1997) Blood 89:3919-3924) to estimate B-ALL-initiating cell frequency in wild-type (1:244 cells) and Ts1Rhr bone marrow (1:60 cells). FIG. 6C shows cell surface phenotype of leukemias arising in wild-type or Ts1Rhr bone marrow cells. All leukemias were B220+CD43+, consistent with a precursor-B cell acute lymphoblastic leukemia (Morse et al. (2002) Blood 100: 246-258), and shown are the percentages of cells with surface immunophenotypes equivalent to normal Hardy A, B, and C fractions from individual leukemias (p=0.003 for the difference in Hardy C/B ratio between wild-type and Ts1Rhr by a two-sided exact Wilcoxon rank sum test). FIG. 6D shows the probability of B-ALL-free survival in wild-type recipients of 10³ wild-type or Ts1Rhr sorted Hardy fraction A, B, or C bone marrow cells transduced with a BCR-ABL-expressing retrovirus (n=15 per genotype, n=5 per Hardy fraction, compared by log-rank tests).

FIG. 7 shows that recipients of Ts1Rhr bone marrow transduced with BCR-ABL have more significant hematologic abnormalities after 3 weeks compared to recipients of wild-type bone-marrow. Peripheral blood analysis 3 weeks after transplantation of 10⁵ or 10⁴ wild-type+BCR-ABL or Ts1Rhr+BCR-ABL bone marrow cells is shown (n=5 mice per dose per genotype). White blood cell counts (WBC), hemoglobin (HB), and platelet (PLT) counts are shown. BCR-ABL positivity is expressed as the percentage of peripheral blood mononuclear cells (%) or the absolute number (Absolute=GFP+percentage×total WBC) per μL. Groups were compared by Student t test.

FIG. 8 shows a schematic of Hardy fraction sorting followed by BCR-ABL transduction and transplantation experiment. Hardy fraction A, B, and C cells from wild-type or Ts1Rhr B220+CD43+ bone marrow cells were sorted, transduced with MSCV-BCR-ABL-ires-GFP, and 10³ cells were transplanted into lethally irradiated wild-type recipients (see FIG. 2A for the Hardy fraction flow sorting strategy).

FIGS. 9A-9J show that trisomy and tetrasomy 21 retinal pigment epithelium (RPE) cells generated by microcell-mediated chromosome transfer (MMCT) do not have differences in DNA repair after I-SceI or RAG-induced cleavage. FIG. 9A shows single nucleotide polymorphism (SNP) array data for a tetrasomy 21 RPE clone (tetra 21-1), two trisomy 21 (tri21-2 and tri21-3) clones, and a diploid clone are shown across the entire genome (top) or chromosome 21 (bottom). FIG. 9B shows representative fluorescence in situ hybridization for human chr.21 in trisomy 21 and tetrasomy 21 RPE cells (red=chr.21 probe, blue=DAPI). FIG. 9C shows representative G-banding karyotype for a tetrasomy 21 RPE cell line. FIG. 9D shows that the DR-GFP construct was targeted to the p84 locus in RPE cells containing 2 or more copies of chr.21. A single double-strand DNA break induced by I-SceI can be repaired by multiple pathways. FIG. 9E shows that repair after I-SceI cleavage in cells lacking classical nonhomologous end-joining (NHEJ) factors (e.g. KU70/80, XRCC4/LIG4) is characterized by higher rates of homologous recombination and more extensive deletions at NHEJ junctions (Pierce et al. (2001) Genes Dev. 15:3237-42). However, the frequencies of homologous recombination (shown as percent GFP-positive) induced by I-SceI do not significantly differ between disomic (Di) and trisomy 21 (Tri) RPE clones. Two clones from each genotype were assayed on two occasions in triplicate. FIG. 9F show that the phenotype of nonhomologous end-joining induced by I-SceI did not significantly differ between disomic and trisomy 21 RPE clones. The number of base pairs deleted at junctions formed by NHEJ from two clones from each genotype is shown. FIG. 9G shows that the DR-GFP-CE construct targeted to the p84 locus can be used to assess repair after RAG cleavage. Cleavage at the paired RAG recognition signal sequences (white and black triangles) results in removal of the intervening sequence (in yellow) and nonhomologous end joining (NHEJ) between the double-strand break ends. FIG. 9H shows that PCR shows no difference in the frequencies of the RAG-induced deletion between diploid and tetrasomy 21 cells. Two biologic replicates are shown for each genotype. FIG. 9I shows that repair junctions after RAG cleavage in cells lacking classical NHEJ factors (e.g., KU70/80, XRCC4/LIG4) typically have longer deletions and more extensive use of short stretches of homology than in wild-type cells (Weinstock et al. (2006) Mol. Cell. Biol. 26:131-139). However, the number of base pairs deleted after cleavage by RAG and NHEJ did not significantly differ between disomic and tetrasomy 21 cells (n=2 clones per genotype). FIG. 9J shows junction sequences for disomic (n=27) and tetrasomy 21 (n=70) RPE clones. A single nucleotide insertion is shown in Tetra-1 #B-3-7 (yellow).

FIG. 10 shows that RNA-seq expression of the triplicated genes in Ts1Rhr compared to wild-type B cells. RNA sequencing of Ts1Rhr and wild-type B cells (n=3 mice per genotype) yielded relative expression levels among the 25 expressed triplicated genes (absolute fragments per kilobase per million reads [FPKM]>0.1), and the flanking centromeric and telomeric regions.

FIG. 11 shows the absolute expression of DSCR genes in wild-type and Ts1Rhr B cells by RNAseq. Fragments per kilobase per million reads (FPKM) values for wild-type and Ts1Rhr passage 1 B cells are plotted (n=3 independent biologic replicates per genotype).

FIGS. 12A-12G show that polysomy 21 B-ALL is associated with the overexpression of PRC2 targets. FIG. 12A shows a heat map of human genes orthologous to the 150 most upregulated genes from Ts1Rhr B cells in primary human pediatric B-ALLs. Unsupervised hierarchical clustering by gene revealed the “core Ts1Rhr” gene set (boxed). FIG. 12B shows GSEA plots for the full and core Ts1Rhr gene sets in the AIEOP data set. ES, enrichment score. FIG. 12C shows a GSEA plot of the core Ts1Rhr gene set in an independent ICH validation cohort. FIG. 12D shows a network enrichment map of MSigDB gene sets enriched (FDR<0.05) in the Ts1Rhr expression signature. FIG. 12E shows unsupervised hierarchical clustering of H3K27me3-marked genes from the MIKKELSEN_MEF_H3K27me3 gene set in the AIEOP pediatric B-ALL cohort (karyotype shown). FIG. 12F shows GSEA plots of the top 100 genes from three PRC2/H3K27me3 gene sets as defined in the AIEOP patient cohort in the ICH validation cohort. FIG. 12G shows quantitative histone MS for H3K27-K36 peptides (*P<0.05, n=3 samples per group per genotype).

FIGS. 13A-13E shows that DS-ALL is associated with overexpression of PRC2 targets and genes marked by H3K27me3, Ts1Rhr and PRC2′H3K27me3 gene signatures distinguish non-DS-ALL with somatic gain of chromosome 21 or iAMP21, and Ts1Rhr B-ALLs are associated with H3K27 hypomethylation. FIG. 13A shows heat maps of all genes comprising three of the top five scoring target gene sets enriched in the core Ts1Rhr signature in DS-ALLs and non-DS-ALLs. FIG. 13B shows unsupervised clustering results of a validation cohort of 30 non-DS pediatric B-ALL gene expression signatures (the AIEOP-2 cohort) using a 100-gene SUZ12 target gene set. Four patients with somatic gain of chr.21 and two with iAMP21 cluster within a distinct group with 5 additional cases (P=0.001 by Fisher's exact test). FIG. 13C shows GSEA plots of the Ts1Rhr gene set and the top 100 discriminating genes in the Mikkelsen NPC and MEF H3K27me3 gene sets from the AIEOP cohort, queried in the primary human B-ALLs in the AIEOP-2 cohort containing cases with somatic +21 and iAMP21. ES indicates enrichment score. FIG. 13D shows unsupervised hierarchical clustering results of histone H3 post-translational modifications in splenocytes from mice with Ts1Rhr and wild-type BCR-ABL B-ALLs quantitated by mass spectrometry (blue-red=low-to-high relative amount of each listed peptide, n=3 independent leukemias for each genotype). Peptides containing H3K27me3 with lower abundance in Ts1Rhr B-ALLs are indicated by arrows. FIG. 13E shows Western blotting results in sorted CD19+ Ts1Rhr and wild-type B-ALLs (n=5 independent leukemias for each genotype, distinct from those in panel D).

FIGS. 14A-14H show that Ts1Rhr B cells have reduced H3K27me3 that results in overexpression of bivalently marked genes. FIG. 14A shows gene tracks showing occupancy of histone marks at the Plod2 promoter (one of the 50 core Ts1Rhr genes) in reads per million per base pair (rpm/bp). FIG. 14B shows levels of H3K27me3 in Ts1Rhr and wild-type B cells at regions enriched for H3K27me3 in wild-type cells (***P<1e-16). FIG. 14C shows histone marks at the promoters of genes that are upregulated or downregulated in Ts1Rhr vs. wild-type cells (**P<1e-5). FIG. 14D shows chromatin marks in wild-type B cells present at promoters of all genes (left) or genes that are upregulated in Ts1Rhr B cells (right, ***P<0.00 compared to all genes by Chi-square with Yates' correction). FIG. 14E shows colony counts in the presence of DMSO or GSK-J4 (n=3 biological replicates per genotype, *P<0.05 compared to DMSO for same genotype). FIG. 14F shows colony counts in the presence of GSK-126 or after withdrawal at passage 5 (*P<0.05 compared to GSK-126 for same genotype, #P<0.05 compared to other genotype or no withdrawal). Arrow indicates GSK-126 withdrawal. FIG. 14G shows Western blotting results of passage 2 colonies after 14 total days in culture with DMSO, 1 μM GSK-J4, or 1 μM GSK-126. FIG. 14H shows Western blotting results of colonies one passage (7 days) after continuation (4) or removal (−) of GSK-126.

FIGS. 15A-15H show that ChIP-seq and CHIP-qPCR exhibit decreased H3K27me3 at promoters in Ts1Rhr B cells, the Ts1Rhr gene set is enriched for E2A/TCF3 and LEF1 targets, and DS-ALLs are sensitive to GSK-J4. FIG. 15A shows ChIP for H3K27me3 (left), H3K27me3 (right), or control rabbit IgG followed by quantitative PCR on a representative set of genes from the Ts1Rhr signature in an independent validation set of wild-type and Ts1Rhr mice (n=3 mice per genotype, one representative of two independent experiments). Data represented as fold enrichment over input relative to a negative control intergenic region on chr.5 (Chr 5 IN) (**P<0.01, *P<0.05). FIG. 15B shows H3K27me3 enriched regions in wild-type B cells. The promoter region is defined as the 5 kb flanking annotated transcription start sites. Overlap of H3K27me3 regions with the promoter region was significant in comparison to a random background model of the genome (P<10⁻¹⁰). FIG. 15C shows a Venn diagram showing the number and overlap between H3K27me3 enriched regions in wild-type (WT) or Ts1Rhr B cells. FIG. 15D shows the log₂ fold difference in density of H3K27me3 at promoters between Ts1Rhr and wild-type B cells is shown. FIG. 15E shows the top three ranked transcription factors with predicted binding sites among promoters of genes in the listed sets as queried in MSigDB “c3.tft” defined in the TRANSFAC database (version 7.4, available on the World Wide Web at gene-regulation.com). FIG. 15F shows the relative fraction of genes that have proximal E2A/TCF3 occupancy among all genes (7129 of 20671), genes with only H3K27me3 (557 of 1994) or H3K4me3 (4032 of 9360) at the promoter in wild-type B cells, or genes in the Ts1Rhr gene set (85 of 150) (**P<0.01. ***P<0.0001 versus the Ts1Rhr gene set by Chi-square with Yates' correction). FIG. 15G shows that expression of genes in the Ts1Rhr and Core Ts1Rhr sets are increased compared to all probesets in wild-type B cell progenitors as compared to E2A^{-/-} (expression data from²⁸; ***P<0.0001 by Student t-test, center bars=median, box=25-75% confidence interval, whiskers=10-90% confidence interval). FIG. 15H shows the IC₅₀ for five DS-ALLs treated in vitro with GSK-J4 (error bars represent 95% confidence intervals).

FIGS. 16A-16B show the sensitivity of murine and human B-cell ALL to GSK-J4. FIG. 16A shows that a subset of murine B-cell acute lymphoblastic leukemias that harbor triplication of the Down Syndrome Critical Region (lower panel) are 100-fold more sensitive to GSK-J4 compared to leukemias that lack triplication (upper panel). FIG. 16B shows that a human primary B-cell ALL xenograft from a patient with Down Syndrome is 10-100-fold more sensitive to GSK-J4 compared to a similar xenograft that lacks an extra copy of chromosome 21.

FIGS. 17A-17E show that HMGN1 overexpression decreases H3K27me3 and promotes transformed B cell phenotypes. FIG. 17A shows Western blotting results of Ba/F3 cells transduced with empty virus or murine HMGN1 (n=3 independent biological replicates). FIG. 17B shows relative shRNA representation over passages 1-3. Each line represents an individual shRNA (n=155 total). The five shRNAs targeting Hmgn1 are indicated. FIG. 17C shows GSEA plots for the full and core Ts1Rhr gene sets in HMGN1_OE transgenic B cells. FIG. 17D show B cell colonies during repassaging of WT and HMGN1_OE BM (n=6 biological replicates per genotype in two independent experiments, *P<0.05). FIG. 17E shows leukemia-free survival of recipient mice after transplantation of wild-type or HMGN1_OE bone marrow transduced with BCR-ABL (aggregate of three independent experiments, n=20 [WT] or n=28 [HMGN1_OE] per group, curves compared by log-rank test).

FIGS. 18A-18G show that HMGN1 overexpression alone results in multiple B cell phenotypes observed with triplication of the entire 21q22 orthologous region. FIG. 18A shows relative quantitation of H3K27me3 and HMGN1 in BaF3 lymphoblasts transduced with empty vector of mouse HMGN1. FIG. 18B shows a heat map showing RNA expression of the 31 triplicated genes in passages 1, 3, and 6 in triplicate Ts1Rhr cultures (blue-red=low to high log₂ FPKM values, genes listed in genomic order). FIG. 18C shows a schematic of the primary B cell shRNA experiment. Passage 1 B cells from Ts1Rhr or wild-type bone marrow were pooled after infection with individual lentiviral shRNAs targeting either a triplicated gene (5 shRNA/gene) or a control (n=30). DNA was collected post-infection (baseline) and after each passage (indicated by arrows), and the relative representation of each shRNA was quantitated by next generation sequencing. Data represent the average of independent biological replicates from wild-type (n=3) and Ts1Rhr (n=4) animals. FIG. 18D show normalized quantitation of negative (non-targeting) and positive (known to be toxic) control shRNAs in passage 6 Ts1Rhr colonies relative to input (left) demonstrates preferential loss of positive control shRNAs. Neither positive nor negative control shRNAs were preferentially lost from Ts1Rhr passage 3 cells compared to wild-type (right, Tukey box and whiskers plots, horizontal bar is the median and plus is the mean; *P<0.05; NS, not significant). FIG. 18E show Western blotting results of BaF3 lymphoblasts confirming knockdown of HMGN1. Antibodies are: A (Abcam), B (Aviva), mHMGN1 (affinity purified murine HMGN1 antibody). FIG. 18F show Western blotting results of HMGN1 in B cell colonies from wild-type and HMGN1_OE mice using the Abcam HMGN1 antibody. “Endo” represents endogenous mouse HMGN1 and “Tg” represents transgenic human HMGN1. FIG. 18G shows Hardy B cell subfractions as percentages of bone marrow cells from wild-type (black) and HMGN1_OE (orange) littermates (n=4 per group, *P<0.05).

FIG. 19 shows a schematic of B-cell developmental lineages and associated molecular markers according to murine genetics nomenclature.

### BRIEF DESCRIPTION OF TABLES

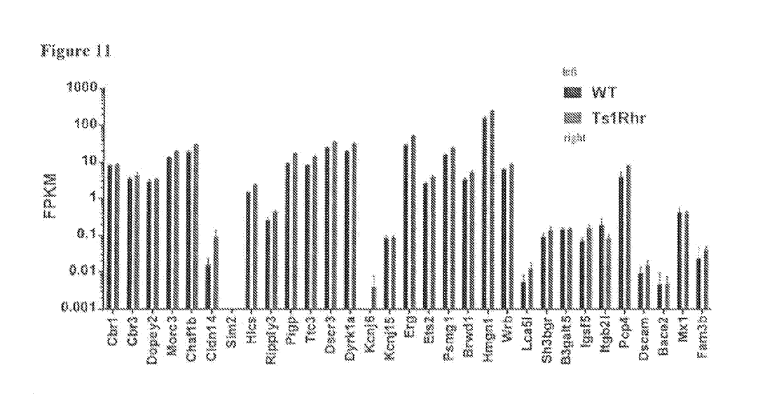

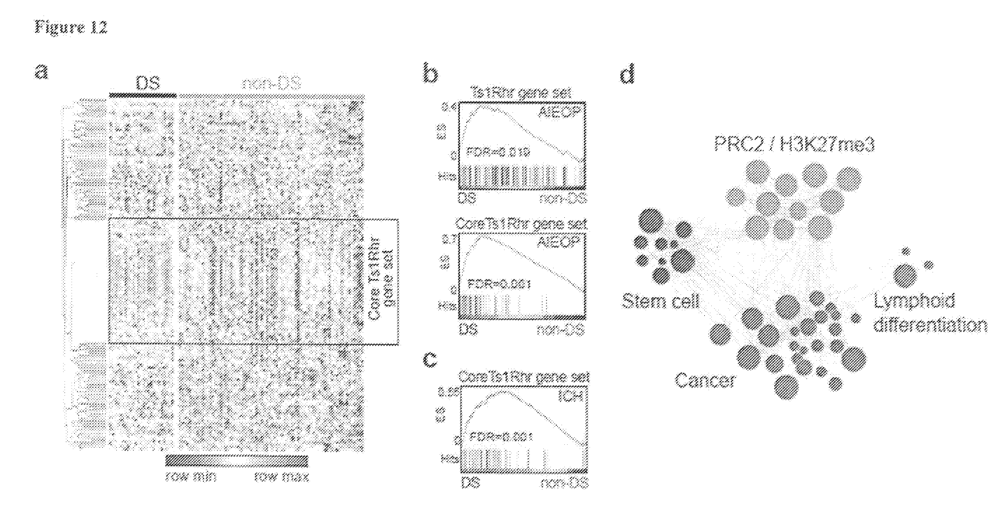

Table 1 shows genes differentially expressed in Ts1Rhr as compared to wild-type B cells. The top 150 higher (UP) and lower (DOWN) expressed genes in Ts1Rhr relative to wild-type passage 1 B cells by RNAseq and EdgeR analysis (p<0.05, false discovery rate <0.25) is shown (n=3 independent biologic replicates per genotype). Differential expression is annotated as log₂ fold change in Ts1Rhr relative to wild-type. The 50 UP genes that constitute the Core Ts1Rhr gene set (FIG. 12A) are annotated.

Table 2 shows the results of a query of the top 150 Ts1Rhr UP gene set against the Molecular Signatures Database (MSigDB) ‘c1’ positional dataset.

Table 3 shows the results of gene set enrichment and network enrichment mapping for Ts1Rhr B cells.

Table 4 shows the results of a query of the 50 Core Ts1Rhr gene set against the Molecular Signatures Database (MSigDB) ‘c2 cgp’ chemical and genetic perturbations dataset.

Table 5 shows the top 100 differentially expressed genes in the SUZ12 target gene, Mikkelsen MEF and NPC H3K27me3 signatures between DS-ALLs and non-DS-ALLs.

Table 6 shows shRNAs used in the competitive growth assay targeting DSCR genes. Gene symbols for DSCR genes (tab 1 “TEST”) and controls (tab 2 “CONTROLS”) are shown, with clone names in The RNAi Consortium (TRC) database, target sequence, and location of the target sequence within the gene. Data are the normalized ratio of the quantitation of each shRNA in Ts1Rhr to wild-type B cells during passaging relative to input within each genotype.

### DETAILED DESCRIPTION OF THE INVENTION

The present invention is based, at least in part, on the novel discovery of gene profiles useful for distinguishing among cancer subtypes (e.g., lymphoid cancers, such as leukemia) and for predicting the clinical outcome of such cancer subtypes to therapeutic regimens, particularly to modulators of histone methylation (e.g., H3K27me3). Thus, agents such as miRNAs, miRNA analogues, small molecules, RNA interference, aptamer, peptides, peptidomimetics, antibodies that specifically bind to one or more biomarkers of the invention (e.g., biomarkers listed in Tables 1-5 and/or described in the Examples, such as H3K27 demethylases, PRC2 complexes, EZH2, and HMGN1) and fragments thereof can be used to identify, diagnose, prognose, assess, prevent, and treat cancers (e.g., lymphoid cancers, such as leukemia). In addition, the present invention is based, at least in part, on the novel discovery that contacting lymphoid progenitor cells (e.g., wild type and/or genomically altered cells) with an agent that inhibits polycomb repressor complex 2 (PRC2) activity or reduces H3K27me3 levels can increase the number of lymphoid progenitor cells (e.g., increase self-renewal and cell proliferation) from the initial population of such lymphoid progenitor cells.

### 1. Definitions

The articles “a” and “an” are used herein to refer to one or to more than one (i.e. to at least one) of the grammatical object of the article. By way of example, “an element” means one element or more than one element.

The term “allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor. The term “mismatched allogeneic” refers to deriving from, originating in, or being members of the same species having non-identical major histocompatability complex (MHC) antigens (i.e., proteins) as typically determined by standard assays used in the art, such as serological or molecular analysis of a defined number of MHC antigens. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members. These terms contrast with the term “xenogeneic,” which refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor. The term “syngeneic” refers to deriving from, originating in, or being members of the same species that are genetically identical, particularly with respect to antigens or immunological reactions. These include identical twins having matching MHC types. Thus, a “syngeneic transplant” refers to transfer of cells or organs from a donor to a recipient who is genetically identical to the donor.

The term “altered amount” of a marker or “altered level” of a marker refers to increased or decreased copy number of the marker and/or increased or decreased expression level of a particular marker gene or genes in a cancer sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample.

The “amount” of a marker, e.g., expression or copy number of a marker or minimal common region (MCR), or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker. In some embodiments, the amount of the marker in the subject can be considered “significantly” higher or lower than the normal amount if the amount is 10%°, 15%, 20%, 25%, 30%, 35%, 40%, 45%, 50% or more, higher or lower, respectively, than the normal amount of the marker.

The term “altered level of expression” of a marker refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

The term “altered subcellular localization” of a marker refers to the mislocalization of the marker within a cell relative to the normal localization within the cell e.g., within a healthy and/or wild-type cell. An indication of normal localization of the marker can be determined through an analysis of subcellular localization motifs known in the field that are harbored by marker polypeptides.

Unless otherwise specified herein, the terms “antibody” and “antibodies” broadly encompass naturally-occurring forms of antibodies (e.g. IgG, IgG, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger. P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov. S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “antisense” nucleic acid polypeptide comprises a nucleotide sequence which is complementary to a “sense” nucleic acid encoding a protein, e.g., complementary to the coding strand of a double-stranded cDNA polypeptide, complementary to an mRNA sequence or complementary to the coding strand of a gene. Accordingly, an antisense nucleic acid polypeptide can hydrogen bond to a sense nucleic acid polypeptide.

The term “autologous” refers to deriving from or originating in the same subject or patient. An “autologous transplant” refers to the harvesting and reinfusion or transplant of a subject's own cells or organs. Exclusive or supplemental use of autologous cells can eliminate or reduce many adverse effects of administration of the cells back to the host, particular graft versus host reaction.

The term “biochip” refers to a solid substrate comprising an attached probe or plurality of probes of the invention, wherein the probe(s) comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more probes. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder. The probes may be attached to the biochip in a wide variety of ways, as will be appreciated by those in the art. The probes may either be synthesized first, with subsequent attachment to the biochip, or may be directly synthesized on the biochip. The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing. The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics. The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide. The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, vomit).

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenstr\vec{o}m's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioecndotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myclocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present invention is used in the treatment, diagnosis, and/or prognosis of lymphoma or its subtypes, including, but not limited to, lymphocyte-rich classical Hodgkin lymphoma, mixed cellularity classical Hodgkin lymphoma, lymphocyte-depleted classical Hodgkin lymphoma, nodular sclerosis classical Hodgkin lymphoma, anaplastic large cell lymphoma, diffuse large B-cell lymphomas, MLL′ pre B-cell ALL) based upon analysis of markers described herein.

The term “classifying” includes “to associate” or “to categorize” a sample with a disease state. In certain instances, “classifying” is based on statistical evidence, empirical evidence, or both. In certain embodiments, the methods and systems of classifying use of a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment. It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The term “diagnosing cancer” includes the use of the methods, systems, and code of the present invention to determine the presence or absence of a cancer or subtype thereof in an individual. The term also includes methods, systems, and code for assessing the level of disease activity in an individual.

As used herein, the term “diagnostic marker” includes markers described herein which are useful in the diagnosis of cancer, e.g., over- or under-activity, emergence, expression, growth, remission, recurrence or resistance of tumors before, during or after therapy. The predictive functions of the marker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers types or cancer samples; (2) its presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its presence or absence in clinical subset of subjects with cancer (e.g., those responding to a particular therapy or those developing resistance). Diagnostic markers also include “surrogate markers.” e.g., markers which are indirect markers of cancer progression. Such diagnostic markers may be useful to identify populations of subjects amenable to treatment with modulators of H3K27me3 levels (e.g., subjects having Down syndrome-type ALL as described herein) and to thereby treat such stratified patient populations.

The term “Down syndrome” or “DS” refers to a condition caused by trisomy for human chromosome 21 (Hsa21) and is the most common genetic cause of mental retardation in humans. DS occurs in I in 800-1000 live births and results in over 80 different clinical phenotypes, including craniofacial abnormalities, a small hypocellular brain with a disproportionately small cerebellum, Alzheimer-like histopathology, and an elevated risk for congenital heart defects, Hirschsprung's disease, and leukemia. DS is associated with two contrary cancer-related phenotypes. The first observation of a patient with leukemia and DS was made in 1930, and an increased risk of leukemia among individuals with DS was established by 1955. Acute megakaryoblastic leukemia (AMKL) occurs approximately 500-fold more frequently in individuals with DS than in the general population. AMKL almost always occurs in concert with a somatic mutation in the GATAI transcription factor. Several genetic mouse models of DS exist. The most widely-used of these models is the Ts65Dn mouse, which is trisomic for orthologs of approximately half of the 261 protein coding genes on Hsa21 (Patterson and Costa (2005) Nat. Rev. Genet. 6:137-147; Davisson (2005) Drug Disc. Today: Disease Models 2:103-109). This mouse recapitulates in detail several phenotypes of DS, including impairments in learning and memory degeneration of basal forebrain cholinergic neurons with aging, small cerebellum, fewer granule cell neurons and reduced cell proliferation in the dentate gyrus, and dysmorphology of the craniofacial skeleton, mandible and cranial vault. The Ts1Rlr mouse has segmental trisomy for a subset of the genes represented in Ts65Dn which correspond to a “critical region” on Hsa21 which harbors genes sufficient to cause a number of DS phenotypes. In addition, the Dp(16)1Yu mouse harbors an extra copy of all of the segments on mouse chromosome 16 that are syntenic to human chromosome 21 and such mice display learning, memory, and heart defects comparable to those observed in human DS (Li et al. (2007) Hum. Mol. Genet. 16:1359-66). In humans, studies of partial trisomy 21 (“Down Syndrome Critical Region” (DSCR) indicate that only parts of the chromosome are necessary to recapitulate the Down syndrome phenotype (Patterson and Costa (2005) Nat. Rev. Genet. 6:137-147; Olson et al. (2004) Science 306:687-690). The Ts1Rhr mouse is trisomic only for the region of mouse chromosome 16 that is comparable to the DSCR.

The term “expansion” in the context of cells refers to increase in the number of a characteristic cell type, or cell types, from an initial population of cells, which may or may not be identical. The initial cells used for expansion need not be the same as the cells generated from expansion. For instance, the expanded cells may be produced by growth and differentiation of the initial population of cells. Excluded from the term expansion are limiting dilution assays used to characterize the differentiation potential of cells.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

The term “gene expression data” or “gene expression level” as used herein refers to information regarding the relative or absolute level of expression of a gene or set of genes in a cell or group of cells. The level of expression of a gene may be determined based on the level of RNA, such as mRNA, encoded by the gene. Alternatively, the level of expression may be determined based on the level of a polypeptide or fragment thereof encoded by the gene. Gene expression data may be acquired for an individual cell, or for a group of cells such as a tumor or biopsy sample. Gene expression data and gene expression levels can be stored on computer readable media, e.g., the computer readable medium used in conjunction with a microarray or chip reading device. Such gene expression data can be manipulated to generate gene expression signatures.

The term “gene expression signature” or “signature” as used herein refers to a group of coordinately expressed genes. The genes making up this signature may be expressed in a specific cell lineage, stage of differentiation, or during a particular biological response. The genes can reflect biological aspects of the tumors in which they are expressed, such as the cell of origin of the cancer, the nature of the non-malignant cells in the biopsy, and the oncogenic mechanisms responsible for the cancer. For example, the gene expression signatures described herein stratify Down Syndrome-ALL (DS-ALL) from general ALL conditions that are especially amenable to treatment with modulators of H3K27me3 levels.

The term “hematological cancer” refers to cancers of cells derived from the blood. In some embodiments, the hematological cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), Hodgkin's lymphoma, mantle cell lymphoma (MCL), follicular lymphoma, Waldenstrom's macroglobulinemia (WM), B-cell lymphoma and diffuse large B-cell lymphoma (DLBCL). NHL may include indolent Non-Hodgkin's Lymphoma (iNHL) or aggressive Non-Hodgkin's Lymphoma (aNHL).

The term “hematopoietic stem cell” or “HSC” refers to a clonogenic, self-renewing pluripotent cell capable of ultimately differentiating into all cell types of the hematopoietic system, including B cells T cells, NK cells, lymphoid dendritic cells, myeloid dendritic cells, granulocytes, macrophages, megakaryocytes, and erythroid cells. As with other cells of the hematopoietic system, HSCs are typically defined by the presence of a characteristic set of cell markers.

The term “homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody,” as used herein, is intended to include antibodies made by a non-human cell having variable and constant regions which have been altered to more closely resemble antibodies that would be made by a human cell, for example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

As used herein, the term “inhibit” includes the decrease, limitation, or blockage, of, for example a particular action, function, or interaction. For example, cancer is “inhibited” if at least one symptom of the cancer, such as hyperproliferative growth, is alleviated, terminated, slowed, or prevented. As used herein, cancer is also “inhibited” if recurrence or metastasis of the cancer is reduced, slowed, delayed, or prevented.

As used herein, the term “interaction,” when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules. Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

An “isolated antibody,” as used herein, is intended to refer to an antibody that is substantially free of other antibodies having different antigenic specificities. Moreover, an isolated antibody may be substantially free of other cellular material and/or chemicals.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations, in which compositions of the invention are separated from cellular components of the cells from which they are isolated or recombinantly produced. In one embodiment, the language “substantially free of cellular material” includes preparations of having less than about 30%, 20%, 10%, or 5% (by dry weight) of cellular material. When an antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g. a package or container) comprising at least one reagent, e.g. a probe, for specifically detecting or modulating the expression of a marker of the invention. The kit may be promoted, distributed, or sold as a unit for performing the methods of the present invention.

The term “leukemia” refers to a group of diseases that are cancers of the marrow and blood, where the malignant cells are white blood cells (leukocytes). The two major groups are lymphatic, and myeloid leukemia. Both groups are considered as either acute or chronic depending on various factors. Also included are lymphoid leukemias. Leukemias can thus be divided into four main types: acute lymphocytic leukemia, acute myelogenous leukemia, chronic lymphocytic leukemia and chronic myelogenous leukemia. Acute and chronic leukemias are usually studied as groups separated by the cells which are affected. These heterogeneous groups are usually considered together and are considered as a group of diseases characterized by infiltration of the bone marrow and other tissues by the cells of the hematopoietic system. The infiltration is called neoplastic, meaning new growth of cells, but all of the cells seen in the marrow, and peripheral circulation in leukemia are normal in a normal bone marrow, except for one structure, seen in myelocytic leukemia called Auer rods. These structures are repeated in this kind of leukemia, and are unknown as to structure, and relationship to any other material. Acute lymphoblastic leukemia (ALL) is also referred to as acute lymphocytic leukemia and acute lymphoid leukemia and is a form of leukemia characterized by excess lymphoblasts. Malignant, immature white blood cells continuously multiply and are overproduced in the bone marrow. ALL causes damage and death by crowding out normal cells in the bone marrow, and by spreading (infiltrating) to other organs. ALL is most common in childhood with a peak incidence at 2-5 years of age, and another peak in old age. Standard of care for treating ALL focuses on treatment of different phases in order to control bone marrow and systemic (whole-body) disease as well as to prevent leukemic cells from spreading to other sites, particularly the central nervous system (CNS), e.g., monthly lumbar punctures: a) induction chemotherapy is used to bring about bone marrow remission. For adults, standard induction plans include prednisone, vincristine, and an anthracycline drug; other drug plans may include L-asparaginase or cyclophosphamide. For children with low-risk ALL, standard therapy usually consists of three drugs (prednisone, L-asparaginase, and vincristine) for the first month of treatment; b) consolidation therapy or intensification therapy eliminates any remaining leukemia cells. There are many different approaches to consolidation, but it is typically a high-dose, multi-drug treatment that is undertaken for a few months. Patients with low- to average-risk ALL receive therapy with antimetabolite drugs such as methotrexate and 6-mercaptopurine (6-MP). High-risk patients receive higher drug doses of these drugs, plus additional drugs; c) CNS prophylaxis (preventive therapy) stops the cancer from spreading to the brain and nervous system in high-risk patients. Standard prophylaxis may include radiation of the head and/or drugs delivered directly into the spine; and/or d) maintenance treatments with chemotherapeutic drugs prevent disease recurrence once remission has been achieved. Maintenance therapy usually involves lower drug doses, and may continue for up to three years. Alternatively, allogeneic bone marrow transplantation may be appropriate for high-risk or relapsed patients. Chronic lymphocytic leukemia (also known as “chronic lymphoid leukemia” or “CLL”), is a leukemia of the white blood cells (lymphocytes) that affects a particular lymphocyte, the B cell, which originates in the bone marrow, develops in the lymph nodes, and normally fights infection. In CLL, the DNA of a B cell is damaged, so that it cannot fight infection, but grows out of control and crowds out the healthy blood cells that can fight infection. CLL is an abnormal neoplastic proliferation of B cells. The cells accumulate mainly in the bone marrow and blood. Although not originally appreciated, CLL is now thought to be identical to a disease called small lymphocytic lymphoma (SLL), a type of non-Hodgkin's lymphoma which presents primarily in the lymph nodes. Most people are diagnosed without symptoms as the result of a routine blood test that returns a high white blood cell count, but as it advances, CLL results in swollen lymph nodes, spleen, and liver, and eventually anemia and infections. Early CLL is not usually treated, and late CLL is treated with chemotherapy and monoclonal antibodies. Survival varies from 5 years to more than 25 years. It is now possible to diagnose patients with short and long survival more precisely by examining the DNA mutations, and patients with slowly-progressing disease can be reassured and may not need any treatment in their lifetimes [Chiorazzi et al., (2005) N. Engl. J. Med. 352(8):804-815]. Chronic myelogenous leukemia (CML), also known as chronic granulocytic leukemia (CGL), is a neoplastic disorder of the hematopoietic stem cell. In its early phases, this disease is characterized by leukocytosis, the presence of increased numbers of immature granulocytes in the peripheral blood, splenomegaly and anemia. These immature granulocytes include basophils, eosinophils, and neutrophils. The immature granulocytes also accumulate in the bone marrow, spleen, liver, and occasionally in other tissues. Patients presenting with this disease characteristically have more than 75,000 white blood cells per microliter, and the count may exceed 500,000/ul. Cytologically, CML is characterized by a translocation between chromosome 22 and chromosome 9. This translocation juxtaposes a purported proto-oncogene with tyrosine kinase activity, a circumstance that apparently leads to uncontrolled cell growth. The resulting translocated chromosome is sometimes referred to as the Philadelphia chromosome.

The term “lymphocytes” refers to cells of the immune system which are a type of white blood cell. Lymphocytes include, but are not limited to, T-cells (cytotoxic and helper T-cells), B-cells and natural killer cells (NK cells).

The term “lymphoid progenitor cell” refers to an oligopotent or unipotent progenitor cell capable of ultimately developing into any of the terminally differentiated cells of the lymphoid lineage, such as T cell, B cell, NK cell, or lymphoid dendritic cells, but which do not typically differentiate into cells of the myeloid lineage. As with cells of the myeloid lineage, different cell populations of lymphoid progenitors are distinguishable from other cells by their differentiation potential, and the presence of a characteristic set of cell markers. Similarly, the term “common lymphoid progenitor cell” or “CLP” refers to an oligopotent cell characterized by its capacity to give rise to B-cell progenitors (BCP), T-cell progenitors (TCP), NK cells, and dendritic cells. These progenitor cells have little or no self-renewing capacity, but are capable of giving rise to T lymphocytes, B lymphocytes, NK cells, and lymphoid dendritic cells. By contrast, the term “myeloid progenitor cell” refers to a multipotent or unipotent progenitor cell capable of ultimately developing into any of the terminally differentiated cells of the myeloid lineage, but which do not typically differentiate into cells of the lymphoid lineage. Hence, “myeloid progenitor cell” refers to any progenitor cell in the myeloid lineage. Committed progenitor cells of the myeloid lineage include oligopotent common myeloid progenitor cells, granulocyte monocyte progenitor cells, and megakaryocyte/erythroid cells, but also encompass unipotent erythroid progenitor, megakaryocyte progenitor, granulocyte progenitor, and macrophage progenitor cells. Different cell populations of myeloid progenitor cells are distinguishable from other cells by their differentiation potential, and the presence of a characteristic set of cell markers. Similarly, the term “common myeloid progenitor cell” or “CMP” refers to a cell characterized by its capacity to give rise to granulocyte/monocyte (GMP) progenitor cells and megakaryocyte/erythroid (MEP) progenitor cells. These progenitor cells have limited or no self-renewing capacity, but are capable of giving rise to myeloid dendritic, myeloid erythroid, erythroid, megakaryocytes, granulocyte/macrophage, granulocyte, and macrophage cells.

The term “lymphoma” refers to cancers that originate in the lymphatic system. Lymphoma is characterized by malignant neoplasms of lymphocytes-B lymphocytes and T lymphocytes (i.e., B-cells and T-cells). Lymphoma generally starts in lymph nodes or collections of lymphatic tissue in organs including, but not limited to, the stomach or intestines. Lymphoma may involve the marrow and the blood in some cases. Lymphoma may spread from one site to other parts of the body. Lymphomas include, but are not limited to, Hodgkin's lymphoma, non-Hodgkin's lymphoma, cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma and mantle zone lymphoma and low grade follicular lymphoma.

A “marker” or “biomarker” includes a nucleic acid or polypeptide whose altered level of expression in a tissue or cell from its expression level in a control (e.g., normal or healthy tissue or cell) is associated with a disease state, such as a cancer or subtype thereof (e.g., lymphoid cancers, such as leukemia). A “marker nucleic acid” is a nucleic acid (e.g., mRNA, cDNA, mature miRNA, pre-miRNA, pri-miRNA, miRNA*, anti-miRNA, or a miRNA binding site, or a variant thereof and other classes of small RNAs known to a skilled artisan) encoded by or corresponding to a marker of the invention. Such marker nucleic acids include DNA (e.g., cDNA) comprising the entire or a partial sequence of any of the nucleic acid sequences set forth in Tables 1-5 and Examples or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” includes a protein encoded by or corresponding to a marker of the invention. A marker protein comprises the entire or a partial sequence of any of the sequences set forth in Tables 1-5 and Examples or the Examples. The terms “protein” and “polypeptide” are used interchangeably. In some embodiments, specific combinations of biomarkers are preferred. For example, a combination or subgroup of one or more of the biomarkers selected from the group consisting of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

The term “marker phenotyping” in the context of cell identification refers to identification of markers or antigens on cells for determining their phenotype (e.g., differentiation state and/or cell type). This may be done by immunophenotyping, which uses antibodies that recognize antigens present on a cell. The antibodies may be monoclonal or polyclonal, but are generally chosen to have minimal crossreactivity with other cell markers. It is to be understood that certain cell differentiation or cell surface markers are unique to the animal species from which the cells are derived, while other cell markers will be common between species. These markers defining equivalent cell types between species are given the same marker identification even though there are species differences in structure (e.g., amino acid sequence). Cell markers include cell surfaces molecules, also referred to in certain situations as cell differentiation (CD) markers, and gene expression markers. The gene expression markers are those sets of expressed genes indicative of the cell type or differentiation state. In part, the gene expression profile will reflect the cell surface markers, although they may include non-cell surface molecules.

As used herein, the term “modulate” includes up-regulation and down-regulation, e.g., enhancing or inhibiting a response.

The “normal” or “control” level of expression of a marker is the level of expression of the marker in cells of a subject, e.g., a human patient, not afflicted with a cancer. An “over-expression” or “significantly higher level of expression” of a marker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the marker in a control sample (e.g., sample from a healthy subject not having the marker associated disease) and preferably, the average expression level of the marker in several control samples. A “significantly lower level of expression” of a marker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a healthy subject not having the marker associated disease) and preferably, the average expression level of the marker in several control samples.

The term “peripheral blood cell subtypes” refers to cell types normally found in the peripheral blood including, but is not limited to, eosinophils, neutrophils, T cells, monocytes, NK cells, granulocytes, and B cells.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., lymphoid cancers, such as leukemia), development of one or more clinical factors, development of intestinal cancer, or recovery from the disease.

The term “response to cancer therapy” or “outcome of cancer therapy” relates to any response of the hyperproliferative disorder (e.g., cancer) to a cancer therapy, preferably to a change in tumor mass and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Response may also be assessed by caliper measurement or pathological examination of the tumor after biopsy or surgical resection for solid cancers. Responses may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to copy number, level of expression, level of activity, etc. of one or more biomarkers listed in Tables 1-5 and Examples or the Examples that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom the measurement values are known. In certain embodiments, the same doses of cancer therapeutic agents are administered to each subject. In related embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker threshold values that correlate to outcome of a cancer therapy can be determined using methods such as those described in the Examples section. Outcomes can also be measured in terms of a “hazard ratio” (the ratio of death rates for one patient group to another; provides likelihood of death at a certain time point), “overall survival” (OS), and/or “progression free survival.” In certain embodiments, the prognosis comprises likelihood of overall survival rate at 1 year, 2 years, 3 years, 4 years, or any other suitable time point. The significance associated with the prognosis of poor outcome in all aspects of the present invention is measured by techniques known in the art. For example, significance may be measured with calculation of odds ratio. In a further embodiment, the significance is measured by a percentage. In one embodiment, a significant risk of poor outcome is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. Thus, the present invention further provides methods for making a treatment decision for a cancer patient, comprising carrying out the methods for prognosing a cancer patient according to the different aspects and embodiments of the present invention, and then weighing the results in light of other known clinical and pathological risk factors, in determining a course of treatment for the cancer patient. For example, a cancer patient that is shown by the methods of the invention to have an increased risk of poor outcome by combination chemotherapy treatment can be treated with more aggressive therapies, including but not limited to radiation therapy, peripheral blood stem cell transplant, bone marrow transplant, or novel or experimental therapies under clinical investigation.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., chemotherapeutic or radiation therapy. In some embodiments, normal cells are not affected to an extent that causes the normal cells to be unduly injured by the cancer therapy (e.g., chemotherapy or radiation therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M. Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Wetsenthal L M, Contrib Gynecol Obstet 1994; 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 5-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “synergistic effect” refers to the combined effect of two or more anticancer agents or chemotherapy drugs can be greater than the sum of the separate effects of the anticancer agents or chemotherapy drugs alone.

The term “subject” refers to any healthy animal, mammal or human, or any animal, mammal or human afflicted with a condition of interest (e.g., cancer). The term “subject” is interchangeable with “patient.”

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

The term “substantially pure cell population” refers to a population of cells having a specified cell marker characteristic and differentiation potential that is at least about 50%, preferably at least about 75-80%, more preferably at least about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

As used herein, the term “survival” includes all of the following: survival until mortality, also known as overall survival (wherein said mortality may be either irrespective of cause or tumor related): “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

A “transcribed polynucleotide” or “nucleotide transcript” is a polynucleotide (e.g. an mRNA, hnRNA, cDNA, mature miRNA, pre-miRNA, pri-miRNA, miRNA*, anti-miRNA, or a miRNA binding site, or a variant thereof or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors.” In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with cancer) and preferably, the average expression level or copy number of the marker in several control samples.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for a fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Tables 1-5 and Examples) are well known in the art and readily available on publicly available databases, such as the National Center for Biotechnology Information (NCBI). For example, exemplary nucleic acid and amino acid sequences derived from publicly available sequence databases are provided below.

The nucleic acid and amino acid sequences of a representative human KDM6A biomarker (also known as UTX or MGC141941 or bA386N14.2 or DKFZp686A03225) is available to the public at the GenBank database under NM_21140.2 and NP_0.0066963.2. Nucleic acid and polypeptide sequences of KDM6A orthologs in organisms other than humans are well known and include, for example, mouse KDM6A (NM009483.1 and NP_033509.1), rat KDM6A (XM_002730185.2 and XP_002730231.1), chimpanzee KDM6A (XM_002806207.1 and XP_002806253.1), chicken KDM6A (XM_416762.3 and XP_416762.3), fruit fly KDM6A (NM_001201844.1 and NP_001188773.1), and worm KDM6A (NM_077049.3 and NP_509450.1).

The nucleic acid and amino acid sequences of a representative human KDM6B biomarker (also known as JMJD3 or KIAA0346) is available to the public at the GenBank database under NM_001080424.1 and NP_001073893.1. Nucleic acid and polypeptide sequences of KDM6B orthologs in organisms other than humans are well known and include, for example, dog KDM6B (XM_546599.3 and XP_546599.2), mouse KDM6B (NM_001017426.1 and NP 001017426.1), rat KDM6B (NM_001108829.1 and NP_001102299.1), and zebrafish KDM6B (XM_003198938.1 and XP_003198986.1 and NM_001030178.1 and NP_001025349.1).

At least five splice variants encoding five human EZH2 isoforms exist. The sequence of human EZH2 transcript variant 1 is the canonical sequence, all positional information described with respect to the remaining isoforms are determined from this sequence, and the sequences are available to the public at the GenBank database under NM_004456.4 and NP_004447.2. The sequences of human EZH2 transcript variant 2 can be found under NM_152998.2 and NP_694543.1 and the encoded protein replaces the residues HP of positions 297-298 of the canonical sequences with HRKCNYS. The sequences of human EZH2 transcript variant 3 can be found under NM_001203247.1 and NP_001190176.1 and the encoded protein deletes residues 83-121 of the canonical sequence. The sequences of human EZH2 transcript variant 4 can be found under NM_001203248.1 and NP_001190177.1 and the encoded protein deletes residues 74-82 of the canonical sequence. The sequences of human EZH2 transcript variant 5 can be found under NM_O001203249.1 and NP_001190178.1 and the encoded protein deletes residues 74-82 of the canonical sequence, as well as replaces the residues DGSSNHVYNYQPCDHPRQPCDSSCPCVIAQNFCEKFCQCSSEC of positions 511-553 with G. The catalytic site of EZH2 is believed to reside in a conserved domain of the protein known as the SET domain. The amino acid sequence of the SET domain of EZH2 is provided by the following partial sequence spanning amino acid residues 613-726 of human EZH2 isoform 1 described above and as follows: HLLLAPSDVAGWGIFIKDPVQKNEFISEYCGEIISQDEADRRGKVYDKYMCSPLFNLNNDFVVD ATRKGNKIRFANHSVNPNCYAKVMMVNGDHRIGIFAKRAIQTGEELFFDY. Additional sequences and structural information is publicly available in the art (e.g., U.S. Pat. Publ. 2013-0040906). Nucleic acid and polypeptide sequences of EZH2 orthologs in organisms other than humans are well known and include, for example, mouse EZH2 (NM_007071.2 and NP_031997.2 and NM_001146689.1 and NP_001140161), chimpanzee EZH2 (NM_001266503.1 and NP_001253432.1), cow EZH2 (NM_001193024.1 and NP_001179953.1), and rat EZH2 (NM_001134979.1 and NP_001128451.1).

The nucleic acid and amino acid sequences of a representative human HMGN1 biomarker is available to the public at the GenBank database under NM_004965.6 and NP_004956.5. Nucleic acid and polypeptide sequences of HMGN1 orthologs in organisms other than humans are well known and include, for example, monkey HMGN1 (XM_01113912.2 and XP_001113912.1), chimpanzee HMGN1 (XM_514899.4 and XP_514899.2), and cow HMGN1 (XM_002697394.1 and ZP_002697440).

In addition, eukaryotes have chromatin arranged around proteins in the form of nucleosomes, which are the smallest subunits of chromatin and includes approximately 146-147 base pairs of DNA wrapped around an octamer of core histone proteins (two each of H2A, H2B, H3, and H4). Trimethylation of histone H3 on Lys 27 (H3K27me3) is key for cell fate regulation. Mammalian cells have three known sequence variants of histone H3 proteins, denoted H3.1, H3.2 and H3.3, that are highly conserved differing in sequence by only a few amino acids. As used herein, the term “histone H3” can refer to H3.1, H3.2, or H3.3 individually or collectively. The sequences are as follows:

Histone H3.1:
MARTKQTARKSTGGKAPRKQLATKAARKSAPATGGVKKPHRYRPGTVALR
EIRRYQKSTE
Histone H3.2:
MARTKQTARKSTGGKAPRKQLATKAARKSAPATGGVKKPHRYRPGTVALR
EIRRYQKSTE
Histone H3.3:
MARTKQTARKSTGGKAPRKQLATKAARKSAPSTGGVKKPHRYRPGTVALR
EIRRYQKSTE

These amino acid sequences include a methionine as residue No. 1 that is cleaved off when the protein is processed, hence what is lysine 28 in the amino acid sequences above corresponds to lysine (K) 27. These three protein variants are encoded by at least fifteen different genes/transcripts. Sequences encoding the histone H3.1 variant arm publicly available as HIST1H3A (NM_003529.2; NP_003520.1), HIST1H3B (NM_003537.3; NP_003528.1), HIST1H3C (NM_003531.2; NP_003522.1), HIST1H3D (NM_003530.3; NP_003521.2), HIST1H3E (NM_003532.2; NP_003523.1), HIST1H3F (NM_021018.2; NP_066298.1),
HIST1H3G (NM_003534.2; NP_003525.1), HIST1H3H (NM_003536.2; NP_003527.1), HIST1H3I (NM_003533.2; NP_003524.1), and HIST1H3J (NM_003535.2; NP_003526.1), Sequences encoding the histone H3.2 variant are publicly available as HIST2H3A (NM_001005464.2; NP_001005464.1). HIST2H3C (NM_021059.2; NP_066403.2), and HIST2H3D (NM_001123375.1; NP_001116847.1). Sequences encoding the histone H3.3 variant are publicly available as H3F3A (NM_002107.3; NP_002098.1) and H3F3B (NM_005324.3; NP_005315.1). See U.S. Pat. Publ. 2012/0202843 for additional details. Antibodies for the detection of H3K27me3 and methods for making them are known in the art.

| Human KDM6A cDNA Sequence | |
|---|---|
| SEQ ID NO: 1 | |
| 1 | atgaaatcct gcggagtgtc gctcgctacc gccgccgctg ccgccgccgc tttcggtgat |
| 61 | gaggaaaaga aaatggcggc gggaaaagcg agcggcgaga gcgaggaggc gtcccccagc |
| 121 | ctgacagccg aggagaggga ggcgctcggc ggactggaca gccgcctctt tgggttcgtg |
| 181 | agatttcatg aagatggcgc caggacgaag gccctactgg gcaaggctgt tcgctgctat |
| 241 | gaatctctaa tcttaaaagc tgaaggaaaa gtggagtctg atttcttttg tcaattaggt |
| 301 | cacttcaacc tcttattgga agattatcca aaagcattat ctgcatacca gaggtactac |
| 361 | agtttacagt ctgactactg gaagaatgct gcctttttat atggtcttgg tttggtctac |
| 421 | ttccattata atgcatttca gtgggcaatt aaagcatttc aggaggtgct ttatgttgat |
| 481 | cccagctttt gtcgagccaa ggaaattcat ttacgacttg ggcttatgtt caaagtgaac |
| 541 | acagactatg agtctagttt aaagcatttt cagttagctt tggttgactg taatccctgc |
| 601 | actttgtcca atgctgaaat tcaatttcac attgcccact tatatgaaac ccagaggaaa |
| 661 | tatcattctg caaaagaagc ttatgaacaa cttttgcaga cagagaatct ttctgcacaa |
| 721 | gtaaaagcaa ctgtcttaca acagttaggt tggatgcatc acactgtaga tctcctggga |
| 781 | gataaagcca ccaaggaaag ctcatgctatt cagtatctcc aaaagtcctt ggaagcagat |
| 841 | cctaattctg gccagtcctg gtatttcctc ggaaggtgct attcaagtat tgggaaagtt |
| 901 | caggatgcct ttatatctta caggcagtct attgataaat cagaagcaag tgcagataca |
| 961 | tggtgttcaa taggtgtgct atatcagcag caaaatcagc ccatggatgc tttacaggcc |
| 1021 | tatatttgtg ctgtacaatt ggaccatggc catgctgcag cctggatgga cctaggcact |
| 1081 | ctctatgaat cctgcaacca gcctcaggat gccattaaat gctacttaaa tgcaactaga |
| 1141 | agcaaaagtt gtagtaatac ctctgcactt gcagcacgaa ttaagtattt acaggctcag |
| 1201 | ttgtgtaacc ttccacaagg tagtctacag aataaaacta aattacttcc tagtattgag |
| 1261 | gaggcgtgga gcctaccaat tcccgcagag cttacctcca ggcagggtgc catgaacaca |
| 1321 | gcacagcaga atacttctga caattggagt ggtggacatg ctgtgtcaca tcctccagta |
| 1381 | cagcaacaag ctcattcatg gtgtttgaca ccacagaaat tacagcactt ggaacagctc |
| 1441 | cgcgcaaata gaaataattt aaatccagca cagaaactga tgctggaaca gctggaaagt |
| 1501 | cagtttgtct taatgcaaca acaccaaatg agaccaacag gagttgcaca ggtacgatct |
| 1561 | actggaattc ctaatgggcc aacagctgac tcatcactgc ctacaaactc agtctctggc |
| 1621 | cagcagccac agcttgctct gaccagagtg cctagcgtct ctcagcctgg agtccgtcct |
| 1681 | gcctgccctg ggcagccttt ggccaatgga cccttttctg caggccatgt tccctgtagc |
| 1741 | acatcaagaa cgctgggaag tacagacact attttgatag gcaataatca tataacagga |
| 1801 | agtggaagta atggaaacgt gccttacctg cagcgaaacg cactcactct acctcataac |
| 1861 | cgcacaaacc tgaccagcag cgcagaggag ccgtggaaaa accaactatc taactccact |
| 1921 | caggggcttc acaaaggtca gagttcacat tcggcaggtc ctaatggtga acgacctctc |
| 1981 | tcttccactg ggccttccca gcatctccag gcagctggct ctggtattca gaatcagaac |
| 2041 | ggacatccca ccctgcctag caattcagta acacaggggg ctgctctcaa tcacctctcc |
| 2101 | tctcacactg ctacctcagg tggacaacaa ggcattacct taaccaaaga gagcaagcct |
| 2161 | tcaggaaaca tattgacggt gcctgaaaca agcaggcaca ctggagagac acctaacagc |
| 2221 | actgccagtg tcgagggact tcctaatcat gtccatcaga tgacggcaga tgctgtttgc |
| 2281 | agtcctagcc atggagattc taagtcacca ggtttactaa gttcagacaa tcctcagctc |
| 2341 | tctgccttgt tgatgggaaa agccaataac aatgtgggta ctggaacctg tgacaaagtc |
| 2401 | aataacatcc acccagctgt tcatacaaag actgataact ctgttgcctc ttcaccatct |
| 2461 | tcagccattt caacagcaac accttctcca aaatccactg agcagacaac cacaaacagt |
| 2521 | gttaccagcc ttaacagccc tcacagtggg ctacacacaa ttaatggaga agggatggaa |
| 2581 | gaatctcaga gccccatgaa aacagatctg cttctggtta accacaaacc tagtccacag |
| 2641 | atcataccat caatgtctgt gtccatatac cccagctcag cagaagttct gaaggcatgc |
| 2701 | aggaatctag gtaaaaatgg cttatctaac agtagcattt tgttggataa atgtccacct |
| 2761 | ccaagaccac catcttcacc ataccctccc ttgccaaagg acaagttgaa tccacctaca |
| 2821 | cctagtattt acttggaaaa taaacgtgat gctttctttc ctccattaca tcaattttgt |
| 2881 | acaaatccga acaaccctgt tacagtaata cgtggccttg ctggagctct taagttagac |
| 2941 | ctgggacttt tctctactaa aactttggtg gaagctaaca atgaacatat ggtagaagtg |
| 3001 | aggacacagt tgttgcagcc agcagatgaa aactgggatc ccactggaac aaagaaaatc |
| 3061 | tggcattgtg aaagtaatag atctcatact acaattgcta aatatgcaca gtaccaggcc |
| 3121 | tcctcattcc aggaatcatt gagagaagaa aatgaaaaaa gaagtcatca taaagaccac |
| 3181 | tcagatagtg aatctacatc gtcagataat tctgggagga ggaggaaagg accctttaaa |
| 3241 | accataaagt ttgggaccaa tattgaccta tctgatgaca aaaagtggaa gttgcagcta |
| 3301 | catgagctga ctaaacttcc tgcttttgtg cgtgtcgtat cagcaggaaa tcttctaagc |
| 3361 | catgttggtc ataccatatt gggcatgaac acagttcaac tatacatgaa agttccaggg |
| 3421 | agcagaacac caggtcatca ggaaaataac aacttctgtt cagttaacat aaatattggc |
| 3481 | ccaggtgact gtgaatggtt tgttgttcct gaaggttact ggggtgttct gaatgacttc |
| 3541 | tgtgaaaaaa ataatttgaa tttcctaatg ggttcttggt ggcccaatct tgaagatctt |
| 3601 | tatgaagcaa atgttccagt gtataggttt attcagcgac ctggagattt ggtctggata |
| 3661 | aatgcaggca ctgttcattg ggttcaggct attggctggt gcaacaacat tgcttggaat |
| 3721 | gttggtccac ttacagcctg ccagtataaa ttggcagtgg aacggtacga atggaacaaa |
| 3781 | ttgcaaagtg tgaagtcaat agtacccatg gttcatcttt cctggaatat ggcacgaaat |
| 3841 | atcaaggtct cagatccaaa gctttttgaa atgattaagt attgtcttct aagaactctg |
| 3901 | aagcaatgtc agacattgag ggaagctctc attgctgcag gaaaagagat tatatggcat |
| 3961 | gggcggacaa aagaagaacc agctcattac cgtagcattt gtgaagtgga ggtttttgat |
| 4021 | ctgctttttg tcactaatga gagtaattca cgaaagacct acatagtaca ttgccaagat |
| 4081 | tgtgcacgaa aaacaagcgg aaacttggaa aactttgtgg tgctagaaca gtacaaaatg |
| 4141 | gaggacctga tgcaagtcta tgaccaattt acattagctc ctccattacc atccgcctca |
| 4201 | tcttga |
| Human KDM6A Amino Acid Sequence | |
| SEQ ID NO: 2 | |
| 1 | mkscgvslat aaaaaaafgd eekkmaagka sgeseeasps ltaeerealg gldsrlfgfv |
| 61 | rfhedgartk allgkavrcy eslilkaegk vesdffcqlg hfnllledyp kalsayqryy |
| 121 | slqsdywkna aflyglglvy fhynafqwai kafqevlyvd psfcrakeih lrlglmfkvn |
| 181 | tdyesslkhf qlalvdcnpc tlsnaeiqfh iahlyetqrk yhsakeayeq llqtenlsaq |
| 241 | vkatvlqqlg wmhhtvdllg dkatkesyai qylqkslead pnsqgswyfl grcyssigkv |
| 301 | qdafisyrqs idkseasadt wcsigvlyqq qnqpmdalqa yicavqldhg haaawmdlgt |
| 361 | lyescnqpqd aikcylnatr akscsntsal aarikylqaq lcnlpqgslq nktkllpsie |
| 421 | eawslpipae ltsrqgamnt aqqntsdnws gghavshppv qqqahswclt pqklqhleql |
| 481 | ranrnnlnpa qklmleqles qfvlmqqhqm rptgvaqvrs tgipngptad sslptnsvsg |
| 541 | qqpqlaltrv psvsqpgvrp acpgqplang pfsaghvpcs tsrtlgstdt ilignnhitg |
| 601 | sgsngnvpyl qrnaltlphn rtnltssaee pwknqlsnst qglhkgqssh sagpngerpl |
| 661 | sstgpsqhlq aagsgiqnqn ghptlpsnsv tqgaalnhls shtatsggqq gitltkeskp |
| 721 | sgniltvpet srhtgetpns tasveglpnh vhqmtadavc spshgdsksp gllssdnpql |
| 781 | sallmgkann nvgtgtcdkv nnihpavhtk tdnsvassps saistatpsp ksteqtttns |
| 841 | vtslnsphsg lhtingegme esqspmktdl llvnhkpspq iipsmsvsiy pssaevlkac |
| 901 | rnlgknglsn ssilldkcpp prppsspypp lpkdklnppt psiylenkrd affpplhqfc |
| 961 | tnpnnpvtvi rglagalkld lglfstktlv eannehmvev rtqllqpade nwdptgtkki |
| 1021 | whcesnrsht tiakyaqyqa ssfqeslree nekrshhkdh sdsestssdn sgrrrkgpfk |
| 1081 | tikfgtnidl sddkkwklql heltklpafv rvvsagnlls hvghtilgmn tvqlymkvpg |
| 1141 | srtpghqenn nfcsvninig pgdcewfvvp egywgvlndf ceknnlnflm gswwpnledl |
| 1201 | yeanvpvyrf iqrpgdlvwi nagtvhwvqa igwcnniawn vgpltacqyk laveryewnk |
| 1261 | lqsvksivpm vhlswnmarn ikvsdpklfe mikycllrtl kqcqtlreal iaagkeiiwh |
| 1321 | grtkeepahy csicevevfd llfvtnesns cktyivhcqd carktsgnle nfvvlaqykm |
| 1381 | edlmqvydqf tlapplpsas s |
| Mouse KDM6A cDNA Sequence | |
| SEQ ID NO: 3 | |
| 1 | atgaaatcct gcggagtgtc gctcgctacc gccgccgccg ccgccgccgc cgccgctttc |
| 61 | ggtgatgagg aaaagaaaat ggcggcggga aaagcgagcg gcgagagcga ggaggcgtcc |
| 121 | cccagcctga cagcggagga gagggaggcg ctcggcggac tggacagccg ccttttcggg |
| 181 | ttcgtgaggt ttcatgaaga tggcgccagg atgaaggccc tgctgggcaa ggctgttcgc |
| 241 | tgctacgaat ctctaatctt aaaagctgaa gggaaagtgg agtctgattt cttttgtcaa |
| 301 | ttaggtcact tcaacctctt attggaagat tatccaaaag cattatctgc ataccagagg |
| 361 | tactacagtt tacagtctga ttactggaag aatgctgcct ttttatatgg tcttggtttg |
| 421 | gtctacttcc attacaatgc atttcagtgg gctattaaag catttcagga ggtgctttat |
| 481 | gtcgatccca gcttttgtcg agccaaggaa attcatttac gacttgggct tatgttcaaa |
| 541 | gtgaacacag actatgagtc tagtttaaag cattttcagt tagctttggt tgactgtaat |
| 601 | ccctgcactt tgtccaatgc tgaaattcag tttcacattg cccacttata tgaaacccag |
| 661 | aggaagtatc attctgcaaa agaagcttat gagcaacttt tgcagacaga aaacctttct |
| 721 | gcacaagtaa aagcaactat tttacaacaa ttaggttgga tgcatcacac tgtggatctc |
| 781 | ctgggagata aggccaccaa ggaaagttat gctattcagt atctccagaa gtccttggaa |
| 841 | gcagatccaa attctggcca gtcctggtat ttccttggaa ggtgctattc aagtattggg |
| 901 | aaagttcagg atgcctttat atcttacagg caatctattg ataaatcaga agcaagtgca |
| 961 | gatacatggt gttcaatagg tgtgctctat caacagcaaa atcagcctat ggatgctttg |
| 1021 | caagcttata tttgtgctgt acaattggac cacggtcatg ctgcagcctg gatggatcta |
| 1081 | ggcactctct atgaatcctg caaccaacct caggatgcta ttaaatgcta tttaaatgca |
| 1141 | actagaagca aaaattgtag taatacctct ggacttgcag cacgaattaa gtatttacag |
| 1201 | gctcagttgt gtaaccttcc acaaggtagt ctacagaata aaactaaatt acttcctagt |
| 1261 | attgaggagg catggagcct accaatcccc gcagagctta cctccaggca gggtgccatg |
| 1321 | aacacagcac agcagaatac ttctgataat tggagtggtg gcaatgcacc acctccagta |
| 1381 | gaacaacaaa ctcattcatg gtgtttgaca ccacagaaat tacagcactt ggaacagctc |
| 1441 | cgagcaaaca gaaataattt aaatccagca cagaaactaa tgctggaaca gctggaaagt |
| 1501 | cagtttgtct taatgcagca acaccaaatg agacaaacag gagttgcaca ggtacggcct |
| 1561 | actggaattc ttaatgggcc aacagttgac tcatcactgc ctacaaactc agtttctggc |
| 1621 | cagcagccac agcttcctct gaccagaatg cctagtgtct ctcagcctgg agtccacact |
| 1681 | gcctgtccta ggcagacttt ggccaatgga cccttttctg caggccatgt tccctgtagc |
| 1741 | acatcaagaa cactgggaag tacagacact gttttgatag gcaataatca tgtaacagga |
| 1801 | agtggaagta atggaaacgt gccttacctg cagcgaaacg cacccactct acctcataac |
| 1861 | cgcacaaacc tgaccagcag cacagaggag ccgtggaaaa accaactatc taactccact |
| 1921 | caggggcttc acaaaggtcc gagttcacat ttggcaggtc ctaatggtga acgacctcta |
| 1981 | tcttccactg ggccctccca gcatctccag gcagctggct ctggtattca gaatcagaat |
| 2041 | ggacatccca ccctgcctag caattcagta acacaggggg ctgctctcaa tcacctctcc |
| 2101 | tctcacactg ctacctcagg tggacaacaa ggcattacct taaccaaaga gagcaagcct |
| 2161 | tcaggaaaca cattgacggt gcctgaaaca agcaggcaaa ctggagagac acctaacagc |
| 2221 | actgccagtg ttgagggact tcctaatcat gtccatcagg tgatggcaga tgctgtttgc |
| 2281 | agtcctagcc atggagattc taagtcacca ggtttactaa gttcagacaa tcctcagctc |
| 2341 | tctgccttgt tgatgggaaa agctaataac aatgtgggtc ctggaacctg tgacaaagtc |
| 2401 | aataacatcc acccaactgt ccatacaaag actgataatt ctgttgcctc ttcaccatct |
| 2461 | tcagccattt ccacagcaac accttctcct aagtccactg aacagacaac cacaaacagt |
| 2521 | gttaccagcc ttaacagccc tcacagtggg ctgcacacaa ttaatggaga aggaatggaa |
| 2581 | gaatctcaga gccccattaa aacagatctg cttctagtta gccacagacc tagtcctcag |
| 2641 | atcataccat caatgtctgt gtccatatat cccagctcag cagaagttct gaaagcttgc |
| 2701 | aggaatctag gtaaaaacgg cctgtctaat agtagcattc tgttggataa atgtccgcct |
| 2761 | ccaagaccac catcctcacc ataccctccc ttgccaangg acaagttgaa tccacctaca |
| 2821 | cctagtattt atttggaaaa taaacgtgat gctttctttc ctccattaca tcaattttgt |
| 2881 | acaaacccaa acaaccctgt tacagtaata cgtggccttg ctggagctct taaattagac |
| 2941 | ttgggacttt tctctactaa aactttggtg gaagctaaca atgaacatat ggtagaagtg |
| 3001 | aggacacagt tgttacaacc agcagatgaa aattgggacc ctactggaac caagaaaatc |
| 3061 | tggcactgtg aaagtaatag atctcatact acaattgcta aatatgctca gtaccaggcc |
| 3121 | tcctcattcc aagaatcatt gagagaagaa aatgagaaaa gaagtcacca taaagaccac |
| 3181 | tcagacagtg aatctacatc atcagataat tctgggaaaa gaagaaaagg accctttaaa |
| 3241 | accattaagt ttgggaccaa cattgacctg tctgatgaca aaaagtggaa gttacagcta |
| 3301 | catgagctga ctaaacttcc tgccttcgtg agagttgtat ctgcaggaaa tcttttaagc |
| 3361 | cacgttggtc atactatact gggcatgaac acagttcaac tatacatgaa agttccagga |
| 3421 | agcagaacac caggtcatca agaaaataac aacttctgtt cagttaatat aaatattggc |
| 3481 | ccaggtgact gtgaatggtt tgttgttcct gaaggctact ggggtgtttt gaatgacttc |
| 3541 | tgtgaaaaaa ataatttgaa tttcttaatg ggttcttggt ggcccaacct tgaagatcta |
| 3601 | tatgaagcaa atgttccagt gtataggttt attcagcgac ctggagatct ggtctggata |
| 3661 | aatgctggca ctgttcattg ggttcaagct attggctggt gcaacaatat tgcttggaat |
| 3721 | gttggtccac ttacagcctg tcagtataag ttagcagkgg aacgttatga atggaacaag |
| 3781 | ttgcaaaatg taaagtcaat agtacccatg gttcatcttt cctggaatat ggcacgaaat |
| 3841 | atcaaggttt cagatccaaa gctttttgaa atgattaagt attgtcttct gagaacgctg |
| 3901 | aagcaatgtc agacattgag ggaagctcta attgctgcag gaaaagagat catatggcac |
| 3961 | gggcggacaa aagaagaacc agctcattat tgtagtattt gtgaggtgga ggtttttgat |
| 4021 | ctgctttttg tcactaatga gagtaattct cgaaaaacct acatagtaca ttgccaagat |
| 4081 | tgtgcacgaa aaacaagtgg gaatctggaa aattttgtgg tgctagaaca gtacaaaatg |
| 4141 | gaggatctga tgcaagtcta tgaccaattt acattagtaa gtgaaatcaa catgctcctc |
| 4201 | cattaccatc cgcctcatct tgatattgtt ccatggacat taaacatgag accttttctg |
| 4261 | ctattcagaa agtaa |
| Mouse KDM6A Amino Acid Sequence | |
| SEQ ID NO: 4 | |
| 1 | mkscgvslat aaaaaaaaaf gdeakkmaag kasgeseeas psltaeerea lggldsrlfg |
| 61 | fvrfhedgar mkallgkavr cyeslilkae gkvesdffcq lghfnllled ypkalsayqr |
| 121 | yyslqsdywk naaflyglgl vyfhynafqw aikafqsvly vdpsfcrake ihlrlglmfk |
| 181 | vntdyesslk hfqlalvdcn pctlsnaeiq fhiahlyetq rkyhsakeay eqllqtenls |
| 241 | aqvkatilqq lgwmbhtvdl lgdkatkesy aiqylqksle adpnsgqswy flgrcyssig |
| 301 | kvqdafisyr qsidkaeasa dtwcsigvly qqqnqpmdal qayicavqld hghaaawmdl |
| 361 | gtlyescnqp qdaikcylna trskncsnts glaarikylq aqlcnlpqgs lqnktkllps |
| 421 | ieeawslpip aeltsrqgam ntaqqntsdn wsggnapppv eqqthswclt pqklqhleql |
| 481 | ranrnnlnpa qklmleqles qfvlmqqhqm cqtgvaqvrp tgilngptvd sslptnsvsg |
| 541 | qqpqlpltrm psvsqpgvht acprqtlang pfsaghvpcs tsrtlgstdt vlignnhvtg |
| 601 | sgsngnvpyl qrnaptlphn rtnltsstee pwknqlsnst qglhkgpssh lagpngerpl |
| 661 | sstgpaqhlq aagsgiqnqn ghptlpsnsv tqgaalnhls shtatsggqq gitltkaskp |
| 721 | sgntltvpet srqtgetpns tasveglpnh vhqvmadavc spshgdsksp gllssdnpql |
| 781 | sallmgkann nvgpgtcdkv nnihptvhtk tdnsvassps saistatpsp ksteqtttns |
| 841 | vtslnsphsg lhtingegme esqspiktdl llvshrpspq iipsmsvsiy pssaevlkac |
| 901 | rnlgknglsn ssilldkcpp prppsspypp lpkdklnppt psiylenkrd affpplhqfc |
| 961 | tnpnnpvtvi rglagalkld lglfstktlv eannehmvev rtqllqpade nwdptgtkki |
| 1021 | whcesnrsht tiakyaqyqa ssfqeslree nektshhkdh sdsestssdn sgkrrkgpfk |
| 1081 | tikfgtnidl sddkkwklql heltklpafv rvvsagnlls hvghtilgmn tvqlymkvpg |
| 1141 | srtpghqenn nfcsvninig pgdcewfvvp egywgvlndf ceknnlnflm gswwpnledl |
| 1201 | yeanvpvyrf iqrpgdlvwi nagtvhwvqa igwcnniawn vgpltacqyk laveryswnk |
| 1261 | lqnvksivpm vhlswnmarn ikvsdpklfe mikycllrtl kqcqtlreal iaagkeiiwh |
| 1321 | grtkeepahy cslcevevfd llfvtnesns rktyivhcqd carktagnle nfvvleqykm |
| 1381 | edlmqvydqf tlvseinmll hyhpphldiv pwtlnmtpfl lfrk |
| Human KDM6B cDNA Sequence | |
| SEQ ID NO: 5 | |
| 1 | atgcatcggg cagtggaccc tccaggggcc cgcgctgcac gggaagcett tgcccttggg |
| 61 | ggcctgagct gtgctggggc ctggagctcc tgcccgcctc atccccctcc tcgtagcgca |
| 121 | tggctgcctg gaggcagatg ctcagccagc attgggcagc ccccgcttcc tgctccccta |
| 181 | cccccttcac atggcagtag ttctgggcac cccagcaaac catattatgc tccaggggcg |
| 241 | cccactccaa gacccctcca tgggaagctg gaatccctgc atggctgtgt gcaggcattg |
| 301 | ctccgggagc cagcccagcc agggctttgg gaacagcttg ggcaactgta cgagtcagag |
| 361 | cacgatagtg aggaggccac acgctgctac cacagcgccc ttcgatacgg aggaagcttc |
| 421 | gctgagctgg ggccccgcat tggccgactg cagcaggccc agctctggaa ctttcatact |
| 481 | ggctcctgcc agcaccgagc caaggtcctg cccccactgg agcaagtgtg gaacttgcta |
| 541 | caccttgagc acaaacggaa ctatggagcc aagcggggag gtcccccggt gaagcgagct |
| 601 | gctgaacccc cagtggtgca gcctgtgccc cctgcagcac tctcaggccc ctcaggggag |
| 661 | gagggcctca gccctggagg caagcgaagg agaggctgca actctgaaca gactggcctt |
| 721 | cccccagggc tgccactgcc tccaccacca ttaccaccac caccaccacc accaccacca |
| 781 | ccaccaccac ccctgcctgg cctggctacc agccccccat ttcagctaac caagccaggg |
| 841 | ctgcggagta ccctgcatgg agatgcctgg ggcccagagc gcaagggttc agcaccccca |
| 901 | gagcgccagg agcagcggca ctcgctgcct cacccatatc catacccagc tccagcgtac |
| 961 | accgcgcacc cccctggcca ccggctggtc ccggctgctc ccccaggccc aggcccccgc |
| 1021 | cccccaggag cagagagcca tggctgcctg cctgccaccc gtccccccgg aagtgacctt |
| 1081 | agagagagca gagttcagag gtcgcggatg gactccagcg tttcaccagc agcaaccacc |
| 1141 | gcctgcgtgc cttacgcccc ttcccggccc cctggcctcc ccggcaccac caccagcagc |
| 1201 | agcagtagca gcagcagcaa cactggtctc cggggcgtgg agccgaaccc aggcattccc |
| 1261 | ggcgctgacc attaccaaac tcccgcgctg gaggtctctc accatggccg cctggggccc |
| 1321 | tcggcacaca gcagtcggaa accgttcttg ggggctcccg ctgccactcc ccacctatcc |
| 1381 | ctgccacctg gaccttcctc accccctcca cccccctgtc cccgcctctt acgcccccca |
| 1441 | ccaccccctg cctggttgaa gggtccggcc tgccgggcag cccgagagga tggagagatc |
| 1501 | ttagaagagc tcttctttgg gactgaggga cccccccgcc ctgccccacc acccctcccc |
| 1561 | catcgcgagg gcttcttggg gcctccggcc tcccgctttt ctgtgggcac tcaggattct |
| 1621 | cacacccctc ccactccccc aaccccaacc accagcagta gcaacagcaa cagtggcagc |
| 1681 | cacagcagca gccctgctgg gcctgtgtcc tttcccccac caccctatct ggccagaagt |
| 1741 | atagaccccc ttccccggcc tcccagccca gcacagaacc cccaggaccc acctcttgta |
| 1801 | cccctgactc ttgccctgcc tccagcccct ccttcctcct gccaccaaaa tacctcagga |
| 1861 | agcttcaggc gcccggagag cccccggccc agggtctcct tcccaaagac ccccgaggtg |
| 1921 | gggccggggc cacccccagg ccccctgagt aaagcccccc agcctgtgcc gcccggggtt |
| 1981 | ggggagctgc ctgcccgagg ccctcgactc tttgattttc cccccactcc gctggaggac |
| 2041 | cagtttgagg agccagccga attcaagatc ctacctgatg ggctggccaa catcatgaag |
| 2101 | atgctggacg aatccattcg caaggaagag gaacagcaac aacacgaagc aggcgtggcc |
| 2161 | ccccaacccc cgctgaagga gccctttgca tctctgcagt ctcctttccc caccgacaca |
| 2221 | gcccccacca ctactgctcc tgctgtcgcc gtcaccacca ccaccaccac caccaccacc |
| 2281 | accacggcca cccaggaaga ggagaagaag ccaccaccag ccctaccacc accaccgcct |
| 2341 | ctagccaagt tccctccacc ctctcagcca cagccaccac cacccccacc ccccagcccg |
| 2401 | gccagcctgc tcaaatcctt ggcctccgtg ctggagggac aaaagtactg ttatcggggg |
| 2461 | actggagcag ctgtttccac ccggcctggg cccttgccca ccactcagta ttcccctggc |
| 2521 | cccccatcag gtgctaccgc cctgccgccc acctcagcgg cccctagcgc ccagggctcc |
| 2581 | ccacagccct ctgcttcctc gtcatctcag ttctctacct caggcgggcc ctgggcccgg |
| 2641 | gagcgcaggg cgggcgaaga gccagtcccg ggccccatga cccccaccca accgccccca |
| 2701 | cccctatctc tgccccctgc tcgctctgag tctgaggtgc tagaagagat cagccgggct |
| 2761 | tgcgagaccc ttgtggagcg ggtgggccgg agtgccactg acccagccga cccagtggac |
| 2821 | acagcagagc cagcggacag tgggactgag cgactgctgc cccccgcaca ggccaaggag |
| 2881 | gaggctggcg gggtggcggc agtgtcaggc agctgtaagc ggcgacagaa ggagcatcag |
| 2941 | aaggagcatc ggcggcacag gcgggcctgt aaggacagtg tgggtcgtcg gccccgtgag |
| 3001 | ggcagggcaa aggccaaggc caaggtcccc aaagaaaaga gccgccgggt gctggggaac |
| 3061 | ctggacctgc agagcgagga gatccagggt cgtgagaagt cccggcccga tcttggcggg |
| 3121 | gcctccaagg ccaagccacc cacagctcca gcccctccat cagctcctgc accttctgcc |
| 3181 | cagcccacac ccccgtcagc ctctgtccct ggaaagaagg ctcgggagga agccccaggg |
| 3241 | ccaccgggtg tcagccgggc cgacatgctg aagctgcgct cacttagtga ggggcccccc |
| 3301 | aaggagctga agatccggct catcaaggta gagagtggtg acaaggagac ctttatcgcc |
| 3361 | tctgaggtgg aagagcggcg gctgcgcatg gcagacctca ccatcagcca ctgtgctgct |
| 3421 | gacgtcgtgc gcgccagcag gaatgccaag gtgaaaggga agtttcgaga gtcctacctt |
| 3481 | tcccctgccc agtctgtgaa accgaagatc aacactgagg agaagctgcc ccgggaaaaa |
| 3541 | ctcaaccccc ctacacccag catctatctg gagagcaaac gggatgcctt ctcacctgtc |
| 3601 | ctgctgcagt tctgtacaga ccctcgaaat cccatcacag tgatccgggg cctggcgggc |
| 3661 | tccctgcggc tcaacttggg cctcttctcc accaagaccc tggtggaagc gagtggcgaa |
| 3721 | cacaccgtgg aagttcgcac ccaggtgcag cagccctcag atgagaactg ggatctgaca |
| 3781 | ggcactcggc agatctggcc ttgtgagagc tcccgttccc acaccaccat tgccaagtac |
| 3841 | gcacagtacc aggcctcatc cttccaggag tctctgcagg aggagaagga gagtgaggat |
| 3901 | gaggagtcag aggagccaga cagcaccact ggaacccctc ctagcagcgc accagacccg |
| 3961 | aagaaccatc acatcatcaa gtttggcacc aacatcgact tgtctgatgc taagcggtgg |
| 4021 | aagccccagc tgcaggagct gctgaagctg cccgccttca tgcgggtaac atccacgggc |
| 4081 | aacatgctga gccacgtggg ccacaccatc ctgggcatga acacggtgca gctgtacatg |
| 4141 | aaggtgcccg gcagccgaac gccaggccac caggagaata acaacttctg ctccgtcaac |
| 4201 | atcaacactg gcccaggcga ctgcgagtgg ttcgcggtgc acgagcacta ctgggagacc |
| 4261 | atcagcgctt tctgtgatcg gcacggcgtg gactacttga cgggttcctg gtggccaatc |
| 4321 | ctggatgatc tctatgcatc caatattcct gtgtaccgct tcgtgcagcg acccggagac |
| 4381 | ctcgtgtgga ttaatgcggg gactgtqcac tgggtgcagq ccaccggctg gtgcaacaac |
| 4441 | attgcctgga acgtggggcc cctcaccgcc tatcagtacc agctggccct ggaacgatac |
| 4501 | gagtggaatg aggtgaagaa cgtcaaatcc atcgtgccca tgattcacgt gtcatggaac |
| 4561 | gtggctcgca cggtcaaaat cagcgacccc gacttgttca agatgatcaa gttctgcctg |
| 4621 | ctgcagtcca tgaagcactg ccaggtgcaa cgcgagagcc tggtgcgggc agggaagaaa |
| 4681 | atcgcttacc agggccgtgt caaggacgag ccagcctact actgcaacga gtgcgatgtg |
| 4741 | gaggcgttta acatcctgtt cgtgacaagt gagaatggca gccgcaacac gtacctggta |
| 4801 | cactgcgagg gctgtgcccg gcgccgcagc gcaggcctgc agggcgtggt ggtgctggag |
| 4861 | cagtaccgca ctgaggagct ggctcaggcc tacgacgcct tcacgctggt gagggcccgg |
| 4921 | cgggcgcgcg ggcagcggag gagggcactg gggcaggctg cagggacggg cttcgggagc |
| 4981 | ccggccgcgc ctttccctga gcccccgccg gctttctccc cccaggcccc agccagcacg |
| 5041 | tcgcgatga |
| Human KDM6B Amino Acid Sequence | |
| SEQ ID NO: 6 | |
| 1 | mhravdppga raareafalg glscagawss qpphpppraa wlpggrcsas igqpplpapl |
| 61 | ppshgsssgh pskpyyapga ptprplhgkl eslhgcvqal lrepaqpglw eqlgqlyese |
| 121 | hdseeatrcy hsalryggsf aelgprigrl qqaqlwnfbt gscqhrakvl ppleqvwnll |
| 181 | hlehkrnyga krggppvkra aeppvvqpvp paalsgpsge eglspggkrr rgcnseqtgl |
| 241 | ppglplpppp lppppppppp pppplpglat sppfqltkpg lwstlhgdaw gperkgsapp |
| 301 | erqeqrhslp hpypypapay tahppghrlv paappgpgpr ppgaeshgcl patrppgsdl |
| 361 | resrvqrsrm dssvspaatt acvpyapsrp pglpgtttss ssssssntgl rgvepnpgip |
| 421 | gadhyqtpal evshhgrlgp sahssrkpfl gapaatphls lppgpssppp ppcprllrpp |
| 481 | pppawlkgpa craaredgei leelffgteg pprpappplp hregflgppa srfsvgtqds |
| 541 | htpptpptpt tsssnsnsgs hssspagpvs fppppylars idplprppsp aqnpqdpplv |
| 601 | pltlalppap psschqntsg sfrrpesprp rvsfpktpev gpgpppgpls kapqpvppgv |
| 661 | gelpargprl fdfpptpled qfeepaefki lpdglanimk mldesirkee eqqqheagva |
| 721 | pqpplkepfa slqspfptdt aptttapava vttttttttt ttatqeeekk pppalppppp |
| 781 | lakfpppsqp qpppppppsp asllkslasv legqkycyrg tgaavstrpg plpttqyspg |
| 841 | ppsgatalpp tsaapsaqgs pqpsassssq fstsggpwar errageepvp gpmtptqppp |
| 901 | plslpparse sevleeisra cetlvervgr satdpadpvd taepadsgte rllppaqake |
| 961 | eaggvaavsg sckrrqkehq kehrrhrrac kdsvgrrpre grakakakvp keksrrvlgn |
| 1021 | ldlqseeiqg reksrpdlgg askakpptap appsapapsa qptppsasvp gkkareeapg |
| 1081 | ppgvsradml klrslsegpp kelkirlikv esgdketfia seveerrlrm adltishcaa |
| 1141 | dvvrasrnak vkgkfresyl spaqsvkpki nteeklprek lnpptpsiyl eskrdafspv |
| 1201 | llqfctdprn pitvirglag slrlnlglfs tktlveasge htvevrtqvq qpsdenwdlt |
| 1261 | gtrqiwpces srshttiaky aqyqassfqe slqeekesed eeseepdstt gtppssapdp |
| 1321 | knhhiikfgt nidlsdakrw kpqlqellkl pafmrvtstg nmlshvghti lgmntvqlym |
| 1381 | kvpgsrtpgh qennnfcsvn inigpgdcew favhehywet isafcdrhgv dyltgswwpi |
| 1441 | lddlyasnip vyrfvqrpgd lvwinagtvh wvqatgwcnn iawnvgplta yqyqlalery |
| 1501 | ewnevknvks ivpmihvawn vartvkisdp dlfkmikfcl lqsmkhcqvq reslvragkk |
| 1561 | iayqgrvkde payycnecdv avfnilfvts engarntylv hcegcarrrs aglqgvvvle |
| 1621 | qyrteelaqa ydaftlvrar rargqrrral gqaagtgfgs paapfpeppp afspqapast |
| 1681 | sr |
| Mouse KDM6B cDNA Sequence | |
| SEQ ID NO: 7 | |
| 1 | atgcatcggg cagtggaccc tccaggggcc cgctctgcac gggaagcctt tgcccttggg |
| 61 | ggcttgagct gtgctggggc ttggagctcc tgcccacccc atcctcctcc ccgaagctca |
| 121 | tggctgcccg gaggcagatg ctctgccagc gttgggcagc ccccactctc agctccttta |
| 181 | cccccatctc atggcagtag ctccgggcac cctaacaaac cctattatgc tcctgggaca |
| 241 | cccaccccaa gaccccttca cgggaagttg gaatccctac atggctgtgt ccaggcattg |
| 301 | ctccgggagc cagcgcagcc agggttgtgg gaacagcttg gacagtcgta tgaatcagag |
| 361 | cacgacagtg aggaagccgt atgctgctac catagggccc ttcgctatgg aggaagcttc |
| 421 | gccgagctgg gaccccggat tggccgcttg cagcaggccc agctctggaa ctttcatgcc |
| 481 | ggttcctgtc agcacagagc caaggtcctg cctcccctgg agcaagtctg gaatttgctg |
| 541 | caccttgagc acaaacggaa ctatggggct aagcgagggg gccctccagt gaagagatct |
| 601 | gctgaacccc ccgtggtcca gcctatgcct cctgcagccc tctcaggccc ctcaggagag |
| 661 | gagggcctta gccctggagg caagcgcagg agaggctgca gctctgaaca ggctggcctt |
| 721 | cccccaggtc tgccactccc tccaccaccc ccacccccac cgcctccacc accaccacca |
| 781 | ccccctccac caccaccgct gcctggcctg gctattagcc ccccatttca gctgactaag |
| 841 | ccagggctgt ggaataccct gcatggagat gcttggggcc ccgagcgcaa gggttcagcg |
| 901 | ccgccagagc gccaggagca gcggcactcg atgcctcatt catatccata cccagctccc |
| 961 | gcctactccg ctcatccgcc cagccatcgg ctggtcccca acacacccct tggtccaggt |
| 1021 | ccccgacccc caggagcaga gagccatggc tgcctgcctg ccacccgtcc ccccggaagt |
| 1081 | gaccttagag agagcagagt tcagaggtcg cggatggact ccagcgtttc accagcagca |
| 1141 | tctaccgcct gcgtgcctta cgccccttcc cggccccctg gcctccccgg caccagcagc |
| 1201 | agcagcagca gcagcagtag cagtaacaac actggtcttc ggggtgtgga gccaagccca |
| 1261 | ggcattcctg gcgctgacca ttaccaaaac cctgcgctgg agatatcccc tcaccaggcc |
| 1321 | cgcctgggtc cctccgcaca cagcagtcgg aaaccattct tgacggcccc tgctgccacg |
| 1381 | ccccacttat ccctaccccc tgggacccca tcatcccctc cacccccatg tcctcgcctc |
| 1441 | ttgcgccctc caccgccccc tgcttggatg aagggctcag cctgccgtgc agcccgagag |
| 1501 | gatggagaga tcttagggga gctcttcttt ggtgctgagg gacctccccg tcctcctccc |
| 1561 | ccaccccttc cccaccgtga tggcttcttg gggcctccaa acccccgctt ttctgtgggc |
| 1621 | actcaggatt cgcataaccc tcccactccc ccaaccacca ccagcagcag cagcagcagc |
| 1681 | aacagccaca gcagtagtcc tactgggccg gtgccctttc caccaccctc ctatctggcc |
| 1741 | agaagtatag accccctccc caggccatcc agcccaacct tgagccccca ggacccacct |
| 1801 | cttccaccac tgactcttgc cctgcctcca gcccctccct cctcctgcca ccaaaatacc |
| 1861 | tcaggaagct tcaggcgctc ggagagcccc cggcccaggg tctccttccc aaagaccccc |
| 1921 | gaggtggggc aggggccacc cccaggccct gtgagtaaag ccccccagcc tgtgccacct |
| 1981 | ggggttggag agctgcctgc ccgaggcccg aggctctttg atttcccacc cactccgctg |
| 2041 | gaggaccagt ttgaagagcc agccgaattc aagatcctac ctgatgggct ggcaaacatc |
| 2101 | atgaagatgc tggatgaatc cattcggaag gaggaggagc agcagcagca gcaggaggca |
| 2161 | ggcgtggctc ccccaccccc actcaaagag ccctttgcat ctctacagcc tccatttccc |
| 2221 | agtgacacag ccccagccac caccactgct gcccccacca ccgccaccac caccacaacc |
| 2281 | accaccacca ccaccaccca agaagaggag aagaagccac caccagccct accaccacca |
| 2341 | ccgcctctag ccaagtttcc tccacctccc cagccacagc ccccaccacc tccaccagcc |
| 2401 | agcccagcca gcctgctcaa atcgttggcc tctgttcttg agggacaaaa gtactgttac |
| 2461 | cgggggactg gagcagccgt ctcaaccagg cccgggtccg tgcccgccac tcagtattcc |
| 2521 | cctagtcctg catcaggtgc taccgcccca ccacccactt cagtggcccc tagtgcccag |
| 2581 | ggctccccca agccctcggt ttcctcgtca tctcagttct ctacctcagg cgggccttgg |
| 2641 | gcccgggagc acagggcggg tgaagagcca gcaccaggcc ccgtgacccc tgcccagttg |
| 2701 | cccccacctc tgccgctgcc ccctgctcgt tctgagtctg aggtgctaga agaaatcagt |
| 2761 | cgggcttgtg agacccttgt agagcgggtg ggccggagtg ccatcaaccc agtggacacg |
| 2821 | gcagacccag tggacagtgg gactgagcca cagccgccgc ctgcgcaggc caaggaggag |
| 2881 | agtggggggg tggcggtagc agcagcaggt ccaggtagtg gcaagcgtcg tcaaaaggag |
| 2941 | catcggcggc acaggcgggc ctgtagggac agtgtgggtc gacgaccccg cgaggggagg |
| 3001 | gccaaggcca aggccaaggc tcccaaagaa aaaagccgaa gggtgctggg gaacctcgac |
| 3061 | ttgcagagtg aggagatcca gggccgggag aaggcccggc ccgatgtcgg tggggtttcc |
| 3121 | aaagtcaaga cacccacagc tccagcaccc ccgcctgctc ctgcacccgc tgctcagcca |
| 3181 | acacccccat cagctcctgt ccctgggaag aagactcgtg aggaggctcc ggggcctcca |
| 3241 | ggtgtgagcc gggcagatat gctgaagctc cggtcactta gtgaggggcc tcccaaggag |
| 3301 | ctgaagatca ggctcatcaa ggtggaaagt ggggacaagg agacctttat cgcctctgag |
| 3361 | gtggaagagc ggcggctgcg catggcagac ctcaccatca gccactgtgc cgccgatgtc |
| 3421 | atgcgtgcca gcaagaatgc caaggtgaaa gggaaattcc gagagtccta cctgtcccct |
| 3481 | gcccagtctg tgaaacccaa gatcaacact gaggagaagc tgccccggga aaaactcaac |
| 3541 | ccccctaccc ccagcatcta tttggagagc aaacgagatg ccttctcgcc ggtcctgcta |
| 3601 | cagttctgta cagacccccg gaaccccatc accgtcatca ggggcctggc tggttcactt |
| 3661 | cggctcaact taggcctttt ctccaccaag actctggtgg aggcgagcgg tgaacatacg |
| 3721 | gtggaggtcc gtacccaagt acagcagccc tcagacgaga actgggacct gacaggtacc |
| 3781 | agacaaatct ggccctgtga gagctcccgt tcccacacca ccatcgctaa atacgcacag |
| 3841 | taccaggcct cgtccttcca ggagtcactg caggaggaga gggagagtga ggatgaggaa |
| 3901 | tccgaggaac cagacagcac tacaggaacc tctcccagca gtgcaccgga ccccaagaac |
| 3961 | catcacatca tcaagtttgg cactaacatc gacctgtctg atgccaagag gtggaagcca |
| 4021 | cagctacagg agctgctgaa actgcccgcc ttcatgcggg taacatccac aggcaacatg |
| 4081 | ctcagccacg tgggccacac catcctgggc atgaacaccg tgcagctata catgaaggtc |
| 4141 | cctggcagcc gaacgccagg ccaccaagag aataacaatt tctgctcagt caacatcaac |
| 4201 | attggccctg gggactgcga gtggttcgcg gtacatgagc actattggga gaccatcagc |
| 4261 | gccttctgcg accggcatgg tgtggactac ttgactggtt cctggtggcc aatcttggat |
| 4321 | gacctctatg cgtccaatat tcctgtttac cgcttcgtgc agcgccctgg agaccttgtg |
| 4381 | tggattaatg cagggactgt acattgggtg caggctaccg gctggtgcaa caacattgcc |
| 4441 | tggaacgtgg ggcccctcac cgcctatcag taccagctgg ccctggagcg atatgagtgg |
| 4501 | aacgaggtga agaacgtcaa gtccattgtg cccatgattc atgtgtcctg gaacgtcgct |
| 4561 | cgaacggtca agatcagcga tcctgacttg ttcaagatga tcaagttctg cctcctgcag |
| 4621 | tcaatgaagc actgtcaggt acagcgggag agcctggtgc gggcagggaa gaagatcgct |
| 4681 | taccaaggcc gtgtcaaaga cgagcctgcc tactactgca acgaatgcga cgtggaggtg |
| 4741 | ttcaacatcc tgttcgttac aagtgagaat ggcagccgaa acacgtacct ggtgcactgc |
| 4801 | gagggctgtg cgcgccgtcg cagcgcgggc ctacagggcg tggtggtgct agagcagtac |
| 4861 | cgcacggagg agctggcgca ggcctacgat gccttcacac tggctcccgc cagcacgtct |
| 4921 | cgatga |
| Mouse KDM6B Amino Acid Sequence | |
| SEQ ID NO: 8 | |
| 1 | mhravdppga rsareafalg glscagawss cpphppprss wlpggrcsas vgqpplsapl |
| 61 | ppshgsssgh pnkpyyapgt ptprplhgkl eslhgcvqal lrepaqpglw eqlgqlyese |
| 121 | hdseeavccy hralryggsf aelgprigrl qqaqlwnfha gscqhrakvl ppleqvwnll |
| 181 | hlehkrnyga krggppvkrs aeppvvqpmp paalsgpsge eglspggkrr rgcsseqagl |
| 241 | ppglplpppp pppppppppp pppppplpgl aisppfqltk pglwntlhgd awgpcrkgsa |
| 301 | pperqeqrhs mphsypypap aysahppshr lvpntplgpg prppgaeshg clpatrppgs |
| 361 | dlresrvqrs rmdssvspaa stacvpyaps rppglpgtss ssssssssnn tglrgvepsp |
| 421 | gipgadhyqn paleisphqa rlgpsahssr kpfltapaat phlslppgtp ssppppcprl |
| 481 | lrpppppawm kgsacraare dgeilgelff gaegpprppp pplphrdgfl gppnprfsvg |
| 541 | tqdshnppip ptttssssss nshsssptgp vpfpppsyla rsidplprps sptlspqdpp |
| 601 | lppltlalpp appsschqnt sgsfrrsesp rprvsfpktp evgqgpppgp vskapqpvpp |
| 661 | gvgelpargp rlfdfpptpl edqfeepaef kilpdglani mkmldesirk ceeqqqqqea |
| 721 | gvapppplke pfaslqppfp sdtapattta apttattttt ttttttqeee kkpppalppp |
| 781 | pplakfpppp qpqppppppa spasllksla svlegqkycy rgtgaavstr pgsvpatqys |
| 841 | pspasgatap pptsvapsaq gspkpsvsss sqfstsggpw arehrageep apgpvcpaql |
| 901 | ppplplppar sesevleeis racetlverv grsainpvdt adpvdsgtep qpppaqakee |
| 961 | sggvavaaag pgsgkrrqke hrrhrracrd svgrrpregr akakakapke ksrrvlgnld |
| 1021 | lqseeiqgre karpdvggvs kvktptapap ppapapaaqp tppsapvpgk ktreeapgpp |
| 1081 | gvsradmlkl rslsegppke lkirlikves gdketfiase veerrlrmad ltishcaadv |
| 1141 | mrasknakvk gkfresylsp aqsvkpkint eeklprekln pptpsiyles krdafspvll |
| 1201 | qfctdprnpi tvirglagsl rlnlglfstk tlveasgeht vevrtqvqqp sdenwdltgt |
| 1261 | rqiwpcessr shttiakyaq yqassfqesl qeeresedee seepdsttgt spssapdpkn |
| 1321 | hhiikfgtni dlsdakrwkp qlqellklpa fmrvtstgnm lshvghtilg mntvqlymkv |
| 1381 | pgsrtpghqe nnnfcsvnin igpgdcewfa vhehywetis afcdrhgvdy ltgswwpild |
| 1441 | dlyasnipvy rfvqrpgdlv wlnagtvhwv qatgwcnnia wnvgpltayq yqlaleryew |
| 1501 | nevknvksiv pmihvswnva rtvkisdpdl fkmikfcllq smkhcqvqre slvragkkia |
| 1561 | yqgrvkdepa yycnecdvev fnilfvtsen gsrntylvhc egcarrrsag lqgvvvleqy |
| 1621 | rteelaqayd aftlapasts r |
| Human EZH2 (isoform 1) cDNA Sequence | |
| SEQ ID NO: 9 | |
| 1 | atgggccaga ctgggaagaa atctgagaag ggaccagttt gttggcggaa gcgtgtaaaa |
| 61 | tcagagtaca tgcgactgag acagctcaag aggttcagac gagctgatga agtaaagagt |
| 121 | atgtttagtt ccaatcgtca gaaaattttg gaaagaacgg aaatcttaaa ccaagaatgg |
| 181 | aaacagcgaa ggatacagcc tgtgcacatc ctgacttctg tgagctcatt gcgcgggact |
| 241 | agggagtgtt cggtgaccag tgacttggat tttccaacac aagtcacccc attaaagact |
| 301 | ctgaatgcag ttgcttcagt acccataatg tattcttggt ctcccctaca gcagaatttt |
| 361 | atggtggaag atgaaactgt tttacataac attccttata tgggagatga agttttagat |
| 421 | caggatggta ctttcattga agaactaata aaaaattatg atgggaaagt acacggggat |
| 481 | agagaatgtg ggtttataaa tgatgaaatt tttgtggagt tggtgaatgc ccttggtcaa |
| 541 | tataatgatg atgacgatga tgatgatgga gacgatcctg aagaaagaga agaaaagcag |
| 601 | aaagatctgg aggatcaccg agatgataaa gaaagccgcc cacctcggaa atttccttct |
| 661 | gataaaattt ttgaagccat ttcctcaatg tttccagata agggcacagc agaagaacta |
| 721 | aaggaaaaat ataaagaact caccgaacag cagctcccag gcgcacttcc tcctgaatgt |
| 781 | acccccaaca tagatggacc aaatgctaaa tctgttcaga gagagcaaag cttacactcc |
| 841 | tttcatacgc tttcctgtag gcgatgtttt aaatatgact gcttcctaca tcgtaagtgc |
| 901 | aattattctt ttcatgcaac acccaacact tataagcgga agaacacaga aacagctcta |
| 961 | gacaacaaac cttgtggacc acagtgttac cagcatttgg agggagcaaa ggagtttgct |
| 1021 | gctgctctca ccgctgagcg gataaagacc ccaccaaaac gtccaggagg ccgcagaaga |
| 1081 | ggacggcttc ccaataacag tagcaggccc agcaccccca ccatcaatgt gctggaatca |
| 1141 | aaggatacag acagtgatag ggaagcaggg actgaaacgg ggggagagaa caatgataaa |
| 1201 | gaagaagaag agaagaaaga tgaaacttcg agctcctctg aagcaaattc tcggtgtcaa |
| 1261 | acaccaataa agatgaagcc aaatattgaa cctcctgaga atgtggagtg gagtggtgct |
| 1321 | gaagcctcaa tgtttagagt cctcattggc acttactatg acaatttctg tgccattgct |
| 1381 | aggttaattg ggaccaaaac atgtagacag gtgtatgagt ttagagtcaa agaatctagc |
| 1441 | atcatagctc cagctcccgc tgaggatgtg gatactcctc caaggaaaaa gaagaggaaa |
| 1501 | caccggttgt gggctgcaca ctgcagaaag atacagctga aaaaggacgg ctcctctaac |
| 1561 | catgtttaca actatcaacc ctgtgatcat ccacggcagc cttgtgacag ttcgtgccct |
| 1621 | tgtgtgatag cacaaaattt ttgtgaaaag ttttgtcaat gtagttcaga gtgtcaaaac |
| 1681 | cgctttccgg gatgccgctg caaagcacag tgcaacacca agcagtgccc gtgctacctg |
| 1741 | gctgtccgag agtgtgaccc tgacctctgt cttacttgtg gagccgctga ccattgggac |
| 1801 | agtaaaaatg tgtcctgcaa gaactgcagt attcagcggg gctccaaaaa gcatctattg |
| 1861 | ctggcaccat ctgacgtggc aggctggggg atttttatca aagatcctgt gcagaaaaat |
| 1921 | gaattcacct cagaatactg tggagagatt atttctcaag atgaagctga cagaagaggg |
| 1981 | aaagtgtatg ataaatacat gtgcagcttt ctgttcaact tgaacaatga ttttgtggtg |
| 2041 | gatgcaaccc gcaagggtaa caaaattcgt tttgcaaatc attcggtaaa tccaaactgc |
| 2101 | tatgcaaaag ttatgatggt taacggtgat cacaggatag gtatttttgc caagagagcc |
| 2161 | atccagactg gcgaagagct gttttttgat tacagataca gccaggctga tgccctgaag |
| 2221 | tatgtcggca tcgaaagaga aatggaaatc ccttga |
| Human EZH2 (isoform 1) Amino Acid Sequence | |
| SEQ ID NO: 10 | |
| 1 | mgqtgkksek gpvcwrkrvk seymrlrqlk rfrradevks mfssnrqkil erteilnqew |
| 61 | kqrriqpvhi ltsvsslrgt recsvtsdld fptqviplkt lnavasvpim yswsplqqnf |
| 121 | mvedetvlhn ipymgdevld qdgtfieeli knydgkvhgd recgfindei fvelvnalgq |
| 181 | yndddddddg ddpeereakq kdledhrddk esrpprkfps dkifeaissm fpdkgtaeel |
| 241 | kekykelteq qlpgalppec tpnidgpnak svqreqslhs fhtlfcrrcf kydcflhrkc |
| 301 | nysfhatpnt ykrkntetal dnkpcgpqcy qhlegakefa aaltaerikt ppkrpggrrr |
| 361 | grlpnnssrp stptinvles kdtdsdreag tetggenndk eeeekkdets ssseansrcq |
| 421 | tpikmkpnie ppenvewsga easmfrvlig tyydnfcaia rligtktcrq vyefrvkess |
| 481 | iiapapaedv dtpprkkkrk hrlwaahcrk iqlkkdgssn hvynyqpcdh prqpcdsscp |
| 541 | cviaqnfcek fcqcssecqn rfpgcrckaq cntkqcpcyl avrecdpdlc ltcgaadhwd |
| 601 | sknvsckncs iqrgskkhll lapsdvagwg ifikdpvqkn efiseyegei isqdeadrrg |
| 661 | kvydkymcsf lfnlnndfvv datrkgnkir fanhsvnpnc yakvmmvngd hrigifakra |
| 721 | iqtgeelffd yrysqadalk yvgieremei p |
| Human EZH2 (isoform 2) cDNA Sequence | |
| SEQ ID NO: 11 | |
| 1 | atgggccaga ctgggaagaa atctgagaag ggaccagttt gttggcggaa gcgtgtaaaa |
| 61 | tcagagtaca tgcgactgag acagctcaag aggttcagac gagctgatga agtaaagagt |
| 121 | atgtttagtt ccaatcgtca gaaaattttg gaaagaacgg aaatcttaaa ccaagaatgg |
| 181 | aaacagcgaa ggatacagcc tgtgcacatc ctgacttctg tgagctcatt gcgcgggact |
| 241 | agggaggtgg aagatgaaac tgttttacat aacattcctt atatgggaga tgaagtttta |
| 301 | gatcaggatg gtactttcat tgaagaacta ataaaaaatt atgatgggaa agtacacggg |
| 361 | gatagagaat gtgggtttat aaatgatgaa atttttgtgg agttggtgaa tgcccttggt |
| 421 | caatataatg atgatgacga tgatgatgat ggagacgatc ctgaagaaag agaagaaaag |
| 481 | cagaaagatc tggaggatca ccgagatgat aaagaaagcc gcccacctcg gaaatttcct |
| 541 | tctgataaaa tttttgaagc catttcctca atgtttccag ataagggcac agcagaagaa |
| 601 | ctaaaggaaa aatataaaga actcaccgaa cagcagctcc caggcgcact tcctcctgaa |
| 661 | tgtaccccca acatagatgg accaaatgct aaatctgttc agagagagca aagcttacac |
| 721 | tcctttcata cgcttttctg taggcgatgt tttaaatatg actgcttcct acatcctttt |
| 781 | catgcaacac ccaacactta taagcggaag aacacagaaa cagctctaga caacaaacct |
| 841 | tgtggaccac agtgttacca gcatttggag ggagcaaagg agtttgctgc tgctctcacc |
| 901 | gctgagcgga taaagacccc accaaaacgt ccaggaggcc gcagaagagg acggcttccc |
| 961 | aataacagta gcaggcccag cacccccacc attaatgtgc tggaatcaaa ggatacagac |
| 1021 | agtgataggg aagcagggac tgaaacgggg ggagagaaca atgataaaga agaagaagag |
| 1081 | aagaaagatg aaactccgag ctcctctgaa gcaaattctc ggtgtcaaac accaataaag |
| 1141 | atgaagccaa atattgaacc tcctgagaat gtggagtgga gtggtgctga agcctcaatg |
| 1201 | tttagagtcc tcattggcac ttactatgac aatttctgtg ccattgctag gttaattggg |
| 1261 | accaaaacat gtagacaggt gtatgagttt agagtcaaag aatctagcat catagctcca |
| 1321 | gctcccgctg aggatgtgga tactcctcca aggaaaaaga agaggaaaca ccggttgtgg |
| 1381 | gctgcacact gcagaaagat acagctgaaa aaggacggct cctctaacca tgtttacaac |
| 1441 | tatcaaccct gtgatcatcc acggcagcct tgtgacagtt cgtgcccttg tgtgatagca |
| 1501 | caaaattttt gtgaaaagtt ttgtcaatgt agttcagagt gtcaaaaccg ctctccggga |
| 1561 | tgccgctgca aagcacagtg caacaccaag cagtgcccgt gctacctggc tgtccgagag |
| 1621 | tgtgaccctg acctctgtct tacttgtgga gccgctgacc attgggacag taaaaatgtg |
| 1681 | tcctgcaaga actgcagtat tcagcggggc tccaaaaagc atctattgct ggcaccatct |
| 1741 | gacgtggcag gctgggggat ttttatcaaa gatcctgtgc agaaaaatga attcatctca |
| 1801 | gaatactgtg gagagattat ttctcaagat gaagctgaca gaagagggaa agtgtatgat |
| 1861 | aaatacatgt gcagctttct gttcaacttg aacaatgatt ttgtggtgga tgcaacccgc |
| 1921 | aagggtaaca aaattcgttt tgcaaatcat tcggtaaatc caaactgcta tgcaaaagtt |
| 1981 | atgatggtta acggtgatca caggataggt atttttgcca agagagccat ccagactggc |
| 2041 | gaagagctgt tttttgatta cagatacagc caggctgatg ccctgaagta tgtcggcatc |
| 2101 | gaaagagaaa tggaaatccc ttga |
| Human EZH2 (isoform 3) cDNA Sequence | |
| SEQ ID NO: 12 | |
| 1 | atgggccaga ctgggaagaa atctgagaag ggaccagttt gttggcggaa gcgtgtaaaa |
| 61 | tcagagtaca tgcgactgag acagctcaag aggttcagac gagctgatga agtaaagagt |
| 121 | atgtttagtt ccaatcgtca gaaaattttg gaaagaacgg aaatcttaaa ccaagaatgg |
| 181 | aaacagcgaa ggatacagcc tgtgcacatc ctgacttctg tgagctcatt gcgcgggact |
| 241 | agggagtgtt cggtgaccag tgacttggat tttccaacac aagtcatccc attaaagact |
| 301 | ctgaatgcag ttgcttcagt acccataatg tattcttggt ctcccctaca gcagaatttt |
| 361 | atggtggaag atgaaactgt tttacataac attccttata tgggagatga agttttagat |
| 421 | caggatggta ctttcattga agaactaata aaaaattatg atgggaaagt acacggggat |
| 481 | agagaatgtg ggtttataaa tgatgaaatt tttgtggagt tggtgaatgc ccttggtcaa |
| 541 | tataatgatg atgacgatga tgatgatgga gacgatcctg aagaaagaga agaaaagcag |
| 601 | aaagatctgg aggatcaccg agatgataaa gaaagccgcc cacctcggaa atttccttct |
| 661 | gataaaattt ttgaagccat ttcctcaatg tttccagata agggcacagc agaagaacta |
| 721 | aaggaaaaat ataaagaact caccgaacag cagctcccag gcgcacttcc tcctgaatgt |
| 781 | acccccaaca tagatggacc aaatgctaaa tctgttcaga gagagcaaag cttacactcc |
| 841 | tttcatacgc ttttctgtag gcgatgtttt aaatatgact gcttcctaca tccttttcat |
| 901 | gcaacaccca acacttataa gcggaagaac acagaaacag ctctagacaa caaaccttgt |
| 961 | ggaccacagt gttaccagca tttggaggga gcaaaggagt ttgctgctgc tctcaccgct |
| 1021 | gagcggataa agaccccacc aaaacgtcca ggaggccgca gaagaggacg gcttcccaat |
| 1081 | aacagtagca ggcccagcac ccccaccatt aatgtgctgg aatcaaagga tacagacagt |
| 1141 | gatagggaag cagggactga aacgggggga gagaacaatg ataaagaaga agaagagaag |
| 1201 | aaagatgaaa cttcgagctc ctctgaagca aattctcggt gtcaaacacc aataaagatg |
| 1261 | aagccaaata ttgaacctcc tgagaatgtg gagtggagtg gtgctgaagc ctcaatgttt |
| 1321 | agagtcctca ttggcactta ctatgacaat ttctgtgcca ttgctaggtt aattgggacc |
| 1381 | aaaacatgta gacaggtgta tgagtttaga gtcaaagaat ctagcatcat agctccagct |
| 1441 | cccgctgagg atgtggatac tcctccaagg aaaaagaaga ggaaacaccg gttgtgggct |
| 1501 | gcacactgca gaaagataca gctgaaaaag gacggctcct ctaaccatgt ttacaactat |
| 1561 | caaccctgtg atcatccacg gcagccttgt gacagttcgt gcccttgtgt gatagcacaa |
| 1621 | aatttttgtg aaaagttttg tcaatgtagt tcagagtgtc aaaaccgctt tccgggatgc |
| 1681 | cgctgcaaag cacagtgcaa caccaagcag tgcccgtgct acctggctgt ccgagagtgt |
| 1741 | gaccctgacc tctgtcttac ttgtggagcc gctgaccatt gggacagtaa aaatgtgtcc |
| 1801 | tgcaagaact gcagtattca gcggggctcc aaaaagcatc tattgctggc accatctgac |
| 1861 | gtggcaggct gggggatttt tatcaaagat cctgtgcaga aaaatgaatt catctcagaa |
| 1921 | tactgtggag agattatttc tcaagatgaa gctgacagaa gagggaaagt gtatgataaa |
| 1981 | tacatgtgca gctttctgtt caacttgaac aatgattttg tggtggatgc aacccgcaag |
| 2041 | ggtaacaaaa ttcgttttgc aaatcattcg gtaaatccaa actgctatgc aaaagctatg |
| 2101 | atggttaacg gtgatcacag gataggtatt tttgccaaga gagccaccca gactggcgaa |
| 2161 | gagctgtttt ttgattacag atacagccag gctgatgccc tgaagtatgt cggcatcgaa |
| 2221 | agagaaatgg aaatcccttg a |
| Human EZH2 (isoform 4) cDNA Sequence | |
| SEQ ID NO: 13 | |
| 1 | atgggccaga ctgggaagaa atctgagaag ggaccagttt gttggcggaa gcgtgtaaaa |
| 61 | tcagagtaca tgcgactgag acagctcaag aggttcagac gagctgatga agtaaagagt |
| 121 | atgtttagtt ccaatcgtca gaaaattttg gaaagaacgg aaatcttaaa ccaagaatgg |
| 181 | aaacagcgaa ggatacagcc tgtgcacatc ctgacttctt gttcggtgac cagtgacttg |
| 241 | gattttccaa cacaagtcat cccattaaag actctgaatg cagttgcttc agtacccata |
| 301 | atgtattctt ggtctcccct acagcagaat tttatggtgg aagatgaaac tgttttacat |
| 361 | aacattcctt atatgggaga tgaagtttta gatcaggatg gtactttcat tgaagaacta |
| 421 | ataaaaaatt atgatgggaa agtacacggg gatagagaat gtgggtttat aaatgatgaa |
| 481 | atttttgtgg agttggtgaa tgcccttggt caatataatg atgatgacga tgatgatgat |
| 541 | ggagacgatc ctgaagaaag agaagaaaag cagaaagatc tggaggatca ccgagatgat |
| 601 | aaagaaagcc gcccacctcg gaaatttcct tctgataaaa tttttgaagc catttcctca |
| 661 | atgtttccag ataagggcac agcagaagaa ctaaaggaaa aatataaaga actcaccgaa |
| 721 | cagcagctcc caggcgcact tcctcctgaa tgtaccccca acatagatgg accaaatgct |
| 781 | aaatctgttc agagagagca aagcttacac tcctttcata cgcttttctg taggcgatgt |
| 841 | tttaaatatg actgcttcct acatcctttt catgcaacac ccaacactta taagcggaag |
| 901 | aacacagaaa cagctctaga caacaaacct tgtggaccac agtgttacca gcatttggag |
| 961 | ggagcaaagg agtttgctgc tgctctcacc gctgagcgga taaagacccc accaaaacgt |
| 1021 | ccaggaggcc gcagaagagg acggcttccc aataacagta gcaggcccag cacccccacc |
| 1081 | attaatgtgc tggaatcaaa ggatacagac agtgataggg aagcagggac tgaaacgggg |
| 1141 | ggagagaaca atgataaaga agaagaagag aagaaagatg aaacttcgag ctcctctgaa |
| 1201 | gcaaattctc ggtgtcaaac accaataaag atgaagccaa atattgaacc tcctgagaat |
| 1261 | gtggagtgga gtggtgctga agcctcaatg tttagagtcc tcattggcac ttactatgac |
| 1321 | aatctctgtg ccattgctag gttaattggg accaaaacat gtagacaggt gtatgagttt |
| 1381 | agagtcaaag aatctagcat catagctcca gctcccgctg aggatgtgga tactcctcca |
| 1441 | aggaaaaaga agaggaaaca ccggttgtgg gctgcacact gcagaaagat acagctgaaa |
| 1501 | aaggacggct cctctaacca tgtttacaac tatcaaccct gtgatcatcc acggcagcct |
| 1561 | tgtgacagtt cgtgcccttg tgtgatagca caaaattttt gtgaaaagtt ttgtcaatgt |
| 1621 | agttcagagt gtcaaaaccg ctttccggga tgccgctgca aagcacagtg caacaccaag |
| 1681 | cagtgcccgt gctacctggc tgtccgagag tgtgaccctg acctctgtct tacttgtgga |
| 1741 | gccgctgacc attgggacag taaaaatgtg tcctgcaaga actgcagtat tcagcggggc |
| 1801 | tccaaaaagc atctattgct ggcaccatct gacgtggcag gctgggggat ttttatcaaa |
| 1861 | gatcctgtgc agaaaaatga attcatctca gaatactgtg gagagattat ttctcaagat |
| 1921 | gaagctgaca gaagagggaa agtgtatgat aaatacatgt gcagctttct gttcaacttg |
| 1981 | aacaatgatt ttgtggtgga tgcaacccgc aagggtaaca aaattcgttt tgcaaatcat |
| 2041 | tcggtaaatc caaactgcta tgcaaaagct atgatggtta acggtgatca caggataggt |
| 2101 | atttttgcca agagagccat ccagactggc gaagagctgt tttttgatta cagatacagc |
| 2161 | caggctgatg ccctgaagta tgtcggcatc gaaagagaaa tggaaatccc ttga |
| Human EZH2 (isoform 5) cDNA Sequence | |
| SEQ ID NO: 14 | |
| 1 | atgggccaga ctgggaagaa atctgagaag ggaccagttt gttggcggaa gcgtgtaaaa |
| 61 | tcagagtaca tgcgactgag acagctcaag aggttcagac gagctgatga agtaaagagt |
| 121 | atgtttagtt ccaatcgtca gaaaattttg gaaagaacgg aaatcttaaa ccaagaatgg |
| 181 | aaacagcgaa ggatacagcc tgtgcacatc ctgacttctt gttcggtgac cagtgacttg |
| 241 | gattttccaa cacaagtcat cccattaaag actctgaatg cagttgcttc agtacccata |
| 301 | atgtattctt ggtctcccct acagcagaat tttatggtgg aagatgaaac tgttttacat |
| 361 | aacattcctt atatgggaga tgaagtttta gatcaggatg gtactttcat tgaagaacta |
| 421 | ataaaaaatt atgatgggaa agtacacggg gatagagaat gtgggtttat aaatgatgaa |
| 481 | atttttgtgg agttggtgaa tgcccttggt caatataatg atgatgacga tgatgatgat |
| 541 | ggagacgatc ctgaagaaag agaagaaaag cagaaagatc tggaggatca ccgagatgat |
| 601 | aaagaaagcc gcccacctcg gaaatttcct tctgataaaa tttttgaagc catttcctca |
| 661 | atgtttccag ataagggcac agcagaagaa ctaaaggaaa aatataaaga actcaccgaa |
| 721 | cagcagctcc caggcgcact tcctcctgaa tgtaccccca acatagatgg accaaatgct |
| 781 | aaatctgttc agagagagca aagcttacac tcctttcata cgcttttctg taggcgatgt |
| 841 | tttaaatatg actgcttcct acatcctttt catgcaacac ccaacactta taagcggaag |
| 901 | aacacagaaa cagctctaga caacaaacct tgtggaccac agtgttacca gcatttggag |
| 961 | ggagcaaagg agtttgctgc tgctctcacc gctgagcgga taaagacccc accaaaacgt |
| 1021 | ccaggaggcc gcagaagagg acggcttccc aataacagta gcaggcccag cacccccacc |
| 1081 | attaatgtgc tggaatcaaa ggatacagac agtgataggg aagcagggac tgaaacgggg |
| 1141 | ggagagaaca atgataaaga agaagaagag aagaaagatg aaacttcgag ctcctctgaa |
| 1201 | gcaaattctc ggtgtcaaac accaataaag atgaagccaa atattgaacc tcctgagaat |
| 1261 | gtggagtgga gtggtgctga agcctcaatg tttagagtcc tcattggcac ttactatgac |
| 1321 | aatttctgtg ccattgctag gttaattggg accaaaacat gcagacaggt gtatgagttt |
| 1381 | agagtcaaag aatctagcat catagctcca gctcccgctg aggatgtgga tactcctcca |
| 1441 | aggaaaaaga agaggaaaca ccggttgtgg gctgcacact gcagaaagat acagctgaaa |
| 1501 | aagggtcaaa accgctttcc gggatgccgc tgcaaagcac agtgcaacac caagcagtgc |
| 1561 | ccgtgctacc tggctgtccg agagtgtgac cctgacctct gtcttacttg tggagccgct |
| 1621 | gaccattggg acagtaaaaa tgtgtcctgc aagaactgca gtattcagcg gggctccaaa |
| 1681 | aagcatctat tgctggcacc atctgacgtg gcaggctggg ggatttttat caaagatccc |
| 1741 | gtgcagaaaa atgaattcat ctcagaatac tgtggagaga ttatttctca agatgaagct |
| 1801 | gacagaagag ggaaagtgta tgataaatac atgtgcagct ttctgttcaa cttgaacaat |
| 1861 | gattttgtgg tggatgcaac ccgcaagggt aacaaaattc gttttgcaaa tcattcggta |
| 1921 | aatccaaact gctatgcaaa agttatgatg gttaacggtg atcacaggat aggtattttt |
| 1981 | gccaagagag ccatccagac tggcgaagag ctgttttttg attacagata cagccaggct |
| 2041 | gatgccctga agtatgtcgg catcgaaaga gaaatggaaa tcccttga |
| Mouse EZH2 (isoform 1) cDNA Sequence | |
| SEQ ID NO: 15 | |
| 1 | atgggccaga ctgggaagaa atctgagaag ggaccggttt gttggcggaa gcgtgtaaaa |
| 61 | tcagagtaca tgagactgag acagctcaag aggttcagaa gagctgatga agtaaagact |
| 121 | atgtttagtt ccaatcgtca gaaaattttg gaaagaactg aaaccttaaa ccaagagtgg |
| 181 | aagcagcgga ggatacagcc tgtgcacatc atgacttctg tgagctcatt gcgcgggact |
| 241 | agggagtgtt cagtcaccag tgacttggat tttccagcac aagtcatccc gttaaagacc |
| 301 | ctgaatgcag tcgcctcggt gectataatg tactcttggt cgcccttaca acagaatttt |
| 361 | atggtggaag acgaaactgt tttacataac attccttata tgggggatga agttctggat |
| 421 | caggatggca ctttcattga agaactaata aaaaattatg atggaaaagt gcatggtgac |
| 481 | agagaatgtg gatttataaa tgatgaaatt tttgtggagt tggtaaatgc tcttggtcaa |
| 541 | tataatgatg atgatgatga cgatgatgga gatgatccag atgaaagaga agaaaaacag |
| 601 | aaagatctag aggataatcg agatgataaa gaaacttgee cacctcggaa atttcctgct |
| 661 | gataaaatat ttgaagccat ttcctcaatg tttccagata agggcaccgc agaagaactg |
| 721 | aaagaaaaat ataaagaact cacggagcag cagctcccag gtgctctgcc tcctgaatgt |
| 781 | actccaaaca tcgatggacc aaatgccaaa tctgttcaga gggagcaaag cttgcattca |
| 841 | tttcatacgc tcttctgtcg acgatgtttt aagtatgact gcttcctaca tcccttccat |
| 901 | gcaacaccca acacatataa gaggaagaac acagaaacag ctttggacaa caagccttgt |
| 961 | ggaccacagt gttaccagca tctggaggga gctaaggagt ttgctgctgc tcttactgct |
| 1021 | gagcgtataa agacaccacc taaacgccca gggggccgca gaagaggaag acttccgaat |
| 1081 | aacagtagca gacccagcac ccccaccatc agtgtgctgg agtcaaagga tacagacagt |
| 1141 | gacagagaag cagggactga aactggggga gagaacaatg ataaagaaga agaagagaaa |
| 1201 | aaagatgaga cgtccagctc ctctgaagca aattctcggt gtcaaacacc aataaagatg |
| 1261 | aagccaaata ttgaacctcc tgagaatgtg gagtggagtg gtgctgaagc ctccatgttt |
| 1321 | agagtcctca ttggtactta ctacgataac ttttgtgcca ttgctaggct aattgggacc |
| 1381 | aaaacatgta gacaggtgta tgagtttaga gtcaaggagt ccagtatcat agcacctgtt |
| 1441 | cccactgagg atgtagacac tcctccaaga aagaagaaaa ggaaacatcg gttgtgggct |
| 1501 | gcacactgca gaaagataca actgaaaaag gacggctcct ctaaccatgt ttacaactat |
| 1561 | caaccctgtg accatccacg gcagccttgt gacagttcgt gcccttgtgt gatagcacaa |
| 1621 | aatttttgtg aaaagttttg tcaatgtagt tcagagtgtc aaaaccgctt tcctggatgt |
| 1681 | cggtgcaaag cacaatgeaa caccaaacag tgtccatgct acctggctgt ccgagagtgt |
| 1741 | gaccctgacc tctgtctcac gtgtggagct gctgaccatt gggacagtaa aaatgtatcc |
| 1801 | tgtaagaact gtagcattca gcggggctct aaaaagcact tactgctggc accgtctgat |
| 1861 | gtggcaggct ggggcatctt tatcaaagat cctgtacaga aaaatgaatt catctcagaa |
| 1921 | tactgtgggg agattatttc tcaggatgaa gcagacagaa gaggaaaagt gtatgacaaa |
| 1981 | tacatgtgca gctttctgtt caacttgaac aatgattttg tggtggatgc aacccgaaag |
| 2041 | ggcaacaaaa ttcgttttgc taatcattca gtaaatccaa actgctatgc aaaagttatg |
| 2101 | atggttaatg gtgaccacag gataggcatc tttgctaaga gggctatcca gactggtgaa |
| 2161 | gagttgtttt ttgattacag atacagccag gctgatgccc tgaagtatgt gggcatcgaa |
| 2221 | cgagaaatgg aaatcccttg a |
| Mouse EZH2 (isoform 1) Amino Acid Sequence | |
| SEQ ID NO: 16 | |
| 1 | mgqtgkksek gpvcwrkrvk seymrlrqlk rfrradevkt mfssnrqkil ertetlnqew |
| 61 | kqrriqpvhi mtsvsslrgt recsvtsdld fpaqviplkt lnavasvpim yswsplqqnf |
| 121 | mvedetvlhn ipymgdevld qdgtfieeli knydgkvhgd recgfindai fvelvnalgq |
| 181 | yndddddddg ddpdereekq kdlednrddk etcpprkfpa dkifeaissm fpdkgtaeel |
| 241 | kekykelteq qlpgalppec tpnidgpnak svqreqslhs fhtlfcrrcf kydcflhpfh |
| 301 | atpntykrkn tetaldnkpc gpqcyqhleg akefaaalta eriktppkrp ggrrrgrlpn |
| 361 | nssrpstpti svleskdtds dreagtetgg enndkeeeek kdetssssea nsrcqtpikm |
| 421 | kpnieppenv ewsgaeasmf rvligtyydn fcaiarligt ktcrqvyefr vkessiiapv |
| 481 | ptedvdtppr kkkrkhrlwa ahcrkiqlkk dgssnhvyny qpcdhprqpc dsscpcviaq |
| 541 | nfcekfcqcs secqnrfpgc rckaqcntkq cpcylavrec dpdlcltcga adhwdsknvs |
| 601 | ckncsiqrgs kkhlllapsd vagwgifikd pvqknefise ycgeiisqde adrrgkvydk |
| 661 | ymcsflfnln ndfvvdatrk gnkirfanhs vnpncyakvm ravngdhrigi fakraiqtge |
| 721 | elffdyrysq adalkyvgie remeip |
| Mouse EZH2 (isoform 2) cDNA Sequence | |
| SEQ ID NO: 17 | |
| 1 | atgggccaga ctgggaagaa atctgagaag ggaccggttt gttggcggaa gcgtgtaaaa |
| 61 | tcagagtaca tgagactgag acagctcaag aggttcagaa gagctgatga agtaaagact |
| 121 | atgtttagtt ccaatcgtca gaaaattttg gaaagaactg aaaccttaaa ccaagagtgg |
| 181 | aagcagcgga ggatacagcc tgtgcacatc atgacttctt gttcagtcac cagtgacttg |
| 241 | gattttccag cacaagtcat cccgttaaag accctgaatg cagtcgcctc ggtgcctata |
| 301 | atgtactctt ggtcgccctt acaacagaat tttatggtgg aagacgaaac tgttttacat |
| 361 | aacattcctt atatggggga tgaagttctg gatcaggatg gcactttcat tgaagaacta |
| 421 | ataaaaaatt atgatggaaa agtgcatggt gacagagaat gtggatttat aaatgatgaa |
| 481 | atttttgtgg agttggtaaa tgctcttggt caatataatg atgatgatga tgacgatgat |
| 541 | ggagatgatc cagatgaaag agaagaaaaa cagaaagatc tagaggataa tcgagatgat |
| 601 | aaagaaactt gcccacctcg gaaatttcct gctgataaaa tatttgaagc catttcctca |
| 661 | atgtttccag ataagggcac cgcagaagaa ctgaaagaaa aatataaaga actcacggag |
| 721 | cagcagctcc caggtgctct gcctcctgaa tgtactccaa acatcgatgg accaaatgcc |
| 781 | aaatctgttc agagggagca aagcttgcat tcatttcata cgctcttctg tcgacgatgt |
| 841 | tttaagtatg actgcttcct acategtaag tgcagttatt ccttccatgc aacacccaac |
| 901 | acatataaga ggaagaacac agaaacagct ttggacaaca agccttgtgg accacagtgt |
| 961 | taccagcatc tggagggagc taaggagttt gctgctgctc ttactgctga gcgtataaag |
| 1021 | acaccaccta aacgcccagg gggccgcaga agaggaagac ttccgaataa cagtagcaga |
| 1081 | cccagcaccc ccaccatcag tgtgctggag tcaaaggata cagacagtga cagagaagca |
| 1141 | gggactgaaa ctgggggaga gaacaatgat aaagaagaag aagagaaaaa agatgagacg |
| 1201 | tccagctcct ctgaagcaaa ttctcggtgt caaacaccaa taaagatgaa gccaaatatt |
| 1261 | gaacctcctg agaatgtgga gtggagtggt gctgaagcct ccatgtttag agtcctcatt |
| 1321 | ggtacttact acgataactt ttgtgccatt gctaggctaa ttgggaccaa aacatgtaga |
| 1381 | caggtgtatg agtttagagt caaggagtcc agtatcatag cacctgttcc cactgaggat |
| 1441 | gtagacactc ctccaagaaa gaagaaaagg aaacatcggt tgtgggctgc acactgcaga |
| 1501 | aagatacaac tgaaaaagga cggctcctct aaccatgttt acaactatca accctgtgac |
| 1561 | catccacggc agccttgtga cagttcgtgc ccttgtgtga tagcacaaaa tttttgtgaa |
| 1621 | aagttttgtc aatgtagttc agagtgtcaa aaccgctttc ctggatgtcg gtgcaaagca |
| 1681 | caatgcaaca ccaaacagtg tccatgctac ctggctgtcc gagagtgtga ccctgacctc |
| 1741 | tgtctcacgt gtggagctgc tgaccattgg gacagtaaaa atgtatcctg taagaactgt |
| 1801 | agcattcagc ggggctctaa aaagcactta ctgctggcac cgtctgatgt ggcaggctgg |
| 1861 | ggcatcttta tcaaagatcc tgtacagaaa aatgaattca tctcagaata ctgtggggag |
| 1921 | attatttctc aggatgaagc agacagaaga ggaaaagtgt atgacaaata catgtgcagc |
| 1981 | tttctgttca acttgaacaa tgattttgtg gtggatgcaa cccgaaaggg caacaaaatt |
| 2041 | cgctttgcta atcattcagt aaatccaaac tgctatgcaa aagttatgat ggttaatggt |
| 2101 | gaccacagga taggcatctt tgctaagagg gctatccaga ctggtgaaga gttgtttttt |
| 2161 | gattacagat acagccaggc tgatgccctg aagtatgtgg gcatcgaacg agaaatggaa |
| 2221 | atcccttga |
| Mouse EZH2 (isoform 2) Amino Acid Sequence | |
| SEQ ID NO: 18 | |
| 1 | mgqtgkksek gpvcwrkrvk seymrlrqlk rfrradevkt mfssnrqkil ertetlnqew |
| 61 | kqrriqpvhi mtscsvtsdl dfpaqviplk tlnavasvpi myswsplqqn fmvedetvlh |
| 121 | nipymgdevl dqdgtfieel iknydgkvhg drecgfinde ifvelvnalg qynddddddd |
| 181 | gddpdereek qkdlednrdd ketcpprkfp adkifeaiss mfpdkgtaee lkekykelte |
| 241 | qqlpgalppe ctpnidgpna ksvqreqslh sfhtlfcrrc fkydcflhrk csysfhatpn |
| 301 | tykrknteta ldnkpcgpqc yqhlegakef aaaltaerik tppkrpggcr rgrlpnnssr |
| 361 | pstptisvle skdtdsdrea gtetggennd keeeekkdet sssseansrc qtpikmkpni |
| 421 | eppenvewsg aeasmfrvli gtyydnfcai arligtktcr qvyefrvkes siiapvpted |
| 481 | vdtpprkkkr khrlwaahcr kiqlkkdgss nhvynyqpcd hprqpcdssc pcviaqnfce |
| 541 | kfcqcssecq nrfpgcrcka qcntkqcpcy lavrecdpdl cltcgaadhw dsknvacknc |
| 601 | siqrgskkhl llapsdvagw gifikdpvqk nefiseycge iisqdeadrr gkvydkymcs |
| 661 | flfnlnndfv vdatrkgnki rfanhsvnpn cyakvmmvng dhrigifakr aiqtgeelff |
| 721 | dyrysqadal kyvgiereme ip |
| Human HMGN1 cDNA Seauence | |
| SEQ ID NO: 19 | |
| 1 | atgcccaaga ggaaggtcag ctccgccgaa ggcgccgcca aggaagagcc caagaggaga |
| 61 | tcggcgcggt tgtcagctaa acctcctgca aaagtggaag cgaagccgaa aaaggcagca |
| 121 | gcgaaggata aatcttcaga caaaaaagtg caaacaaaag ggaaaagggg agcaaaggga |
| 181 | aaacaggccg aagtggctaa ccaagaaact aaagaagact tacctgcgga aaacggggaa |
| 241 | acgaagactg aggagagtcc agcctctgat gaagcaggag agaaagaagc caagtctgat |
| 301 | taa |
| Human HMGN1 Amino Acid Sequence | |
| SEQ ID NO: 20 | |
| 1 | mpkrkvssae gaakeepkrr sarlsakppa kveakpkkaa akdkssdkkv qtkgkrgakg |
| 61 | kqaevanqet kedlpaenge tkteespasd eagekeaksd |
| Rhesus Monkey HMGN1 cDNA Sequence | |
| SEQ ID NO: 21 | |
| 1 | atgcccaaga ggaaggtcag ctccgccgaa ggggccgcca aggaagagcc caaaaggaga |
| 61 | tcggcgcggt tgtcagctaa acctcctgcc aaagtggaag cgaagccgaa aaaggcagca |
| 121 | gcgaaggata aatcttcaga caaaaaagtg caaacaaaag ggaaaagggg agcaaaggga |
| 181 | aaacaggccg aagtggctaa ccaagaaact aaagaagatt tacctgcaga aaacggggaa |
| 241 | acgaaaactg aggagagtcc agcctctgat gaagcaggag agaaagaagc caagtctgat |
| 301 | taa |
| Rhesus Monkey HMGN1 Amino Acid Sequence | |
| SEQ ID NO: 22 | |
| 1 | mpkrkvssae gaakeepkrr sarlsakppa kveakpkkaa akdkssdkkv qtkgkrgakg |
| 61 | kqaevanqet kedlpaenge tkteespasd eagekeaksd |

### II. Agents and Compositions

Agents and compositions of the present invention are provided for us in the diagnosis, prognosis, prevention, and treatment of cancer (e.g., lymphoid cancers, such as leukemia) and cancer subtypes thereof. Such agents and compositions can detect and/or modulate, e.g., up- or down-regulate, expression and/or activity of gene products or fragments thereof encoded by biomarkers of the invention, including the biomarkers listed in Tables 1-5 and Examples. Exemplary agents include antibodies, small molecules, peptides, peptidomimetics, natural ligands, and derivatives of natural ligands, that can either bind and/or activate or inhibit protein biomarkers of the invention, including the biomarkers listed in Tables 1-5 and Examples, or fragments thereof; RNA interference, antisense, nucleic acid aptamers, etc. that can downregulate the expression and/or activity of the biomarkers of the invention, including the biomarkers listed in Tables 1-5 and Examples, or fragments thereof.

In one embodiment, isolated nucleic acid molecules that specifically hybridize with or encode one or more biomarkers listed in Tables 1-5 and Examples or biologically active portions thereof. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecules corresponding to the one or more biomarkers listed in Tables 1-5 and Examples can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (i.e., a leukemic cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having the nucleotide sequence of one or more biomarkers listed in Tables 1-5 and Examples or a nucleotide sequence which is at least about 50%, preferably at least about 60%, more preferably at least about 70%, yet more preferably at least about 80%, still more preferably at least about 90%, and most preferably at least about 95% or more (e.g., about 98%) homologous to the nucleotide sequence of one or more biomarkers listed in Tables 1-5 and Examples or a portion thereof (i.e., 100, 200, 300, 400, 450, 500, or more nucleotides), can be isolated using standard molecular biology techniques and the sequence information provided herein. For example, a human cDNA can be isolated from a human cell line (from Stratagene, La Jolla, Calif., or Clontech, Palo Alto, Calif.) using all or portion of the nucleic acid molecule, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of the nucleotide sequence of one or more biomarkers listed in Tables 1-5 and Examples or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence, or fragment thereof, can be isolated by the polymerase chain reaction using oligonucleotide primers designed based upon the sequence of the one or more biomarkers listed in Tables 1-5 and Examples, or fragment thereof, or the homologous nucleotide sequence. For example, mRNA can be isolated from muscle cells (i.e., by the guanidinium-thiocyanate extraction procedure of Chirgwin et al. (1979) Biochemistry 18: 5294-5299) and cDNA can be prepared using reverse transcriptase (i.e., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed according to well-known methods in the art. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to the nucleotide sequence of one or more biomarkers listed in Tables 1-5 and Examples can be prepared by standard synthetic techniques, i.e., using an automated DNA synthesizer.

Probes based on the nucleotide sequences of one or more biomarkers listed in Tables 1-5 and Examples can be used to detect transcripts or genomic sequences encoding the same or homologous proteins. In preferred embodiments, the probe further comprises a label group attached thereto, i.e., the label group can be a radioisotope, a fluorescent compound, an enzyme, or an enzyme co-factor. Such probes can be used as a part of a diagnostic test kit for identifying cells or tissue which express one or more biomarkers listed in Tables 1-5 and Examples, such as by measuring a level of nucleic acid in a sample of cells from a subject, i.e., detecting mRNA levels of one or more biomarkers listed in Tables 1-5 and Examples.

Nucleic acid molecules encoding proteins corresponding to one or more biomarkers listed in Tables 1-5 and Examples from different species are also contemplated. For example, rat or monkey cDNA can be identified based on the nucleotide sequence of a human and/or mouse sequence and such sequences are well known in the art. In one embodiment, the nucleic acid molecule(s) of the invention encodes a protein or portion thereof which includes an amino acid sequence which is sufficiently homologous to an amino acid sequence of one or more biomarkers listed in Tables 1-5 and Examples, such that the protein or portion thereof modulates (e.g., enhance), one or more of the following biological activities: a) binding to the biomarker; b) modulating the copy number of the biomarker; c) modulating the expression level of the biomarker; and d) modulating the activity level of the biomarker.

As used herein, the language “sufficiently homologous” refers to proteins or portions thereof which have amino acid sequences which include a minimum number of identical or equivalent (e.g., an amino acid residue which has a similar side chain as an amino acid residue in one or more biomarkers listed in Tables 1-5 and Examples, or fragment thereof) amino acid residues to an amino acid sequence of the biomarker, or fragment thereof, such that the protein or portion thereof modulates (e.g., enhance) one or more of the following biological activities: a) binding to the biomarker, b) modulating the copy number of the biomarker; c) modulating the expression level of the biomarker; and d) modulating the activity level of the biomarker.

In another embodiment, the protein is at least about 50%, preferably at least about 60%, more preferably at least about 70%, 75%, 80%, 85%, 90%, 91%, 92%, 93%, 94%, 95%, 96%, 97%, 98%, 99% or more homologous to the entire amino acid sequence of the biomarker, or a fragment thereof.

Portions of proteins encoded by nucleic acid molecules of the one or more biomarkers listed in Tables 1-5 and Examples are preferably biologically active portions of the protein. As used herein, the term “biologically active portion” of one or more biomarkers listed in Tables 1-5 and Examples is intended to include a portion, e.g., a domain/motif, that has one or more of the biological activities of the full-length protein.

Standard binding assays, e.g., immunoprecipitations and yeast two-hybrid assays, as described herein, or functional assays, e.g., RNAi or overexpression experiments, can be performed to determine the ability of the protein or a biologically active fragment thereof to maintain a biological activity of the full-length protein.

The invention further encompasses nucleic acid molecules that differ from the nucleotide sequence of the one or more biomarkers listed in Tables 1-5 and Examples, or fragment thereof due to degeneracy of the genetic code and thus encode the same protein as that encoded by the nucleotide sequence, or fragment thereof. In another embodiment, an isolated nucleic acid molecule of the invention has a nucleotide sequence encoding a protein having an amino acid sequence of one or more biomarkers listed in Tables 1-5 and Examples, or fragment thereof, or a protein having an amino acid sequence which is at least about 70%, 75%, 80%, 85%, 90%, 91%, 92%, 93%, 94%, 95%, 96%, 97%, 98%, 99% or more homologous to the amino acid sequence of the one or more biomarkers listed in Tables 1-5 and Examples, or fragment thereof. In another embodiment, a nucleic acid encoding a polypeptide consists of nucleic acid sequence encoding a portion of a full-length fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

It will be appreciated by those skilled in the art that DNA sequence polymorphisms that lead to changes in the amino acid sequences of the one or more biomarkers listed in Tables 1-5 and Examples may exist within a population (e.g., a mammalian and/or human population). Such genetic polymorphisms may exist among individuals within a population due to natural allelic variation. As used herein, the terms “gene” and “recombinant gene” refer to nucleic acid molecules comprising an open reading frame encoding one or more biomarkers listed in Tables 1-5 and Examples, preferably a mammalian, e.g., human, protein. Such natural allelic variations can typically result in 1-5% variance in the nucleotide sequence of the one or more biomarkers listed in Tables 1-5 and Examples. Any and all such nucleotide variations and resulting amino acid polymorphisms in the one or more biomarkers listed in Tables 1-5 and Examples that are the result of natural allelic variation and that do not alter the functional activity of the one or more biomarkers listed in Tables 1-5 and Examples are intended to be within the scope of the invention. Moreover, nucleic acid molecules encoding one or more biomarkers listed in Tables 1-5 and Examples from other species.

In addition to naturally-occurring allelic variants of the one or more biomarkers listed in Tables 1-5 and Examples sequence that may exist in the population, the skilled artisan will further appreciate that changes can be introduced by mutation into the nucleotide sequence, or fragment thereof, thereby leading to changes in the amino acid sequence of the encoded one or more biomarkers listed in Tables 1-5 and Examples, without altering the functional ability of the one or more biomarkers listed in Tables 1-5 and Examples. For example, nucleotide substitutions leading to amino acid substitutions at “non-essential” amino acid residues can be made in the sequence, or fragment thereof. A “non-essential” amino acid residue is a residue that can be altered from the wild-type sequence of the one or more biomarkers listed in Tables 1-5 and Examples without altering the activity of the one or more biomarkers listed in Tables 1-5 and Examples, whereas an “essential” amino acid residue is required for the activity of the one or more biomarkers listed in Tables 1-5 and Examples. Other amino acid residues, however, (e.g., those that are not conserved or only semi-conserved between mouse and human) may not be essential for activity and thus are likely to be amenable to alteration without altering the activity of the one or more biomarkers listed in Tables 1-5 and Examples.

The term “sequence identity or homology” refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1. Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a protein homologous to one or more biomarkers listed in Tables 1-5 and Examples, or fragment thereof, can be created by introducing one or more nucleotide substitutions, additions or deletions into the nucleotide sequence, or fragment thereof, or a homologous nucleotide sequence such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), branched side chains (e.g., threonine, valine, isoleucine) and aromatic side chains (e.g., tyrosine, phenylalanine, tryptophan, histidine). Thus, a predicted nonessential amino acid residue in one or more biomarkers listed in Tables 1-5 and Examples is preferably replaced with another amino acid residue from the same side chain family. Alternatively, in another embodiment, mutations can be introduced randomly along all or part of the coding sequence of the one or more biomarkers listed in Tables 1-5 and Examples, such as by saturation mutagenesis, and the resultant mutants can be screened for an activity described herein to identify mutants that retain desired activity. Following mutagenesis, the encoded protein can be expressed recombinantly according to well-known methods in the art and the activity of the protein can be determined using, for example, assays described herein.

The levels of one or more biomarkers listed in Tables 1-5 and Examples levels may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In preferred embodiments, the levels of one or more biomarkers listed in Tables 1-5 and Examples levels are ascertained by measuring gene transcript (e.g., mRNA), by a measure of the quantity of translated protein, or by a measure of gene product activity. Expression levels can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In a particular embodiment, the mRNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomezynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding one or more biomarkers listed in Tables 1-5 and Examples. Other suitable probes for use in the diagnostic assays of the invention are described herein. Hybridization of an mRNA with the probe indicates that one or more biomarkers listed in Tables 1-5 and Examples is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrixr™ gene chip array. A skilled artisan can readily adapt known mRNA detection methods for use in detecting the level of the One or more biomarkers listed in Tables 1-5 and Examples mRNA expression levels.

An alternative method for determining mRNA expression level in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA. 88:189-193), self-sustained sequence replication (Guatelli et al., 1990. Proc. Natl Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in slur methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to the One or more biomarkers listed in Tables 1-5 and Examples mRNA.

As an alternative to making determinations based on the absolute expression level, determinations may be based on the normalized expression level of one or more biomarkers listed in Tables 1-5 and Examples. Expression levels are normalized by correcting the absolute expression level by comparing its expression to the expression of a non-biomarker gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of a protein corresponding to one or more biomarkers listed in Tables 1-5 and Examples can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express the biomarker of interest.

The present invention further provides soluble, purified and/or isolated polypeptide forms of one or more biomarkers listed in Tables 1-5 and Examples, or fragments thereof. In addition, it is to be understood that any and all attributes of the polypeptides described herein, such as percentage identities, polypeptide lengths, polypeptide fragments, biological activities, antibodies, etc. can be combined in any order or combination with respect to any biomarker listed in Tables 1-5 and Examples and combinations thereof.

In one aspect, a polypeptide may comprise a full-length amino acid sequence corresponding to one or more biomarkers listed in Tables 1-5 and Examples or a full-length amino acid sequence with 1 to about 20 conservative amino acid substitutions. An amino acid sequence of any described herein can also be at least 50, 55, 60, 65, 70, 75, 80, 85, 90, 91, 92, 93, 94, 95, 96, 97, 98, 99, or 99.5% identical to the full-length sequence of one or more biomarkers listed in Tables 1-5 and Examples, which is either described herein, well known in the art, or a fragment thereof. In another aspect, the present invention contemplates a composition comprising an isolated polypeptide corresponding to one or more biomarkers listed in Tables 1-5 and Examples polypeptide and less than about 25%, or alternatively 15%, or alternatively 5%, contaminating biological macromolecules or polypeptides.

The present invention further provides compositions related to producing, detecting, characterizing, or modulating the level or activity of such polypeptides, or fragment thereof, such as nucleic acids, vectors, host cells, and the like. Such compositions may serve as compounds that modulate the expression and/or activity of one or more biomarkers listed in Tables 1-5 and Examples. For example, HMGN1 polypeptides can be used to reduce H3K27me3 and thereby allow lymphoid cells, such as lymphoid progenitors, to proliferate or, alternatively, agents that reduce HMGN1 polypeptide levels or activity can be used to stop proliferation of lymphoid cell (e.g., DS-ALL cells).

An isolated polypeptide or a fragment thereof (or a nucleic acid encoding such a polypeptide) corresponding to one or more biomarkers of the invention, including the biomarkers listed in Tables 1-5 and Examples or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

For example, a polypeptide immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well-known protocols used for fusing lymphocytes and immortalized cell lines can be applied for the purpose of generating a monoclonal antibody against one or more biomarkers of the invention, including the biomarkers listed in Tables 1-5 and Examples, or a fragment thereof (see, e.g., Galfre, G. et al. (1977) Nature 266:550-52; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-A4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628: Gram et al. (1992) Proc. Natl. Acad Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125.023; Better et al. (1988) Science 240:1041-1043; Liu et al. (1987) Proc. Nat. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw at al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art. e.g., as described in U.S. Pat. No. 5,565,332, 5,871,907, or 5,733,743. The use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson. J. R. (1993) Proc. Natl. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan. L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Hiochem. Biophys. Rev. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

Additionally, fully human antibodies could be made against biomarkers of the invention, including the biomarkers listed in Tables 1-5 and Examples, or fragments thereof. Fully human antibodies can be made in mice that are transgenic for human immunoglobulin genes, e.g. according to Hogan, et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA. 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers of the invention, including one or more biomarkers listed in Tables 1-5 and Examples, or a fragment thereof. In one embodiment, the bispecific antibody could specifically bind to both a polypeptide or a fragment thereof and its natural binding partner(s) or a fragment(s) thereof.

In another aspect of this invention, peptides or peptide mimetics can be used to antagonize or promote the activity of one or more biomarkers of the invention, including one or more biomarkers listed in Tables 1-5 and Examples, or a fragment(s) thereof. In one embodiment, variants of one or more biomarkers listed in Tables 1-5 and Examples which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see. e.g., Narang. S. A. (1983) Tetrahedron 39:3; Itakura et al. (1984) Annu. Rev. Biochemn. 53:323: Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers of the invention, including one or more biomarkers listed in Tables 1-5 and Examples, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere, J. (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983). Vol. 1, Issue 3, “Peptide Backbone Modifications” (general review); Morley, J. S. (1980) Trends Pharm. Sci. pp. 463-468 (general review); Hudson, D. et al. (1979) Int. J. Pep. Prol. Res. 14:177-185 (—CH2NH—, CH2CH2-); Spatola, A. F. et al. (1986) Life Sci. 38:1243-1249 (—CH2-S): Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982) (—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivitization (e.g., labeling) of peptidomimetics should not substantially interfere with the desired biological or pharmacological activity of the peptidomimetic.

Also encompassed by the present invention are small molecules which can modulate (either enhance or inhibit) interactions, e.g., between biomarkers listed in Tables 1-5 and Examples and their natural binding partners, or inhibit activity. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145). In some embodiments, chemical inhibitors of one or more histone H3K27 demethylases (e.g., KMD6A and/or KMD6B) are useful. Such inhibitors are well known in the art and include GSK-J4 (ethyl 3-((6-(4,5-dihydro-1H-benzo[d]azepin-3(2H)-yl)-2-(pyridin-2-yl)pyrimidin-4-yl)amino)propanoate), which has the chemical formula:

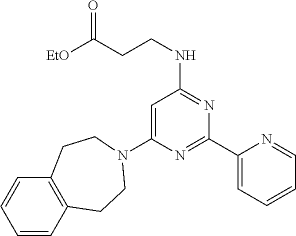 [SMILES: CCOC(=O)CCNc1cc(N2CCc3ccccc3CC2)nc(-c2ccccn2)n1]

(see, the World Wide Web at xcessbio.com/index.php/new-products-14/gsk-j4.html)

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Agnew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990), Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

The invention also relates to chimeric or fusion proteins of the biomarkers of the invention, including the biomarkers listed in Tables 1-5 and Examples, or fragments thereof. As used herein, a “chimeric protein” or “fusion protein” comprises one or more biomarkers of the invention, including one or more biomarkers listed in Tables 1-5 and Examples, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers of the invention, including one or more biomarkers listed in Tables 1-5 and Examples, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g. bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can enhance or upregulate one or more biological activities associated with the corresponding wild-type, naturally occurring, or synthetic small nucleic acids. In another embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers of the invention, including one or more biomarkers listed in Tables 1-5 and Examples, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin. In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee at al. (2002), Nat. Biotechnol. 20:500-505; and Paul et al. (2002), Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in viva. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Tables 1-5 and Examples). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, R. (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′,3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al. (1988) BioTechniques 6:958-976) or intercalating agents. (See, e.g., Zon (1988), Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil. (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford. Ill., USA), Glen Research (Sterling, Va., USA), ChemGenes (Ashland, Mass., USA), Cruachem (Glasgow, UK), and Exiqon (Vedback, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene, in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods and compositions presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp. 2nd edition (July 1999).

The modulatory agents described herein (e.g. antibodies, small molecules, peptides, fusion proteins, or small nucleic acids) can be incorporated into pharmaceutical compositions and administered to a subject in vivo. The compositions may contain a single such molecule or agent or any combination of agents described herein. Based on the genetic pathway analyses described herein, it is believed that such combinations of agents is especially effective in diagnosing, prognosing, preventing, and treating cancer. Thus, “single active agents” described herein can be combined with other pharmacologically active compounds (“second active agents”) known in the art according to the methods and compositions provided herein. It is believed that certain combinations work synergistically in the treatment of particular types of cancer. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Typical large molecule active agents are biological molecules, such as naturally occurring or artificially made proteins. Proteins that are particularly useful in this invention include proteins that stimulate the survival and/or proliferation of hematopoietic precursor cells and immunologically active poietic cells in vitro or in vivo. Others stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alpha-n1, interferon alpha-n3, interferon beta-Ia, and interferon gamma-Ib; GM-CF and GM-CSF; and EPO.

Particular proteins that can be used in the methods and compositions provided herein include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.). Recombinant and mutated forms of GM-CSF can be prepared as described in U.S. Pat. Nos. 5,391,485; 5,393,870; and 5,229,496, all of which are incorporated herein by reference. Recombinant and mutated forms of G-CSF can be prepared as described in U.S. Pat. Nos. 4,810,643; 4,999,291; 5,528,823; and 5,580,755; all of which are incorporated herein by reference.

Antibodies that can be used in combination form include monoclonal and polyclonal antibodies. Examples of antibodies include, but are not limited to, trastuzumab (Herceptin®), rituximab (Rituxan®), bevacizumab (Avastin®), pertuzumab (Omnitarg®), tositumomab (Bexxar®), edrecolomab (Panorex®), and G250. Compounds of the invention can also be combined with, or used in combination with, anti-TNF-α antibodies. Large molecule active agents may be administered in the form of anti-cancer vaccines. For example, vaccines that secrete, or cause the secretion of, cytokines such as IL-2, G-CSF, and GM-CSF can be used in the methods, pharmaceutical compositions, and kits provided herein. See, e.g., Emens, L. A., et al., Curr. Opinion Mol. Ther. 3(1):77-84 (2001).

Second active agents that are small molecules can also be used to in combination as provided herein. Examples of small molecule second active agents include, but are not limited to, anti-cancer agents, antibiotics, immunosuppressive agents, and steroids.

In some embodiments, well known “combination chemotherapy” regimens can be used. In one embodiment, the combination chemotherapy comprises a combination of two or more of cyclophosphamide, hydroxydaunorubicin (also known as doxorubicin or adriamycin), oncovorin (vincristine), and prednisone. In another preferred embodiment, the combination chemotherapy comprises a combination of cyclophosphamide, oncovorin, prednisone, and one or more chemotherapeutics selected from the group consisting of anthracycline, hydroxydaunorubicin, epirubicin, and motixantrone.

Examples of other anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer carboplatin; carmustine: carubicin hydrochloride; carzelesin; cedefingol; celecoxib (COX-2 inhibitor): chlorambucil; cirolemycin; cisplatin; cladribine; crisnatol mesylate; cyclophosphamide; cytarabine; dacarbazine; dactinomycin; daunorubicin hydrochloride: decitabine; dexormaplatin; dezaguanine; dezaguanine mesylate diaziquone; docetaxel; doxorubicin; doxorubicin hydrochloride: droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin: mitocromin; mitogillin; mitomalcin; mitomycin; mitosper; mitotane; mitoxantrone hydrochloride; mycophenolic acid; nocodazole: nogalamycin; ormaplatin; oxisuran; paclitaxel; pegaspargase; peliomycin; pentamustine; peplomycin sulfate; perfosfamide; pipobroman; piposulfan; piroxantrone hydrochloride: plicamycin; plomestane; porfimer sodium; porfiromycin; prednimustine; procarbazine hydrochloride; puromycin; puromycin hydrochloride; pyrazofurin; riboprine; safingol; safingol hydrochloride: semustine; simtrazene; sparfosate sodium: sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate: triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate: vinglycinate sulfate: vinleurosine sulfate; vinorelbine tartrate; vinrosidine sulfate: vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists: altretamine; ambamustine; amidox; amifostine; aminolevulinic acid: amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-aletheine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol: cryptophycin 8; cryptophycin A derivatives: curacin A; cyclopentanthraquinones; cycloplatam; cyclosporin A; cypemycin; cytarabine ocfosfate; cytolytic factor, cytostatin; dacliximab; decitabine; dchydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam: heregulin; hexamethylene bisacetamide; hypericin: ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide: kahalalide F; lamellarin-N triacetate; lanreotide: leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline: lytic peptides; maitansine; mannostatin A: marimastat; masoprocol; maspin; matrilysin inhibitors; matrix metalloproteinase inhibitors; menogaril; merbarone; meterelin; methioninase; metoclopramide; MIF inhibitor, mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); o6-benzylguanine: octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors, purpurins; pyrazoloacridine; pyridoxylated hemoglobin polyoxyethylene conjugate; raf antagonists: raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine: superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors: tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, chlorambucil, fludarabine, dexamethasone (Decadron®), hydrocortisone, methylprednisolone, cilostamide, doxorubicin (Doxil®), forskolin, rituximab, cyclosporin A, cisplatin, vincristine, PDE7 inhibitors such as BRL-50481 and IR-202, dual PDE4/7 inhibitors such as IR-284, cilostazol, meribendan, milrinone, vesnarionone, enoximone and pimobendan, Syk inhibitors such as fostamatinib disodium (R406/R788), R343, R-112 and Excellair® (ZaBeCor Pharmaceuticals, Bala Cynwyd, Pa.).

### III. Methods of Selecting Agents and Compositions

Another aspect of the invention relates to methods of selecting agents (e.g., antibodies, fusion proteins, peptides, small molecules, or small nucleic acids) which bind to, upregulate, downregulate, or modulate one or more biomarkers of the invention listed in Tables 1-5 and Examples and/or a cancer (e.g., a lymphoid cancer, such as leukemia). Such methods utilize can use screening assays, including cell based and non-cell based assays.

In one embodiment, the invention relates to assays for screening candidate or test compounds which bind to or modulate the expression or activity level of, one or more biomarkers of the invention, including one or more biomarkers listed in Tables 1-5 and Examples, or a fragment thereof. Such compounds include, without limitation, antibodies, proteins, fusion proteins, nucleic acid molecules, and small molecules.

In one embodiment, an assay is a cell-based assay, comprising contacting a cell expressing one or more biomarkers of the invention, including one or more biomarkers listed in Tables 1-5 and Examples, or a fragment thereof, with a test compound and determining the ability of the test compound to modulate (e.g. stimulate or inhibit) the level of interaction between the biomarker and its natural binding partners as measured by direct binding or by measuring a parameter of cancer.

For example, in a direct binding assay, the biomarker polypeptide, a binding partner polypeptide of the biomarker, or a fragment(s) thereof, can be coupled with a radioisotope or enzymatic label such that binding of the biomarker polypeptide or a fragment thereof to its natural binding partner(s) or a fragment(s) thereof can be determined by detecting the labeled molecule in a complex. For example, the biomarker polypeptide, a binding partner polypeptide of the biomarker, or a fragment(s) thereof, can be labeled with ¹²⁵I, ³⁵S, ¹⁴C, or ³H, either directly or indirectly, and the radioisotope detected by direct counting of radioemmission or by scintillation counting. Alternatively, the polypeptides of interest a can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to modulate the interactions between one or more biomarkers of the invention, including one or more biomarkers listed in Tables 1-5 and Examples, or a fragment thereof, and its natural binding partner(s) or a fragment(s) thereof, without the labeling of any of the interactants (e.g., using a microphysiometer as described in McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, determining the ability of the blocking agents (e.g. antibodies, fusion proteins, peptides, nucleic acid molecules, or small molecules) to antagonize the interaction between a given set of polypeptides can be accomplished by determining the activity of one or more members of the set of interacting molecules. For example, the activity of one or more biomarkers of the invention, including one or more biomarkers listed in Tables 1-5 and Examples, or a fragment thereof, can be determined by detecting induction of cytokine or chemokine response, detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the biomarker or a fragment thereof (e.g., modulations of biological pathways identified herein, such as modulated proliferation, apoptosis, cell cycle, and/or E2F transcription facto binding activity). Determining the ability of the blocking agent to bind to or interact with said polypeptide can be accomplished by measuring the ability of an agent to modulate immune responses, for example, by detecting changes in type and amount of cytokine secretion, changes in apoptosis or proliferation, changes in gene expression or activity associated with cellular identity, or by interfering with the ability of said polypeptide to bind to antibodies that recognize a portion thereof.

In yet another embodiment, an assay of the present invention is a cell-free assay in which one or more biomarkers of the invention, including one or more biomarkers listed in Tables 1-5 and Examples or a fragment thereof, e.g. a biologically active fragment thereof, is contacted with a test compound, and the ability of the test compound to bind to the polypeptide, or biologically active portion thereof, is determined. Binding of the test compound to the biomarker or a fragment thereof, can be determined either directly or indirectly as described above. Determining the ability of the biomarker or a fragment thereof to bind to its natural binding partner(s) or a fragment(s) thereof can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological polypeptides. One or more biomarkers polypeptide or a fragment thereof can be immobilized on a BIAcore chip and multiple agents, e.g., blocking antibodies, fusion proteins, peptides, or small molecules, can be tested for binding to the immobilized biomarker polypeptide or fragment thereof. An example of using the BIA technology is described by Fitz et al. (1997) Oncogene 15:613.

The cell-free assays of the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In one or more embodiments of the above described assay methods, it may be desirable to immobilize either the biomarker polypeptide, the natural binding partner(s) polypeptide of the biomarker, or fragments thereof, to facilitate separation of complexed from uncomplexed forms of one or both of the proteins, as well as to accommodate automation of the assay. Binding of a test compound in the assay can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-base fusion proteins, can be adsorbed onto glutathione Sepharose beads (Sigma Chemical, St. Louis, Mo.) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

In an alternative embodiment, determining the ability of the test compound to modulate the activity of one or more biomarkers of the invention, including one or more biomarkers listed in Tables 1-5 and Examples, or a fragment thereof, or of natural binding partner(s) thereof can be accomplished by determining the ability of the test compound to modulate the expression or activity of a gene, e.g., nucleic acid, or gene product, e.g., polypeptide, that functions downstream of the interaction. For example, inflammation (e.g., cytokine and chemokine) responses can be determined, the activity of the interactor polypeptide on an appropriate target can be determined, or the binding of the interactor to an appropriate target can be determined as previously described.

In another embodiment, modulators of one or more biomarkers of the invention, including one or more biomarkers listed in Tables 1-5 and Examples, or a fragment thereof, are identified in a method wherein a cell is contacted with a candidate compound and the expression or activity level of the biomarker is determined. The level of expression of biomarker mRNA or polypeptide or fragments thereof in the presence of the candidate compound is compared to the level of expression of biomarker mRNA or polypeptide or fragments thereof in the absence of the candidate compound. The candidate compound can then be identified as a modulator of biomarker expression based on this comparison. For example, when expression of biomarker mRNA or polypeptide or fragments thereof is greater (statistically significantly greater) in the presence of the candidate compound than in its absence, the candidate compound is identified as a stimulator of biomarker expression. Alternatively, when expression of biomarker mRNA or polypeptide or fragments thereof is reduced (statistically significantly less) in the presence of the candidate compound than in its absence, the candidate compound is identified as an inhibitor of biomarker expression. The expression level of biomarker mRNA or polypeptide or fragments thereof in the cells can be determined by methods described herein for detecting biomarker mRNA or polypeptide or fragments thereof.

In other embodiments, activity of histone methyl modifying proteins (e.g., enzymes) are evaluated. The effect of a test compound can be evaluated, for example, by measuring methylation of a substrate in the presence of a stimulating agent at the beginning of a time course, and then comparing such levels after a predetermined time (e.g., 0.1, 0.25, 0.5, 1, 1.5, 2, 2.5, 3, or more hours) in a reaction that includes the test compound and in a parallel control reaction that does not include the test compound. This is one example of a method for determining the effect of a test compound on enzyme activity in vitro using a stimulating agent as provided by the present disclosure. In general, an assay involves preparing a reaction mixture of a histone methyl modifying enzyme, a substrate, a stimulating agent, and one or more test compounds under conditions and for a time sufficient to allow components to interact. Methylation can be evaluated directly or indirectly. For example, H3K27 mono-, di-, and/or tri-methylation or the relative proportions or relative changes from one species to another over time, can be assessed. In some embodiments, a component of an assay reaction mixture (e.g., a substrate) is anchored onto a solid phase. A component anchored on the solid phase can be detected at the end of a reaction, e.g., a methylase reaction. Any vessel suitable reactants can be used. Examples of suitable vessels include microtiter plates, test tubes, and micro-centrifuge tubes.

Activity of methyl modifying enzymes can be evaluated by any available means. In some embodiments, a methylation state of a substrate is evaluated by mass spectrometric analysis of a substrate. In some embodiments, methylation of a substrate is evaluated with an antibody specific for a methylated or demethylated substrate. Such antibodies are commercially available (e.g., from Upstate Group, NY, or Abcam Ltd., UK). Suitable immunoassay techniques for detecting methylation state of a substrate include immunoblotting, ELISA, and immunoprecipitation. Methylation reactions can be carried out in the presence of a labeled methyl donor (e.g., a S-adenosyl-[methyl-¹⁴C]-L-methionine, or 5-adenosyl-[methyl-³H]-L-methionine), allowing detection of label into a methylase substrate, or release of label from a demethylase substrate. In some embodiments, activity of a methyl modifying enzyme is evaluated using fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on a ‘donor’ (e.g., a DNA molecule of a nucleosome) is selected such that its emitted fluorescent energy will be absorbed by a fluorescent label on an ‘acceptor’ (e.g., an antibody specific for a histone methyl modification of interest), which in turn is able to fluoresce due to the absorbed energy. A reaction can be carried out using an unlabeled substrate, and histone modification is determined by detecting antibody binding using a fluorimeter (see, U.S. Pat. Pub. 2008/0070257).

In some embodiments, demethylation is evaluated by direct or indirect detection of release of a reaction product such as formaldehyde and/or succinate. In some embodiments, release of formaldehyde is detected. Release of formaldehyde can be detected using a formaldehyde dehydrogenase assay in which formaldehyde dehydrogenase converts released formaldehyde to formic acid using NAD+ as electron acceptor. Reduction of NAD+ can be detected spectrophotometrically (Lizcano et al., Anal. Biochem. 286:75-79, 2000). In some embodiments, release of formaldehyde is detected by converting formaldehyde to 3,5-diacethyl-1,4-dihydrolutidine (DDL) and detecting the DDL, for example, by detecting radiolabeled DDL (e.g., ³H-DDL). A substrate can be labeled so that a labeled reaction product is released (e.g., formaldehyde and/or succinate) by a demethylation reaction. In some embodiments, a substrate is methylated with ³H-SAM (S-adenosylmethionine), demethylation of which releases ³H-formaldehyde, which can detected directly, or which can be converted to ³H-DDL, which is detected. Methods of detecting reaction products such as formaldehyde and/or succinate include mass spectrometry, gas chromatography, liquid chromatography, immunoassay, electrophoresis, and the like, and combinations thereof. Demethylase assays are also described in Shi et al., Cell 119:941-953, 2004. An alternative means for detecting demethylase activity employs analysis of release of radioactive carbon dioxide (see, e.g., Pappalardi et al. (2008) Biochem. 47:11165-11167 and Supporting Information, which describes use of a radioactive assay in which capture of ¹⁴CO₂ is captured and detected following release from α[1-¹⁴C]-ketoglutaric acid coupled to hydroxylation reactions). Such methods can also be employed for detection of demethylation. Detection of enzyme activity can include use of fluorescent, radioactive, scintillant, or other type of reagents. In some embodiments, a scintillation proximity assay is used for evaluating enzyme activity. Such assays can involve use of an immobilized scintillant (e.g., immobilized on a bead or microplate) and a radioactive methyl donor. In some embodiments, a scintillation proximity assay employs scintillant-coated microplates such as FlashPlates® (Perkin Elmer). In some embodiments, components of an assay reaction mixture are conjugated to biotin and streptavidin. Biotinylated components (e.g., biotinylated substrate or biotinylated stimulating agent) can be prepared, e.g., using biotin-NHS (N-hydroxy-succinimide) according to known techniques (e.g., biotinylation kit, Pierce Chemicals, Rockford, Ill.). Biotinylated components can be captured using streptavidin-coated beads or immobilized in the wells of streptavidin-coated plates (Pierce Chemical). As would be appreciated by those of skill in the art, assays can also employ any of a number of standard techniques for preparation and/or analysis of enzymatic activity, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York); and immunoprecipitation (see, for example, Ausubel, F. et al., eds. (1999) Current Protocols in Molecular Biology, J. Wiley: New York). Such resins and chromatographic techniques are known to one skilled in the art (see. e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect activity of histone methyl modifying enzymes.

In yet another aspect of the invention, a biomarker of the invention, including one or more biomarkers listed in Tables 1-5 and Examples, or a fragment thereof, can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other polypeptides which bind to or interact with the biomarker or fragments thereof and are involved in activity of the biomarkers. Such biomarker-binding proteins are also likely to be involved in the propagation of signals by the biomarker polypeptides or biomarker natural binding partner(s) as, for example, downstream elements of one or more biomarkers-mediated signaling pathway.

The two-hybrid system is based on the modular nature of most transcription factors, which consist of separable DNA-binding and activation domains. Briefly, the assay utilizes two different DNA constructs. In one construct, the gene that codes for one or more biomarkers polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” polypeptides are able to interact, in vivo, forming one or more biomarkers-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with one or more biomarkers polypeptide of the invention, including one or more biomarkers listed in Tables 1-5 and Examples or a fragment thereof.

In another aspect, the invention pertains to a combination of two or more of the assays described herein. For example, a modulating agent can be identified using a cell-based or a cell-free assay, and the ability of the agent to modulate the activity of one or more biomarkers polypeptide or a fragment thereof can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### III. Uses and Methods of the Invention

The biomarkers of the invention described herein, including the biomarkers listed in Tables 1-5 and Examples or fragments thereof, can be used in one or more of the following methods: a) screening assays; b) predictive medicine (e.g., diagnostic assays, prognostic assays, and monitoring of clinical trials); and c) methods of treatment (e.g., therapeutic and prophylactic, e.g., by up- or down-modulating the copy number, level of expression, and/or level of activity of the one or more biomarkers).

The isolated nucleic acid molecules of the invention can be used, for example, to (a) express one or more biomarkers of the invention, including one or more biomarkers listed in Tables 1-5 and Examples or a fragment thereof (e.g., via a recombinant expression vector in a host cell in gene therapy applications or synthetic nucleic acid molecule), (b) detect biomarker mRNA or a fragment thereof (e.g., in a biological sample) or a genetic alteration in one or more biomarkers gene, and/or (c) modulate biomarker activity, as described further below. The biomarker polypeptides or fragments thereof can be used to treat conditions or disorders characterized by insufficient or excessive production of one or more biomarkers polypeptide or fragment thereof or production of biomarker polypeptide inhibitors. In addition, the biomarker polypeptides or fragments thereof can be used to screen for naturally occurring biomarker binding partner(s), to screen for drugs or compounds which modulate biomarker activity, as well as to treat conditions or disorders characterized by insufficient or excessive production of biomarker polypeptide or a fragment thereof or production of biomarker polypeptide forms which have decreased, aberrant or unwanted activity compared to biomarker wild-type polypeptides or fragments thereof (e.g., cancers, including lymphoid cancers, such as leukemia).

A. Screening Assays

In one aspect, the present invention relates to a method for preventing in a subject, a disease or condition associated with an unwanted, more than desirable, or less than desirable, expression and/or activity of one or more biomarkers described herein. Subjects at risk for a disease that would benefit from treatment with the claimed agents or methods can be identified, for example, by any one or combination of diagnostic or prognostic assays known in the art and described herein (see, for example, agents and assays described in III. Methods of Selecting Agents and Compositions).

B. Predictive Medicine

The present invention also pertains to the field of predictive medicine in which diagnostic assays, prognostic assays, and monitoring of clinical trials are used for prognostic (predictive) purposes to thereby treat an individual prophylactically. Accordingly, one aspect of the present invention relates to diagnostic assays for determining the expression and/or activity level of biomarkers of the invention, including biomarkers listed in Tables 1-5 and Examples or fragments thereof, in the context of a biological sample (e.g., blood, serum, cells, or tissue) to thereby determine whether an individual is afflicted with a disease or disorder, or is at risk of developing a disorder, associated with aberrant or unwanted biomarker expression or activity. The present invention also provides for prognostic (or predictive) assays for determining whether an individual is at risk of developing a disorder associated with biomarker polypeptide, nucleic acid expression or activity. For example, mutations in one or more biomarkers gene can be assayed in a biological sample.

Such assays can be used for prognostic or predictive purpose to thereby prophylactically treat an individual prior to the onset of a disorder characterized by or associated with biomarker polypeptide, nucleic acid expression or activity.

Another aspect of the invention pertains to monitoring the influence of agents (e.g., drugs, compounds, and small nucleic acid-based molecules) on the expression or activity of biomarkers of the invention, including biomarkers listed in Tables 1-5 and Examples, or fragments thereof, in clinical trials. These and other agents are described in further detail in the following sections.

1. Diagnostic Assays

The present invention provides, in part, methods, systems, and code for accurately classifying whether a biological sample is associated with a cancer or a clinical subtype thereof (e.g., lymphoid cancers, such as leukemia). In some embodiments, the present invention is useful for classifying a sample (e.g., from a subject) as a cancer sample using a statistical algorithm and/or empirical data (e.g., the presence or level of one or biomarkers described herein).

An exemplary method for detecting the level of expression or activity of one or more biomarkers of the invention, including one or more biomarkers listed in Tables 1-5 and Examples or fragments thereof, and thus useful for classifying whether a sample is associated with cancer or a clinical subtype thereof (e.g., lymphoid cancers, such as leukemia), involves obtaining a biological sample from a test subject and contacting the biological sample with a compound or an agent capable of detecting the biomarker (e.g., polypeptide or nucleic acid that encodes the biomarker or fragments thereof) such that the level of expression or activity of the biomarker is detected in the biological sample. In some embodiments, the presence or level of at least one, two, three, four, five, six, seven, eight, nine, ten, fifty, hundred, or more biomarkers of the invention are determined in the individual's sample. In certain instances, the statistical algorithm is a single learning statistical classifier system. Exemplary statistical analyses are presented in the Examples and can be used in certain embodiments. In other embodiments, a single learning statistical classifier system can be used to classify a sample as a cancer sample, a cancer subtype sample, or a non-cancer sample based upon a prediction or probability value and the presence or level of one or more biomarkers described herein. The use of a single learning statistical classifier system typically classifies the sample as a cancer sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the cancer classification results to a clinician, e.g., an oncologist or hematologist.

In another embodiment, the method of the present invention further provides a diagnosis in the form of a probability that the individual has a cancer or a clinical subtype thereof. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having cancer or a clinical subtype thereof. In yet another embodiment, the method of the present invention further provides a prognosis of cancer in the individual. For example, the prognosis can be surgery, development of a clinical subtype of the cancer (e.g., subtype of leukemia), development of one or more symptoms, development of malignant cancer, or recovery from the disease. In some instances, the method of classifying a sample as a cancer sample is further based on the symptoms (e.g., clinical factors) of the individual from which the sample as obtained. The symptoms or group of symptoms can be, for example, those associated with the IPI. In some embodiments, the diagnosis of an individual as having cancer or a clinical subtype thereof is followed by administering to the individual a therapeutically effective amount of a drug useful for treating one or more symptoms associated with cancer or the cancer.

In some embodiments, an agent for detecting biomarker mRNA, genomic DNA, or fragments thereof is a labeled nucleic acid probe capable of hybridizing to biomarker mRNA, genomic DNA, or fragments thereof. The nucleic acid probe can be, for example, full-length biomarker nucleic acid, or a portion thereof, such as an oligonucleotide of at least 15, 30, 50, 100, 250 or 500 nucleotides in length and sufficient to specifically hybridize under stringent conditions well known to a skilled artisan to biomarker mRNA or genomic DNA. Other suitable probes for use in the diagnostic assays of the invention are described herein.

A preferred agent for detecting one or more biomarkers listed in Tables 1-5 and Examples or a fragment thereof is an antibody capable of binding to the biomarker, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, and biological fluids isolated from a subject, as well as tissues, cells, and fluids present within a subject. That is, the detection method of the invention can be used to detect biomarker mRNA, polypeptide, genomic DNA, or fragments thereof, in a biological sample in vitro as well as in vivo. For example, in vitro techniques for detection of biomarker mRNA or a fragment thereof include Northern hybridizations and in sin hybridizations. In vivo techniques for detection of biomarker polypeptide include enzyme linked immunosorbant assays (ELISAs), Western blots, immunoprecipitations and immunofluorescence. In vitro techniques for detection of biomarker genomic DNA or a fragment thereof include Southern hybridizations. Furthermore, in vive techniques for detection of one or more biomarkers polypeptide or a fragment thereof include introducing into a subject a labeled anti-biomarker antibody. For example, the antibody can be labeled with a radioactive marker whose presence and location in a subject can be detected by standard imaging techniques.

In one embodiment, the biological sample contains polypeptide molecules from the test subject. Alternatively, the biological sample can contain mRNA molecules from the test subject or genomic DNA molecules from the test subject. A preferred biological sample is a hematological tissue (e.g., a sample comprising blood, plasma, B cell, bone marrow, etc.) sample isolated by conventional means from a subject.

In another embodiment, the methods further involve obtaining a control biological sample from a control subject, contacting the control sample with a compound or agent capable of detecting polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof of one or more biomarkers listed in Tables 1-5 and Examples such that the presence of biomarker polypeptide, mRNA, genomic DNA, or fragments thereof, is detected in the biological sample, and comparing the presence of biomarker polypeptide, mRNA, eDNA, small RNAs, mature miRNA, pro-miRNA, pri-miRNA, miRNA*, anti-miRNA, or a miRNA binding site, or a variant thereof, genomic DNA, or fragments thereof in the control sample with the presence of biomarker polypeptide, mRNA, cDNA, small RNAs, mature miRNA, pre-miRNA, pri-miRNA, miRNA, anti-miRNA, or a miRNA binding site, or a variant thereof, genomic DNA, or fragments thereof in the test sample.

The invention also encompasses kits for detecting the presence of a polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, of one or more biomarkers listed in Tables 1-5 and Examples in a biological sample. For example, the kit can comprise a labeled compound or agent capable of detecting one or more biomarkers polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, in a biological sample; means for determining the amount of the biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, in the sample; and means for comparing the amount of the biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof, in the sample with a standard. The compound or agent can be packaged in a suitable container. The kit can further comprise instructions for using the kit to detect the biomarker polypeptide, mRNA, cDNA, small RNAs, mature miRNA, pre-miRNA, pri-miRNA, miRNA*, anti-miRNA, or a miRNA binding site, or a variant thereof, genomic DNA, or fragments thereof.

In some embodiments, therapies tailored to treat stratified patient populations based on the described diagnostic assays are further administered.

2. Prognostic Assays

The diagnostic methods described herein can furthermore be utilized to identify subjects having or at risk of developing a disease or disorder associated with aberrant expression or activity of one or more biomarkers of the invention, including one or more biomarkers listed in Tables 1-5 and Examples, or a fragment thereof. As used herein, the term “aberrant” includes biomarker expression or activity levels which deviates from the normal expression or activity in a control.

The assays described herein, such as the preceding diagnostic assays or the following assays, can be utilized to identify a subject having or at risk of developing a disorder associated with a misregulation of biomarker activity or expression, such as in a cancer (e.g., lymphoid cancers, such as leukemia). Alternatively, the prognostic assays can be utilized to identify a subject having or at risk for developing a disorder associated with a misregulation of biomarker activity or expression. Thus, the present invention provides a method for identifying and/or classifying a disease associated with aberrant expression or activity of one or more biomarkers of the invention, including one or more biomarkers listed in Tables 1-5 and Examples, or a fragment thereof. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with aberrant biomarker expression or activity. For example, such methods can be used to determine whether a subject can be effectively treated with an agent for a cancer (e.g., lymphoid cancers, such as leukemia). Thus, the present invention provides methods for determining whether a subject can be effectively treated with an agent for a disease associated with aberrant biomarker expression or activity in which a test sample is obtained and biomarker polypeptide or nucleic acid expression or activity is detected (e.g., wherein a significant increase or decrease in biomarker polypeptide or nucleic acid expression or activity relative to a control is diagnostic for a subject that can be administered the agent to treat a disorder associated with aberrant biomarker expression or activity). In some embodiments, significant increase or decrease in biomarker expression or activity comprises at least 2 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher or lower, respectively, than the expression activity or level of the marker in a control sample.

The methods of the invention can also be used to detect genetic alterations in one or more biomarkers of the invention, including one or more biomarkers listed in Tables 1-5 and Examples or a fragment thereof, thereby determining if a subject with the altered biomarker is at risk for cancer (e.g., lymphoid cancers, such as leukemia) characterized by aberrant biomarker activity or expression levels. In preferred embodiments, the methods include detecting, in a sample of cells from the subject, the presence or absence of a genetic alteration characterized by at least one alteration affecting the integrity of a gene encoding one or more biomarkers polypeptide, or the mis-expression of the biomarker. For example, such genetic alterations can be detected by ascertaining the existence of at least one of 1) a deletion of one or more nucleotides from one or more biomarkers gene, 2) an addition of one or more nucleotides to one or more biomarkers gene, 3) a substitution of one or more nucleotides of one or more biomarkers gene, 4) a chromosomal rearrangement of one or more biomarkers gene, 5) an alteration in the level of a messenger RNA transcript of one or more biomarkers gene, 6) aberrant modification of one or more biomarkers gene, such as of the methylation pattern of the genomic DNA, 7) the presence of a non-wild type splicing pattern of a messenger RNA transcript of one or more biomarkers gene, 8) a non-wild type level of one or more biomarkers polypeptide, 9) allelic loss of one or more biomarkers gene, and 10) inappropriate post-translational modification of one or more biomarkers polypeptide. As described herein, there are a large number of assays known in the art which can be used for detecting alterations in one or more biomarkers gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in one or more biomarkers gene (see Abravaya et al. (1995) Nucleic Acids Res. 23:675-682). This method can include the steps of collecting a sample of cells from a subject, isolating nucleic acid (e.g., genomic DNA, mRNA, cDNA, small RNA, mature miRNA, pre-miRNA, pri-miRNA, miRNA*, anti-miRNA, or a miRNA binding site, or a variant thereof) from the cells of the sample, contacting the nucleic acid sample with one or more primers which specifically hybridize to one or more biomarkers gene of the invention, including the biomarker genes listed in Tables 1-5 and Examples, or fragments thereof, under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more biomarkers gene of the invention, including one or more biomarkers listed in Tables 1-5 and Examples, or a fragment thereof, from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more biomarkers gene of the invention, including a gene listed in Tables 1-5 and Examples, or a fragment thereof, can be identified by hybridizing a sample and control nucleic acids, e.g., DNA, RNA, mRNA, small RNA, cDNA, mature miRNA, pre-miRNA, pri-miRNA, miRNA*, anti-miRNA, or a miRNA binding site, or a variant thereof, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in one or more biomarkers can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence one or more biomarkers gene of the invention, including a gene listed in Tables 1-5 and Examples, or a fragment thereof, and detect mutations by comparing the sequence of the sample biomarker gene with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in one or more biomarkers gene of the invention, including a gene listed in Tables 1-5 and Examples, or fragments thereof, include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker genes of the invention, including genes listed in Tables 1-5 and Examples, or fragments thereof, obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in biomarker genes of the invention, including genes listed in Tables 1-5 and Examples, or fragments thereof. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA. In some embodiments, the hybridization reactions can occur using biochips, microarrays, etc., or other array technology that are well known in the art.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving one or more biomarkers of the invention, including one or more biomarkers listed in Tables 1-5 and Examples, or fragments thereof.

3. Monitoring of Effects During Clinical Trials

Monitoring the influence of agents (e.g., drugs) on the expression or activity of one or more biomarkers of the invention, including one or more biomarkers listed in Tables 1-5 and Examples, or a fragment thereof (e.g., the modulation of a cancer state) can be applied not only in basic drug screening, but also in clinical trials. For example, the effectiveness of an agent determined by a screening assay as described herein to increase expression and/or activity of one or more biomarkers of the invention, including one or more biomarkers listed in Tables 1-5 and Examples or a fragment thereof, can be monitored in clinical trials of subjects exhibiting decreased expression and/or activity of one or more biomarkers of the invention, including one or more biomarkers of the invention, including one or more biomarkers listed in Tables 1-5 and Examples, or a fragment thereof, relative to a control reference. Alternatively, the effectiveness of an agent determined by a screening assay to decrease expression and/or activity of one or more biomarkers of the invention, including one or more biomarkers listed in Tables 1-5 and Examples, or a fragment thereof, can be monitored in clinical trials of subjects exhibiting decreased expression and/or activity of the biomarker of the invention, including one or more biomarkers listed in Tables 1-5 and Examples or a fragment thereof relative to a control reference. In such clinical trials, the expression and/or activity of the biomarker can be used as a “read out” or marker of the phenotype of a particular cell.

In some embodiments, the present invention provides a method for monitoring the effectiveness of treatment of a subject with an agent (e.g., an agonist, antagonist, peptidomimetic, polypeptide, peptide, nucleic acid, small molecule, or other drug candidate identified by the screening assays described herein) including the steps of (i) obtaining a pre-administration sample from a subject prior to administration of the agent; (ii) detecting the level of expression and/or activity of one or more biomarkers of the invention, including one or more biomarkers listed in Tables 1-5 and Examples or fragments thereof in the preadministration sample: (iii) obtaining one or more post-administration samples from the subject; (iv) detecting the level of expression or activity of the biomarker in the post-administration samples; (v) comparing the level of expression or activity of the biomarker or fragments thereof in the pre-administration sample with the that of the biomarker in the post administration sample or samples; and (vi) altering the administration of the agent to the subject accordingly. For example, increased administration of the agent may be desirable to increase the expression or activity of one or more biomarkers to higher levels than detected (e.g., to increase the effectiveness of the agent.) Alternatively, decreased administration of the agent may be desirable to decrease expression or activity of the biomarker to lower levels than detected (e.g., to decrease the effectiveness of the agent). According to such an embodiment, biomarker expression or activity may be used as an indicator of the effectiveness of an agent, even in the absence of an observable phenotypic response.

D. Methods of Treatment

The present invention provides for both prophylactic and therapeutic methods of treating a subject at risk of (or susceptible to) a disorder characterized by insufficient or excessive production of biomarkers of the invention, including biomarkers listed in Tables 1-5 and Examples or fragments thereof, which have aberrant expression or activity compared to a control. Moreover, agents of the invention described herein can be used to detect and isolate the biomarkers or fragments thereof, regulate the bioavailability of the biomarkers or fragments thereof, and modulate biomarker expression levels or activity.

1. Prophylactic Methods

In one aspect, the invention provides a method for preventing in a subject, a disease or condition associated with an aberrant expression or activity of one or more biomarkers of the invention, including one or more biomarkers listed in Tables 1-5 and Examples or a fragment thereof, by administering to the subject an agent which modulates biomarker expression or at least one activity of the biomarker. Subjects at risk for a disease or disorder which is caused or contributed to by aberrant biomarker expression or activity can be identified by, for example, any or a combination of diagnostic or prognostic assays as described herein. Administration of a prophylactic agent can occur prior to the manifestation of symptoms characteristic of the biomarker expression or activity aberrancy, such that a disease or disorder is prevented or, alternatively, delayed in its progression.

2. Therapeutic Methods

Another aspect of the invention pertains to methods of modulating the expression or activity or interaction with natural binding partner(s) of one or more biomarkers of the invention, including one or more biomarkers listed in Tables 1-5 and Examples or fragments thereof, for therapeutic purposes. The biomarkers of the invention have been demonstrated to correlate with cancer (e.g., lymphoid cancers, such as leukemia). Accordingly, the activity and/or expression of the biomarker, as well as the interaction between one or more biomarkers or a fragment thereof and its natural binding partner(s) or a fragment(s) thereof can be modulated in order to modulate the immune response.

Modulatory methods of the invention involve contacting a cell with one or more biomarkers of the invention, including one or more biomarkers of the invention, including one or more biomarkers listed in Tables 1-5 and Examples or a fragment thereof or agent that modulates one or more of the activities of biomarker activity associated with the cell. An agent that modulates biomarker activity can be an agent as described herein, such as a nucleic acid or a polypeptide, a naturally-occurring binding partner of the biomarker, an antibody against the biomarker, a combination of antibodies against the biomarker and antibodies against other immune related targets, one or more biomarkers agonist or antagonist, a peptidomimetic of one or more biomarkers agonist or antagonist, one or more biomarkers peptidomimetic, other small molecule, or small RNA directed against or a mimic of one or more biomarkers nucleic acid gene expression product.

An agent that modulates the expression of one or more biomarkers of the invention, including one or more biomarkers of the invention, including one or more biomarkers listed in Tables 1-5 and Examples or a fragment thereof is, e.g., an antisense nucleic acid molecule, RNAi molecule, shRNA, mature miRNA, pre-miRNA, pri-miRNA, miRNA*, anti-miRNA, or a miRNA binding site, or a variant thereof, or other small RNA molecule, triplex oligonucleotide, ribozyme, or recombinant vector for expression of one or more biomarkers polypeptide. For example, an oligonucleotide complementary to the area around one or more biomarkers polypeptide translation initiation site can be synthesized. One or more antisense oligonucleotides can be added to cell media, typically at 200 μg/ml, or administered to a patient to prevent the synthesis of one or more biomarkers polypeptide. The antisense oligonucleotide is taken up by cells and hybridizes to one or more biomarkers mRNA to prevent translation. Alternatively, an oligonucleotide which binds double-stranded DNA to form a triplex construct to prevent DNA unwinding and transcription can be used. As a result of either, synthesis of biomarker polypeptide is blocked. When biomarker expression is modulated, preferably, such modulation occurs by a means other than by knocking out the biomarker gene.

Agents which modulate expression, by virtue of the fact that they control the amount of biomarker in a cell, also modulate the total amount of biomarker activity in a cell.

In one embodiment, the agent stimulates one or more activities of one or more biomarkers of the invention, including one or more biomarkers listed in Tables 1-5 and Examples or a fragment thereof. Examples of such stimulatory agents include active biomarker polypeptide or a fragment thereof and a nucleic acid molecule encoding the biomarker or a fragment thereof that has been introduced into the cell (e.g., cDNA, mRNA, shRNAs, siRNAs, small RNAs, mature miRNA, pre-miRNA, pri-miRNA, miRNA*, anti-miRNA, or a miRNA binding site, or a variant thereof, or other functionally equivalent molecule known to a skilled artisan). In another embodiment, the agent inhibits one or more biomarker activities. In one embodiment, the agent inhibits or enhances the interaction of the biomarker with its natural binding partner(s). Examples of such inhibitory agents include antisense nucleic acid molecules, anti-biomarker antibodies, biomarker inhibitors, and compounds identified in the screening assays described herein.

These modulatory methods can be performed in vitro (e.g., by contacting the cell with the agent) or, alternatively, by contacting an agent with cells in vivo (e.g., by administering the agent to a subject). As such, the present invention provides methods of treating an individual afflicted with a condition or disorder that would benefit from up- or down-modulation of one or more biomarkers of the invention listed in Tables 1-5 and Examples or a fragment thereof, e.g., a disorder characterized by unwanted, insufficient, or aberrant expression or activity of the biomarker or fragments thereof. In one embodiment, the method involves administering an agent (e.g., an agent identified by a screening assay described herein), or combination of agents that modulates (e.g., upregulates or downregulates) biomarker expression or activity. In another embodiment, the method involves administering one or more biomarkers polypeptide or nucleic acid molecule as therapy to compensate for reduced, aberrant, or unwanted biomarker expression or activity.

Stimulation of biomarker activity is desirable in situations in which the biomarker is abnormally downregulated and/or in which increased biomarker activity is likely to have a beneficial effect. Likewise, inhibition of biomarker activity is desirable in situations in which biomarker is abnormally upregulated and/or in which decreased biomarker activity is likely to have a beneficial effect.

In addition, these modulatory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer (e.g., lymphoid cancers, such as leukemia), being treated, the extent of the disease and other factors familiar to the physician of skill in the art and can be determined by the physician.

E. Methods of Expanding Lymphoid Progenitor Cell Populations

In another aspect, the present invention provides methods of increasing the number of lymphoid progenitor cells from an initial population of lymphoid progenitor cells comprising contacting the lymphoid progenitor cells with an agent that inhibits polycomb repressor complex 2 (PRC2) activity to thereby increase the number of lymphoid progenitor cells.

1. Cell Types for Expansion

As described herein, lymphoid progenitor cells and cellular sources comprising same can be used. Descriptions of cells herein are well known to the skilled artisan and are further described with the understanding that these descriptions reflect the current state of knowledge in the art and the invention is not limited thereby to only those phenotypic markers described herein.

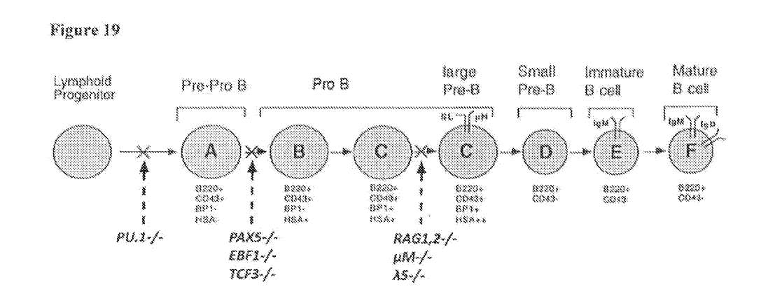

Hematopoietic stem cells give rise to lymphoid or myeloid progenitor cells. A “lymphoid progenitor cell” refers to a cell capable of differentiating into any of the terminally differentiated cells of the lymphoid lineage. Encompassed within the lymphoid progenitor cells are the common lymphoid progenitor cells (CLP), a cell population characterized by limited or non-self-renewal capacity but which is capable of cell division to form T lymphocyte and B lymphocyte progenitor cells, NK cells, and lymphoid dendritic cells. The marker phenotypes useful for identifying CLPs will be those commonly known in the art. For example, for CLP cells of mouse, the cell population is characterized by the presence of markers as described in Kondo et al. (1997) Cell 91:661-672, while for human CLPs, a marker phenotype of CD34+ CD38+ CD10+IL7R+may be used (Galy et al. (1995) Immunity, 3:459-473; Akashi et al. (1999) Int. J. Hematol. 69:217-226). Additional illustrations of B cell lineage development and associated molecular markers defining each cell stage in mouse models are provided in FIG. 19 (Iritani et al. (1997) EMBO J. 16:7019-7031; Hardy and Hayakawa (2001) Ann. Rev. Immunol. 19:595-621).

By contrast, committed myeloid progenitor cells refer to cell populations capable of differentiating into any of the terminally differentiated cells of the myeloid lineage. Encompassed within the myeloid progenitor cells are the common myeloid progenitor cells (CMP), a cell population characterized by limited or non-self-renewal capacity but which is capable of cell division to form granulocyte/macrophage progenitor cells (GMP) and megakaryocyte/erythroid progenitor cells (MEP). Non-self-renewing cells refers to cells that undergo cell division to produce daughter cells, neither of which have the differentiation potential of the parent cell type, but instead generates differentiated daughter cells. The marker phenotypes useful for identifying CMPs include those commonly known in the art. For CMP cells of murine origin, the cell population is characterized by the marker phenotype c-Kit(high) (CD117) CD16(low) CD34(low) Sca-1(neg) Lin(neg) and further characterized by the marker phenotypes FcγR(lo) IL-7Rα(neg) (CD127). The murine CMP cell population is also characterized by the absence of expression of markers that include B220, CD4, CD8, CD3, Ter119, Gr-1 and Mac-1. For CMP cells of human origin, the cell population is characterized by CD34+CD38+ and further characterized by the marker phenotypes CD123+ (IL-3Rα) CD4SR(neg). The human CMP cell population is also characterized by the absence of cell markers CD3, CD4, CD7, CD8, CD10, CD11b, CD14, CD19, CD20, CD56, and CD234a. Descriptions of marker phenotypes for various myeloid progenitor cells are described in, for example, U.S. Pat. Nos. 6,465,247 and 6,761,883; Akashi (2000) Nature 404:193-197. Another committed progenitor cell of the myeloid lineage is the granulocyte/macrophage progenitor cell (GMP). The cells of this progenitor cell population are characterized by their capacity to give rise to granulocytes (e.g., basophils, eisinophils, and neutrophils) and macrophages. Similar to other committed progenitor cells, GMPs lack self-renewal capacity. Murine GMPs are characterized by the marker phenotype c-Kit(hi) (CD117) Sca-1(neg) Fc (CD116) IL-7Rγ(neg) CD34(pos). Murine GMPs also lack expression of markers B220, CD4, CD8, CD3, Gr-1, Mac-1, and CD90. Human GMPs are characterized by the marker phenotype CD34+ CD38+ CD123+ CD45RA+. Human GMP cell populations are also characterized by the absence of markers CD3, CD4, CD7, CD8, CD10, CD11b, CD14, CD19, CD20, CD56, and CD235a. In addition, megakaryocyte/erythroid progenitor cells (MEP), which are derived from the CMPs, are characterized by their capability of differentiating into committed megakaryocyte progenitor and erythroid progenitor cells. Mature megakaryocytes are polyploid cells that are precursors for formation of platelets, a developmental process regulated by thrombopoietin. Erythroid cells are formed from the committed erythroid progenitor cells through a process regulated by erythropoietin, and ultimately differentiate into mature red blood cells. Murine MEPs are characterized by cell marker phenotype c-Kit(hi) and IL-7R and further characterized by marker phenotypes Fc and CD34(low). Murine MEP cell populations are also characterized by the absence of markers B220, CD4, CD8, CD3, Gr-1, and CD90. Another exemplary marker phenotype for mouse MEPs is c-kit(high) Sca-1(neg) Lin (neg/low) CD16 (low) CD34(low). Human MEPs are characterized by marker phenotypes CD34+ CD38+ CD1123(neg) CD45RA(neg). Human MEP cell populations are also characterized by the absence of markers CD3, CD4, CD7, CD8, CD10, CD11b, CD14, CD19, CD20, CD56, and CD235a. Further restricted progenitor cells in the myeloid lineage are the granulocyte progenitor, macrophage progenitor, megakaryocyte progenitor, and erythroid progenitor. Granulocyte progenitor cells are characterized by their capability to differentiate into terminally differentiated granulocytes, including eosinophils, basophils, neutrophils. The GPs typically do not differentiate into other cells of the myeloid lineage. With regards to the megakaryocyte progenitor cell (MKP), these cells are characterized by their capability to differentiate into terminally differentiated megakaryocytes but generally not other cells of the myeloid lineage (see, e.g., WO 2004/024875).

In some embodiments, the cells to be expanded are comprised within tissues or other cellular sources, such as bone marrow, peripheral blood, cord blood, and the like. Peripheral and cord blood is a rich source of HSCs and progenitor cells. Cells are obtained using methods known and commonly practiced in the art. For example, methods for preparing bone marrow cells are described in Sutherland et al., Bone Marrow Processing and Purging: A Practical Guide (Gee, A. P. ed.), CRC Press Inc. (1991)). Umbilical cord blood or placental cord blood is typically obtained by puncture of the umbilical vein, in both term or preterm, before or after placental detachment (see, e.g., Turner, C. W. et al., Bone Marrow Transplant. 10:89 (1992); Bertolini, F. et al., J. Hematother. 4:29 (1995)).

In other embodiments, the starting cells to be expanded are isolated cells. Such cells can further be selected and purified, which can include both positive and negative selection methods, to obtain a substantially pure population of cells. In one aspect, fluorescence activated cell sorting (FACS), also referred to as flow cytometry, is used to sort and analyze the different cell populations. Cells having the cellular markers specific for a lymphoid progenitor cell population are tagged with an antibody, or typically a mixture of antibodies, that bind the cellular markers. Each antibody directed to a different marker is conjugated to a detectable molecule, particularly a fluorescent dye that can be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and progenitor cells is described in, among others, U.S. Pat. Nos. 5,137,809, 5,750,397, 5,840,580; 6,465,249; Manz, M. G. et al., Proc. Natl. Acad. Sci. USA 99:11872-11877 (2002); and Akashi, K. et al., Nature 404(6774):193-197 (2000)). General guidance on fluorescence activated cell sorting is described in, for example, Shapiro, H. M., Practical Flow Cytometry, 4th Ed., Wiley-Liss (2003) and Ormerod, M. G., Flow Cytometry: A Practical Approach, 3rd Ed., Oxford University Press (2000).

Another method of isolating the initial cell populations uses a solid or insoluble substrate to which as bound antibodies or ligands that interact with specific cell surface markers. In immunoadsorption techniques, cells are contacted with the substrate (e.g., column of beads, flasks, magnetic particles) containing the antibodies and any unbound cells removed. Immunoadsorption techniques can be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads can be readily isolated by a magnetic separator (see, e.g., Kato, K. and Radbruch, A., Cytometry 14(4):384-92 (1993); CD34+ direct isolation kit, Miltenyi Biotec, Bergisch, Gladbach, Germany). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations.

FACS and most batch wise immunoadsorption techniques can be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that can be employed is use of antibody/complement treatment or immunotoxins to remove unwanted cells.

It is to be understood that the purification of cells also includes combinations of the methods described above. A typical combination may comprise an initial procedure that is effective in removing the bulk of unwanted cells and cellular material, for example leukapharesis. A second step may include isolation of cells expressing a marker common to one or more of the progenitor cell populations by immunoadsorption on antibodies bound to a substrate. For example, magnetic beads containing anti-B2204 antibodies are able to bind and capture lymphoid progenitors that commonly express the B220 antigen. An additional step providing higher resolution of different cell types, such as FACS sorting with antibodies to a set of specific cellular markers, can be used to obtain substantially pure populations of the desired cells. Another combination may involve an initial separation using magnetic beads bound with anti-B220 antibodies followed by an additional round of purification with FACS.

Where applicable, stem cells and lymphoid progenitor cells can be mobilized from the bone marrow into the peripheral blood by prior administration of cytokines or drugs to the subject (see, e.g., Lapidot, T. et al., Exp. Hematol. 30:973-981 (2002)). Cytokines and chemokines capable of inducing mobilization include, by way of example and not limitation, granulocyte colony stimulating factor (G-CSF), granulocyte macrophage colony stimulating factor (GM-CSF), erythropoietin (Kiessinger. A. et al., Exp. Hematol. 23:609-612 (1995)), stem cell factor (SCF), AMD3100 (AnorMed, Vancouver, Canada), interleukin-8 (IL-8), and variants of these factors (e.g., pegfilgastrim, darbopoietin). Combinations of cytokines and/or chemokines, such as G-CSF and SCF or GM-CSF and G-CSF, can act synergistically to promote mobilization and may be used to increase the number of lymphoid progenitor cells in the peripheral blood, particularly for subjects who do not show efficient mobilization with a single cytokine or chemokine (Morris, C. et al., J. Haematol. 120:413-423 (2003)). Cytoablative agents can also be used at inducing doses (i.e., cytoreductive doses) to mobilize lymphoid progenitor cells, and are useful either alone or in combination with cytokines. This mode of mobilization is applicable when the subject is to undergo mycloablative treatment, and is carried out prior to the higher dose chemotherapy. Cytoreductive drugs for mobilization, include, among others, cyclophosphamide, ifosfamide, etoposide, cytosine arabinoside, and carboplatin (Montillo, M. et al., Leukemia 18:57-62 (2004); Dasgupta, A. et al., J. Infusional Chemother. 6:12 (1996); Wright, D. E. et al., Blood 97:(8):2278-2285 (2001)).

Determining the differentiation potential of cells, and thus the type of stem cells or progenitor cells isolated, is typically conducted by exposing the cells to conditions that permit development into various terminally differentiated cells. These conditions generally comprise a mixture of cytokines and growth factors in a culture medium permissive for development of the lymphoid lineage. Colony forming culture assays rely on culturing the cells in vitro via limiting dilution and assessing the types of cells that arise from their continued development. A common assay of this type is based on methylcellulose medium supplemented with cytokines (e.g., MethoCult, Stem Cell Technologies, Vancouver, Canada; Kennedy, M. et al., Nature 386:488-493 (1997)). Cytokine and growth factor formulations permissive for differentiation in the hematopoietic pathway are described in Manz et al., Proc. Natl. Acad. Sci. USA 99(18):11872-11877 (2002); U.S. Pat. No. 6,465,249; and Akashi, K. et al., Nature 404(6774):193-197 (2000)). Cytokines include SCF, FLT-3 ligand. GM-CSF, IL-3, TPO, and EPO. Another in vitro assay is long-term culture initiating cell (LTC-IC) assay, which typically uses stromal cells to support hematopoiesis (see, e.g., Ploemacher, R. E. et al., Blood. 74:2755-2763 (1989); and Sutherland, H. J. et al., Proc. Natl. Acad. Sci. USA 87:3745 (1995)).

Another type of assay suitable for determining the differentiation potential of isolated cells relies upon in vivo administration of cells into a host animal and assessment of the repopulation of the hematopoietic system. The recipient is immunocompromised or immunodeficient to limit rejection and permit acceptance of allogeneic or xenogeneic cell transplants. A useful animal system of this kind is the NOD/SCID (Pflumio, F. et al., Blood 88:3731 (1996); Szilvassym S. J. et al., “Hematopoietic Stem Cell Protocol,” in Methods in Molecular Medicine, Humana Press (2002); Greiner, D. L. et al., Stem Cells 16(3):166-177 (1998); Piacibello, W. et al., Blood 93:(11):3736-3749 (1999)) or Rag2 deficient mouse (Shinkai, Y. et al., Cell 68:855-867 (1992)). Cells originating from the infused cells are assessed by recovering cells from the bone marrow, spleen, or blood of the host animal and determining presence of cells displaying specific cellular markers, (i.e., marker phenotyping) typically by FACS analysis. Detection of markers specific to the transplanted cells permits distinguishing between endogenous and transplanted cells. For example, antibodies specific to human forms of the cell markers (e.g., HLA antigens) identify human cells when they are transplanted into suitable immunodeficient mouse (see, e.g., Piacibello. W. et al., supra).

The initial populations of cells obtained by the methods above are used directly for expansion or frozen for use at a later date. A variety of mediums and protocols for freezing cells are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig, C. et al., Bone Marrow Transplant. 34(6):531-6 (2004)), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well known and described in the art (see, e.g., Broxmeyer et al. (2003) Proc. Natl. Acad. Sci. USA 100:645-650). Cells are preserved at a final temperature of less than about −135° C.

Expansion of lymphoid progenitor cells is carried out in a basal medium, which can be supplemented with the mixture of cytokines and growth factors described herein, sufficient to support expansion of lymphoid progenitor cells. The basal medium will comprise amino acids, carbon sources (e.g., pyruvate, glucose, etc.), vitamins, serum proteins (e.g., albumin), inorganic salts, divalent cations, antibiotics, buffers, and other preferably defined components that support expansion of myeloid progenitor cells. Suitable basal mediums include, by way of example and not limitation. RPMI medium. Iscove's medium, minimum essential medium, Dulbeccos Modified Eagles Medium, and others known in the art (see, e.g., U.S. Pat. No. 6,733,746). Commercially available basal mediums include, by way of example and not limitation, Stemline™ (Sigma Aldrich), StemSpan™ (StemCell Technologies, Vancouver, Canada), Stempro™ (Life Technologies, Gibco BRL, Gaithersburg, Md., USA) HPGM™ ((Cambrex, Walkersville, Md., USA), QBSF™ (Quality Biological, Gaithersburg, Md., USA), X-VIVO (Cambrex Corp., Walkersville, Md., USA) and Mesencult™ (StemCell Technologies, Vancouver, Canada). The formulations of these and other mediums will be apparent to the skilled artisan.

The initial population of cells are contacted with the mixture of cytokines and growth factors in the basal medium, and cultured to expand the population of myeloid progenitor cells. Expansion is done for from about 2 days to about 14 days, preferably from about 4 days to 10 days, more preferably about 4 days to 8 days and/or until the indicated fold expansion and the characteristic cell populations are obtained.

In one embodiment, the final cell culture preparation is characterized by a lymphoid progenitor cell population that is expanded at least about 0.5 fold, about 1 fold, about 5 fold, about 10 fold, about 20 fold, or more. In the final culture, the lymphoid progenitor cell population can comprise at least about 60%, 65%, 70%, 75%, 80%, 85%, 90%, 95%, 99%, or more of the total cells in the culture.

Variations on the basic culture techniques described herein readily understood by the skilled artisan are included within the scope of the present invention. For example, feeder cell cultures can be used to alter the growth media environment (Feugier, P. et al., J Hematother Stem Cell Res 11(1): 127-38 (2002)). Similarly, co-cultures of various cell populations can be created. Cells expanded by the methods described herein can be used without further purification, or can be isolated into different cell populations by various techniques known in the art, such as by immunoaffinity chromatography, immunoadsorption, FACS sorting, or other procedures as described above. Preferably. FACS sorting or immunoadsorption is used. For example, a FACS gating strategy has an initial selection for live cells based on characteristic forward scatter (cell size) and side scatter (cell density) parameters, and a second selection for expression of cell markers for lymphoid progenitor cells or non-lymphoid cells.

2. Agents to Inhibit Polycomb Repressor Complex 2 (PRC2) Catalytic Activity

The PRC2 complex directs histone methyltransferase activity. Although the compositions of the complexes isolated by different groups are slightly different, they generally contain EED, EZH2, SUZ12, and RbAp48 or Drosophila homologs thereof. However, a reconstituted complex comprising only EED, EZH2, and SUZ12 retains histone methyltransferase activity (e.g., mono-through tri-methylation) for lysine 27 of histone H3 (e.g., H3K27me3; see U.S. Pat. No. 7,563,589; Cardoso et al. (2000) Eur. J. Hum. Genet. 8:174-180). The PRC2 complex may also interact with DNMT1, DNMT3A, DNMT3B and PHF1 via the EZH2 subunit and with SIRT1 via the SUZ12 subunit. Of the various proteins making up PRC2 complexes, EZH2 (Enhancer of Zeste Homolog 2) is the catalytic subunit (Vire et al. (2006) Nature 439:871-874). The catalytic site of EZH2 in turn is present within a SET domain, a highly conserved sequence motif (named after Su(var)3-9. Enhancer of Zeste, Trithorax) that is found in several chromatin-associated proteins, including members of both the Trithorax group and Polycomb group. The SET domain is characteristic of all known histone lysine methyltransferases except the H3-K79 methyltransferase DOT1.

Any agent that disrupts the catalytic methyltransferase activity of PRC2 can be used according to the methods described herein. Such agents include small molecules, antisense nucleic acids, interfering RNA, shRNA, siRNA, aptamers, ribozymes, and dominant-negative protein binding partners. For example, knockout or knockdown of EZH2 or other PRC2 complex components, such as through reduction of mRNA or protein, will reduce H3K27me3 methylation. Similarly, functional knockout or knockdown of PRC2 H3K27me3 activity can be achieved by disrupting the protein-protein interactions necessary for the PRC2 to form and/or maintain catalytic activity. For example, dominant negative proteins, such as EZH2 lacking a functional catalytic domain and/or having reduced histone methyltransferase activity, but maintaining the ability to bind to PRC2 complex binding partner(s) will reduce PRC2 H3K27me3 activity. In some embodiments, chemical (e.g., small molecule) inhibitors of PRC2 activity, such as small molecule inhibitors of EZH2, are particularly useful because expansion of cell populations can be easily reversed by withdrawal of the compound. Such chemical inhibitors are well known in the art and are described, for example, in US Pat. Publs. 2013-0059849, 2013-0053397, 2013-0053383, 2013-0040906, 2012-0264734, 2012-0071418, as well as McCabe et al. (2012) Nature 492:108-112. In one embodiment, a chemical inhibitor of EZH2 is used, such as GSK-126 (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide) having the structure:

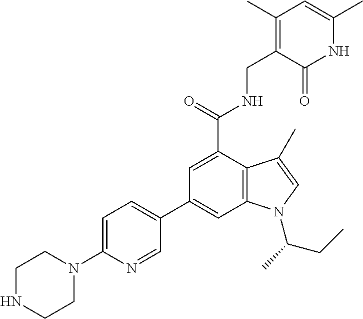 [SMILES: CC[C@H](C)n1cc(C)c2c(C(=O)NCc3c(C)cc(C)[nH]c3=O)cc(-c3ccc(N4CCNCC4)nc3)cc21]

(see, the World Wide Web at xcessbio.com/index.php/home-page-products/gsk 126.html)

3. Uses of Expanded Lymphoid progenitor Cells

Expanded cell populations prepared by the methods described herein are useful for the treatment of various disorders and applicable for many biomedical and biotechnological situations. As used herein, “treatment” can refer to therapeutic or prophylactic treatment, or a suppressive measure for a disease, disorder or undesirable condition. Treatment encompasses administration of the subject cells in an appropriate form prior to the onset of disease symptoms and/or after clinical manifestations, or other manifestations of the disease or condition to reduce disease severity, halt disease progression, or eliminate the disease. Prevention of the disease includes prolonging or delaying the onset of symptoms of the disorder or disease, preferably in a subject with increased susceptibility to the disorder. The amount of the cells needed for achieving a therapeutic effect will be determined empirically in accordance with conventional procedures for the particular purpose. Generally, for administering the cells for therapeutic purposes, the cells are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms or manifestations of the disorder or disease.

Cell populations expanded in vivo will already be comprised within a subject's body for use therein. Cells for infusion, such as those prepared in vitro or ex viva, include expanded cell populations without additional purification, or isolated cell populations having defined cell marker phenotype and characteristic differentiation potential as described herein. Expanded cells may be derived from a single subject, where the cells are autologous or allogeneic to the recipient. It is to be understood that cells isolated directly from a donor subject without expansion in culture may be used for the same therapeutic purposes as the expanded cells. Preferably, the isolated cells are a substantially pure population of cells. These unexpanded cells may be autologous, where the cells to be infused are obtained from the recipient, such as before treatment with cytoablative agents. In another embodiment, the unexpanded cells are allogeneic to the recipient, where the cells have a complete match, or partial or full mismatch with the MHC of the recipient. As described above, the isolated unexpanded cells are preferably obtained from different donors to provide a mixture of allogeneic lymphoid cells.

Transplantation of cells into an appropriate host can be accomplished by methods generally used in the an. The preferred method of administration is intravenous infusion. The number of cells transfused will take into consideration factors such as sex, age, weight, the types of disease or disorder, stage of the disorder, the percentage of the desired cells in the cell population (e.g., purity of cell population), and the cell number needed to produce a therapeutic benefit. Generally, the numbers of expanded cells infused may be from about 1×10⁴ to about 1×10⁵ (cells/kg, from about 1×10⁵ to about 10×10⁶ cells/kg, preferably about 1×10⁶ cells to about 5×10⁵ cells/kg of body weight, or more as necessary. In some embodiments, the cells are in a pharmaceutically acceptable carrier at about 1×10 to about 1×10⁹ cells. Cells can be administered in one infusion, or through successive infusions over a defined time period sufficient to generate a therapeutic effect. Different populations of cells may be infused when treatment involves successive infusions. A pharmaceutically acceptable carrier, as further described below, may be used for infusion of the cells into the patient. These will typically comprise, for example, buffered saline (e.g., phosphate buffered saline) or unsupplemented basal cell culture medium, or medium as known in the art.

Conditions suitable for treatment include genetic and/or acquired immunodeficiency or autoimmune diseases where, for example, patients have decreased numbers of lymphocytes leading to susceptibility to infection and shortened lifespan. Exemplary, non-limiting genetic immunodeficiencies include combined immunodeficiencies (SCID), such as ADA-deficiency (adenosine deaminase), X-SCID (X linked SCID), ZAP-70 deficiency, Rag 1/2 deficiency, Jak3 deficiency, IL7RA deficiency or CD3 deficiencies; primary immunodeficiencies, such as the acquired immunodeficiency syndrome (AIDS), DiCGeorge's (velocardiofacial) syndrome, adenosine deaminase (ADA) deficiency, reticular dysgenesis, Wiskott/Aldrich syndrome, ataxia-telangiectasia, severe combined immunodeficiency; and secondary immunodeficiencies, such as energy from tuberculosis, drug-induced leukopenia, non-HIV viral illnesses leukopenia, radiation poisoning, toxin exposure, malnutrition, and the like.

Expanded lymphoid cell populations are also useful for various transplantation conditions, such as transplantation of stem cells, bone marrow, and/or umbilical cord blood. Lymphoid progenitors expanded in vitro, ex vivo, or in vivo can shorten the time to immune reconstitution, thereby decreasing the likelihood of infectious complications.

The ability to expand lymphoid cell populations has numerous additional applications to biotechnological and biomedical research in addition to or outside the context of treating subjects. For example, lymphocytes that produce antibodies can be expanded in order to improved immune responses in vivo or to improve the yields of diagnostic or therapeutic antibodies produced in vitro or ex vivo. Similarly, B cells or other lymphoid cells, such as those useful for research purposes that have been genetically modified, could be indefinitely cultured to perpetuate clonal cell populations.

### IV. Pharmaceutical Compositions

In another aspect, the present invention provides pharmaceutically acceptable compositions which comprise a therapeutically-effective amount of an agent that modulates (e.g., increases or decreases) PRC2 activity and/or H3K27me3 levels, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes: (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) PRC2 activity and/or H3K27me3 levels, or expression and/or activity of the complex, or composition comprising an agent that modulates (e.g., inhibits) PRC2 activity and/or H3K27me3 levels, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline: (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) PRC2 activity and/or H3K27me3 levels, or expression and/or activity of the complex encompassed by the invention. These salts can be prepared in situ during the final isolation and purification of the respiration uncoupling agents, or by separately reacting a purified respiration uncoupling agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) PRC2 activity and/or H3K27me3 levels, or expression and/or activity of the complex. These salts can likewise be prepared in situ during the final isolation and purification of the respiration uncoupling agents, or by separately reacting the purified respiration uncoupling agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like: (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1% to about 99% of active ingredient, preferably from about 5% to about 70%, most preferably from about 0%/o to about 30%.

Methods of preparing these formulations or compositions include the step of bringing into association an agent that modulates (e.g., increases or decreases) PRC2 activity and/or H3K27me3 levels, with the carrier and, optionally, one or more accessory ingredients. In general, the formulations are prepared by uniformly and intimately bringing into association a respiration uncoupling agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid, (2) binders, such as, for example, carboxymethylcellulose, alginates, gelatin, polyvinyl pyrrolidone, sucrose and/or acacia (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., increases or decreases) PRC2 activity and/or H3K27me3 levels include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., increases or decreases) PRC2 activity and/or H3K27me3 levels, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., increases or decreases) PRC2 activity and/or H3K27me3 levels, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a respiration uncoupling agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more respiration uncoupling agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., increases or decreases) PRC2 activity and/or H3K27me3 levels, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the respiration uncoupling agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### V. Administration of Agents

The cancer diagnostic, prognostic, prevention, and/or treatment modulating agents of the invention are administered to subjects in a biologically compatible form suitable for pharmaceutical administration in vivo, to either enhance or suppress immune cell mediated immune responses. By “biologically compatible form suitable for administration in vivo” is meant a form of the protein to be administered in which any toxic effects are outweighed by the therapeutic effects of the protein. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of a blocking antibody may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The agents of the invention described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent of the invention (e.g., an antibody, peptide, fusion protein or small molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by, and directly dependent on, (a) the unique characteristics of the active compound and the particular therapeutic effect to be achieved, and (b) the limitations inherent in the art of compounding such an active compound for the treatment of sensitivity in individuals.

In one embodiment, an agent of the invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays. In addition, an antibody of the invention can also be administered in combination therapy with, e.g., chemotherapeutic agents, hormones, antiangiogens, radiolabelled, compounds, or with surgery, cryotherapy, and/or radiotherapy. An antibody of the invention can also be administered in conjunction with other forms of conventional therapy, either consecutively with, pre- or post-conventional therapy. For example, the antibody can be administered with a therapeutically effective dose of chemotherapeutic agent. In another embodiment, the antibody can be administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular immune disorder, e.g., Hodgkin lymphoma, being treated, the extent of the disease and other factors familiar to the physician of skill in the art and can be determined by the physician.

In addition, the agents of the invention described herein can be administered using nanoparticle-based composition and delivery methods well known to the skilled artisan. For example, nanoparticle-based delivery for improved nucleic acid (e.g., small RNAs) therapeutics are well known in the art (Expert Opinion on Biological Therapy 7:1811-1822).

### EXEMPLIFICATION

This invention is further illustrated by the following examples, which should not be construed as limiting.

### Example 1

### Materials and Methods for Example 2

### A. Mice

All animal experiments were performed with approval of the Dana-Farber Cancer Institute (DFCI) Institutional Animal Care And Use Committee (IACUC). All experiments were performed in an FVB×C57BL6 F1 background, unless otherwise specified. Ts1Rhr (B6.129S6-Dp(16Cbr1-ORF9)1Rhr/J; stock #005838) and Ts65Dn (B6EiC3Sn.BLiA-Ts(17¹⁶)65Dn/DnJ; stock #005252) mice were obtained from Jackson Laboratories. HMGN_1OE mice were described in Bustin et al. (1995) DNA Cell Biol. 14:997-1005. Pax5^{+/-} mice (Urbanek et al. (1994) Cell 79:901-912) backcrossed to C57BL/6 were obtained from M. Busslinger. Eμ-CRLF2 and Eμ-JAK2 R683G were generated by subcloning cDNAs expressing human CRLF2 or mouse JAK2 R683G (Mullighan et al. (2009) Nat. Genet. 41:1243-1246; Yoda et al. (2010) Proc. Natl. Acad. Sci. U.S.A. 107:252-257) downstream of the immunoglobulin heavy chain enhancer (Eμ) and generating transgenic founders in FVB fertilized eggs as described in Dildrop et al. (1989) EMBO J. 8:1121-1128. Controls for Ts1Rhr were wild-type littermates from crosses with either C57Bl/6 (Jackson; #000664) or FVB (Jackson; #001800) mice as indicated. Controls for Ts65Dn were littermates from the colony (B6EiC3Sn.BLiAF1/J; Jackson; #003647). HMGN1_OE mice (Bustin ei al. (1995) DNA Cell Biol. 14:997-105) had been backcrossed >10 generations to C57BL/6 (Abuhatzira et al. (2011) J. Biol. Chem. 286:42051-42062). Controls for HMGN1_OE were wild-type littermates after crossing with FVB mice. Donors for competitive transplantation were congenic CD45.1+B6.SJL-Ptprc^{a} Pepc^{b}/BoyJ (Jackson; stock #002014) crossed with FVB (CD45.1), CS7BL/6×FVB F1 (CD45.1/2), or Ts1Rhr (C57BL/6) crossed with FVB F1 (CD45.1/2). Recipients for competitive transplant, and BCR/ABL and Ik6 bone marrow transplants were C57BL/6×FVB F1 female mice. No randomization was performed for experiments involving mice or samples collected from animals.

### B. Antibodies

Western blotting antibodies were against HMGN1 (Aviva Systems Biology, #ARP38532_P050, rabbit polyclonal), HMGN1 (Abcam, #ab5212, rabbit polyclonal), mouse HMGN1 (affinity purified rabbit polyclonal) (Birger et al. (2003) EMBO J. 22:1665-1675; Bustin et al. (1990) J. Biol. Chem. 265:20077-20080), H3K27me3 (Cell Signaling Technologies, #9733, rabbit polyclonal), total Histone H3 (Cell Signaling Technologies, #9715, rabbit polyclonal), and α-tubulin (Sigma, #T9026, mouse monoclonal). Flow cytometry antibodies were B220-Pacific Blue (BD Pharmingen, #558108, clone RA3-6B2), CD43-APC (BD, #560663, clone S7) or CD43-FITC (BD, #561856, clone S7), CD24-PE-Cy7 (BD, #560536, clone M1/69), BP1-PE (eBiosciences, 12-5891, clone 6C3) or BP1-FITC (eBiosciences, 11-5891, clone 6C3), CD45.1-PE-Cy7 (eBiosciences, 25-0453, clone A20), and CD45.2-APC (eBiosciences, 17-0454, clone 104). ChIP-seq antibodies were H3K27me3 (Cell Signaling Technologies, #9733), H3K4me3 (Abcam, #ab8580), and H3K27ac (Abcam, #ab4729).

### C. Flow Cytometry for Bone Marrow B Cells

Whole bone marrow was harvested from femurs and tibias of 6-8-week-old mice. After red blood cell lysis (Qiagen, #158904), B cell progenitors were stained using antibodies and flow cytometry was performed as described in Hardy et al. (1991) J. Exp. Med. 173:1213-1225. Analysis was performed on a BD FACSCanto II.

### D. Competitive Bone Marrow Transplantation

Whole bone marrow was pooled from femurs and tibias of two 8-week-old donor mice. Donor cells were wild-type or Ts1Rhr CD45.1+/CD45.2+C57BL/6×FVB F1 (test) and CD45.1+B6.SJL×FVB F1 (competitor), and were mixed in a 1:1 ratio. Recipients were lethally irradiated (550 cGy×2, spaced >4 hours apart). B6SJL×FVB F1 mice received 10⁶ total cells (5×10⁵ cells each of test and competitor) via lateral tail vein injection. Bone marrow was harvested 16 weeks after transplantation and analyzed by flow cytometry.

### E. Methylcellulose Colony Forming Assays

Whole bone marrow was harvested from 6-8-week-old mice, and red blood cells were lysed. Cells were plated in B cell (Methocult M3630, Stem Cell Technologies) or myeloid (Methocult M3434) methylcelluose media in gridded 35 mm dishes. Myeloid colonies were plated at 2×10⁴ cells/ml per passage. B cell colonies were plated at 2×10⁵ cells/ml in passage 1, and at 5×10⁴ cells/ml per subsequent passage. Colonies were counted at 7 days, and colonies were then pooled and replated in the same manner.

### F. BMT Models

For BCR-ABL transplantations (Krause et al. (2006) Nat. Med. 12:1175-1180), 10⁵ transduced cells were transplanted with 10⁶ wild-type untransduced bone marrow cells for radioprotection. For generation of BCR-ABL B-ALLs derived from Hardy B cells, 5×10⁴ Hardy B cells from 6 week-old mice were sorted on a BD FACSAria II SORP, spinoculation was performed as described above, and 10³ cells were transplanted into lethally irradiated wild-type recipients with 10⁶ bone marrow cells for radioprotection. Dominant negative Ikaros experiments were performed similarly, except 10⁶ cells spinfected with an MSCV retrovirus expressing GFP alone, or coexpressing GFP and Ik6 (Iacobucci et al. (2×008) Blood 112:3847-3855; Trageser et al. (1991) J. Exp. Med. 206:1739-1753), were transplanted. Mice were followed daily for clinical signs of leukemia and were sacrificed when moribund. Investigators were not blinded to the experimental groups. Ten mice were used per arm for 80% power to detect a 60% difference in survival at a specific time point with alpha of 0.05. No animals were excluded from analysis.

### G. Cell Culture

Ba/F3 experiments were performed as described in Yoda et al. (2010) Proc. Natl. Acad. Sci. U.S.A. 107:252-257. shRNAs targeting Hmgn1 are described below (competitive shRNA assay), and cDNA expressing HMGN1 was described in Rochman et al. (2011) Nucl. Acids Res. 39: 4076-4087). One week after selection in puromycin, retroviral eDNA or lentiviral shRNA-transduced cells were harvested for Western blotting. hTERT-RPE1 cells were cultured in DMEM/F-12. Mouse A9 cells containing a single human chromosome 21 tagged with neomycin-resistant gene (a gift from Dr. M. Oshimura, Tottori University, Japan) were cultured in DMEM. All medium was supplemented with 10% FBS, 100 IU/ml penicillin and 100 μg/ml streptomycin.

### H. Immunoblotting and Quantitation

Western blotting was performed as described in Yoda et al. (2010) Proc. Natl. Acad. Sci. U.S.A. 107:252-257. Image J (available on the World Wide Web at imagej.nih.gov/ij) was used for quantitation of immunoblots, with band intensity normalized to total H3.

### I. Microcell-Mediated Chromosome Transfer (MMCT)

MMCT was performed as described in Yang and Shen (2011) Methods Mol. Biol. 325:59-66 with modifications. A9 cells were cultured to approximately 70% confluence, and treated with 75 ng/ml colcemid for 48 hours. Cells were collected and resuspended in 1:1 DMEM:Percoll (GE Healthcare Biosciences) with 10 μg/ml Cytochalasin B (Sigma-Aldrich), and spun at 17,000 rpm for 75 minutes in a Beckman JA17 rotor. Supernatant was collected and filtered through 10 and 5 μm filters. Approximately 2×10⁶ RPE1 cells were collected and mixed with filtered microcells, treated with 100 μg/ml PHA-P (Sigma-Aldrich) for 30 minutes, and fused by PEG 1500 (Sigma-Aldrich) in solution. Hybrid cells were plated and cultured for 48 hours, and selected with 500 μg/ml Geneticin (Life Technologies) for 12-14 days. Standard G-band analysis was performed at Karyologic, Inc. SNP array was performed at the DFCI microarray core, using the Human Mapping 250k-Nsp platform. Fluorescent in sir hybridization was performed with the Vysis LSI 21 SpectrumOrange probe (Abbott Molecular) according to the manufacturer's instructions.

### J. DR-GFP and DR-GFP-CE Reporter Targeting

Generating and screening of targeted clones were performed as described in Fung and Weinstock (2011) PloS One 6:e20514, with the following modifications. 10⁶ RPE1 cells with 2, 3, or 4 copies of chromosome 21 were nucleofected with 2 μg pAAVS1-DRGFP or pAAVS1-DRGFPCE plasmid together with 2 μg pZFN-AAVS1, using program X-001 of the Amaxa nucleofector II (Lonza). Targeting of individual clones was confirmed by PCR using the Accuprime GC-rich DNA polymerase (Life Technologies). The presence of a single integrant was determined by qPCR.

### K. DNA Repair Assays Using DR-GFP Reporter Cell Lines

Assays for homologous recombination and imprecise non-homologous end-joining were performed as described in Weinstock et al. (2006) Methods Enzymol. 409:524-540 with the following modifications. Transfections were performed with the Neon transfection system (Life Technologies) using 1600V, 20 ms, and 1 pulse. 4×10⁵ DR-GFP cells were transfected with 10 μg I-SceI expression vector (pCBASce) or empty vector (pCAGGS), and plated in 6-well plates. pmCherry-C1 vector (Clontech) was transfected in parallel to confirm equal transfection efficiency. Cells were cultured for 7 days and analyzed by FACS using FACSCalibur (BD Biosciences) for homology-directed repair. The remaining cells were used to extract genomic DNA. One μg DNA was digested with 20 U I-SceI (Roche) overnight, purified, and amplified with a two-step PCR protocol. Accuprime GC-rich polymerase was used for the first step PCR (20 cycles), and Taq polymerase (Qiagen) was used for the second step PCR (20 cycles). PCR products were cloned with the TOPO TA cloning kit for sequencing (Life Technologies). For DR-GFP-CE, pCAGGS-RAG1 and pCAGGS-RAG2 vectors were co-transfected. One μg genomic DNA was digested with 10 U MfeI and 10 U NdeI (NEB) overnight to exclude templates that had not been cleaved by RAG-1 and RAG-2 before PCR amplification.

### L. PCR Primers Used in DNA Repair Assays

The following primers sequences were designed and synthesized to amplified the indicated amplicon for the indicated use:

| Amplicon | | Primers (Forward then Reverse) |
|---|---|---|
| AAVS1 targeting | 5′ junction | 5′-CCAGCTCCCATAGCTCAGTC |
| | | 5′-CTTCATGCAATTGTCGGTCA |
| | 3′ junction | 5′-GCTGCCTCACAAACTTCACA |
| | | 5′-TGAGTTTGCCAAGCAGTCAC |
| qPCR for integrants | DR-GFP and DR-GFP-CE | 5′-AATGCCCTGGCTCACAAATACCAC |
| | constructs | 5′-TGTCCTTCCGAGTGAGAGACACAA |
| | Reference amplicon near | 5′-TGGCCAGGCTGAAAGGATAGGATT |
| | AAVS1 | 5′-AGAATCCAGGTCCAGGGCTGATTT |
| Sequencing of repair | First step | 5′-TTTGGCAAAGAATTCAGATCC |
| products | | 5′-CAAATGTGGTATGGCTGATTATG |
| | Second step | 5′-AAGTAGAAGACCCACGAGGCAACA |
| | | 5′-TGTGGCGGATCTTGAAGTTCACCT |

M. Competitive shRNA Assay in Primary B Cells

shRNAs targeting triplicated Ts1Rhr genes and controls were obtained from The RNAi Consortium (available on the World Wide Web at broadinstitute.org/rnai/trc) as pLKO lentiviral supernatants (Ashton et al. (2012) Cell Stem Cell 11:359-372) (n=185 total shRNAs; see Table 5 for clone ID# and target sequences). Wild-type or Ts1Rhr passage 1 B cell colonies were collected and plated at 5×10⁴ cells per well of a 96 well plate in 100 μl of RPMI with 20% FBS, and 10 ng/ml each of murine IL-7, stem cell factor, and FLT3 ligand (all from R&D Systems), with 8 μg/ml polybrene. Ten μl of lentiviral supernatant was added and the plate was centrifuged at 1000×g for 30 minutes, and then placed in a 37° C. incubator for 24 hours. Wells were pooled, 10⁶ cells were saved for input shRNA analysis, and 2×10′ cells were plated in 6 ml M3630 methylcellulose with 0.05 μg/ml puromycin in a 10 cm non-tissue culture treated dish. At this density of plating, after 7 days of growth there were at least 4×10′ colonies per plate which would represent >200 colonies per individual shRNA on average. After each passage, genomic DNA was harvested from 10⁶ cells (Qiagen QIAmp kit), and 2×10⁶ cells were replated in the same manner. Repassaging continued until cultures stopped forming new colonies (3-4 passages for wild-type) or until 6 passages were completed. The entire assay was repeated in n=3 (wild-type) and n=4 (Ts1Rhr) independent biological replicates.

The shRNA encoded in the genomic DNA was amplified using two rounds of PCR. Primary PCR reactions were performed using up to 10 μg of genomic DNA in 100 μl reactions consisting of 10 μl buffer, 8 μl dNTPs (2.5 mM each), 10 μl of 5 μM primary PCR primer mix (see below) and 1.5 μl Takara exTaq. For the secondary PCR amplification the reaction was performed as described in Ashton et al. (2012) Cell Stem Cell 11:359-372 using modified forward primers, which incorporated Illumina adapters and 6-nucleotide barcodes. Secondary PCR reactions were pooled and run on a 2% agarose gel. The bands were normalized and pooled based on relative intensity. Equal amount of sample was run on a 2% agarose gel and gel purified. Samples were sequenced using a custom sequencing primer on an Illumina Hi-Seq and quantitated as described in Ashton et al. (2012) Cell Stem Cell 11:359-372. The following PCR primer sequences were used:

Primary PCR Primers
5′ primer: AATGGACTATCATATGCTTACCGTAACTTGAAAGTATTTCG
3′ primer: CTTTAGTTTGTATGTCTGTTGCTATTATGTCTACTATTCTTTCCC
Secondary PCR Primers
5′ 6nt Bar-coded PCR primer:
5′-
AATGATACGGCGACCACCGACCGTAACTTGAAAGTATTTCGATTTCTTGGCTT
TATATATCNNNNNNAAAGGAC-3′
3′ Universal PCR primer:
5′-CAAGCAGAAGACGGCATACGAGCTCTTCCGATCTTGTGGATGAATACTGCCA
TTTGTCTC-3′
Custom Illumina sequencing primer:
CCGTAACTTGAAAGT/i6diPr/TTTCGATTTCTTGGCTTT/i6diPr/T/i6diPr/TATC

### N. RNA Sequencing and Data Processing

Total RNA was harvested from B cell colonies (n=3 independent biologic replicates per genotype per passage). RNA sequencing was performed at The Center for Cancer Computational Biology at the Dana-Farber Cancer Institute (DFCI). Quality control of total RNA was performed using the RNA Qubit Assay (Invitrogen) and the Bioanalyzer RNA Nano 6000 Chip Kit (Agilent). At least 100 ng of total RNA and a Bioanalyzer RNA Integrity Number of >7.0 were required. Library construction was performed using a TruSeq RNA Library Prep Kit (Illumina). Final library quality control was performed using the DNA High Sensitivity Qubit Kit (Invitrogen), the Bioanalyzer High Sensitivity Chip Kit (Agilent) and the 7900HT Fast qPCR machine (Applied Biosystems). qPCR was performed using the Illumina Universal Library Quantification Kit from KAPA Biosystems. RNASeq libraries were then normalized to 2 nM, pooled for multiplexing in equal volumes, and sequenced at 10 pM on the Illumina HiSeq 2000. Sequencing was performed as 2×50 paired-end reads using the 100 cycles per lane Sanger/Illumina 1.9 deep sequencing protocol. The raw sequence data were subjected to data quality control checks based on per base sequence quality scores, per sequence quality scores, per sequence GC content, sequence length distribution, and overrepresented sequences, which are implemented in the FastQC tool (available on the World Wide Web at bioinformatics.babraham.ac.ukiprojects/fastqc/). Reads that passed quality control filters were aligned against the mouse reference genome by using the ultra-high-throughput long read aligner Bowtie2 (Langmead and Salzberg (2012) Nature Methods 9:357-359) available through TopHat 2.0.7 (Trapnell et al. (2012) Nat. Protocols 7:562-578) (available on the World Wide Web at tophat.cbcb.umd.edu). Mapping results were further analyzed with TopHat to identify splice junctions between exons. Genomic annotations in gene transfer format (GTF) were obtained from Ensembl mouse genome GRCm38 (available on the World Wide Web at useast.ensembl.org/Mus_musculus/Info/Index). Gene-level expression measurements for 23,021 Ensembl mouse genes were reported in fragments per kilobase per million reads (FPKM) by Cufflinks 2.0.0 (Trapnell et al. (2010) Nat. Biotech. 28:511-515) (available on the World Wide Web at cufflinks.cbcb.umd.edu/). An FPKM filtering cutoff of 1.0 in at least one of the sample was used to determine expressed transcripts.

### O. Differential Analysis for RNA-Seq Transcript Expression

Differential analysis was performed by applying the EdgeR method (Robinson et al. (2010) Bioinformatics 26:139-140) implemented in the EdgeR library in Bioconductor v2.11 (available on the World Wide Web at bioconductor.org/). EdgeR uses empirical Bayes estimation and exact tests based on the negative binomial distribution model of the genome-scale count data. EdgeR estimates the gene-wise dispersions by conditional maximum likelihood, conditioning on the total count for that gene. The gene-wise dispersion is “normalized” by shrinking towards a consensus value based on an empirical Bayes procedure (Robinson and Smyth (2007) Bioinformatics 23:2881-2887). The differential expression is estimated separately for each gene based on an exact test analogous to Fisher's exact test adopted for over-dispersed data (Robinson and Smyth (2008) Biostatistics 9:321-332).

### P. Gene Expression Profiling (GEP) and Gene Set Enrichment Analysis (GSEA)

The series matrix file for two DS-ALL datasets (AIEOP and ICH) were downloaded from GEO (GEO accession number GSE17459) (Hertzberg et al. (2010) Blood 115:1006-1017), as were the Rag1^{-/-} and E2A/Tcf3^{-/-} B cell progenitors (GSE21978) (Lin et al. (2010) Nat. Immunol. 11:635-643). RNA from HMGN1 transgenic (HMGN1_OE) or wild-type littermate B cell colonies was processed and hybridized to Affymetrix Mouse Gene 2.0 ST array at the DFCI Microarray Core per the manufacturer's instructions. Raw probe-level data from the AIEOP-2 non-DS-ALL cohort and the mouse HMGN1_OE GEP were summarized using the Robust Multiarray Average (RMA) (Irizarry et al (2003) Nucl. Acids Res. 31:e15) and Brainarray custom chip identification files based on Entrez IDs (Version 17) (Dai et al. (2005) Nucl. Acids Res. 33:e175) using the ExpressionFileCreator module in Gene Pattern (Reich et al. (2005) Nat. Genet. 38:500-501). For GSEA the expression file was converted to human gene orthologs using BioMart (Kinsella et al. (2011) Database 2011:bar030). GSEA of the Ts1Rhr, the core Ts1Rhr, and the PRC2 gene sets was performed as described in Subramanian et al. (2005) Proc. Natl. Acad. U.S.A. 102:15545-15550 using GSEA v2.0.10 (available on the World Wide Web at broadinstitute.org/gsea/). The Ts1Rhr gene set was tested for its enrichment in the c (positional), c2.cgp (chemical and genetic perturbation), c3.tft (transcription factor targets), and c6 (oncogenic signatures) gene sets deposited in the Molecular Signature Database MSigDB v3.1 (Broad Institute; available on the World Wide Web at broadinstitute.org/gsea/msigdb). The analysis was performed by applying the 2-tailed Fisher test method, as implemented in the Investigate_GeneSets module at MSigDB. To define the Ts1Rhr B cell gene set, the top 150 most differentially expressed protein coding genes with an adjusted p-value below 0.25 were selected. Hierarchical clustering of this signature in DS-ALL vs. non-DS-ALL revealed a subset of genes most contributing to the distinguishing phenotype and this branch defined the “Core” Ts1Rhr gene set. Full gene sets for BENPORATH_SUZ12_TARGETS, MIKKELSEN_MEF_HCP_WITH_H3K27ME3, and MIKKELSEN_MEF_NPC_WITH_H3K27ME3 were obtained from MSigDB v3.1. The 100 most differentially expressed genes between the DS-ALLs and the non-DS-ALLs were determined using the MarkerSelectionModule in GenePattern. For E2A target gene expression, RAG1−/− proB cells were compared to E2A−/− preproB cells to generate probesets with >1.5-fold change and P<0.05 between conditions, exactly as had been done by the authors (Lin et al. (2010) Nat. Immunol. 11:635-643). The Ts1Rhr and core gene sets were compared to all probesets for their relative expression in E2A wild-type (RAG1−/− proB) vs E2A−/− cells.

### Q. Network Enrichment Mapping

The gene sets with significant enrichment in genes up-regulated in Ts1Rhr by GSEA were selected based on the maximum cut-off value 0.05 for P-value and FDR, and visualized with Enrichment Map software (Merico et al. (2010) PLoS One 5:e13984). This software organizes the significant gene sets into a network, where nodes correspond to gene sets and the edges reflect significant overlap between the nodes according to a Fisher's test. The size of the nodes is proportional to the number of genes in the gene set. The hubs correspond to collections of genes sets with significant pair-wise overlap which have a unifying functional description according to GO biological processes. The node color is associated to the functional description of the hub. The clusters provided by the Enrichment Map are described in Table 3.

### R. Visualization of Gene Expression and Mass Spectrometry Data

RNASeq-derived expression data from Ts1Rhr and wild-type B cells, B-ALL gene expression data, and histone mass spectrometry data were visualized as heat maps using GENE-E (available on the World Wide Web at broadinstitute.org/cancer/software/GENE-E/).

S. BCR-ABL B-ALL model

Generation of B-ALLs by transduction of wild-type or Ts1Rhr bone marrow with p210 BCR-ABL in an MSCV-ires-GFP retrovirus was performed as previously described (Krause et al. Nat. Med. 12:1175-1180), with modifications. For limiting dilution transplantations, 10⁵ or 10⁴ spinoculated cells were transplanted with 10⁶ wild-type untransduced bone marrow cells for radioprotection. 10⁶ spinoculated cells were transplanted without additional radioprotective cells. Mice were followed daily for clinical signs of leukemia and were sacrificed when moribund. Complete blood count analysis was performed with a Hemavet 950 (Drew Scientific). For calculation of leukemia-initiating cell frequency, L-Calc software from Stem Cell Technologies (available on the World Wide Web at stemcell.com/en/Products/All-Products/LCalc-Software.aspx) was used and transplanted BCR/ABL+ cells were calculated by multiplying the number of cells transplanted by the % GFP+ cells at the time of transplant (limiting dilution curves compared by chi-squared test) (Wang et al. Blood 89:3919-3924). For generation of BCR-ABL B-ALLs derived from Hardy A, Hardy B or Hardy C cells, staining for Hardy fractions in wild-type or Ts1Rhr 6-8-week-old bone marrow was performed as described above, and 5×10⁴ cells from each subpopulation were sorted on a BD FACSAria II SORP. Spinoculation with BCR-ABL retrovirus was performed as described above, and 10 cells were transplanted into lethally irradiated wild-type recipients with 10⁶ bone marrow cells for radioprotection.

### T. Column Purification of Mouse B-ALLs

For Western blotting of mouse B-ALLs, cryopreserved B-ALL splenocytes were enriched using anti-CD19 antibody conjugated to magnetic microbeads (#130-052-201) and an MS MACS column (#130-042-201), both from Miltenyi Biotec.

U. Histone mass spectrometry

Mass spectrometry for global histone H3 post-translational modifications was performed as described in Peach et al. (2012) Mol. Cell. Proteom. 11:128-137 using wild-type or Ts1Rhr passage 1 B cells and BCR-ABL B-ALLs. H3K27 modifications are presented in conjunction with H3K36, as both are present in the same measured peptides because of their close proximity.

### V. Drug Treatment

GSK-J4 (KDM6A/UTX and KDM6B/JMJD3 inhibitor, catalog #M60063-2) (Kruidenier et al. (2012) Nature 488:404-408) and GSK-126 (EZH2 inhibitor, catalog #M60071-2) (McCabe et al. (2012) Nature 492:108-112) were purchased from Xcessbio. For methylcellulose experiments, at each passage DMSO, GSK-J4, or GSK-126 were added to cultures a final concentration of 1 μM. DS-ALLs (deidentified specimens obtained with informed consent under DFCI IRB protocol 05-001) were treated In vitro in quadruplicate with GSK-J4 at two-fold dilutions from 40 nM to 10 μM in RPMI with 20% calf serum supplemented with 10 ng/mL IL3, IL7, SCF, FLT3 ligand, and 50 μM beta-mercaptoethanol. After 3 days, viability was measured using CellTiter-Glo reagent and normalized to DMSO control (Promega).

### W. In Vitro GSK-J4 Assays

Leukemia cells were murine BCR/ABL-positive B-ALLs as described above, or human Down syndrome or non-Down syndrome primary xenografted B-ALLs. Viable cells were plated in white opaque 384-well plates (50 μl/well; Corning) using EL406 Combination Washer Dispenser (BioTek) at a density of 0.25×10⁶ cells/ml. GSK-J4 or vehicle (DMSO) were added using a JANUS Automated Workstation (PerkinElmer) at the indicated concentrations. After 72 hours, CellTiter-Glo Luminescent Cell Viability Assay reagent (Promega) was added (25 μl each well) and read by the 2104 EnVision Multilabel Reader (PerkinElmer) per the manufacturers' instructions. Each data point was quantified in quadruplicate. Dose-response curves and plots were generated with GraphPad Prism software.

### X. ChIP Analyses

B cell colonies (>5,000 colonies per genotype) from 3 wild-type and 3 Ts1Rhr animals were pooled after 7 days in methylcellulose culture. ChIP was performed as described in Verzi et al. (2010) Dev. Cell 19:713-726. Libraries for sequencing were prepared following the Illumina TruSeq DNA Sample Preparation v2 kit protocol. After end-repair and A-tailing, immunoprecipitated DNA (10-50 ng) or whole cell extract DNA (50 ng) was ligated to a 1:50 dilution of Illumina Adaptor Oligo Mix assigning one of 24 unique indexes in the kit to each sample. Following ligation, libraries were amplified by 18 cycles of PCR using the HiFi NGS Library Amplification kit from KAPA Biosystems. Amplified libraries were then size-selected using a 2% gel cassette in the Pippin Prep system from Sage Science set to capture fragments between 200 and 400 bp. Libraries were quantified by qPCR using the KAPA Biosystems Illumina Library Quantification kit according to kit protocols. Libraries with distinct TruSecq indexes were multiplexed by mixing at equimolar ratios and running together in a lane on the Illumina HiSeq 200 for 40 bases in single read mode. Alignment to mouse genome assembly NCBI37/mm9 and normalization were performed as described in Lin et al. (2012) Cell 151:56-67. Regions of modified histones enriched in wild type and Ts1Rhr cells were identified using MACS peak calling algorithm at a P-value of 1e-9 (Zhang et al. (2008) Genome Biol. 9:R137). Location analysis of ChIP-target enriched regions was performed using the CEAS software suite developed by the Liu lab at DFCI (Shin et al. (2009) Bioinformatics 25:2605-2606). Promoters states were classified by the presence of H3K4me3, H3K27me3, or both (bivalent) ChIP-seq enriched regions in the +/−1 kb region relative to the transcriptional start site (TSS). ChIP-qPCR was performed on two independent sets of pooled B cell colonies from 3 wild-type and 3 Ts1Rhr mice. For analysis of upregulated genes in Ts1Rhr B cells, the 31 triplicated genes in Ts1Rhr mice were excluded. Data are presented as boxplots designating median (black line). 1 SD (box), and 2 SD (whiskers). E2A ChIP-Seq data from Rag1^{-/-} proB cells were obtained from GEO (GSE21978) (Lin et al. (2010) Nat. Immunol. 11:635-643) and mapped to the genome as above. Regions of enriched E2A genomic occupancy were defined using the MACS algorithm as above. Genes were considered associated with E2A if their gene body overlapped an E2A enriched region, or if their TSS was within 50 kb of an E2A enriched region, as was performed in Loven et al. (2013) Cell 153:320-334.

### Y. Statistical Analyses

Pairwise comparisons are represented as means+/−SEM by two-tailed Student t test, except where otherwise specified. Categorical variables were compared using a Fisher's exact test. Kaplan-Meier survival curves were compared using the log-rank test. In addition, RNA-seq. ChIP-seq, and microarray expression data are deposited with GEO under GEO accession number GSE48555.

### Example 2

### Analysis of DSCR Triplication Effects

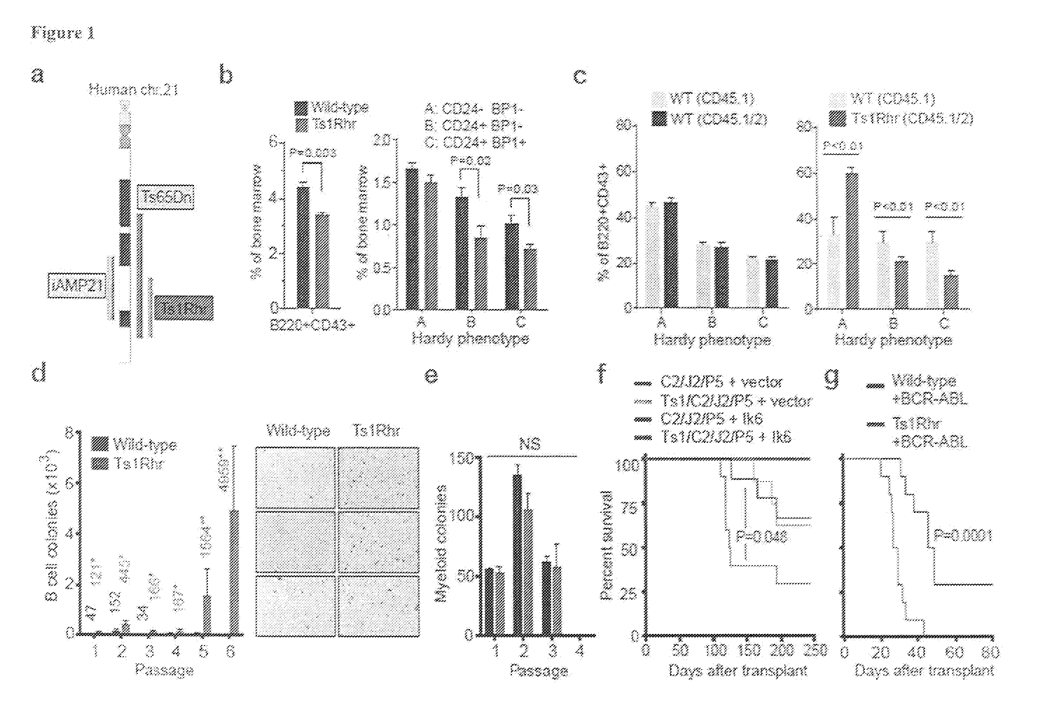

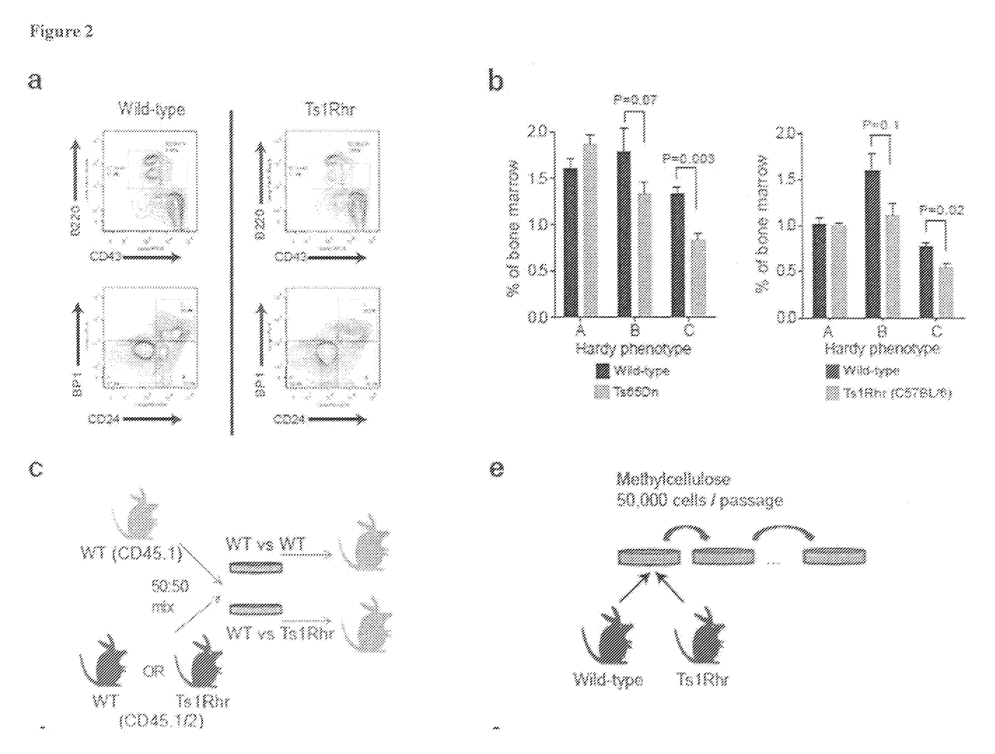

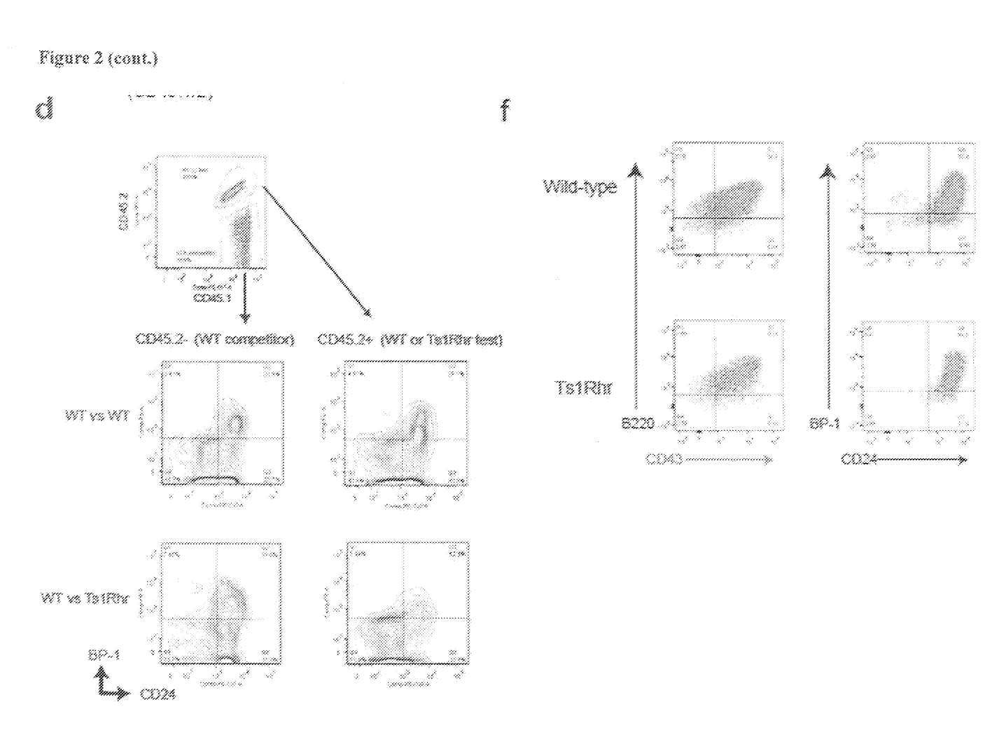

In order to directly interrogate the effects of polysomy 21, B cell development in Ts1Rhr mice (FIG. 1A), which harbor a triplication of 31 genes and one non-coding RNA on mouse chr.16 orthologous to human chr.21q22 (Olson et al. (2004) Science 306:687-690), was assayed. Bone marrow from 6-week-old Ts1Rhr mice had fewer total progenitor (B220+CD43+) B and pro-B (Hardy B and C) (Hardy et al. (1991) J. Exp. Med. 173:1213-1225) cells than wild-type littermates, while the pre-pro-B (Hardy A) fraction was unaffected (FIGS. 1B and 2A). CS7BL/6 Ts1Rhr, FVBxC57BL/6 F1 Ts1Rhr and Ts65Dn mice (Reeves et al. (1995) Nat. Genet. 11:177-184), which harbor a larger triplication (FIG. 1A), all had similar reductions in pro-B cells (FIG. 2B). This differentiation defect essentially phenocopies human fetal livers with trisomy 21, which have reduced pre-pro-B (CD34+CD19+CD10−) and pro-B cells (CD34+CD19+CD10+), as well as other hematopoietic defects (Roy et al. (2012) Proc. Natl. Acad. Sci. U.S.A. 109:17579-17584).

Competitive transplantation was performed using equal mixtures of congenic CD45.1 wild-type bone marrow and CD45.1/CD45.2 bone marrow from either Ts1Rhr or wild-type mice (FIG. 2C). After 16 weeks, recipients of wild-type CD45.1 and CD45.1/45.2 bone marrow had equal representations of both populations in Hardy A, B and C fractions, as well as whole bone marrow (FIGS. 1C and 2D). In contrast, mice that received wild-type CD45.1 mixed with Ts1Rhr CD45.1/45.2 recapitulated the Ts1Rhr defect, with significant reductions in CD45.1/45.2 Hardy B and C fractions (FIGS. 1C and 2D). Thus, the differentiation effect is independent of non-hematopoietic cells.

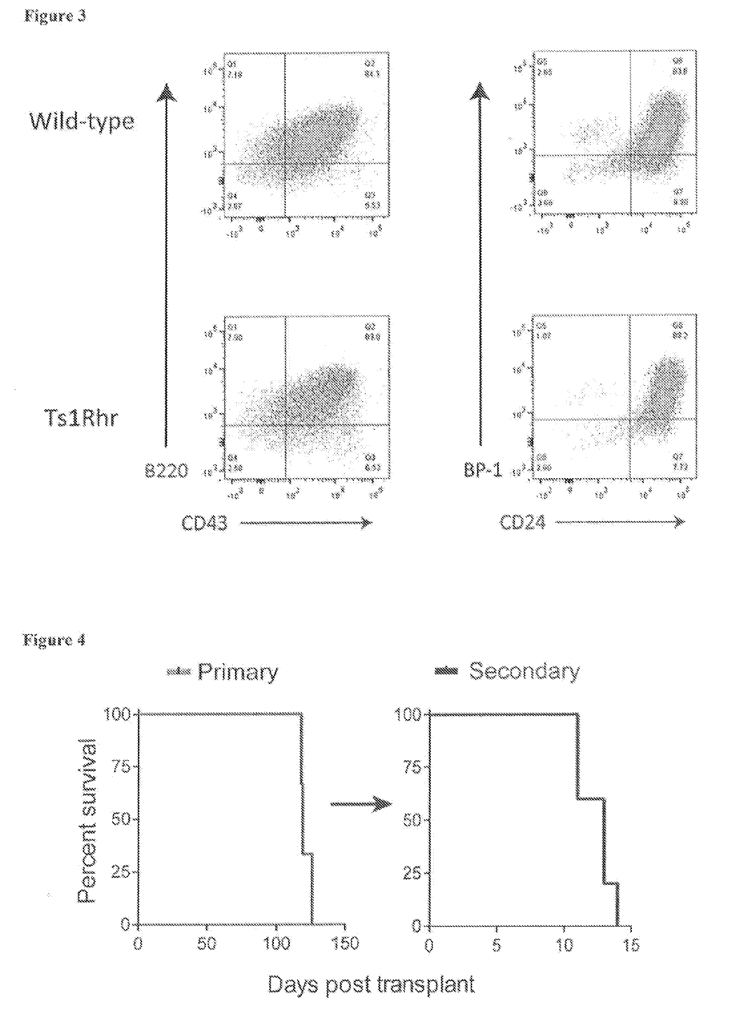

To address whether chr.21q22 directly confers transformed phenotypes like proliferation and self-renewal, progenitor B cell colonies were generated from unselected Ts1Rhr and wild-type bone marrow in three-dimensional cultures with IL7 (FIGS. 2E-2F). Wild-type bone marrow forms colonies (termed ‘passage 1’) under these conditions that can be replated to form new colonies for 1-2 additional passages. In contrast, Ts1Rhr bone marrow generated more colonies in early passages and serially replated indefinitely (FIG. 1D), which indicates self-renewal capacity. Both Ts1Rhr and wild-type colonies from early passages were universally Hardy C (CD24+BP−1+) by flow cytometry (FIG. 3). After passage 2, wild-type cells formed few if any colonies while Ts1Rhr cells obtained from all mice (n=9) expanded exponentially after passages 3 or 4 (FIG. 1D) and continued to repassage for more than 10 platings. In contrast, there were no significant differences between Ts1Rhr and wild-type bone marrow in the number or repassaging potential of myeloid colonies (FIG. 1E). Passage 6 B cells from Ts1Rhr bone marrow were capable of causing fatal lymphoproliferation in vivo upon injection into Nod.Scid.IL2Rγ^{-/-} mice and rapidly lethal B-ALL upon secondary transplantation into immunocompetent recipients (FIG. 4). Thus, DSCR triplication is sufficient to confer B cell self-renewal in vitro and that results in serially transplantable B-ALL in vivo.

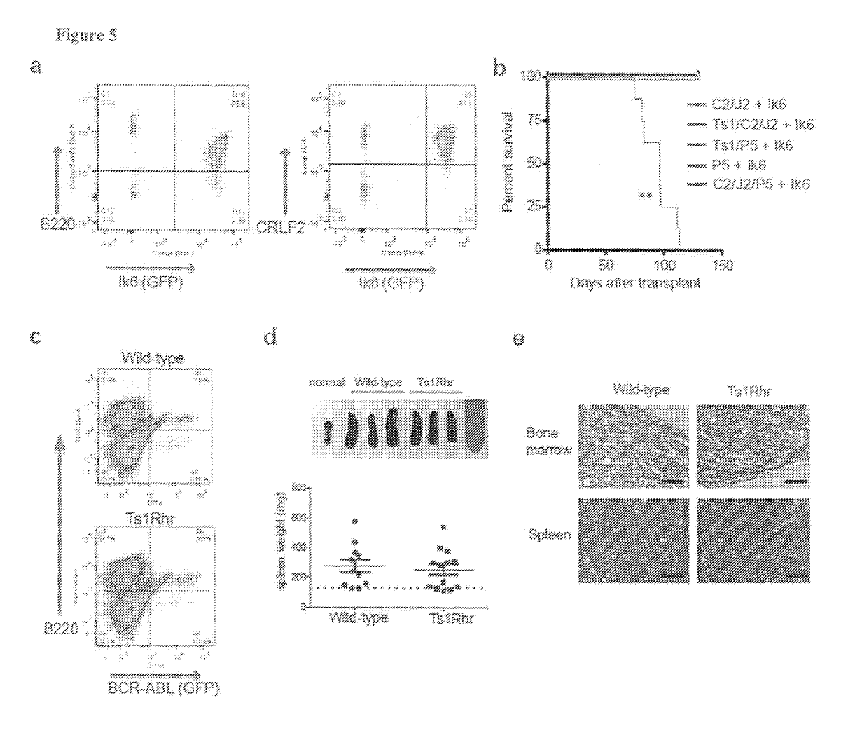

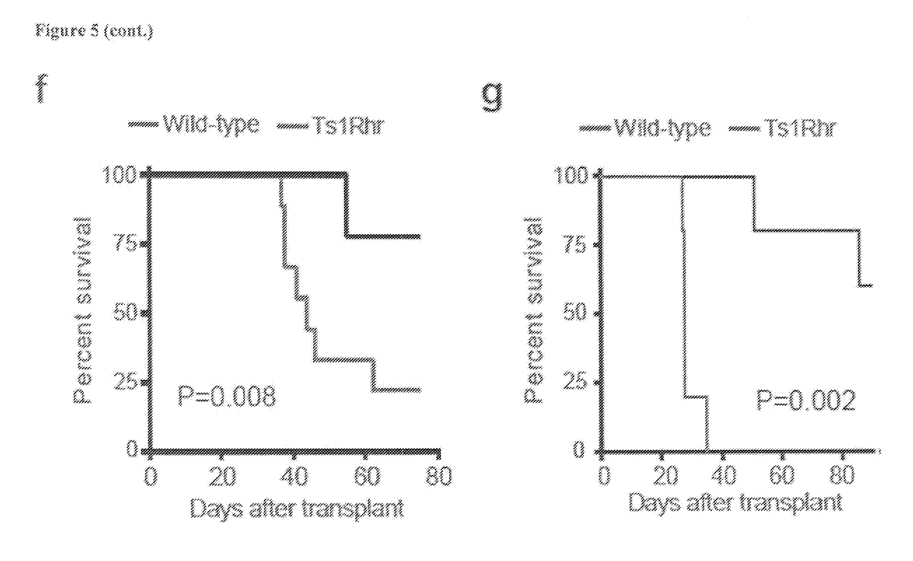

Sixty percent of DS-associated B-ALLs harbor rearrangements of CRLF2 that commonly occur in combination with activating JAK2 mutations (Mullighan et al. (2009) Nat. Genet. 41:1243-1246; Russell et al. (2009) Blood 114:2688-2698; Yoda et al. (2010) Proc. Natl. Acad. Sci. U.S.A. 107:252-257). To model this, Eμ-CRLF2 (hereafter ‘C2’) and Eμ-JAK2 R683G (‘J2’) transgenic mice, which have B-cell restricted transgene expression, were generated. C2/J2 mice did not develop B-ALL by 18 months of age, nor did C2/J2 mice crossed to Pax5^{+/-} mice. Transduction of C2/J2/Pax5^{+/-} bone marrow with a dominant-negative IKZF1 allele (Ik6) (Iacobucci et al. (2008) Blood 112:3847-3855) and transplantation into wild-type recipients resulted in CRLF2-positive B-ALL in all mice by 120 days (FIGS. SA-5B). Control mice lacking C2, J2 or Pax5 heterozygosity did not develop B-ALL with Ik6 (FIG. 5B), thus establishing this transgenic combination as the first model of CRLF2/JAK2-driven B-ALL. To assess the effect from the addition of chr.21q22 triplication. C2/J2/Pax5^{+/-} and Ts1Rhr/C2/J2/Pax5^{+/-} mice were transduced with a lower titer of either empty virus or Ik6 virus. Mice transplanted with Ts1Rhr/C2/J2/Pax5^{+/-} bone marrow transduced with Lk6 developed B-ALL with greater penetrance and reduced latency compared to C2/J2Pax5^{+/-} alone (FIG. 1F). The same genotypes (C2/J2Pax5^{+/-}/Ik6 with or without polysomy 21) occur in high-risk cases of human B-ALL (Mullighan et al. (2009) Proc. Natl. Acad. Sci. U.S.A. 106:9414-9418), supporting the validity of the model.

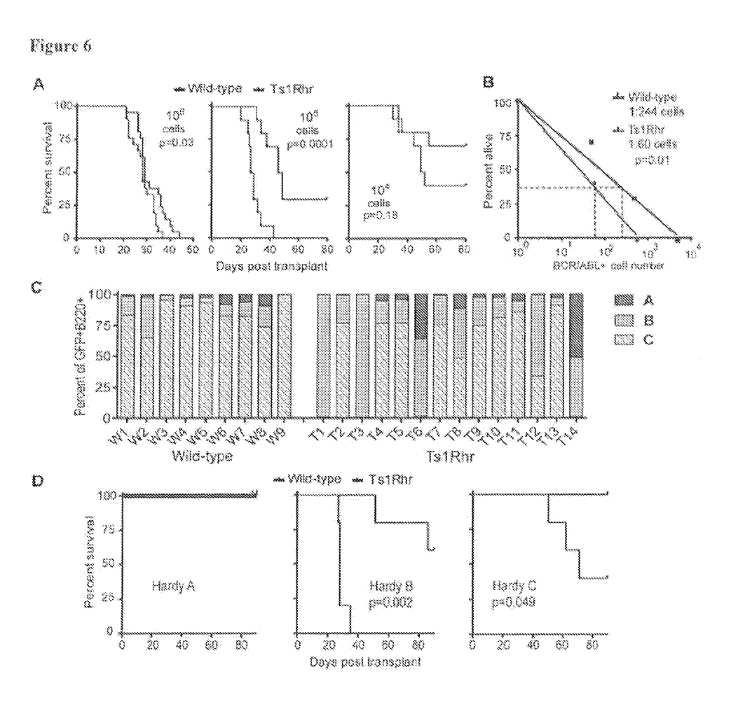

To confirm the contribution of chr.21q22 triplication in a more tractable model, B-ALL was induced by transplanting unselected bone marrow transduced with p210 BCR-ABL (Krause et al. (2006) Nat. Med. 12:1175-1180). Although BCR-ABL ALL is uncommon in children with DS, polysomy 21 is the most common somatic aneuploidy among BCR-ABL ALLs (Wetzler et al. (2004) Br. J. Haematol. 124:275-288). Limiting dilution analysis was performed by transplanting 10⁶, 10⁵ or 10⁴ transduced bone marrow cells from Ts1Rhr mice or wild-type littermates into wild-type recipients (FIG. 6A). Ts1Rhr and wild-type bone marrow had similar transduction efficiencies (FIG. 5C), but mice (CS7BL/6 and FVBxC57BL/6 F1 backgrounds) that received transduced Ts1Rhr bone marrow succumbed to B-ALL with shorter latency and increased penetrance (FIGS. 1G and 5D-5F). Specifically, three weeks after transplantation, mice that received transduced Ts1Rhr bone marrow had higher white blood cell counts and lower hemoglobin concentrations in peripheral blood compared with mice that received transduced wild-type bone marrow (FIG. 7).

Mice transplanted with either wild-type or Ts1Rhr bone marrow succumbed to progenitor (B220+ CD43+) B-ALLs with similar histology that infiltrated the bone marrow and spleen (FIG. 5D-5E). However. B-ALLs in mice transplanted with Ts1Rhr marrow developed with shorter latency and, in cohorts transplanted with 10⁵ or 10⁴ cells, increased penetrance (FIGS. 6A and 5F). Based on a Poisson distribution analysis, the frequency of B-ALL-initiating cells was over 4-fold higher in Ts1Rhr bone marrow (FIG. 6B; 1:244 versus 1:60 transduced cells, p=0.01). B-ALLs (based on GFP+/B220+ phenotype) derived from wild-type bone marrow were homogenous populations of CD24+BP-1+ (equivalent to Hardy C) cells. In contrast, nearly one-half of B-ALLs derived from Ts1Rhr bone marrow were primarily CD24+BP-1− (Hardy B; FIG. 6C, p=0.003 compared to wild-type by Wilcoxon rank sum test), with some cases harboring CD24−BP−1− (Hardy A) cells.

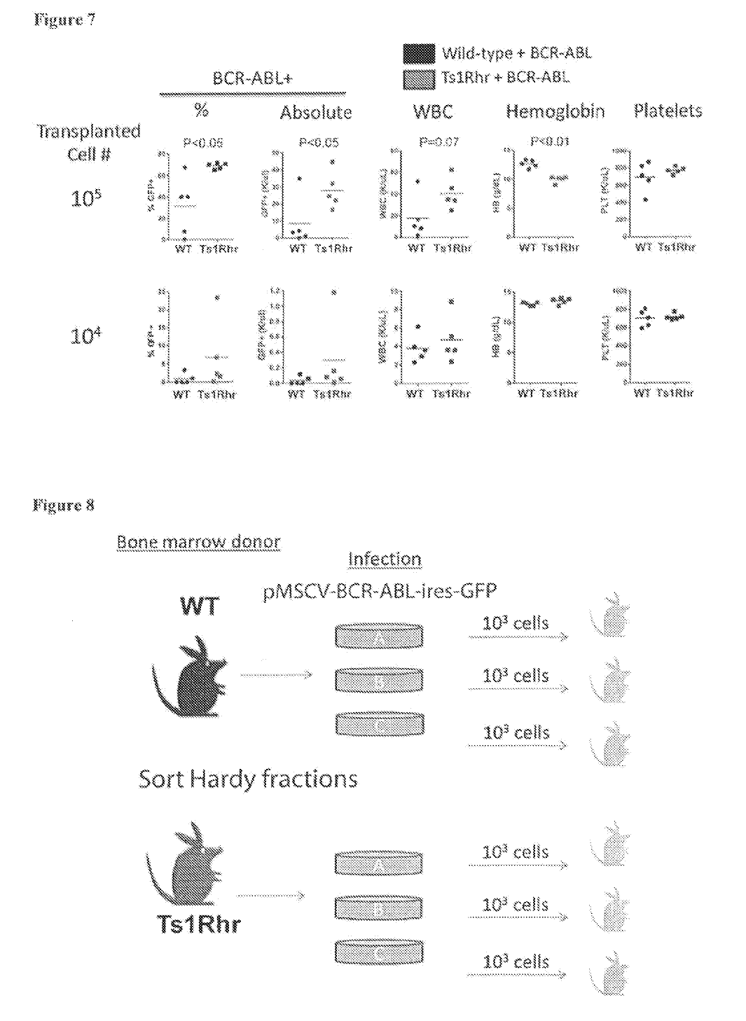

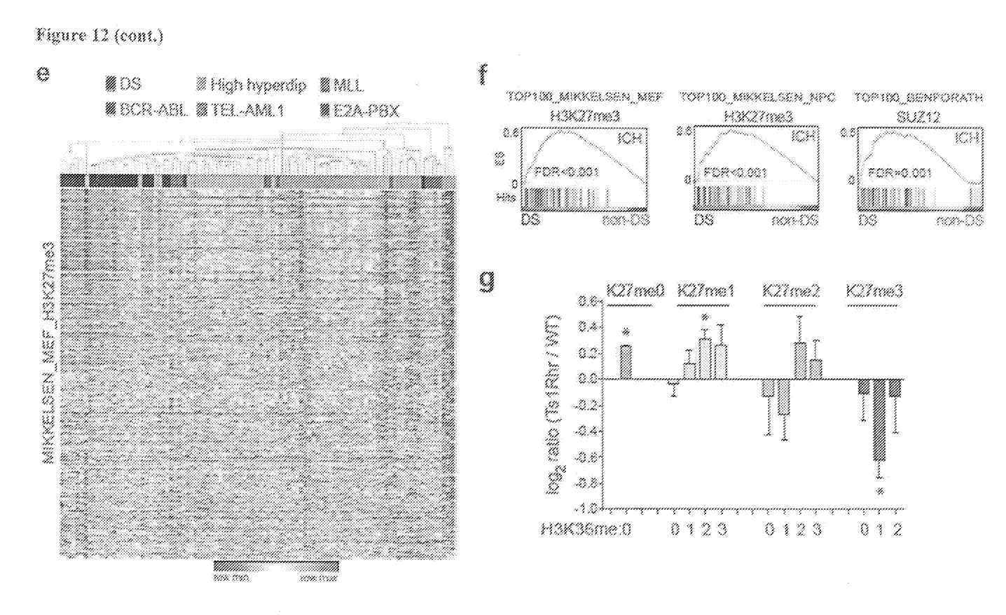

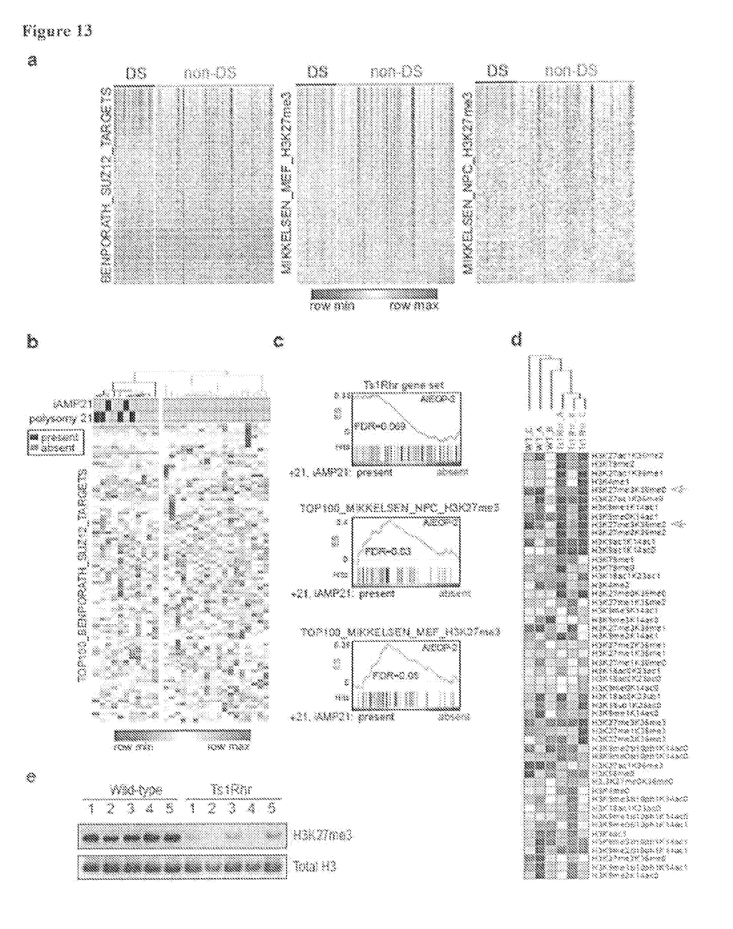

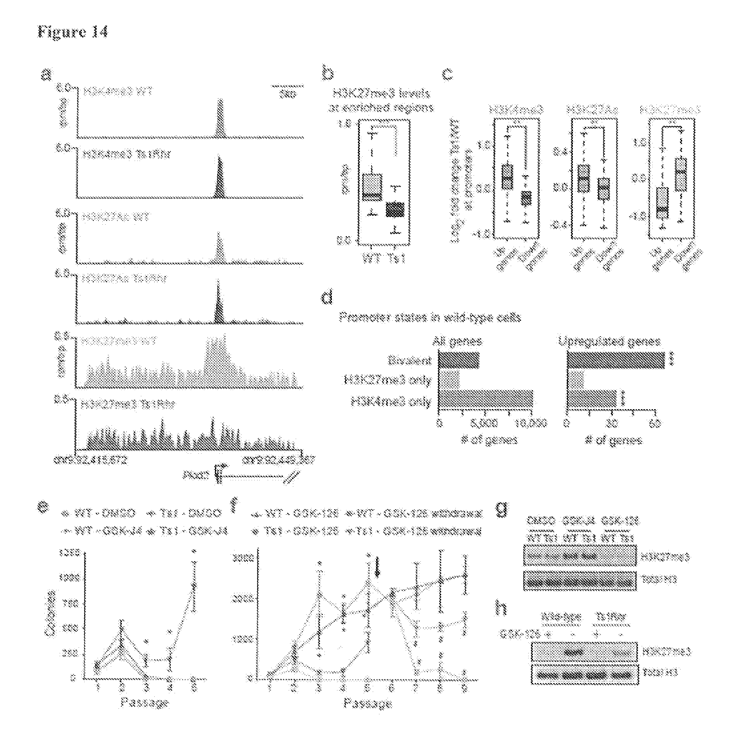

The difference in B-ALL differentiation phenotype raised the possibility that DSCR triplication affects the B cell stage that is transformed by BCR-ABL. To address this, Hardy A, B and C fractions were sorted from Ts1Rhr and wild-type bone marrow, individually transduced with BCR-ABL, and then transplanted 10³ cells into wild-type recipients (FIG. 8). As with unsorted bone marrow (FIG. 6A), B-ALLs developed with greater penetrance and shorter latency among mice transplanted with transduced Ts1Rhr Hardy B cells (p=0.002 by log-rank test: FIG. 6D) compared with transduced wild-type Hardy B cells. B-ALL also developed in mice transplanted with transduced Ts1Rhr Hardy C cells but not wild-type (p=0.049; FIG. 14D), although with longer latency than among mice transplanted with transduced Ts1Rhr Hardy B cells (p=0.002 for Ts1rhr Hardy B versus Hardy C). No mice transplanted with transduced Hardy A cells from either genotype developed B-ALL (FIG. 6D). Thus, DSCR triplication promotes BCR-ABL transformation in both Hardy B and Hardy C fractions, despite the in vivo reduction in absolute numbers of these cells in Ts1Rhr bone marrow (FIG. 1B). These sorting experiments also confirm that the increased leukemogenesis induced with BCR-ABL, like the differentiation abnormality, is a B cell autonomous effect of DSCR triplication.

Transplantation of BCR-ABL-transduced sorted Hardy B cells from Ts1Rhr or wild-type mice recapitulated the same effect (FIG. 5G), indicating that the leukemogenic effect from chr.21q22 triplication is progenitor B-cell autonomous.

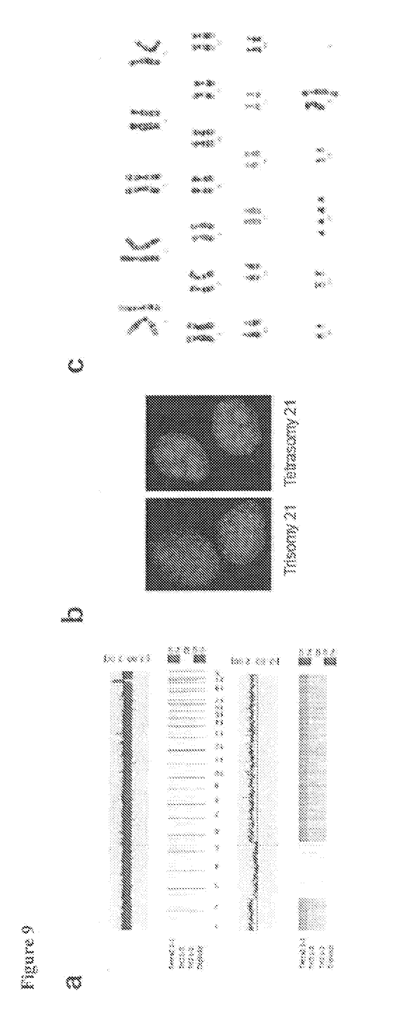

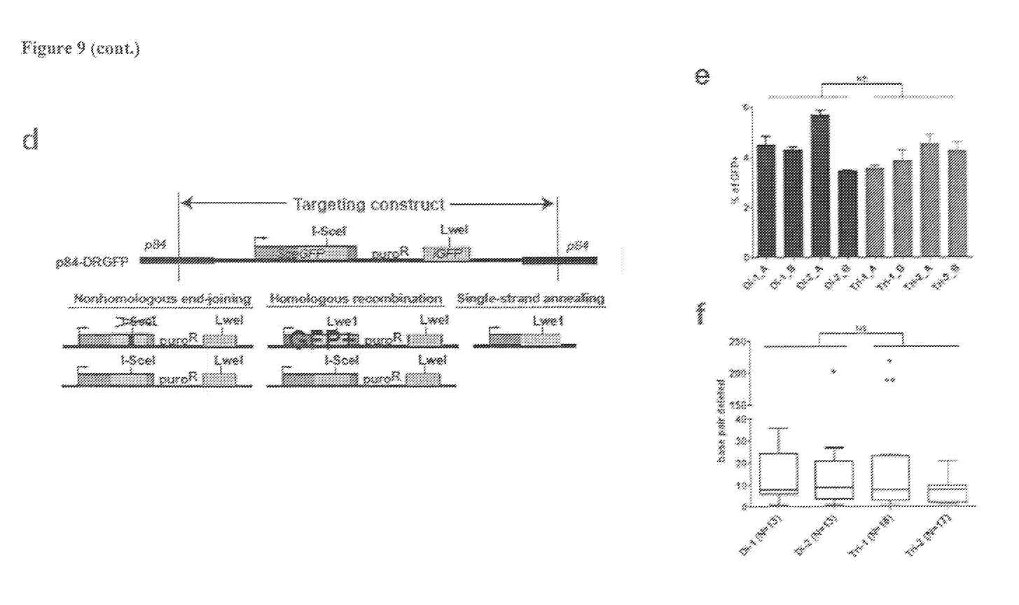

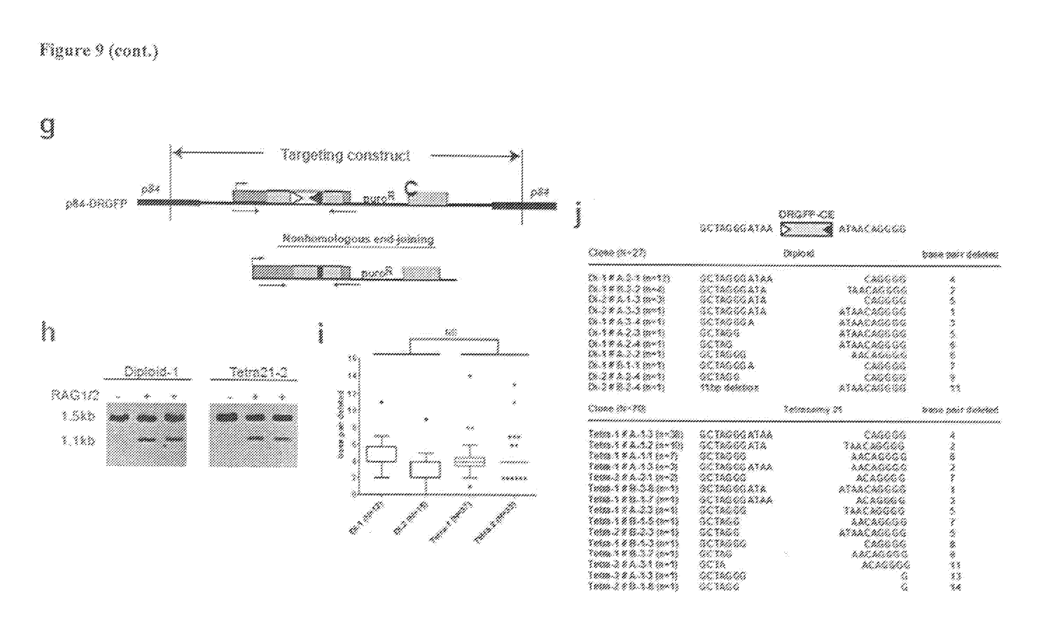

In addition to these direct effects, polysomy 21 could also contribute to B cell transformation by promoting aberrant DNA double-strand break repair (DSBR), which mediates leukemogenic alterations at CRLF2, IKZF1, PAX5 and other loci (Mullighan et al. (2009) Natl. Genet. 41:1243-1246: Russell et al. (2009) Blood 114:2688-2698; Yoda et al. (2010) Proc. Natl. Acad. Sci. U.S.A. 107:252-257). To address this, otherwise isogenic retinal pigment epithelial (RPE) cells that harbor 2, 3 or 4 copies of human chr.21 by microcell-mediated chromosomal transfer were generated (FIGS. 9A-9C). Zinc finger nuclease-mediated recombination was used to target DSBR reporters (Weinstock and Jasin (2006) Mol. Cell Biol. 26:131-139) to the p84 locus of cells with different numbers of chr.21, which avoids confounding locus-specific differences (Smith et al. (2008) Stem Cells 26:496-504). Polysomy 21 had no effect on either homology-directed repair frequency or junction characteristics formed by nonhomologous end-joining, whether DSBs were induced by the I-SceI endonuclease (FIGS. 9D-9F) or by the V(D)J recombinase (FIGS. 9G-9J). Although a subtle defect or one specific to progenitor B cells remains possible, these results indicate for the first time in an isogenic system that polysomy 21 does not drastically affect DSBR phenotype.

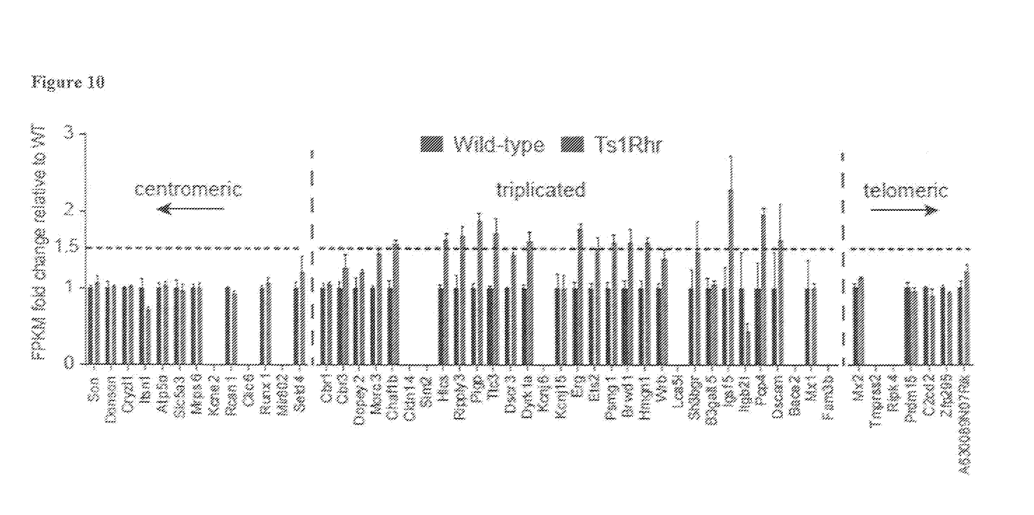

Whole transcriptome sequencing (RNA-seq) of passage 1 B cells was also performed; triplicated loci in Ts1Rhr cells were expressed at approximately 1.5-fold higher levels compared to wild-type cells (FIG. 10) while absolute expression among the 25 genes differed markedly (FIG. 11). A transcriptional “Ts1Rhr gene set” of the 150 most differentially expressed genes compared to wild-type was defined (Table 1). As expected, this signature was highly enriched by gene set enrichment analysis (GSEA) (Subramanian et al. (2005) J. Proc. Natl. Acad. Sci. U.S.A. 102:15545-15550) for human chr.21q22 genes (Table 2), but not other human chromosomal segments, based on a query of the Broad Institute Molecular Signatures Database (MSigDB) “c1” positional dataset (Subramanian et al. (2005) Proc. Natl. Acad. Sci. U.S.A. 102:15545-15550). The Ts1Rhr gene set was next applied to a gene expression dataset of pediatric B-ALLs (AIEOP) (Hertzberg et al. (2010) Blood 115:1006-1017). The Ts1Rhr B cell signature was enriched among DS-ALLs by GSEA (FIGS. 12A-12B; FDR=0.019), indicating that transcriptional differences defined in Ts1Rhr B cells are biologically relevant to human DS-ALL. By hierarchical clustering, a “core Ts1Rhr set” of only 50 genes (Table 1) was observed that distinguished DS-ALLs (FIG. 12A). Although none of the 50 genes are triplicated in Ts1Rhr cells, the core Ts1Rhr set was highly enriched among DS-ALLs in both the AIEOP dataset (FIG. 12B; FDR=0.001) and an independent validation dataset (ICH) (FIG. 12C; FDR=0.001).

To identify pathways perturbed by chr.21q22 triplication, the Ts1Rhr gene set was queried against >3000 functionally defined gene sets in the MSigDB “c2” chemical and genetic perturbations and “c6” oncogenic signatures repositories (Subramanian et al. (2005) Proc. Natl. Acad. Sci. U.S.A. 102:15545-15550). Arranging the significant gene sets in a network enrichment map (Merico et al. (2010) PLoS One 5:e13984) defined 4 clusters (FIG. 12D). The most highly enriched cluster consisted of polycomb repressor complex 2 (PRC2) targets and sites of tri-methylated histone H3K27 (H3K27me3), the repressive mark added by PRC2, that were defined across multiple lineages (Table 3). The additional clusters consisted of gene sets that distinguish either stem cells from lineage-matched differentiated cells, cancer cells from nonmalignant cells, or less differentiated from more differentiated lymphoid cells (Table 3).

It was next asked whether differential expression of PRC2/H3K27me3-classified genes would distinguish DS-ALLs from other B-ALLs. A previous effort using genome-wide expression in the AIEOP cohort failed to define a transcriptional signature specific to DS-ALL (Hertzberg et al. (2010) Blood 115:1006-1017). Strikingly, expression of H3K27me3 targets defined in murine embryonic fibroblasts distinguished DS-ALLs from non-DS-ALLs (FIG. 12E). To validate these findings, the 100 most differentially expressed genes between DS-ALLs and non-DS-ALLs in the AIEOP cohort across three different PRC2/H3K27me3 signatures were determined (FIG. 13A and Table 5). All three signatures were significantly enriched (FDR≦0.001) among DS-ALLs in the ICH validation cohort (FIG. 12F). In a third cohort of non-DS-ALLs (AIEOP-2), cases with either polysomy 21 or iAMP(21) clustered based on expression of PRC2 targets (FIG. 13B, P=0.001 by Fisher's exact test), and the Ts1Rhr and H3K27me3 gene sets were enriched among cases with polysomy 21 or iAMP(21) by GSEA (FIG. 13C).

Genes from PRC2/H3K27me3 gene sets that distinguish DS-ALLs are predominantly overexpressed in DS-ALL (FIGS. 12E and 13A). This indicates that DS-ALL is associated with de-repression of PRC2 targets and reduced H3K27me3. Consistent with the GSEA, histone H3 mass spectrometry demonstrated a global reduction in H3K27me3 peptides in passage 1 Ts1Rhr B cells compared to wild-type cells, with reciprocal increases in unmethylated and monomethylated H3K27 peptides (FIG. 12G). BCR-ABL B-ALLs from Ts1Rhr bone marrow also had reduced H3K27me3 by both mass spectrometry and immunoblotting (FIGS. 13D-13E). Thus, triplication of only 31 genes directly suppresses H3K27me3.

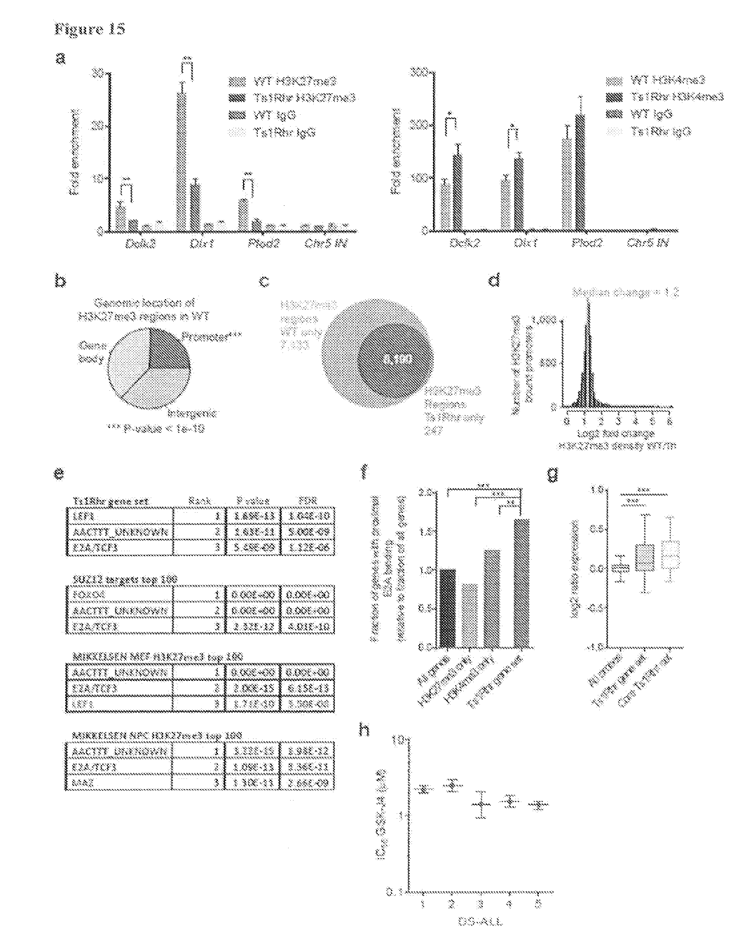

To identify mechanisms that directly link gene triplication, H3K27me3 levels, and gene expression. ChIP-seq of passage 1 Ts1Rhr and wild-type B cells was performed. Ts1Rhr B cells had a genome-wide reduction of H3K27me3 at regions enriched for this mark in wild-type cells (FIGS. 14A-14B) that was confirmed at multiple loci by ChIP followed by quantitative PCR (FIG. 15A). Within Ts1Rhr B cells, H3K27me3 was found almost exclusively at regions enriched for H3K27me3 in wild-type cells, suggesting little or no redistribution but rather a global reduction in the H3K27me3 density (FIGS. 15B-15D). As expected, reciprocal changes in activating (H3K4me3, H13K27ac) and repressive (H3K27me3) marks were observed at promoters of genes differentially expressed in Ts1Rhr B cells (FIG. 14C). However, genes “bivalently marked” with both H3K27me3 and H3K4me3 in wild-type cells were highly enriched among those overexpressed in Ts1Rhr B cells (FIG. 14D; P<0.0001).

Bivalent marks may indicate genes that are modulated during lineage-specific differentiation (Bernstein et al. (2006) Cell 125:315-326). The enrichment of bivalently-marked genes within the Ts1Rhr gene set therefore suggests that the global loss of H3K27me3 from chr.21q22 triplication selectively drives the overexpression of genes defined by a progenitor B cell-specific developmental program. In support of this, the Ts1Rhr and PRC2/H3K27me3 gene sets were highly enriched for predicted binding sites of the master B cell transcription factors E2A/TCF3 and LEF1 (FIG. 15E) (Kruidenier et al. (2012) Nature 488:404-408; McCabe et al. (2012) Nature 492:108-112). To test whether the Ts1Rhr gene set is enriched for functional E2A/TCF3 targets, a previously reported dataset of ChIP-seq and gene expression from wild-type and E2A^{-/-} murine B cell progenitors (Kruidenier et al. (2012) Nature 488:404-408) was analyzed. Genes within the Ts1Rhr gene set had increased proximal occupancy by E2A/TCF3 (FIG. 15F). In addition, the expression of genes within both the Ts1Rhr gene set and the core Ts1Rhr set was preferentially increased in the presence of E2A/TCF3 (FIG. 15G).

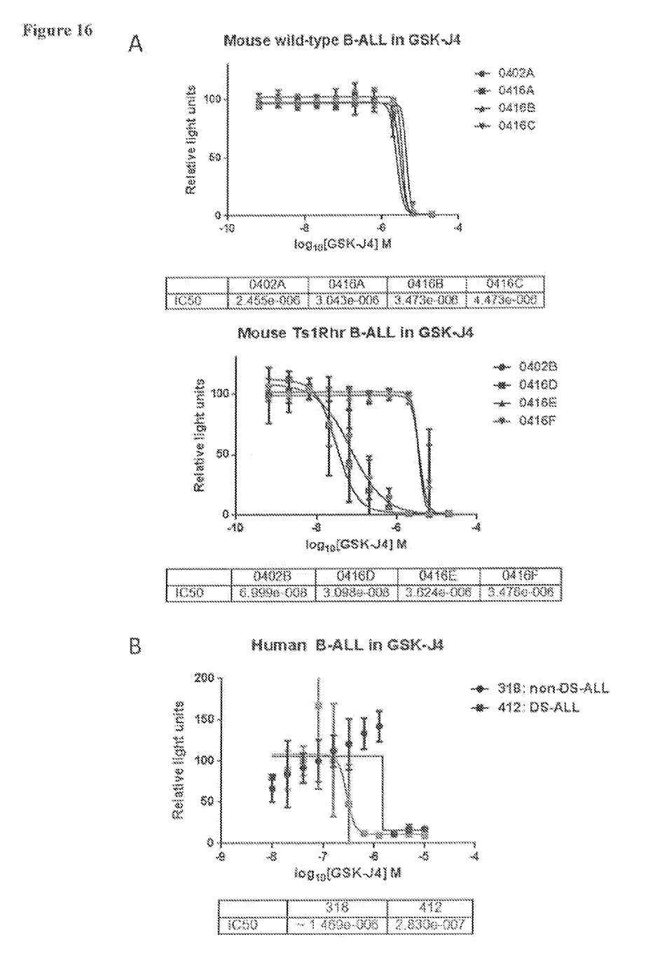

It was next asked whether pharmacologic restoration of H3K27me3 with GSK-J4 (Kruidenier et al. (2012) Nature 488:404-408), a selective inhibitor of H3K27 demethylases, would block Ts1Rhr B cell repassaging. GSK-J4 increased H3K27me3 in Ts1Rhr B cells, decreased colony-forming activity, and blocked indefinite repassaging (FIGS. 14E and 14G). Previous studies demonstrated that 10 μM GSK-J4 reduces lipopolysaccharide-induced proinflammatory cytokine production by human primary macrophages (Kruidenier et al. (2012) Nature 488:404-408). IC₅₀ values for GSK-J4 across a panel of DS-ALLs ranged from 1.4-2.5 μM (FIG. 15H). Treatment with GSK-126²⁵, a selective inhibitor of the PRC2 catalytic subunit EZH2, decreased H3K27me3 and was sufficient to confer indefinite repassaging in wild-type B cells (FIGS. 14F-14G). In addition, murine and human B-cell ALLs harboring increased copies of the Down syndrome critical region were more sensitive to GSK-J4 than to leukemias lacking such increased copies in a limited set of leukemias analyzed (FIG. 16). Both the loss of H3K27me3 and indefinite repassaging were reversible upon withdrawal of GSK-126 from wild-type cells (FIGS. 14F and 14H).

Among the 31 triplicated genes in Ts1Rhr cells is Hmgn1, which encodes a nucleosome binding protein that modulates transcription and promotes chromatin decompaction (Catez et al. (2002) EMBO Rep. 3:760-766; Rattner et al. (2009) Mol. Cell 34:620-626). Modest increases in HMGN1 induce changes in histone H3 modifications and gene expression (Lim et al. (2005) EMBO J. 24:3038-3048: Rochman et al. (2011) Nucl. Acids Res. 39:4076-4087).

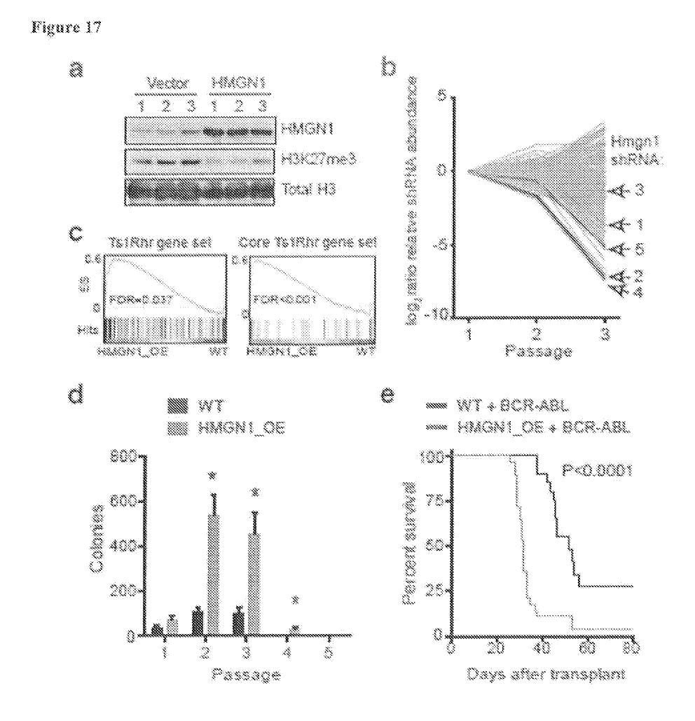

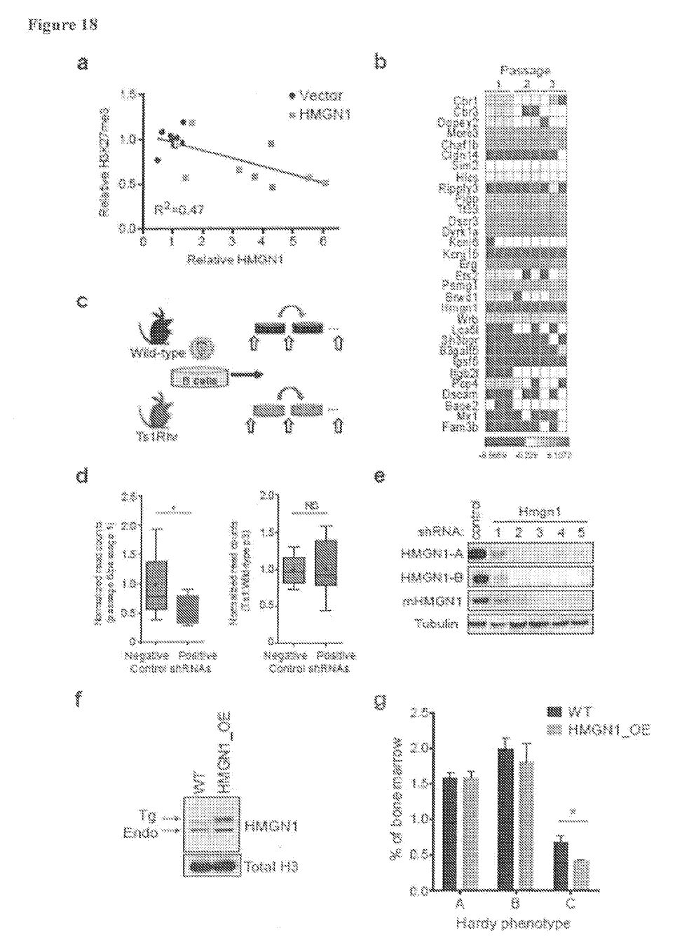

Overexpression of HMGN1 in murine Ba/F3 B cells suppressed H3K27me3 in a dose-dependent fashion (FIGS. 17A and 18A). By RNA-seq, Hmgn1 was one of only seven triplicated genes that maintained >70% of its passage 1 expression level at passages 3 and 6 in all Ts1Rhr replicates (FIG. 18B), indicating that it may be necessary for serial repassaging. To address this, 5 shRNA targeting each of the 31 triplicated genes and controls were individually transduced into Ts1Rhr and wild-type passage 1B cells (FIG. 18C). Transduced cells were pooled and passaged in adequate numbers to ensure that each shRNA was represented, on average, in 2200 colonies at each passage. The relative abundance of each shRNA at each passage was deconvoluted by next-generation sequencing.

As expected, positive control shRNAs that reduce viability across cell types were equally depleted at later passages from Ts1Rhr and wild-type backgrounds (FIG. 18D and Table 6). Among shRNAs against triplicated genes, two of the top four that most selectively depleted Ts1Rhr B cells targeted Hmgn1 and the remaining three shRNAs against Hmgn1 all scored as preferentially toxic in Ts1Rhr B cells (FIG. 17B and Table 6). By passage 6, all 5 shRNAs against Hmgn1 were depleted by an average of >99% across replicates. All five shRNAs reduced HMGN1 protein in Ba/F3 cells (FIG. 18E). Together, these data indicate that HMGN1 contributes to the repassaging phenotype of Ts1Rhr B cells.

To directly address the sufficiency of HMGN1 overexpression for effects observed in Ts1Rhr cells, mice with transgenic overexpression of human HMGN1 (HMGN1_OE) at levels comparable to mouse HMGN1 were analyzed (FIG. 18F) (Bustin et al. (1995) DNA Cell Biol. 14:997-1005). A gene expression signature of HMGN1_OE passage 1 B cells (compared to littermate controls) was highly enriched for the Ts1Rhr and core Ts1Rhr gene sets (FIG. 17C). Compared to control bone marrow, HMGN1_OE bone marrow had reduced Hardy C cells in vivo (FIG. 18G), generated more B cell colonies in passages 1-4 in vitro (FIG. 17D), and resulted in greater penetrance and shorter latency of BCR-ABL-induced B-ALL (FIG. 17E). Thus, overexpression of HMGN1 alone recapitulates transcriptional and phenotypic alterations observed from triplication of all 31 Ts1Rhr genes.

In conclusion, it has been described herein that triplication of chr.21q22 genes confers cell autonomous differentiation and transformation phenotypes in progenitor B cells. By first delineating these biologic consequences of chr.21q22 triplication, human B-ALL datasets were more effectively interrogated and it was demonstrated that DS-ALLs are distinguished by the overexpression of H3K27me3-marked genes. The data also highlight the therapeutic potential of H3K27 demethylase inhibitors for B-ALLs with extra copies of chr.21q22. At the same time, inhibitors of EZH2 are believed to be useful for in vitro or in vivo expansion of precursor B cells. Finally, the nucleosome remodeling protein HMGN1 promotes the in vitro passaging of B cells, suppresses global H3K27me3 and functions as a cooperating oncogene in vivo.

TABLE 1

| Human | | Mouse | | | Triplicated | log2 | | adj. P. Val | Core_ |
|---|---|---|---|---|---|---|---|---|---|
| Symbol | Human Transcript | Symbol | Mouse Transcript | Direction | gene | FC_Ts1vsWT | P. Value | (FDR) | Gene_Set |
| PCDH17 | NM_001040429.2 | Pcdh17 | NM_001013753.2 | Up | no | 1.54637752 | 1.80E−11 | 9.18E−09 | no |
| MYOM2 | NM_003970.2 | Myom2 | NM_008664.2 | Up | no | 1.52163934 | 6.43E−12 | 3.88E−09 | no |
| ACPP | NM_001134194.1 | Acpp | NM_019807.2 | Up | no | 1.46890123 | 1.61E−11 | 8.44E−09 | no |
| FZD6 | NM_003506.3 | Fxd6 | NM_001162494.1 | Up | no | 1.44567633 | 4.67E−11 | 1.86E−08 | no |
| TDRD9 | NM_153046.2 | Tdrd9 | NM_029056.1 | Up | no | 1.44375967 | 7.19E−09 | 1.43E−06 | Core |
| SYTL4 | NM_080737.2 | Sytl4 | NM_013757.1 | Up | no | 1.40346946 | 5.12E−11 | 1.93E−08 | no |
| C15orf48 | NM_197955.2 | AA467197 | NM_001004174.1 | Up | no | 1.39700365 | 5.76E−11 | 2.13E−08 | no |
| RBM44 | NM_001080504.2 | Rbm44 | NM_001033408.4 | Up | no | 1.32364628 | 5.78E−07 | 5.16E−05 | no |
| PXDN | NM_012293.1 | Pxdn | NM_181395.2 | Up | no | 1.3101789 | 4.68E−09 | 1.00E−06 | no |
| SCN4B | NM_001142349.1 | Scn4b | NM_001013390.2 | Up | no | 1.28105823 | 2.14E−10 | 7.36E−08 | Core |
| TMEM132E | NM_207313.1 | Tmem132e | NM_023438.2 | Up | no | 1.26622663 | 9.60E−10 | 2.69E−07 | Core |
| MAGI1 | NM_001033057.1 | Magi1 | NM_001083320.1 | Up | no | 1.20490304 | 1.29E−07 | 1.55E−05 | no |
| C6orf222 | NM_001010903.4 | 4930539E08Rik | NM_172450.3 | Up | no | 1.20124159 | 6.63E−07 | 5.72E−05 | no |
| ACY3 | NM_080658.1 | Acy3 | NM_027857.3 | Up | no | 1.19197028 | 2.78E−08 | 4.94E−06 | no |
| PRDM16 | NM_199454.2 | Prdm16 | NM_001177995.1 | Up | no | 1.18197006 | 4.15E−07 | 3.88E−05 | Core |
| ATP2B2 | NM_001683.3 | Atp2b2 | NM_001036684.2 | Up | no | 1.14564423 | 5.34E−07 | 4.84E−05 | Core |
| TMEM121 | NM_025268.2 | tmem121 | NM_153776.2 | Up | no | 1.13940458 | 1.18E−05 | 0.00054664 | no |
| NKG7 | NM_005601.3 | Nkg7 | NM_024253.4 | Up | no | 1.08533792 | 8.13E−05 | 0.00046959 | no |
| CMTM8 | NM_178868.3 | Cmtm8 | NM_027294.2 | Up | no | 1.07275242 | 1.22E−05 | 0.00066417 | no |
| PCDH11X | NM_032967.2 | Pcdh11x | NM_001081385.1 | Up | no | 1.06458925 | 8.51E−07 | 6.98E−05 | Core |
| CACNA2D1 | NM_000722.2 | Cacna2d1 | NM_001110843.1 | Up | no | 1.04834358 | 2.94E−07 | 3.05E−05 | Core |
| MYLK | NM_053028.3 | Mylk | NM_139300.3 | Up | no | 1.01270212 | 5.25E−06 | 0.00032376 | no |
| PCP4 | NM_006198.2 | Pcp4 | NM_008791.2 | Up | yes | 0.966712 | 7.24E−05 | 0.00043452 | no |
| C11orf63 | NM_024806.3 | 4931429I11Rik | NM_001081121.1 | Up | no | 0.96016163 | 7.95E−06 | 0.00046046 | Core |
| PRG3 | NM_006093.3 | Prg3 | NM_016914.2 | Up | no | 0.94542206 | 0.00011191 | 0.00419953 | no |
| IFI44 | NM_006417.4 | Ifi44 | NM_133871.2 | Up | no | 0.92945842 | 0.00014781 | 0.00529738 | no |
| PTPN14 | NM_005401.4 | Ptpn14 | NM_008976.2 | Up | no | 0.89974845 | 6.36E−06 | 0.00038857 | no |
| IL20RB | NM_144717.3 | Il20rb | NM_001033543.3 | Up | no | 0.89580175 | 6.47E−05 | 0.00268723 | Core |
| CXCR1 | NM_000634.2 | Cxcr1 | NM_178241.4 | Up | no | 0.88760509 | 0.00015691 | 0.00556533 | no |
| DDC | NM_001082971.1 | Ddc | NM_016672.4 | Up | no | 0.8808195 | 4.74E−05 | 0.00210826 | Core |
| NKD2 | NM_033120.3 | Nkd2 | NM_028186.4 | Up | no | 0.86909226 | 7.48E−05 | 0.0030674 | no |
| ZNF354B | NM_058230.2 | Zfp354b | NM_013744.3 | Up | no | 0.86163388 | 0.00065595 | 0.01576727 | no |
| ERG | NM_182918.3 | Erg | NM_133659.2 | Up | yes | 0.85374931 | 1.64E−05 | 0.00084282 | no |
| LRCH2 | NM_020871.3 | Lrch2 | NM_001081173.1 | Up | no | 0.84497707 | 0.00046692 | 0.01279818 | Core |
| STAT4 | NM_003151.3 | Stat4 | NM_011487.4 | Up | no | 0.83655099 | 5.30E−05 | 0.00226171 | no |
| KCNB1 | NM_004975.2 | Kcnb1 | NM_008420.4 | Up | no | 0.82587579 | 0.00099575 | 0.02254804 | Core |
| DOCK9 | NM_001130049.1 | Dock9 | NM_001128308.1 | Up | no | 0.81795827 | 3.67E−05 | 0.00169515 | no |
| COL5A3 | NM_015719.3 | Col5a3 | NM_016919.2 | Up | no | 0.81786946 | 4.40E−05 | 0.00199197 | no |
| ESAM | NM_138961.2 | Esam | NM_027102.3 | Up | no | 0.81524283 | 0.00025927 | 0.00840072 | no |
| NRXN1 | NM_004801.4 | Nrxn1 | NM_177284.2 | Up | no | 0.80477272 | 0.00146234 | 0.02925706 | Core |
| GIMAP4 | NM_018326.2 | Gimap4 | NM_174990.4 | Up | no | 0.80190397 | 7.87E−05 | 0.00319996 | no |
| TTC3 | NM_001001894.1 | Ttc3 | NM_009441.2 | Up | yes | 0.80169789 | 5.23E−05 | 0.00223751 | no |
| SLC17A8 | NM_139319.2 | Slc17a8 | NM_182959.3 | Up | no | 0.79115392 | 0.00155742 | 0.03094187 | Core |
| ANXRD6 | NM_014942.4 | Ankrd6 | NM_001012451.1 | Up | no | 0.78954546 | 0.0001254 | 0.00461048 | no |
| HMGCLL1 | NM_019036.2 | Hmgcll1 | NM_173731.2 | Up | no | 0.78553343 | 0.0001186 | 0.0044061 | Core |
| INSM1 | NM_002196.2 | Insm1 | NM_016889.3 | Up | no | 0.78410064 | 0.00237016 | 0.04067418 | Core |
| SLCO5A1 | NM_030958.2 | Slco5a1 | NM_172841.2 | Up | no | 0.78265899 | 0.00229616 | 0.04067418 | Core |
| BRWD1 | NM_018963.4 | Brwd1 | NM_001103179.1 | Up | yes | 0.77277206 | 0.00013056 | 0.00478241 | no |
| RET | NM_020630.4 | Ret | NM_001080780.1 | Up | no | 0.76987631 | 0.00020458 | 0.00670504 | no |
| BEGAIN | NM_020836.3 | Begain | NM_001163175.1 | Up | no | 0.76616088 | 0.00310366 | 0.05102856 | Core |
| VIPR1 | NM_004624.3 | Vipr1 | NM_011703.4 | Up | no | 0.76537287 | 0.00066426 | 0.01594782 | no |
| SPO11 | NM_198265.1 | Spo11 | NM_001083959.1 | Up | no | 0.76141361 | 0.00085792 | 0.01985564 | Core |
| CLEC4F | NM_173535.2 | Clec4f | NM_016751.3 | Up | no | 0.75903099 | 0.00131662 | 0.02666281 | Core |
| FAM101B | NM_182705.2 | Fam101b | NM_029658.1 | Up | no | 0.75621692 | 0.00033841 | 0.00981736 | no |
| CCDC62 | NM_201435.4 | Ccdc62 | NM_001134767.1 | Up | no | 0.75426929 | 0.00165051 | 0.03249981 | no |
| C14orf45 | NM_025057.2 | 2900006K08Rik | NM_028377.3 | Up | no | 0.74917357 | 0.00290775 | 0.04836616 | no |
| IPCEF1 | NM_015553.2 | Ipcef1 | NM_001033391.2 | Up | no | 0.74291867 | 0.00175554 | 0.03426316 | no |
| FAM198A | NM_001129908.2 | Fam198a | NM_177743.5 | Up | no | 0.73972326 | 0.00290755 | 0.04836616 | Core |
| HEMGN | NM_018437.3 | Hemgn | NM_053149.2 | Up | no | 0.73571915 | 0.00457803 | 0.06596927 | no |
| PIGP | NM_153682.2 | Pigp | NM_001159618.1 | Up | yes | 0.73544614 | 0.00054142 | 0.01451066 | no |
| FGF13 | NM_033642.2 | Fgf13 | NM_010200.2 | Up | no | 0.71939127 | 0.00043521 | 0.01204505 | no |
| SH2D5 | NM_001103161.1 | Sh2d5 | NM_001099631.1 | Up | no | 0.71931902 | 0.00029529 | 0.00929573 | no |
| GPR174 | NM_032553.1 | Gpr174 | NM_001177782.1 | Up | no | 0.71804973 | 0.00450213 | 0.06596927 | no |
| PCDHB8 | NM_019120.3 | Pcdhb16 | NM_053141.3 | Up | no | 0.71273498 | 0.00633963 | 0.08646807 | Core |
| PCYT1B | NM_001163265.1 | Pcyt1b | NM_211138.1 | Up | no | 0.706017 | 0.00109653 | 0.02457884 | Core |
| DYRK1A | NM_001396.3 | Dyrk1a | NM_001113389.1 | Up | yes | 0.70575768 | 0.00035224 | 0.01004147 | no |
| CPM | NM_198320.3 | Cpm | NM_027468.1 | Up | no | 0.70514939 | 0.00040178 | 0.01126908 | no |
| PSMG1 | NM_203433.2 | Psmg1 | NM_019537.2 | Up | yes | 0.70417795 | 0.00045055 | 0.01238354 | no |
| CHAF1B | NM_005441.2 | Chaf1b | NM_028083.4 | Up | yes | 0.70267636 | 0.00040208 | 0.01126908 | no |
| HLCS | NM_000411.6 | Hlcs | NM_139145.4 | Up | yes | 0.7015347 | 0.00057989 | 0.01530537 | no |
| PCDHB15 | NM_018935.2 | Pcdhb22 | NM_053147.3 | Up | no | 0.69810234 | 0.01052186 | 0.1320334 | Core |
| SOX13 | NM_005686.2 | Sox13 | NM_011439.2 | Up | no | 0.69732128 | 0.00270165 | 0.04558484 | no |
| STOX2 | NM_020225.1 | Stox2 | NM_175162.4 | Up | no | 0.69232783 | 0.00646526 | 0.08794082 | no |
| ROGDI | NM_024589.2 | Rogdi | NM_133185.2 | Up | no | 0.6923141 | 0.00049971 | 0.01364069 | no |
| DLX1 | NM_001038493.1 | Dlx1 | NM_010053.1 | Up | no | 0.69136179 | 0.00300246 | 0.04961023 | no |
| STON1 | NM_006872.3 | Ston1 | NM_029858.2 | Up | no | 0.68159498 | 0.0016354 | 0.03226589 | no |
| TMEM91 | NM_001098825.1 | Tmem91 | NM_177102.4 | Up | no | 0.67648307 | 0.00967012 | 0.12320743 | no |
| TLR12P | none | Tlr12 | NM_205823.2 | Up | no | 0.67280447 | 0.00253063 | 0.04295388 | no |
| NID2 | NM_007361.3 | Nid2 | NM_008695.2 | Up | no | 0.66854142 | 0.00498142 | 0.07055068 | no |
| RAPGEF4 | NM_001100397.1 | Rapgef4 | NM_019688.2 | Up | no | 0.66605929 | 0.00286347 | 0.04782923 | Core |
| STC2 | NM_003714.2 | Stc2 | NM_011491.3 | Up | no | 0.66337225 | 0.00433958 | 0.06596927 | Core |
| KBTBD11 | NM_014867.2 | Kbtbd11 | NM_029116.2 | Up | no | 0.65108296 | 0.00114748 | 0.0253502 | no |
| HMGN1 | NM_004965.6 | Hmgn1 | NM_008251.3 | Up | yes | 0.64793074 | 0.0010204 | 0.02305376 | no |
| MGST2 | NM_002413.4 | Mgst2 | NM_174995.2 | Up | no | 0.64697692 | 0.00128698 | 0.026169 | no |
| PIPOX | NM_016518.2 | Pipox | NM_008952.2 | Up | no | 0.64658884 | 0.00204249 | 0.03895288 | Core |
| PYGM | NM_001164716.1 | Pygm | NM_011224.1 | Up | no | 0.64620253 | 0.0026497 | 0.04486033 | no |
| HAAO | NM_012205.2 | Haao | NM_025325.2 | Up | no | 0.64547287 | 0.00207692 | 0.03949124 | no |
| DNASE1L3 | NM_004944.3 | Dnase1l3 | NM_007870.3 | Up | no | 0.64521599 | 0.00144949 | 0.02908758 | Core |
| TMEM40 | NM_018306.2 | Tmem40 | NM_001168258.1 | Up | no | 0.63976237 | 0.01007011 | 0.12756963 | Core |
| TMEM59L | NM_012109.2 | Tmem59l | NM_182991.2 | Up | no | 0.63542748 | 0.00135843 | 0.02745387 | no |
| HIVEP3 | NM_024503.4 | Hivep3 | NM_010657.3 | Up | no | 0.63251424 | 0.00139751 | 0.02812951 | no |
| DST | NM_020388.3 | Dst | NM_133833.3 | Up | no | 0.62582013 | 0.0018951 | 0.0366994 | Core |
| GPR125 | NM_145290.3 | Gpr125 | NM_133911.1 | Up | no | 0.62574216 | 0.00200245 | 0.03836803 | no |
| ETHE1 | NM_014297.3 | Ethe1 | NM_023154.3 | Up | no | 0.62444457 | 0.0017176 | 0.03372086 | no |
| C1orf182 | NM_144627.3 | 1700021C14Rik | NM_029801.2 | Up | no | 0.62298149 | 0.01853356 | 0.2073657 | no |
| MMP16 | NM_022564.3 | Mmp16 | NM_019724.3 | Up | no | 0.61626987 | 0.01047818 | 0.13158043 | no |
| PRKAA2 | NM_006252.3 | Prkaa2 | NM_178143.2 | Up | no | 0.61390393 | 0.00306955 | 0.0505511 | Core |
| SFRP5 | NM_003015.3 | Sfrp5 | NM_018780.3 | Up | no | 0.61204577 | 0.00230812 | 0.04067418 | Core |
| COL27A1 | NM_032888.2 | Col27a1 | NM_025685.3 | Up | no | 0.60459675 | 0.00318061 | 0.05207889 | no |
| AIPL1 | NM_001033055.1 | Aipl1 | NM_053245.2 | Up | no | 0.60258467 | 0.02227333 | 0.23875241 | no |
| ACVR2A | NM_001616.3 | Acvr2a | NM_007396.4 | Up | no | 0.60239866 | 0.00277025 | 0.04662403 | no |
| TNIK | NM_001161563.1 | Tnik | NM_001163009.1 | Up | no | 0.6017156 | 0.01490184 | 0.17436818 | no |
| PLOD2 | NM_182943.2 | plod2 | NM_011961.3 | Up | no | 0.60003662 | 0.00287142 | 0.04792201 | Core |
| HDAC11 | NM_024827.3 | Hdac11 | NM_144919.2 | Up | no | 0.59486226 | 0.00394216 | 0.06309677 | no |
| MAP7 | NM_003980.4 | Mtap7 | NM_008635.2 | Up | no | 0.59390466 | 0.00281581 | 0.04727105 | no |
| MID1 | NM_001098624.2 | Mid1 | NM_010797.2 | Up | no | 0.59225001 | 0.00327879 | 0.05342315 | no |
| EGFL7 | NM_201446.2 | Egfl7 | NM_178444.4 | Up | no | 0.58757946 | 0.00724192 | 0.09704763 | no |
| TMC5 | NM_024780.4 | Tmc5 | NM_028930.3 | Up | no | 0.58571132 | 0.01318212 | 0.1590073 | Core |
| TET1 | NM_030625.2 | Tet1 | NM_027384.1 | Up | no | 0.58406287 | 0.01016637 | 0.12862561 | no |
| FAM167A | NM_053279.2 | Fam167a | NM_177628.4 | Up | no | 0.58373773 | 0.00579914 | 0.08019398 | no |
| ART4 | NM_021071.2 | Art4 | NM_026639.2 | Up | no | 0.57785042 | 0.01421897 | 0.16852615 | Core |
| KIF17 | NM_020816.2 | Kif17 | NM_010623.4 | Up | no | 0.57740651 | 0.0057942 | 0.08018133 | no |
| LGR5 | NM_003667.3 | Lgr5 | NM_010195.2 | Up | no | 0.57638977 | 0.00392876 | 0.06293286 | no |
| DCLK2 | NM_001040260.3 | Dclk2 | NM_001195499.1 | Up | no | 0.5746265 | 0.00553438 | 0.07712137 | Core |
| ETS2 | NM_005239.5 | Ets2 | NM_011809.3 | Up | yes | 0.57141611 | 0.00470171 | 0.06725848 | no |
| ACSBG1 | NM_015162.4 | Acsbg1 | NM_053178.2 | Up | no | 0.56786226 | 0.0159384 | 0.18390532 | Core |
| AXIN2 | NM_004655.3 | Axin2 | NM_015732.4 | Up | no | 0.56612961 | 0.02266692 | 0.24198756 | no |
| IL2RA | NM_000417.2 | Il2ra | NM_008367.3 | Up | no | 0.56556095 | 0.00438075 | 0.06596927 | no |
| FAM78B | NM_001017961.3 | Fam78b | NM_001160262.1 | Up | no | 0.5651145 | 0.009694 | 0.12343275 | no |
| FAM70A | NM_017938.3 | Fam70a | NM_172930.3 | Up | no | 0.56488014 | 0.00687523 | 0.0928203 | Core |
| RELN | NM_173054.2 | Reln | NM_011261.2 | Up | no | 0.5644999 | 0.00593355 | 0.08182548 | Core |
| LPCAT2 | NM_017839.4 | Lpcat2 | NM_173014.1 | Up | no | 0.56313619 | 0.0051067 | 0.07222225 | no |
| SLC6A19 | NM_001003841.2 | Slc6a19 | NM_028878.3 | Up | no | 0.56290538 | 0.01379682 | 0.16492453 | no |
| FCRL6 | NM_001004310.2 | Fcrl6 | NM_001164725.1 | Up | no | 0.5601165 | 0.00795614 | 0.10471732 | no |
| EPCAM | NM_002354.2 | Epcam | NM_008532.2 | Up | no | 0.55965488 | 0.02042117 | 0.22297678 | Core |
| IL33 | NM_033439.3 | Il33 | NM_001164724.1 | Up | no | 0.55711109 | 0.00667495 | 0.09034749 | Core |
| TSPAN6 | NM_003270.2 | Tspan6 | NM_019656.3 | Up | no | 0.55698215 | 0.00734012 | 0.09829745 | Core |
| SLC6A12 | NM_001122847.2 | Slc6a12 | NM_133661.3 | Up | no | 0.55533577 | 0.02111532 | 0.22956224 | Core |
| MORC3 | NM_015358.2 | Morc3 | NM_001045529.3 | Up | yes | 0.55424193 | 0.00502332 | 0.0710936 | no |
| ARNT2 | NM_014862.3 | Arnt2 | NM_007488.3 | Up | no | 0.54443356 | 0.0107445 | 0.13423549 | Core |
| MPZL2 | NM_005797.3 | Mpzl2 | NM_007962.4 | Up | no | 0.54077803 | 0.00655631 | 0.08899698 | Core |
| GIMAP8 | NM_175571.2 | Gimap8 | NM_001077410.1 | Up | no | 0.54047967 | 0.00942084 | 0.12072615 | no |
| TGFB3 | NM_003239.2 | Tgfb3 | NM_009368.3 | Up | no | 0.53827694 | 0.00638922 | 0.0870848 | no |
| AMDHD1 | NM_152435.2 | Amdhd1 | NM_027908.1 | Up | no | 0.53804836 | 0.0197572 | 0.21727467 | Core |
| SCARF1 | NM_145351.1 | Scarf1 | NM_001004157.2 | Up | no | 0.53661411 | 0.00819216 | 0.10732752 | no |
| DSCR3 | NM_006052.1 | Dscr3 | NM_007834.3 | Up | yes | 0.53645247 | 0.00658759 | 0.08929987 | no |
| UBXN11 | NM_001077262.1 | Ubxn11 | NM_026257.3 | Up | no | 0.53507171 | 0.01420038 | 0.16843515 | no |
| ARHGEF5 | NM_005435.3 | Arhgef5 | NM_133674.1 | Up | no | 0.53473914 | 0.01426964 | 0.16880055 | no |
| COL23A1 | NM_173465.3 | Col23a1 | NM_153393.2 | Up | no | 0.53309004 | 0.01433504 | 0.1692265 | no |
| PCDH9 | NM_020403.4 | Pcdh9 | NM_001081377.2 | Up | no | 0.53110781 | 0.01641305 | 0.18777084 | no |
| BFSP2 | NM_003571.2 | Bfsp2 | NM_001002896.2 | Up | no | 0.53085497 | 0.00847884 | 0.11042994 | Core |
| FHOD3 | NM_025135.2 | Fhod3 | NM_175276.3 | Up | no | 0.52900754 | 0.00899301 | 0.11561535 | no |
| LGALSL | NM_014181.2 | 1110067D22Rik | NM_173752.4 | Up | no | 0.52860295 | 0.01199973 | 0.14696904 | no |
| EPG5 | NM_020964.2 | 5430411K18Rik | NM_001195633.1 | Up | no | 0.52791501 | 0.0076841 | 0.10221705 | no |
| ZNF286A | NM_001130842.1 | Zfp286 | NM_138949.3 | Up | no | 0.52013941 | 0.01948528 | 0.21476033 | no |
| RDH10 | NM_172037.4 | Rdh10 | NM_133832.3 | Up | no | 0.51821306 | 0.01089663 | 0.13579555 | no |
| CACNA1E | NM_000721.3 | Cacna1e | NM_009782.3 | Up | no | 0.51253169 | 0.01168466 | 0.14388205 | Core |
| PGR | NM_000926.4 | Pgr | NM_008829.2 | Up | no | 0.51087944 | 0.01122086 | 0.13931341 | Core |
| KIAA1958 | NM_133465.2 | E130308A19Rik | NM_001015681.1 | Up | no | 0.50951234 | 0.01095596 | 0.13644961 | no |
| ENDOU | NM_006025.3 | Endou | NM_001168693.1 | Up | no | 0.50832021 | 0.01144396 | 0.1418183 | Core |

| Human | | Mouse | | | Triplicated | log2 | | adj. P. Val |
|---|---|---|---|---|---|---|---|---|
| Symbol | Human Transcript | Symbol | Mouse Transcript | Direction | gene | FC_Ts1vsWT | P. Value | (FDR) |
| COL2A1 | NM_033150.2 | Col2a1 | NM_031163.3 | Down | no | −2.1440539 | 9.64E−21 | 6.40E−17 |
| PLIN4 | NM_001080400.1 | Plin4 | NM_020568.3 | Down | no | −1.7703454 | 1.72E−12 | 1.14E−09 |
| LGR6 | NM_001017404.1 | Lgr6 | NM_001033409.3 | Down | no | −1.5256476 | 1.26E−08 | 2.36E−06 |
| ARG1 | NM_000045.3 | Arg1 | NM_007482.3 | Down | no | −1.4470417 | 6.74E−08 | 9.26E−06 |
| COL6A1 | NM_001848.2 | Col6a1 | NM_009933.4 | Down | no | −1.4225352 | 7.00E−12 | 4.10E−09 |
| COL6A2 | NM_001849.3 | Col6a2 | NM_146007.2 | Down | no | −1.4147234 | 1.92E−11 | 9.57E−09 |
| SFRP2 | NM_003013.2 | Sfrp2 | NM_009144.2 | Down | no | −1.4131069 | 6.88E−10 | 2.05E−07 |
| DCLK1 | NM_004734.4 | Dclk1 | NM_001111053.1 | Down | no | −1.3412335 | 4.19E−08 | 6.62E−06 |
| VCAM1 | NM_001078.3 | Vcam1 | NM_011693.3 | Down | no | −1.2830992 | 6.99E−07 | 5.81E−05 |
| SFRP1 | NM_003012.4 | Sfrp1 | NM_013834.3 | Down | no | −1.2635869 | 1.42E−08 | 2.64E−06 |
| RHOBTB3 | NM_014899.3 | Rhobtb3 | NM_028493.2 | Down | np | −1.2332277 | 2.88E−06 | 0.0002003 |
| SYT13 | NM_020826.2 | Syt13 | NM_030725.4 | Down | no | −1.2125034 | 1.52E−07 | 1.79E−05 |
| LTBP2 | NM_000428.2 | Ltbp2 | NM_013589.3 | Down | no | −1.2115952 | 4.92E−09 | 1.04E−06 |
| COL4A2 | NM_001846.2 | Col4a2 | NM_009932.3 | Down | no | −1.1996301 | 5.47E−08 | 8.14E−06 |
| CYR61 | NM_001554.4 | Cyr61 | NM_010516.2 | Down | no | −1.1859093 | 1.87E−08 | 3.38E−06 |
| COL12A1 | NM_004370.5 | Col12a1 | NM_007730.2 | Down | no | −1.1835883 | 1.07E−07 | 1.35E−05 |
| ENAH | NM_018212.4 | Enah | NM_001083121.1 | Down | no | −1.1834747 | 4.80E−06 | 0.00029816 |
| COL4A1 | NM_001845.4 | Col4a1 | NM_009931.2 | Down | no | −1.1740128 | 3.46E−08 | 5.67E−06 |
| GALNT9 | NM_001122636.1 | Galnt9 | NM_198306.2 | Down | no | −1.1598093 | 4.75E−06 | 0.00029672 |
| MRC2 | NM_006039.4 | Mrc2 | NM_008626.3 | Down | no | −1.1566648 | 6.14E−07 | 5.46E−05 |
| RTN4RL2 | NM_178570.1 | Rtn4rl2 | NM_199223.1 | Down | no | −1.1565433 | 1.03E−05 | 0.00057517 |
| HMGA2 | NM_003484.1 | Hmga2 | NM_010441.2 | Down | no | −1.1397018 | 6.67E−08 | 9.26E−06 |
| ATOH8 | NM_032827.6 | Atoh8 | NM_153778.3 | Down | no | −1.1280676 | 6.61E−06 | 0.00040008 |
| LTBP1 | NM_206943.2 | Ltbp1 | NM_019919.3 | Down | no | −1.1247489 | 1.51E−06 | 0.00011603 |
| PTRF | NM_012232.5 | Ptrf | NM_008986.2 | Down | no | −1.1165208 | 6.29E−08 | 8.85E−06 |
| IRG1 | XM_001722295.2 | Irg1 | NM_008392.2 | Down | no | −1.1157636 | 7.14E−06 | 0.00043116 |
| SEMA3C | NM_006379.3 | Sema3c | NM_013657.5 | Down | no | −1.1015506 | 1.12E−05 | 0.00061959 |
| SERPING1 | NM_000062.2 | Serping1 | NM_009776.3 | Down | no | −1.096034 | 2.63E−05 | 0.00130118 |
| CXCL12 | NM_199168.3 | Cxcl12 | NM_021704.3 | Down | no | −1.0931492 | 6.37E−05 | 0.00265108 |
| BGN | NM_001711.4 | Bgn | NM_007542.4 | Down | no | −1.0875481 | 6.73E−08 | 9.26E−06 |
| FAT1 | NM_005245.3 | Fat1 | NM_001081286.2 | Down | no | −1.0789981 | 1.32E−06 | 0.0001022 |
| MGP | NM_000900.3 | Mgp | NM_008597.3 | Down | no | −1.0756541 | 1.82E−06 | 0.00013782 |
| STEAP2 | NM_001040666.1 | Steap2 | NM_001103157.1 | Down | no | −1.0720385 | 2.17E−05 | 0.00108305 |
| H19 | none | H19 | NR_001592.1 | Down | no | −1.0706985 | 1.38E−07 | 1.64E−05 |
| BCAR1 | XM_929039.4 | Bcar1 | NM_009954.3 | Down | no | −1.0639587 | 1.72E−05 | 0.00087814 |
| CTGF | NM_001901.2 | Ctgf | NM_010217.2 | Down | no | −1.0636714 | 1.50E−06 | 0.000116 |
| OLFML3 | NM_020190.2 | Olfml3 | NM_133859.2 | Down | no | −1.0615782 | 2.88E−06 | 0.0002003 |
| OLFM1 | NM_006334.3 | Olfm1 | NM_001038612.1 | Down | no | −1.0546816 | 2.18E−06 | 0.00016319 |
| DLC1 | NM_001164271.1 | Dlc1 | NM_015802.3 | Down | no | −1.0379254 | 4.48E−05 | 0.00201537 |
| ST8SIA1 | NM_003034.3 | St8sia1 | NM_011374.2 | Down | no | −1.0370874 | 1.85E−05 | 0.0009382 |
| SHISA2 | NM_001007538.1 | Shisa2 | NM_145463.5 | Down | no | −1.0285894 | 0.00015328 | 0.00545414 |
| SPARC | NM_003118.3 | Sparc | NM_009242.4 | Down | no | −1.025705 | 4.36E−07 | 4.03E−05 |
| TENC1 | NM_198316.1 | Tenc1 | NM_153533.2 | Down | no | −1.006288 | 0.00020188 | 0.00662734 |
| TPH1 | NM_004179.2 | Tph1 | NM_009414.3 | Down | no | −1.0061163 | 0.00014385 | 0.00516477 |
| EPDR1 | NM_017549.4 | Epdr1 | NM_134065.4 | Down | no | −1.0055729 | 1.81E−05 | 0.00091975 |
| NPR2 | NM_003995.3 | Npr2 | NM_173788.3 | Down | no | −1.0017153 | 0.00018491 | 0.00617211 |
| F2RL2 | NM_004101.3 | F2rl2 | NM_010170.4 | Down | no | −0.9892648 | 0.00026881 | 0.00857039 |
| NFATC2 | NM_173091.3 | Nfatc2 | NM_001037177.1 | Down | no | −0.9855506 | 0.00026029 | 0.00840783 |
| EML1 | NM_004434.2 | Eml1 | NM_001043335.1 | Down | no | −0.9811063 | 0.00010526 | 0.00398019 |
| CALD1 | NM_033139.3 | Cald1 | NM_145575.3 | Down | no | −0.9804246 | 1.82E−06 | 0.00013782 |
| CCND1 | NM_053056.2 | Ccnd1 | NM_007631.2 | Down | no | −0.9794642 | 1.07E−06 | 8.67E−05 |
| LAMB1 | NM_002291.2 | Lamb1 | NM_008482.2 | Down | no | −0.9739861 | 9.60E−06 | 0.00054678 |
| ANK3 | NM_020987.3 | Ank3 | NM_170730.2 | Down | no | −0.9707859 | 3.41E−05 | 0.00159599 |
| SMAD6 | NM_001142861.2 | Smad6 | NM_008542.3 | Down | no | −0.9682965 | 2.83E−05 | 0.00136932 |
| GREM1 | NM_013372.6 | Grem1 | NM_011824.4 | Down | no | −0.9632938 | 7.22E−06 | 0.00043452 |
| PGM5 | NM_021965.3 | Pgm5 | NM_175013.2 | Down | no | −0.9585904 | 0.00072686 | 0.0172431 |
| AEBP1 | NM_001129.4 | Aebp1 | NM_009636.2 | Down | no | −0.9540463 | 2.59E−06 | 0.00018135 |
| FOSB | NM_001114171.1 | Fosb | NM_008036.2 | Down | no | −0.9521069 | 2.22E−06 | 0.00016319 |
| EOMES | NM_005442.3 | Eomes | NM_001164789.1 | Down | no | −0.9508195 | 0.0004596 | 0.01261488 |
| VGLL3 | NM_016206.2 | Vgll3 | NM_028572.1 | Down | no | −0.9499697 | 0.00044235 | 0.01220863 |
| IGSF11 | NM_001015887.1 | Igsf11 | NM_170599.2 | Down | no | −0.9362733 | 0.00089916 | 0.02071405 |
| CYP1B1 | NM_000104.3 | Cyp1b1 | NM_009994.1 | Down | no | −0.9353678 | 4.30E−06 | 0.00026975 |
| TNC | NM_002160.3 | Tnc | NM_011607.3 | Down | no | −0.9329682 | 1.86E−05 | 0.00093895 |
| COL1A2 | NM_000089.3 | Col1a2 | NM_007743.2 | Down | no | −0.9260755 | 5.02E−06 | 0.00031053 |
| DDR2 | NM_006182.2 | Ddr2 | NM_022563.2 | Down | no | −0.9238141 | 3.04E−05 | 0.00144216 |
| FERMT2 | NM_006832.2 | Fermt2 | NM_146054.2 | Down | no | −0.9215557 | 0.00030602 | 0.00954298 |
| SDC2 | NM_002998.3 | Sdc2 | NM_008304.2 | Down | no | −0.9186484 | 7.12E−05 | 0.00294434 |
| SRPX2 | NM_014467.2 | Srpx2 | NM_026838.4 | Down | no | −0.9167341 | 0.00012178 | 0.00448562 |
| PARVA | NM_018222.4 | Parva | NM_020606.5 | Down | no | −0.9163991 | 0.00011259 | 0.00421707 |
| CXorf57 | NM_018015.5 | D330045A20Rik | NM_175326.5 | Down | no | −0.9163003 | 0.00023837 | 0.00776155 |
| ANTXR1 | NM_018153.3 | Antxr1 | NM_054041.2 | Down | no | −0.9146805 | 9.93E−05 | 0.00382088 |
| TNFSF13 | NM_172089.3 | Tnfsf13 | NM_001159505.1 | Down | no | −0.9103826 | 6.57E−06 | 0.0003994 |
| AMOTL2 | NM_016201.2 | Amotl2 | NM_019764.2 | Down | no | −0.9067286 | 0.00010427 | 0.0039576 |
| LOXL1 | NM_005576.2 | Loxl1 | NM_010729.3 | Down | no | −0.9052608 | 3.96E−05 | 0.0018184 |
| EDNRB | NM_000115.3 | Ednrb | NM_007904.4 | Down | no | −0.9051174 | 1.28E−05 | 0.0006908 |
| GPX8 | NM_001008397.2 | Gpx8 | NM_027127.2 | Down | no | −0.8996573 | 0.0004076 | 0.01139422 |
| PCOLCE | NM_002593.3 | Pcolce | NM_008788.2 | Down | no | −0.8975007 | 2.94E−05 | 0.0014072 |
| FOS | NM_005252.3 | Fos | NM_010234.2 | Down | no | −0.8968545 | 6.07E−06 | 0.00037197 |
| FOSL1 | NM_005438.3 | Fosl1 | NM_010235.2 | Down | no | −0.8956376 | 7.46E−05 | 0.00306346 |
| CXCL14 | NM_004887.4 | Cxcl14 | NM_019568.2 | Down | no | −0.8886254 | 0.0003037 | 0.00953029 |
| CCDC141 | NM_173648.3 | Ccdc141 | NM_001025576.3 | Down | no | −0.8839218 | 0.00036415 | 0.01035151 |
| ADAMTS2 | NM_014244.4 | Adamts2 | NM_175643.3 | Down | no | −0.883814 | 0.00060386 | 0.01568356 |
| HTR7 | NM_000872.4 | Htr7 | NM_008315.2 | Down | no | −0.8816658 | 0.00247068 | 0.04204374 |
| PTGFRN | NM_020440.2 | Ptgfrn | NM_011197.3 | Down | no | −0.8768295 | 1.29E−05 | 0.0006908 |
| COL3A1 | NM_000090.3 | Col3a1 | NM_009930.2 | Down | no | −0.8712592 | 5.04E−05 | 0.00219092 |
| COL8A1 | NM_001850.4 | Col8a1 | NM_007739.2 | Down | no | −0.8684527 | 0.00029427 | 0.00927844 |
| MAMLD1 | NM_005491.3 | Mamld1 | NM_001081354.2 | Down | no | −0.8670569 | 0.00012013 | 0.00444429 |
| NID1 | NM_002508.2 | Nid1 | NM_010917.2 | Down | no | −0.866624 | 4.48E−05 | 0.00201537 |
| ID1 | NM_181353.2 | Id1 | NM_010495.2 | Down | no | −0.863628 | 2.81E−05 | 0.00136165 |
| COL5A1 | NM_000093.4 | Col5a1 | NM_015734.2 | Down | no | −0.8627565 | 0.00010201 | 0.0038943 |
| CHN2 | NM_001039936.1 | Chn2 | NM_023543.2 | Down | no | −0.8625962 | 0.00015313 | 0.00545414 |
| CTTN | NM_138565.2 | Cttn | NM_007803.5 | Down | no | −0.8582913 | 5.33E−05 | 0.00226437 |
| FGF7 | NM_001719907.2 | Fgf7 | NM_008008.4 | Down | no | −0.8528438 | 0.00016378 | 0.00579706 |
| HSPG2 | NM_005529.5 | Hspg2 | NM_008305.3 | Down | no | −0.8505163 | 9.33E−05 | 0.00370918 |
| SERPINH1 | NM_001235.3 | Serpinh1 | NM_009825.2 | Down | no | −0.8503187 | 2.89E−05 | 0.0013896 |
| DGKG | NM_001346.2 | Dgkg | NM_138650.2 | Down | no | −0.8488873 | 0.00125982 | 0.02564301 |
| ERBB2 | NM_004448.2 | Erbb2 | NM_001003817.1 | Down | no | −0.847096 | 0.00106957 | 0.02408289 |
| PRRX1 | NM_006902.3 | Prrx1 | NM_001025570.1 | Down | no | −0.8463165 | 0.00043829 | 0.01211352 |
| COL18A1 | NM_030582.3 | Col18a1 | NM_001109991.1 | Down | no | −0.8395118 | 0.00026086 | 0.00841116 |
| AK1 | NM_000476.2 | Ak1 | NM_021515.3 | Down | no | −0.8391814 | 9.52E−05 | 0.00370918 |
| WWTR1 | NM_015472.4 | Wwtr1 | NM_001168281.1 | Down | no | −0.8391193 | 0.00091138 | 0.02094705 |
| DLG5 | NM_004747.3 | Dlg5 | NM_001163513.1 | Down | no | −0.8365117 | 0.00028916 | 0.00916707 |
| MSRB3 | NM_198080.3 | Msrb3 | NM_177092.4 | Down | no | −0.8349179 | 0.00159883 | 0.03165369 |
| FN1 | NM_212482.1 | Fn1 | NM_010233.2 | Down | no | −0.8339593 | 3.26E−05 | 0.0015338 |
| THY1 | NM_006288.3 | Thy1 | NM_009382.3 | Down | no | −0.8322354 | 5.45E−05 | 0.00280212 |
| GLIS2 | NM_032575.2 | Glis2 | NM_031184.3 | Down | no | −0.8315623 | 0.00040769 | 0.01139422 |
| IL18RAP | NM_003853.2 | Il18rap | NM_010553.3 | Down | no | −0.8266683 | 0.00117139 | 0.02536111 |
| VSIG8 | NM_001134233.1 | Vsig8 | NM_177723.4 | Down | no | −0.8262109 | 0.0030008 | 0.04961023 |
| SARDH | NM_001134707.1 | Sardh | NM_138665.2 | Down | no | −0.820658 | 0.00411321 | 0.06494764 |
| CCDC80 | NM_199511.1 | Ccdc80 | NM_026439.2 | Down | no | −0.820638 | 0.00087106 | 0.02011321 |
| TMEM158 | NM_015444.2 | Tmem158 | NM_001002267.2 | Down | no | −0.816622 | 0.00034992 | 0.00998978 |
| SLC16A14 | NM_152527.4 | Slc16a14 | NM_027921.1 | Down | no | −0.8149653 | 0.00369195 | 0.05942616 |
| FSCN1 | NM_003088.3 | Fscn1 | NM_007984.2 | Down | no | −0.8127236 | 0.00011535 | 0.00429657 |
| SERPINE1 | NM_001165413.2 | Serpine1 | NM_008871.2 | Down | no | −0.8110293 | 7.44E−05 | 0.00306346 |
| LAMA3 | NM_198129.1 | Lama3 | NM_010680.1 | Down | no | −0.8074403 | 0.00099391 | 0.02253195 |
| PCDH7 | NM_032456.2 | Pcdh7 | NM_001122758.1 | Down | no | −0.8034139 | 0.000136 | 0.00494551 |
| PENK | NM_001135690.2 | Penk | NM_001002927.2 | Down | no | −0.8029919 | 0.00630939 | 0.08611449 |
| UMC5B | NM_170744.4 | Unc5b | NM_029770.2 | Down | no | −0.8018947 | 0.00019716 | 0.00649401 |
| ENPP2 | NM_001130863.2 | Enpp2 | NM_001136077.1 | Down | no | −0.8004852 | 0.00466564 | 0.06693466 |
| TGFB1I1 | NM_001164719.1 | Tgfb1i1 | NM_009365.2 | Down | no | −0.7995912 | 0.00054117 | 0.01451066 |
| TPBG | NM_001166392.1 | Tpbg | NM_011627.4 | Down | no | −0.7994097 | 0.00437925 | 0.06596927 |
| PRSS46 | XM_002342331.2 | Prss46 | NM_183103.2 | Down | no | −0.7973843 | 0.00376867 | 0.06056319 |
| CADM1 | NM_014333.3 | Cadm1 | NM_207675.2 | Down | no | −0.7965875 | 7.59E−05 | 0.0031077 |
| SERPINE2 | NM_006216.3 | Serpine2 | NM_009255.4 | Down | no | −0.7950485 | 0.00019697 | 0.00649401 |
| GPC6 | NM_005708.3 | Gpc6 | NM_001079844.1 | Down | no | −0.7902574 | 0.00414031 | 0.06527217 |
| TMEM119 | NM_181724.2 | Tmem119 | NM_146162.2 | Down | no | −0.7894713 | 0.00269863 | 0.04557248 |
| GPR126 | NM_020455.5 | Gpr126 | NM_001002268.3 | Down | no | −0.7859017 | 0.0017939 | 0.03493183 |
| HOXB4 | NM_024015.4 | Hoxb4 | NM_010459.7 | Down | no | −0.7835573 | 0.0005078 | 0.0138049 |
| FARP1 | NM_001001715.2 | Farp1 | NM_134082.3 | Down | no | −0.7824948 | 0.00319831 | 0.05232569 |
| PDGFRB | NM_002609.3 | Pdgfrb | NM_001146268.1 | Down | no | −0.7803336 | 0.00033727 | 0.00981736 |
| GHR | NM_000163.4 | Ghr | NM_010284.2 | Down | no | −0.7764881 | 0.00399615 | 0.06369813 |
| RBFOX2 | NM_001031695.2 | Rbfox2 | NM_001110827.1 | Down | no | −0.7702902 | 0.00059526 | 0.01560756 |
| GPR114 | NM_153837.1 | Gpr114 | NM_001033468.3 | Down | no | −0.7698557 | 0.0004297 | 0.01190907 |
| FFAR2 | NM_005306.2 | Ffar2 | NM_001168512.1 | Down | no | −0.7693888 | 0.00517767 | 0.07317407 |
| CLU | NM_001171138.1 | Clu | NM_013492.2 | Down | no | −0.7645768 | 0.00754015 | 0.10057072 |
| PLA2G2E | NM_014589.2 | Pla2g2e | NM_012044.2 | Down | no | −0.7644378 | 0.00785548 | 0.10366628 |
| PLCD1 | NM_001130964.1 | Plcd1 | NM_019676.2 | Down | no | −0.7643083 | 0.0020968 | 0.03983127 |
| KDELR3 | NM_016657.1 | Kdelr3 | NM_134090.2 | Down | no | −0.7598954 | 0.00363864 | 0.05875788 |
| NRG1 | NM_001160001.1 | Nrg1 | NM_178591.2 | Down | no | −0.7565278 | 0.00888578 | 0.11431046 |
| EPHB2 | NM_017449.3 | Ephb2 | NM_010142.2 | Down | no | −0.7542521 | 0.00130071 | 0.02641442 |
| SOD3 | NM_003102.2 | Sod3 | NM_011435.3 | Down | no | −0.7537364 | 0.00148125 | 0.02957609 |
| CD300E | NM_181449.2 | Cd300e | NM_172050.2 | Down | no | −0.752897 | 0.0032362 | 0.0528411 |
| HTR2A | NM_000621.4 | Htr2a | NM_172812.2 | Down | no | −0.751165 | 0.0125197 | 0.1523077 |
| MYLK2 | NM_033118.3 | Mylk2 | NM_001081044.2 | Down | no | −0.7501059 | 0.00566126 | 0.0786146 |
| FHL2 | NM_201555.1 | Fhl2 | NM_010212.3 | Down | no | −0.7497906 | 0.0036504 | 0.05890003 |
| GPR4 | NM_005282.2 | Gpr4 | NM_175668.4 | Down | no | −0.7493454 | 0.0046353 | 0.06655693 |
| BMP1 | NM_001199.3 | Bmp1 | NM_033241.1 | Down | no | −0.747607 | 0.00058824 | 0.01550515 |
| GREB1 | NM_014668.3 | Greb1 | NM_015764.4 | Down | no | −0.7417772 | 0.00052579 | 0.01417793 |
| RGS1 | NM_002922.3 | Rgs1 | NM_015811.2 | Down | no | −0.7401076 | 0.00019824 | 0.00651863 |

TABLE 2

| | | | | Genes | Genes in | |
|---|---|---|---|---|---|---|
| Gene set | Category | p value | FDR | in set | category | Overlap |
| Ts1Rhr 150 UP | chr21q22 | 8.80E−07 | 0.00029 | 13 | 261 | PCP4, ERG, TTC3, BRWD1, PIGP, |
| | | | | | | DYRK1A, PSMG1, CHAF1B, HLCS, |
| | | | | | | HMGN1, ETS2, MORC3, DSCR3 |
| Ts1Rhr 150 UP | chr3p22 | 0.045 | 1 | 3 | 86 | CMTM8, VIPR1, FAM198A |
| Ts1Rhr 150 UP | chr3p25 | 0.066 | 1 | 3 | 101 | ATP2B2, TMEM40, HDAC11 |
| Ts1Rhr 150 UP | chr2p14 | 0.077 | 1 | 2 | 50 | NRXN1, LGALSL |
| Ts1Rhr 150 UP | chr8q13 | 0.094 | 1 | 2 | 56 | SLCO5A1, RDH10 |
| Ts1Rhr 150 UP | chr2p | 0.097 | 1 | 1 | 11 | HAAO |
| Ts1Rhr 150 UP | chr11q24 | 0.1 | 1 | 3 | 122 | C11ORF63, ESAM, MPZL2 |

TABLE 3

| Cluster | GeneSet | p value | FDR | Overlap |
|---|---|---|---|---|
| PRC2 | BENPORATH_SUZ12_TARGETS | 1.83E−14 | 4.45E−11 | LGR5, NID2, TSPAN6, ESAM, STYL4, PCDH17, COL27A1, SCN4B, SLO5A1, DLX1, RAPG [symbol] |
| | | | | EF4, ERG, LRCH2, PGR, CACNA1E, SFRP5, TMEM132E, MID1, FGF13, RDH10, INSM1, FA [symbol] |
| | | | | M10A, PCDH11X |
| PRC2 | MEISSNER_BRAIN_HCP_WITH_H3K4ME3_AND_{—} | 3.30E−03 | 1.10E−05 | LGR5, NID2, RELN, CMTM8, PCDH17, COL27A1, SCN4B, SLCO5A1, DLX1, SOX13, PTPN1 [symbol] |
| | H3K27ME3 | | | 4, VIPR1, PLOD2, PRDM16, RET, FZD6, NKD2 |
| PRC2 | BENPORATH_ES_WITH_H3K27ME3 | 1.73E−08 | 1.41E−05 | LGR5, ESAM, PCDH17, COL27A1, SCN4B, SLCOSA1, DLX1, RAPGEF4, LRCH2, PGR, CAC [symbol] |
| | | | | NA1E, SFRP5, TMEM132E, MID1, STC2 |
| PCR2 | BENPORATH_EED_TARGETS | 5.50E−08 | 3.71E−05 | LGR5, TSPAN6, ESAM, RELN, PCDH17, COL27A1, SCN4B, SLCO5A1, DLX1, LRCH2, PGR, |
| | | | | CACNA1E, SFRP5, TMEM132E, CPM, NRXN1, GPR174 |
| PCR2 | BENPORATH_PRC2_TARGETS | 3.48E−07 | 1.37E−04 | LGR5, ESAM, PCDH17, COL27A1, SCN4B, SLCO5A1, DLX1, LRCH2, PGR, CANA1E, SFR [symbol] |
| | | | | P5, TMEM132E |
| PCR2 | MIKKELSEN_NPC_HCP_WITH_H3K27ME3 | 5.92E−06 | 9.71E−04 | SCN4B, PGR, SFRP5, TMEM132E, VIPR1, PRDM16, ATP2B2, TMEM31 |
| PCR2 | NUYTTEN_E2H2_TARGETS_UP | 3.82E−05 | 5.01E−03 | NID2, ETS2, IFI44, TGFB3, PLOD2, STC2, SH2D5, KCNB1, HIVEP3, MORC3, TNIK, AXIN2 [symbol] |
| PCR2 | MIKKELSEN_MEF_HCP_WITH_H3K27ME3 | 4.67E−05 | 5.78E−03 | ESAM, RELN, RAPGEF4, CACNA1E, SFRP5, INSM1, KCNB1, ATP2B2, TMEM31 |
| PCR2 | MIKKELSEN_MCV6_HCP_WITH_H3K27ME3 | 2.41E−04 | 1.76E−02 | RELN, SCN4B, SFRP5, TMEM132E, NKD2, ATP2B2, TMEM31 |
| PCR2 | MEISSNER_NCP_HCP_WITH_H3K4ME3_AND_{—} | 4.77E−04 | 2.68E−02 | SCN4B, TMEM132E, VIPR1, PRDM16, RET, ATP2B2 |
| | H3K27ME3 | | | |
| PCR2 | MEISSNER_NPC_HCP_WITH_H3K4ME2 | 4.37E−04 | 2.70E−02 | MAP1, SLCO5A1, INSM1, F2D6, NKD2, COL23A1, KDNB1 |
| Stem | WONG_ADULT_TISSUE_STEM_MODULE | 0.00E−00 | 0.00E−00 | LGR5, NID2, TSPAN6, ESAM, SYTL4, RELN, ETS2, MYLK, DST, TTC3, PXDN, CACNA2D [symbol] |
| Cell | | | | 1, CMTM8, HOAC11, PYGM, FAM101B, IL2RA, STAT4, IFI44, MAPI, GPR125, ARHGEF5, |
| | | | | PCDKB15 |
| Stem | BOQUEST_STEM_CELL_DN | 1.19E−08 | 1.13E−05 | TSPAN6, ETS2, PCDH17, RAPGEF4, ERG, DOCK9, GIMAP4, MGST2, SCARF1 |
| Cell | | | | |
| Stem | LIM_MAMMARY_STEM_CELL_UP | 1.53E−07 | 8.00E−05 | RELN, MYLK, DST, FAM101B, SCN4B, LRCH2, FHOD3, ACVR2A, COL23A1, TMEM121, |
| Cell | | | | SH2D5 |
| Stem | JAATINEN_HEMATOPOIETIC_STEM_CELL_UP | 6.66E−06 | 7.28E−04 | DST, PXDN, MAP7, GPR125, ERG, 1RCH2, PRDM16, ANKRD6 |
| Cell | | | | |
| Stem | YAUCH_HEDGEHOG_SIGNALLING_PARACRINE_UP | 4.30E−06 | 7.81E−04 | RELN, MAP1, RDH10, RET, GPR174, KEMGN |
| Cell | | | | |
| Stem | ST_WNT_BETA_CATENIN_PATHWAY | 1.21E−04 | 1.10E−02 | NKD2, ANKRD6, AXIN2 |
| Cell | | | | |
| Stem | TAKEDA_TARGETS_OF_NUP38_HOXAS_FUSION_3D_UP | 1.71E−04 | 1.44E−02 | DST, IFI44, MAP7, ERG, PCDH3 |
| Cell | | | | |
| Stem | IVANOVA_HEMATOPOIESIS_STEM_CELL_LONG_TERM | 2.22E−04 | 1.72E−02 | MAP7, DOCK9, PRDM16, SCARF1, PCDH3, PCP4 |
| Cell | | | | |
| Stem | SANSOM_WNT_PATHWAY_REQUIRE_MYC | 5.36E−04 | 3.06E−02 | LGR5, FZD6, AXIN2 |
| Cell | | | | |
| Cancer | TURASHVILI_BREAST_LOBULAR_CARCINOMA_{—} | 2.83E−07 | 1.24E−04 | MYLK, DST, PGR, FHOD3, STC2, AMDHD1 |
| | VS_LOBULAR_NORMAL_UP | | | |
| Cancer | SHETH_LIVER_CANCER_VS_TXNIP_LOSS_PAM4 | 8.19E−07 | 2.58E−04 | CACNA1E, TGFB3, DDC, SLC6A12, DNASE1L3, HAAO, CLEC4F, ETHE1 |
| Cancer | VERHAAK_GLIOBLASTOMA_PRONEURAL | 2.35E−06 | 5.28E−04 | TTC3, SLCO5A1, PCDH11X, FHOD3, PTPN14, MMP16, PIPOX |
| Cancer | KRAS.KIDNEY_UP.VI_UP | 3.68E−06 | 6.85E−04 | RELN, PCHD9, PCP4, NRXN1, MAP7, RAPGEF4 |
| Cancer | RIGGLEWING_SARCOMA_PROGENITOR_UP | 3.85E−06 | 7.81E−04 | RELN, STAT4, IFI44, PCDH17, SLCO5A1, MYOM2, GIMAP8, HIVEP3, PCDH3 |
| Cancer | YOSHIMURA_MAPK6_{——}TARGETS_UP | 4.23E−05 | 7.81E−04 | PYGM, RAPGEF4, PGR, SOX13, RET, DDC, ACVR2A, KCNB1, SLC6A12, DNA, SEIL3, HA [symbol] |
| | | | | AO, EGFLT, DYRK1A, ATP2B2, ACPP |
| Cancer | DODD_NASOPHARYNGEAL_CARCINOMA_UP | 1.44E−05 | 2.13E−03 | TSPAN6, SYTL4, CMTM8, RDH10, DOCK3, ACSBG1, TMEM40, VIPR1, PRKAA2, TME |
| | | | | M121, KCNB1, DNASEIL3, CLEC4F, TMC5, PCDH9, MAGI1, IL20RB |
| Cancer | KRAS.LUNCH_UP.VI_UP | 5.13E−05 | 4.85E−03 | RELN, PRG3, SLCO5A1, EGFLT, PCDH17 |
| Cancer | KRAS.OF.YI_ON | 2.23E−04 | 7.23E−03 | MAP7, ETS2, RET, PTPN14, TGFB3 |
| Cancer | EGFR_UP.VI_UP | 2.24E−04 | 7.23E−03 | PCDH9, ETS2, ARNT2, TMEM121, ETHE1 |
| Cancer | HOSHIDA_LIVER_CANCER_SUBCLASS_S3 | 1.12E−04 | 1.03E−02 | ETS2, MYLK, MGST2, SLC5A12, DNASE1L3, HAAO |
| Cancer | SASAKI_ADULT_T_CELL_LEUKEMIA | 1.46E−04 | 1.25E−02 | DST, IL2RA, GPR125, FZD6, ARNT2 |
| Cancer | RICKMAN_HEAD_AND_NECK, CANCER_A | 1.83E−04 | 1.56E−02 | LGR5, DLX1, ARNT2, ANKRD6 |
| Cancer | BOYLAN_MULTIPLE_MYELOMA_PCA1_UP | 1.36E−04 | 1.58E−02 | STAT4, GIMAP4, NKG7, GIMAP6 |
| Cancer | ACEVEDO_FGFR1_TARGETS_IN_PROSTATE_CANCER_{—} | 2.46E−04 | 1.76E−02 | LGR5, MYLK, CACNA2D1, FHOD3, COL23A1, PCP4 |
| | MODEL_ON | | | |
| Cancer | DOANE_BREAST_CANCER_ESR1_UP | 2.31E−04 | 2.02E−02 | PGR, RET, STC2, TMC5, |
| Cancer | CHARAFE_BREAST_CANCER_LUMINAL_VS_BASAL_ON | 3.16E−04 | 2.10E−02 | ETS2, DST, FAM101B, STAT4, IFI44, FZD6, IL20RB |
| Cancer | KEGG_PATHWAYS_IN_CANCER | 3.44E−04 | 2.21E−02 | FGF13, TGFB3, RET, FZD6, ARNT2, AXIN2 |
| Cancer | CHARAFE_BREAST_CANCER_LUMINAL_VS_{—} | 3.37E−04 | 2.21E−02 | NID2, MYLK, PXDN, FAM101B, MID1, FHOD3, SH2D5 |
| | MESENCHYMAL_ON | | | |
| Cancer | ONKEN_UVEAL_MELANOMA_UP | 3.85E−04 | 2.25E−02 | ETS2, TTC3, PXON, GPR125, RAPGEF4, FGF13, SOX13, DOCK9, MGST2 |
| Cancer | SASAL_RESISTANCE_TO_NEOPLASTIC_{—} | 3.85E−04 | 2.25E−02 | TGFB3, PLOD2, COL5A3 |
| | TRANSFORMATION | | | |
| Cancer | MARTORIATI_MDM4_TARGETS_FETAL_LIVER_U | 4.71E−04 | 2.68E−02 | PTPN14, PLOD2, STC2, PIGP, KBTBD11 |
| Cancer | SMID_BREAST_CANCER_BASAL_UP | 5.12E−04 | 2.75E−02 | LGR5, MYLK, PXON, MIDLPTPN14, PLOD2, PCP4, CHAF1B |
| Cancer | NICKOLSKY_BREAST_CANCER_8Q12_Q22_AMPLICON | 5.42E−04 | 2.88E−02 | SLCO5A1, RDH10, F2D6, MMP16 |
| Differ- | HOFFMANN_PRE_BI_TO_LARGE_PRE_BIL_{—} | 6.17E−05 | 6.75E−03 | STAT4, GIMAP4, MGST2, HEMGN |
| entiation | LYMPHOCYTE_ON | | | |
| Differ- | MATSUDA_NATURAL_KILLER_DIFFERENTIATION | 6.23E−05 | 6.75E−03 | DST, FAM101B, STAT4, SOX13, FZD6, NKG7, MAGI1, CHAF18 |
| entiation | | | | |
| Differ- | HOFFMAN_IMMATURE_TO_MATURE_B_LYMPHOCYTE_UP | 2.46E−04 | 1.76E−02 | FAM101B, STAT4, GIMAP4 |
| entiation | | | | |

[symbol]

indicates data missing or illegible when filed

TABLE 4

| Gene | | | | |
|---|---|---|---|---|
| set | Category | p value | FDR | Overlap |
| Ts1Rhr | BENPORATH_5UZ12_TARGETS | 1.33E−10 | 4.54E−07 | SFRP5, PGR, SCN4B, TMEM132E, CACNA1E, |
| Core Up | | | | SLCO5A1, LRCH2, TSPAN5, RAPGEF4, INS M1, |
| | | | | PCDH11X, FAM70A |
| Ts1Rhr | BENPORATH_EED_TARGETS | 4.00E−08 | 5.80E−05 | SFRP5, PGR, SCN4B, TMEM132E, CACNA1E, |
| Core Up | | | | SLCO5A1, LRCH2, TSPAN6, STC2, RELN |
| Ts1Rhr | BENPORATH_ES_WITH_H3K27ME3 | 6.45E−08 | 7.32E−05 | SFRP5, PGR, SCN4B, TMEM132E, CACNA1E, |
| Core Up | | | | SLCO5A1, LRCH2, RAPGEF4, STC2, NRXN1 |
| Ts1Rhr | MIKKELSEN_NPC_HCP_WITH_H3K27ME3 | 7.73E−07 | 6.57E−04 | SFRP5, PGR, SCN4B, TMEM132E, ATP2B2, |
| Core Up | | | | PRDM16 |
| Ts1Rhr | MIKKELSEN_MEF_HCP_WITH_H3K27ME3 | 1.20E−05 | 8.15E−04 | SFRP5, CACNA1E, RAPGEF4, INSM1, RELN, |
| Core Up | | | | ATP2B2, KCNB1 |
| Ts1Rhr | TURASHVILI_BREAST_LOBULAR_{—} | 1.91E−06 | 1.08E−03 | PGR, STC2, DST, AMDHD1 |
| Core Up | CARCINOMA_VS_LOBULAR_NORMAL_UP | | | |
| Ts1Rhr | BENPORATH_PRC2_TARGET | 2.32E−06 | 1.13E−03 | SFRP5, PGR, SCN4B, TMEM132E, CACNA1E, |
| Core Up | | | | SLCO5A1, LRCH2 |
| Ts1Rhr | SHETH_LIVER_CANCER_VS_TXNIP_LOSS_{—} | 4.59E−06 | 1.95E−03 | CACNA1E, DNASE1L3, CLEC4F, SLC6A12, DD |
| Core Up | PAM4 | | | |
| Ts1Rhr | NAKAMURA_TUMOR_ZONE_PERIPHERAL_VS_{—} | 2.55E−05 | 1.01E−02 | STC2, ARNT2, TMC5, IL2ORB, PLOD2, |
| Core Up | CENTRAL_DN | | | CACNA2D1 |
| Ts1Rhr | SMID_BREAST_CANCER_RELAPSE_IN_{—} | 3.83E−05 | 1.22E−02 | STC2, ARNT2 |
| Core Up | LIVER_DN | | | |
| Ts1Rhr | DODD_NASOPHARYNGEAL_CARCINOMA_UP | 3.93E−05 | 1.22E−02 | TSPAN6, KCNB1, DNASE2L3, CLEC4F, TMC5, |
| Core Up | | | | IL2ORB, PRKAA2, TMEM40, AC5BG1 |
| Ts1Rhr | MIKKELSEN_MCV6_HCP_WITH_H3K27ME3 | 5.32E−05 | 1.51E−02 | SFRP5, SCN4B, TMEM132E, RELN, ATP2B2 |
| Core Up | | | | |
| Ts1Rhr | SCHLESINGER_METHYLATED_DE_NOVO_{—} | 7.92E−05 | 2.07E−02 | SFRP5, PGR, PCDH11X |
| Core Up | IN_CANCER | | | |
| Ts1Rhr | SABATES_COLORECTAL_ADENOMA_DN | 1.60E−04 | 3.67E−02 | INSM1, FAM7CA, NRXN1, PRKAA2 |
| Core Up | | | | |
| Ts1Rhr | DOANE_BREAST_CANCER_ESR1_UP | 1.62E−04 | 3.57E−02 | PGR, STC2, TMC5 |
| Core Up | | | | |
| Ts1Rhr | CERVERA_SDHB_TARGETS_1_UP | 1.89E−04 | 3.86E−02 | SCN4B, SLCO5A1, TMEM40 |
| Core Up | | | | |
| Ts1Rhr | YOSHIMURA_MAPKB_TARGETS_UP | 1.93E−04 | 3.86E−02 | PGR, RAPGEF4, ATP2B2, KCNB1, DNA5E1L3, |
| Core Up | | | | SLC5A12, DDC |
| Ts1Rhr | DAVICIONI_MOLECULAR_ARMS_VS_{—} | 2.64E−04 | 5.00E−02 | RAPGEF4, STC2, DST, PIPOX |
| Core Up | ERMS_UP | | | |

TABLE 5

| ID | Type | Rank |
|---|---|---|
| ESYT3 | SUZ12_targets | 1 |
| TRPC6 | SUZ12_targets | 2 |
| RNF128 | SUZ12_targets | 3 |
| PTCHD1 | SUZ12_targets | 4 |
| RDH10 | SUZ12_targets | 5 |
| GUCY1A2 | SUZ12_targets | 6 |
| ADAM12 | SUZ12_targets | 7 |
| FZD10 | SUZ12_targets | 8 |
| PDZD2 | SUZ12_targets | 9 |
| DOK6 | SUZ12_targets | 10 |
| CSMD1 | SUZ12_targets | 11 |
| NPAS2 | SUZ12_targets | 12 |
| PDE88 | SUZ12_targets | 13 |
| ASCL1 | SUZ12_targets | 14 |
| NIN | SUZ12_targets | 15 |
| DOK6 | SUZ12_targets | 16 |
| SIX1 | SUZ12_targets | 17 |
| FBN1 | SUZ12_targets | 18 |
| CDH13 | SUZ12_targets | 19 |
| GABRA2 | SUZ12_targets | 20 |
| DCC | SUZ12_targets | 21 |
| FOXD3 | SUZ12_targets | 22 |
| ADAMTS5 | SUZ12_targets | 23 |
| FOXE1 | SUZ12_targets | 24 |
| ST8SIA1 | SUZ12_targets | 25 |
| STX8P6 | SUZ12_targets | 26 |
| SLC1A2 | SUZ12_targets | 27 |
| SHOX2 | SUZ12_targets | 28 |
| DKK2 | SUZ12_targets | 29 |
| GIPC2 | SUZ12_targets | 30 |
| NFIA | SUZ12_targets | 31 |
| TBX22 | SUZ12_targets | 32 |
| SLC6A1 | SUZ12_targets | 33 |
| SUSD4 | SUZ12_targets | 34 |
| CWH43 | SUZ12_targets | 35 |
| RARRES1 | SUZ12_targets | 36 |
| HLF | SUZ12_targets | 37 |
| CFTR | SUZ12_targets | 38 |
| LRCH2 | SUZ12_targets | 39 |
| DDAH1 | SUZ12_targets | 40 |
| NPNT | SUZ12_targets | 41 |
| GUCY1A2 | SUZ12_targets | 42 |
| CDC20B | SUZ12_targets | 43 |
| RHOB | SUZ12_targets | 44 |
| RSPO2 | SUZ12_targets | 45 |
| SPATA18 | SUZ12_targets | 46 |
| RGS20 | SUZ12_targets | 47 |
| PTHLH | SUZ12_targets | 48 |
| KCNMA1 | SUZ12_targets | 49 |
| ST8SIA1 | SUZ12_targets | 50 |
| GABBR2 | SUZ12_targets | 51 |
| ZFYV28 | SUZ12_targets | 52 |
| CDH7 | SUZ12_targets | 53 |
| GRIA2 | SUZ12_targets | 54 |
| KCNMA1 | SUZ12_targets | 55 |
| STX3 | SUZ12_targets | 56 |
| SPOCK3 | SUZ12_targets | 57 |
| FOXA1 | SUZ12_targets | 58 |
| CDH6 | SUZ12_targets | 59 |
| FAM19A4 | SUZ12_targets | 60 |
| PGR | SUZ12_targets | 61 |
| EMLS | SUZ12_targets | 62 |
| ZIC1 | SUZ12_targets | 63 |
| PKNOX2 | SUZ12_targets | 64 |
| RTN4RL2 | SUZ12_targets | 65 |
| PTHLH | SUZ12_targets | 66 |
| COL4A5 | SUZ12_targets | 67 |
| ESYT3 | SUZ12_targets | 68 |
| GABRA2 | SUZ12_targets | 69 |
| GATA4 | SUZ12_targets | 70 |
| CACNA1D | SUZ12_targets | 71 |
| LPHN3 | SUZ12_targets | 72 |
| XCNV1 | SUZ12_targets | 73 |
| RAPGEF4 | SUZ12_targets | 74 |
| TRPA1 | SUZ12_targets | 75 |
| MYOSB | SUZ12_targets | 76 |
| EN2 | SUZ12_targets | 77 |
| PAX9 | SUZ12_targets | 78 |
| GPC5 | SUZ12_targets | 79 |
| TLL1 | SUZ12_targets | 80 |
| ADRA1A | SUZ12_targets | 81 |
| PDE4DIP | SUZ12_targets | 82 |
| NRK | SUZ12_targets | 83 |
| TMEFF2 | SUZ12_targets | 84 |
| ADAMTS5 | SUZ12_targets | 85 |
| NEFL | SUZ12_targets | 86 |
| VWA3B | SUZ12_targets | 87 |
| XLHDC1 | SUZ12_targets | 88 |
| PTGFR | SUZ12_targets | 89 |
| TMEM26 | SUZ12_targets | 90 |
| MYO5B | SUZ12_targets | 91 |
| KCNJ3 | SUZ12_targets | 92 |
| CLEC4G | SUZ12_targets | 93 |
| FEZF2 | SUZ12_targets | 94 |
| GUCY1A2 | SUZ12_targets | 95 |
| SRPX2 | SUZ12_targets | 96 |
| SHC4 | SUZ12_targets | 97 |
| TBX3 | SUZ12_targets | 98 |
| SIAH3 | SUZ12_targets | 99 |
| PITX2 | SUZ12_targets | 100 |
| ELAVL2 | MIKKELSEN_MEF_HCP_WITH_K3K27ME3 | 1 |
| KCNB1 | MIKKELSEN_MEF_HCP_WITH_K3K27ME4 | 2 |
| BHMT2 | MIKKELSEN_MEF_HCP_WITH_K3K27ME5 | 3 |
| LIN28A | MIKKELSEN_MEF_HCP_WITH_K3K27ME6 | 4 |
| OLIG3 | MIKKELSEN_MEF_HCP_WITH_K3K27ME7 | 5 |
| GRIK3 | MIKKELSEN_MEF_HCP_WITH_K3K27ME8 | 6 |
| SNAP25 | MIKKELSEN_MEF_HCP_WITH_K3K27ME9 | 7 |
| SLC6A1 | MIKKELSEN_MEF_HCP_WITH_K3K27ME10 | 8 |
| RVR2 | MIKKELSEN_MEF_HCP_WITH_K3K27ME11 | 9 |
| CARTPT | MIKKELSEN_MEF_HCP_WITH_K3K27ME12 | 10 |
| NEUROD1 | MIKKELSEN_MEF_HCP_WITH_K3K27ME13 | 11 |
| VSTM2A | MIKKELSEN_MEF_HCP_WITH_K3K27ME14 | 12 |
| CDHB | MIKKELSEN_MEF_HCP_WITH_K3K27ME15 | 13 |
| GABRAS | MIKKELSEN_MEF_HCP_WITH_K3K27ME16 | 14 |
| FEZF2 | MIKKELSEN_MEF_HCP_WITH_K3K27ME17 | 15 |
| RAPGEF4 | MIKKELSEN_MEF_HCP_WITH_K3K27ME18 | 16 |
| KCNJ10 | MIKKELSEN_MEF_HCP_WITH_K3K27ME19 | 17 |
| BAJ3 | MIKKELSEN_MEF_HCP_WITH_K3K27ME20 | 18 |
| HCN1 | MIKKELSEN_MEF_HCP_WITH_K3K27ME21 | 19 |
| DPP10 | MIKKELSEN_MEF_HCP_WITH_K3K27ME22 | 20 |
| CNTN4 | MIKKELSEN_MEF_HCP_WITH_K3K27ME23 | 21 |
| TTC22 | MIKKELSEN_MEF_HCP_WITH_K3K27ME24 | 22 |
| CWH43 | MIKKELSEN_MEF_HCP_WITH_K3K27ME25 | 23 |
| HOXD4 | MIKKELSEN_MEF_HCP_WITH_K3K27ME26 | 24 |
| CALB1 | MIKKELSEN_MEF_HCP_WITH_K3K27ME27 | 25 |
| POU2F3 | MIKKELSEN_MEF_HCP_WITH_K3K27ME28 | 26 |
| KL | MIKKELSEN_MEF_HCP_WITH_K3K27ME29 | 27 |
| OTP | MIKKELSEN_MEF_HCP_WITH_K3K27ME30 | 28 |
| PAQR5 | MIKKELSEN_MEF_HCP_WITH_K3K27ME31 | 29 |
| ADRA1A | MIKKELSEN_MEF_HCP_WITH_K3K27ME32 | 30 |
| RIMKLA | MIKKELSEN_MEF_HCP_WITH_K3K27ME33 | 31 |
| DMRT1 | MIKKELSEN_MEF_HCP_WITH_K3K27ME34 | 32 |
| KCNV1 | MIKKELSEN_MEF_HCP_WITH_K3K27ME35 | 33 |
| KCNC1 | MIKKELSEN_MEF_HCP_WITH_K3K27ME36 | 34 |
| IGFBPL1 | MIKKELSEN_MEF_HCP_WITH_K3K27ME37 | 35 |
| GABRG3 | MIKKELSEN_MEF_HCP_WITH_K3K27ME38 | 36 |
| GRIN2A | MIKKELSEN_MEF_HCP_WITH_K3K27ME39 | 37 |
| SCN8A | MIKKELSEN_MEF_HCP_WITH_K3K27ME40 | 38 |
| SHISA6 | MIKKELSEN_MEF_HCP_WITH_K3K27ME41 | 39 |
| EPHA6 | MIKKELSEN_MEF_HCP_WITH_K3K27ME42 | 40 |
| GRP | MIKKELSEN_MEF_HCP_WITH_K3K27ME43 | 41 |
| NKX2-3 | MIKKELSEN_MEF_HCP_WITH_K3K27ME44 | 42 |
| ADCYAP1 | MIKKELSEN_MEF_HCP_WITH_K3K27ME45 | 43 |
| C11orf63 | MIKKELSEN_MEF_HCP_WITH_K3K27ME46 | 44 |
| SOX18 | MIKKELSEN_MEF_HCP_WITH_K3K27ME47 | 45 |
| DMGDH | MIKKELSEN_MEF_HCP_WITH_K3K27ME48 | 46 |
| SRD5A2 | MIKKELSEN_MEF_HCP_WITH_K3K27ME49 | 47 |
| TBX20 | MIKKELSEN_MEF_HCP_WITH_K3K27ME50 | 48 |
| NPY5R | MIKKELSEN_MEF_HCP_WITH_K3K27ME51 | 49 |
| ST8SIA3 | MIKKELSEN_MEF_HCP_WITH_K3K27ME52 | 50 |
| TCF21 | MIKKELSEN_MEF_HCP_WITH_K3K27ME53 | 51 |
| CHGB | MIKKELSEN_MEF_HCP_WITH_K3K27ME54 | 52 |
| SLC35F3 | MIKKELSEN_MEF_HCP_WITH_K3K27ME55 | 53 |
| SLC34A2 | MIKKELSEN_MEF_HCP_WITH_K3K27ME56 | 54 |
| DLEU7 | MIKKELSEN_MEF_HCP_WITH_K3K27ME57 | 55 |
| FOXD3 | MIKKELSEN_MEF_HCP_WITH_K3K27ME58 | 56 |
| SIX3 | MIKKELSEN_MEF_HCP_WITH_K3K27ME59 | 57 |
| LRAT | MIKKELSEN_MEF_HCP_WITH_K3K27ME60 | 58 |
| INSM1 | MIKKELSEN_MEF_HCP_WITH_K3K27ME61 | 59 |
| CYP24A1 | MIKKELSEN_MEF_HCP_WITH_K3K27ME62 | 60 |
| SLC6A2 | MIKKELSEN_MEF_HCP_WITH_K3K27ME63 | 61 |
| GATA5 | MIKKELSEN_MEF_HCP_WITH_K3K27ME64 | 62 |
| KCNA7 | MIKKELSEN_MEF_HCP_WITH_K3K27ME65 | 63 |
| PRLR | MIKKELSEN_MEF_HCP_WITH_K3K27ME66 | 64 |
| KCNS2 | MIKKELSEN_MEF_HCP_WITH_K3K27ME67 | 65 |
| DCC | MIKKELSEN_MEF_HCP_WITH_K3K27ME68 | 66 |
| IRF6 | MIKKELSEN_MEF_HCP_WITH_K3K27ME69 | 67 |
| LHFPL5 | MIKKELSEN_MEF_HCP_WITH_K3K27ME70 | 68 |
| NKX2-1 | MIKKELSEN_MEF_HCP_WITH_K3K27ME71 | 69 |
| SEZ6L | MIKKELSEN_MEF_HCP_WITH_K3K27ME72 | 70 |
| GAD1 | MIKKELSEN_MEF_HCP_WITH_K3K27ME73 | 71 |
| ONECUT2 | MIKKELSEN_MEF_HCP_WITH_K3K27ME74 | 72 |
| TACR1 | MIKKELSEN_MEF_HCP_WITH_K3K27ME75 | 73 |
| TFAP2B | MIKKELSEN_MEF_HCP_WITH_K3K27ME76 | 74 |
| NHLH2 | MIKKELSEN_MEF_HCP_WITH_K3K27ME77 | 75 |
| ATP2B2 | MIKKELSEN_MEF_HCP_WITH_K3K27ME78 | 76 |
| ALOX15 | MIKKELSEN_MEF_HCP_WITH_K3K27ME79 | 77 |
| TDH | MIKKELSEN_MEF_HCP_WITH_K3K27ME80 | 78 |
| B3GALT5 | MIKKELSEN_MEF_HCP_WITH_K3K27ME81 | 79 |
| CACNA1E | MIKKELSEN_MEF_HCP_WITH_K3K27ME82 | 80 |
| ALOX12B | MIKKELSEN_MEF_HCP_WITH_K3K27ME83 | 81 |
| SORCS3 | MIKKELSEN_MEF_HCP_WITH_K3K27ME84 | 82 |
| SERTM1 | MIKKELSEN_MEF_HCP_WITH_K3K27ME85 | 83 |
| GRIA2 | MIKKELSEN_MEF_HCP_WITH_K3K27ME86 | 84 |
| KCNH7 | MIKKELSEN_MEF_HCP_WITH_K3K27ME87 | 85 |
| QRFPR | MIKKELSEN_MEF_HCP_WITH_K3K27ME88 | 86 |
| NELL1 | MIKKELSEN_MEF_HCP_WITH_K3K27ME89 | 87 |
| LRFN5 | MIKKELSEN_MEF_HCP_WITH_K3K27ME90 | 88 |
| POU4F3 | MIKKELSEN_MEF_HCP_WITH_K3K27ME91 | 89 |
| C14orf39 | MIKKELSEN_MEF_HCP_WITH_K3K27ME92 | 90 |
| DCLX3 | MIKKELSEN_MEF_HCP_WITH_K3K27ME93 | 91 |
| GNG13 | MIKKELSEN_MEF_HCP_WITH_K3K27ME94 | 92 |
| CPLX2 | MIKKELSEN_MEF_HCP_WITH_K3K27ME95 | 93 |
| DPYS | MIKKELSEN_MEF_HCP_WITH_K3K27ME96 | 94 |
| ALOX12 | MIKKELSEN_MEF_HCP_WITH_K3K27ME97 | 95 |
| ZBTB8B | MIKKELSEN_MEF_HCP_WITH_K3K27ME98 | 96 |
| NXPH1 | MIKKELSEN_MEF_HCP_WITH_K3K27ME99 | 97 |
| FGF12 | MIKKELSEN_MEF_HCP_WITH_K3K27ME100 | 98 |
| SLC6A11 | MIKKELSEN_MEF_HCP_WITH_K3K27ME101 | 99 |
| DSCAM | MIKKELSEN_MEF_HCP_WITH_K3K27ME102 | 100 |
| TRPC6 | MIKKELSEN_NPC_HPC_WITH_H3K27ME3 | 1 |
| NPAS2 | MIKKELSEN_NPC_HCP_WITH_H3K27ME3 | 2 |
| LIN28A | MIKKELSEN_NPC_HCP_WITH_H3K27ME3 | 3 |
| GPR37 | MIKKELSEN_NPC_HCP_WITH_H3K27ME3 | 4 |
| COH8 | MIKKELSEN_NPC_HCP_WITH_H3K27ME3 | 5 |
| FOXD3 | MIKKELSEN_NPC_HCP_WITH_H3K27ME3 | 6 |
| CARTPT | MIKKELSEN_NPC_HCP_WITH_H3K27ME3 | 7 |
| CDH8 | MIKKELSEN_NPC_HCP_WITH_H3K27ME3 | 8 |
| SIM1 | MIKKELSEN_NPC_HCP_WITH_H3K27ME3 | 9 |
| GABRA5 | MIKKELSEN_NPC_HCP_WITH_H3K27ME3 | 10 |
| GALNT13 | MIKKELSEN_NPC_HCP_WITH_H3K27ME3 | 11 |
| SLC38A4 | MIKKELSEN_NPC_HCP_WITH_H3K27ME3 | 12 |
| PROM16 | MIKKELSEN_NPC_HCP_WITH_H3K27ME3 | 13 |
| PGR | MIKKELSEN_NPC_HCP_WITH_H3K27ME3 | 14 |
| NXPH2 | MIKKELSEN_NPC_HCP_WITH_H3K27ME3 | 15 |
| TFAP2B | MIKKELSEN_NPC_HCP_WITH_H3K27ME3 | 16 |
| HCN1 | MIKKELSEN_NPC_HCP_WITH_H3K27ME3 | 17 |
| ST8SIA3 | MIKKELSEN_NPC_HCP_WITH_H3K27ME3 | 18 |
| DPP10 | MIKKELSEN_NPC_HCP_WITH_H3K27ME3 | 19 |
| PHLDA2 | MIKKELSEN_NPC_HCP_WITH_H3K27ME3 | 20 |
| FEZF2 | MIKKELSEN_NPC_HCP_WITH_H3K27ME3 | 21 |
| TBX3 | MIKKELSEN_NPC_HCP_WITH_H3K27ME3 | 22 |
| PITX1 | MIKKELSEN_NPC_HCP_WITH_H3K27ME3 | 23 |
| HOXB8 | MIKKELSEN_NPC_HCP_WITH_H3K27ME3 | 24 |
| POU2F3 | MIKKELSEN_NPC_HCP_WITH_H3K27ME3 | 25 |
| PAPPA | MIKKELSEN_NPC_HCP_WITH_H3K27ME3 | 26 |
| RYR2 | MIKKELSEN_NPC_HCP_WITH_H3K27ME3 | 27 |
| NPAS2 | MIKKELSEN_NPC_HCP_WITH_H3K27ME3 | 28 |
| SLC22A3 | MIKKELSEN_NPC_HCP_WITH_H3K27ME3 | 29 |
| CA1O | MIKKELSEN_NPC_HCP_WITH_H3K27ME3 | 30 |
| DMRT1 | MIKKELSEN_NPC_HCP_WITH_H3K27ME3 | 31 |
| IGF8PL1 | MIKKELSEN_NPC_HCP_WITH_H3K27ME3 | 32 |
| SIM1 | MIKKELSEN_NPC_HCP_WITH_H3K27ME3 | 33 |
| PAPPA | MIKKELSEN_NPC_HCP_WITH_H3K27ME3 | 34 |
| SP8 | MIKKELSEN_NPC_HCP_WITH_H3K27ME3 | 35 |
| SFRP1 | MIKKELSEN_NPC_HCP_WITH_H3K27ME3 | 36 |
| COL14A1 | MIKKELSEN_NPC_HCP_WITH_H3K27ME3 | 37 |
| SCNN1G | MIKKELSEN_NPC_HCP_WITH_H3K27ME3 | 38 |
| CBLN4 | MIKKELSEN_NPC_HCP_WITH_H3K27ME3 | 39 |
| GHSR | MIKKELSEN_NPC_HCP_WITH_H3K27ME3 | 40 |
| CNTN2 | MIKKELSEN_NPC_HCP_WITH_H3K27ME3 | 41 |
| SHISA6 | MIKKELSEN_NPC_HCP_WITH_H3K27ME3 | 42 |
| KIAA1045 | MIKKELSEN_NPC_HCP_WITH_H3K27ME3 | 43 |
| NKX2-3 | MIKKELSEN_NPC_HCP_WITH_H3K27ME3 | 44 |
| HOXD10 | MIKKELSEN_NPC_HCP_WITH_H3K27ME3 | 45 |
| LHX8 | MIKKELSEN_NPC_HCP_WITH_H3K27ME3 | 46 |
| SOX18 | MIKKELSEN_NPC_HCP_WITH_H3K27ME3 | 47 |
| HOXA13 | MIKKELSEN_NPC_HCP_WITH_H3K27ME3 | 48 |
| GPR37 | MIKKELSEN_NPC_HCP_WITH_H3K27ME3 | 49 |
| C8orf42 | MIKKELSEN_NPC_HCP_WITH_H3K27ME3 | 50 |
| PYY | MIKKELSEN_NPC_HCP_WITH_H3K27ME3 | 51 |
| BNC1 | MIKKELSEN_NPC_HCP_WITH_H3K27ME3 | 52 |
| VSX1 | MIKKELSEN_NPC_HCP_WITH_H3K27ME3 | 53 |
| GRIK3 | MIKKELSEN_NPC_HCP_WITH_H3K27ME3 | 54 |
| LIPG | MIKKELSEN_NPC_HCP_WITH_H3K27ME3 | 55 |
| T8X3 | MIKKELSEN_NPC_HCP_WITH_H3K27ME3 | 56 |
| ISL1 | MIKKELSEN_NPC_HCP_WITH_H3K27ME3 | 57 |
| GRIK3 | MIKKELSEN_NPC_HCP_WITH_H3K27ME3 | 58 |
| HOXB8 | MIKKELSEN_NPC_HCP_WITH_H3K27ME3 | 59 |
| BNC1 | MIKKELSEN_NPC_HCP_WITH_H3K27ME3 | 60 |
| ATP2B2 | MIKKELSEN_NPC_HCP_WITH_H3K27ME3 | 61 |
| GABRG3 | MIKKELSEN_NPC_HCP_WITH_H3K27ME3 | 62 |
| HOXB8 | MIKKELSEN_NPC_HCP_WITH_H3K27ME3 | 63 |
| LRAT | MIKKELSEN_NPC_HCP_WITH_H3K27ME3 | 64 |
| BNC2 | MIKKELSEN_NPC_HCP_WITH_H3K27ME3 | 65 |
| GABRA5 | MIKKELSEN_NPC_HCP_WITH_H3K27ME3 | 66 |
| NELL1 | MIKKELSEN_NPC_HCP_WITH_H3K27ME3 | 67 |
| FOXD3 | MIKKELSEN_NPC_HCP_WITH_H3K27ME3 | 68 |
| DVOL2 | MIKKELSEN_NPC_HCP_WITH_H3K27ME3 | 69 |
| ATP2B2 | MIKKELSEN_NPC_HCP_WITH_H3K27ME3 | 70 |
| PAPPA | MIKKELSEN_NPC_HCP_WITH_H3K27ME3 | 71 |
| ST8SIA3 | MIKKELSEN_NPC_HCP_WITH_H3K27ME3 | 72 |
| KCNA7 | MIKKELSEN_NPC_HCP_WITH_H3K27ME3 | 73 |
| DMRT2 | MIKKELSEN_NPC_HCP_WITH_H3K27ME3 | 74 |
| GALNT13 | MIKKELSEN_NPC_HCP_WITH_H3K27ME3 | 75 |
| SCN49 | MIKKELSEN_NPC_HCP_WITH_H3K27ME3 | 76 |
| PAX3 | MIKKELSEN_NPC_HCP_WITH_H3K27ME3 | 77 |
| GRM7 | MIKKELSEN_NPC_HCP_WITH_H3K27ME3 | 78 |
| TBX3 | MIKKELSEN_NPC_HCP_WITH_H3K27ME3 | 79 |
| FGF14 | MIKKELSEN_NPC_HCP_WITH_H3K27ME3 | 80 |
| GABRG3 | MIKKELSEN_NPC_HCP_WITH_H3K27ME3 | 81 |
| NKX2-1 | MIKKELSEN_NPC_HCP_WITH_H3K27ME3 | 82 |
| PAPPA | MIKKELSEN_NPC_HCP_WITH_H3K27ME3 | 83 |
| TBX3 | MIKKELSEN_NPC_HCP_WITH_H3K27ME3 | 84 |
| FEZF2 | MIKKELSEN_NPC_HCP_WITH_H3K27ME3 | 85 |
| HOXA13 | MIKKELSEN_NPC_HCP_WITH_H3K27ME3 | 86 |
| PAPPA | MIKKELSEN_NPC_HCP_WITH_H3K27ME3 | 87 |
| NKX2-1 | MIKKELSEN_NPC_HCP_WITH_H3K27ME3 | 88 |
| C6orf132 | MIKKELSEN_NPC_HCP_WITH_H3K27ME3 | 89 |
| TP73 | MIKKELSEN_NPC_HCP_WITH_H3K27ME3 | 90 |
| HOXC9 | MIKKELSEN_NPC_HCP_WITH_H3K27ME3 | 91 |
| SORCS3 | MIKKELSEN_NPC_HCP_WITH_H3K27ME3 | 92 |
| POU2F3 | MIKKELSEN_NPC_HCP_WITH_H3K27ME3 | 93 |
| KCNS2 | MIKKELSEN_NPC_HCP_WITH_H3K27ME3 | 94 |
| LRFN5 | MIKKELSEN_NPC_HCP_WITH_H3K27ME3 | 95 |
| FGF14 | MIKKELSEN_NPC_HCP_WITH_H3K27ME3 | 96 |
| C8orf42 | MIKKELSEN_NPC_HCP_WITH_H3K27ME3 | 97 |
| POU4F3 | MIKKELSEN_NPC_HCP_WITH_H3K27ME3 | 98 |
| CALCR | MIKKELSEN_NPC_HCP_WITH_H3K27ME3 | 99 |
| SIM2 | MIKKELSEN_NPC_HCP_WITH_H3K27ME3 | 100 |

TABLE 6

| Symbol | TRC.Clone.Name | Annotation | CON/ | Target.Seq | Region | T/W1 | T/W2 | T/W3 |
|---|---|---|---|---|---|---|---|---|
| Kcnj15 | NM_019664.3-398s1c1 | NA | TEST | CGACATGAAGTGGCGATACAA | CDS | 1 | 0.262019 | 0.005753 |
| Hmgn1 | NM_008251.3-863s1c1 | H2 | TEST | TGTGGTCATGGCAGTCCATTT | 3UTR | 1 | 0.311644 | 0.005967 |
| Brwd1 | NM_145125.3-4930s1c1 | NA | TEST | ACTCGGAAGAGAGTCTATTTA | CDS | 1 | 0.737082 | 0.006411 |
| Hmgn1 | NM_008251.3-780s1c1 | H4 | TEST | TTCTATCTGGTCCCGTGTTTC | 3UTR | 1 | 0.323601 | 0.00728 |
| Chaf1b | NM_028083.1-718s1c1 | NA | TEST | TGTGGCTTTCAACATTTCAAA | CDS | 1 | 0.292389 | 0.009844 |
| Psmg1 | NM_019537.1-271s1c1 | NA | TEST | GCGTTTGTTATGAACTCGGGA | CDS | 1 | 1.02253 | 0.014719 |
| Kcnj6 | NM_010606.1-1312s1c1 | NA | TEST | CCATTGATTATTAGCCATGAA | CDS | 1 | 1.372994 | 0.02427 |
| Hmgn1 | NM_008251.3-391s1c1 | H5 | TEST | GAAAGAAGCTAAGTCCGACTA | CDS | 1 | 0.659731 | 0.027741 |
| Brwd1 | NM_145125.3-2514s21c1 | NA | TEST | ACGGACGTGTAGGCGTAAATA | CDS | 1 | 0.532465 | 0.031663 |
| Lca5l | NM_001001492.2-1370s1c1 | NA | TEST | CGAAAGTTTCTTCAACGAAAT | CDS | 1 | 1.097417 | 0.03167 |
| Fam3b | NM_020622.2-217s21c1 | NA | TEST | TCTACAACATCCGAAGCATTG | CDS | 1 | 1.620241 | 0.034654 |
| Dopey2 | NM_026700.2-241s1c1 | NA | TEST | CTCAGTAATCGAGAAGGCGTT | CDS | 1 | 0.485752 | 0.043964 |
| Sh3bgr | NM_015825.1-97s1c1 | NA | TEST | GACTTCAAGGAGCTGGACATA | CDS | 1 | 0.734531 | 0.046528 |
| Pcp4 | NM_008791.1-65s1c1 | NA | TEST | CAGGAGATAATGATGGGCAGA | CDS | 1 | 0.851884 | 0.05338 |
| Cbr3 | NM_173047.3-969s21c1 | NA | TEST | CGTTAGCGGGAGAGATGAATG | 3UTR | 1 | 1.207545 | 0.056268 |
| Cbr1 | NM_007620.2-319s21c1 | NA | TEST | ATCGACAACCCGCAGAGCATT | CDS | 1 | 0.936156 | 0.056326 |
| Dyrk1a | NM_007890.2-2489s21c1 | NA | TEST | ATGGAGCTATGGACGTTAATT | CDS | 1 | 0.953841 | 0.057719 |
| Kcnj15 | NM_019664.3-486s1c1 | NA | TEST | GCCTTTATTCATGGTGACTTA | CDS | 1 | 0.820863 | 0.058278 |
| Cldn14 | NM_019500.3-1131s1c1 | NA | TEST | CCGGAGCTACCACCACGGCTA | CDS | 1 | 0.410745 | 0.068165 |
| Hics | NM_139145.2-1979s1c1 | NA | TEST | CGAACAGTAATCCTACCATTT | CDS | 1 | 0.403183 | 0.073492 |
| Lca5l | NM_001001492.2-1596s21c1 | NA | TEST | AGCCAATCTCACGGTCTTAAA | CDS | 1 | 1.345008 | 0.076901 |
| Ttc3 | NM_009441.2-6239s21c1 | NA | TEST | TGCATCACAAAGCTAACAAAT | 3UTR | 1 | 0.817093 | 0.078927 |
| Hmgn1 | NM_008251.3-239s1c1 | H1 | TEST | GCGGGAAAGGATAAAGCATCA | CDS | 1 | 1.014904 | 0.079599 |
| Hics | NM_139145.2-2745s1c1 | NA | TEST | GCGCTGAGATAGTACAAATAT | 3UTR | 1 | 0.696438 | 0.08285 |
| Mx1 | NM_010846.1-2625s1c1 | NA | TEST | CCCATAACAAACACCAAGTAT | 3UTR | 1 | 0.592746 | 0.084034 |
| Itgb2l | NM_008405.1-2333s1c1 | NA | TEST | CCCTATGACCAATCAGGACAT | 3UTR | 1 | 0.941228 | 0.085309 |
| Dscr3 | NM_007834.3-750s21c1 | NA | TEST | CACTTCCCAAATTCTTCATTA | CDS | 1 | 0.813688 | 0.100073 |
| Dyrk1a | NM_007890.1-1374s1c1 | NA | TEST | GCTGACTACTTGAAGTTCAAA | CDS | 1 | 0.848518 | 0.105306 |
| Mx1 | NM_010846.1-882s1c1 | NA | TEST | AGGCAAGGTCTTGGATGTGAT | CDS | 1 | 0.732846 | 0.110915 |
| Hics | NM_139145.2-1632s1c2 | NA | TEST | GCCGCAGGAAATGGGCTTAAT | CDS | 1 | 0.86791 | 0.111295 |
| Fam3b | NM_020622.2-424s21c1 | NA | TEST | ACATTGCTGTCGTCAACTATG | CDS | 1 | 0.891033 | 0.117798 |
| Dopey2 | NM_026700.2-217s1c1 | NA | TEST | CGATTACAGATACAGAAGCTA | CDS | 1 | 0.865242 | 0.118681 |
| Psmg1 | NM_019537.1-259s1c1 | NA | TEST | GCATTCCTGTCAGCGTTTGTT | CDS | 1 | 1.022196 | 0.122801 |
| Pigp | NM_019543.3-549s21c1 | NA | TEST | GCTGTGTATAAACATCCTAAA | 3UTR | 1 | 1.044511 | 0.123754 |
| Cldn14 | NM_019500.3-989s1c1 | NA | TEST | CCCAGTGGCATGAAGTTTGAA | CDS | 1 | 0.767389 | 0.129964 |
| Psmg1 | NM_019537.1-637s1c1 | NA | TEST | GAACAGCCGAACATTGTGCAT | CDS | 1 | 0.836322 | 0.133372 |
| B3galt5 | NM_033149.2-660s1c1 | NA | TEST | CAGACAGCTTACGTGATGAAA | CDS | 1 | 0.950746 | 0.139898 |
| Cbr3 | NM_173047.3-759s21c1 | NA | TEST | CGGACAGGATTCTGCTCAATG | CDS | 1 | 0.902533 | 0.150334 |
| Ripply3 | NM_133229.1-1098s1c1 | NA | TEST | CCTGAGTTCAATTCTCAGCAA | 3UTR | 1 | 1.098118 | 0.150287 |
| Dopey2 | NM_026700.2-341s1c1 | NA | TEST | CTGAAGTATTCCCTCCTACCA | CDS | 1 | 1.017985 | 0.166687 |
| Dopey2 | NM_026700.2-445s1c1 | NA | TEST | CTACGAGATCATCTTCAAGAT | CDS | 1 | 0.662505 | 0.181425 |
| Dscam | NM_031174.2-3121s1c1 | NA | TEST | CCTCCCGAGATTGAGATCAAA | CDS | 1 | 0.855194 | 0.184066 |
| Chaf1b | NM_028083.4-795s1c1 | NA | TEST | CGACAGCATGAAGTCGTTCTT | CDS | 1 | 1.113682 | 0.197147 |
| Cbr3 | NM_173047.3-471s21c1 | NA | TEST | GCACTGAGTTACTGCCTATAA | CDS | 1 | 0.502186 | 0.209943 |
| Wrb | NM_207301.1-776s1c1 | NA | TEST | CGTCTGATGTAGGTCTGGATT | 3UTR | 1 | 0.783118 | 0.215621 |
| Kcnj6 | NM_010606.1-512s1c1 | NA | TEST | CTAACGTCTTGGAAGGCGATT | CDS | 1 | 0.341558 | 0.218176 |
| Lca5l | NM_001001492.2-944s21c1 | NA | TEST | TTCGACAGCTCCTCCGGAAAT | CDS | 1 | 0.71527 | 0.220497 |
| Igsf5 | NM_028078.2-991s21c1 | NA | TEST | TCACCGGAGCTGATGGTTAAT | 3UTR | 1 | 0.887147 | 0.24106 |
| Dscam | NM_031174.2-6672s1c1 | NA | TEST | GCGCAAAGACTACTCTGCTTT | 3UTR | 1 | 1.35574 | 0.241612 |
| Cbr1 | NM_007620.2-404s21c1 | NA | TEST | GCATCGCCTTCAAGGTCAATG | CDS | 1 | 1.27317 | 0.246949 |
| Dscr3 | NM_007834.1-1333s1c1 | NA | TEST | GCTCTCTGATTGAGTCTGTAA | 3UTR | 1 | 0.398041 | 0.247807 |
| B3galt5 | NM_033149.2-1110s1c1 | NA | TEST | GCACTGGAGAACTCGAAAGAA | CDS | 1 | 0.940939 | 0.250766 |
| Ttc3 | NM_009441.2-3398s21c1 | NA | TEST | CGAGTATGTTGTCCGAAATAA | CDS | 1 | 1.545515 | 0.270314 |
| Fam3b | NM_020622.1-579s1c1 | NA | TEST | CAAACTGAAGGCTCAAGCAAA | CDS | 1 | 0.792782 | 0.279329 |
| Dscr3 | NM_007834.3-343s21c1 | NA | TEST | CCAGGGAGTCTCTTTGACAAT | CDS | 1 | 0.700519 | 0.282225 |
| B3galt5 | NM_033149.2-998s1c1 | NA | TEST | GCACACCAAACAGACCTTCTT | CDS | 1 | 1.115935 | 0.285619 |
| Cbr3 | NM_173047.3-994s21c1 | NA | TEST | CTGGTGTGGTCTGATTCTTTC | 3UTR | 1 | 1.246149 | 0.286052 |
| Mx2 | NM_013606.1-1434s1c1 | NA | TEST | CCAGGGTTTGTGAATTACAAA | CDS | 1 | 0.723516 | 0.293613 |
| Morc3 | NM_001045529.2-886s21c1 | NA | TEST | GTGATGTTTACCGACCTAAAT | CDS | 1 | 1.288987 | 0.297922 |
| Dyrk1a | NM_007890.2-1801s21c1 | NA | TEST | ACTCGGATTCAACCTTATTAT | CDS | 1 | 1.037012 | 0.29793 |
| Igsf5 | NM_028078.2-865s21c1 | NA | TEST | GAAATGTGACTTTAGTGTAAT | CDS | 1 | 0.995933 | 0.301536 |
| Morc3 | NM_001045529.2-190s21c1 | NA | TEST | CAGTGATTAGTGACCATATAT | CDS | 1 | 1.238075 | 0.304265 |
| Mx2 | NM_013606.1-146s1c1 | NA | TEST | AGGCGTTGATTCAGTCAACTT | CDS | 1 | 0.892911 | 0.307939 |
| Sim2 | NM_011377.1-1599s1c1 | NA | TEST | CCTTGACCTGAAGCTCATATT | CDS | 1 | 1.042186 | 0.312377 |
| Kcnj15 | NM_019664.3-411s1c1 | NA | TEST | CGATACAAGCTCACCCTATTT | CDS | 1 | 0.71167 | 0.313412 |
| Kcnj6 | NM_010606.1-1032s1c1 | NA | TEST | CGCCTTCATGGTAGGATGTAT | CDS | 1 | 0.878819 | 0.322629 |
| Ets2 | NM_011809.2-888s21c1 | NA | TEST | CAACACCGTCAATGTCAATTA | CDS | 1 | 1.577949 | 0.342707 |
| Chaf1b | NM_028083.4-307s21c1 | NA | TEST | GCTGTCAATGTTGTACGCTTT | CDS | 1 | 0.681101 | 0.344537 |
| Wrb | NM_207301.1-398s1c1 | NA | TEST | GCGCTGATGATCTCGCTCATT | CDS | 1 | 1.043596 | 0.351375 |
| Pcp4 | NM_008791.1-63s1c1 | NA | TEST | GTCAGGAGATAATGATGGGCA | CDS | 1 | 0.739351 | 0.3675 |
| Setd4 | NM_145482.1-595s1c1 | NA | TEST | GCTGATGAGCAAAGCATCGTT | CDS | 1 | 1.644677 | 0.37638 |
| Hmgn1 | NM_008251.3-721s21c1 | H3 | TEST | AGTATACTAAATGGCAATTTG | 3UTR | 1 | 1.136239 | 0.38443 |
| Dscr3 | NM_007834.3-921s21c1 | NA | TEST | CCACAGAGATTCAGAATATTC | CDS | 1 | 0.773284 | 0.388011 |
| Morc3 | XM_128334.2-1060s1c1 | NA | TEST | GCCTACATTGAACGTGATGTT | CDS | 1 | 0.940087 | 0.393441 |
| Kcnj15 | NM_019664.3-1158s1c1 | NA | TEST | CCTGTGGTTTCTCTCTCCAAA | CDS | 1 | 1.084377 | 0.402942 |
| Morc3 | NM_001045529.2-2835s21c1 | NA | TEST | AGCGAGATCAGCAGTACTTAA | CDS | 1 | 1.200395 | 0.40593 |
| Chaf1b | NM_028083.1-691s1c1 | NA | TEST | GCGGATCTATAATACCCAGAA | CDS | 1 | 0.694971 | 0.406916 |
| Mx1 | NM_010846.1-1129s1c1 | NA | TEST | CCACTATTGGAAGATCAAATA | CDS | 1 | 1.111896 | 0.417384 |
| Cldn14 | NM_019500.3-1295s1c1 | NA | TEST | GACCAATGATGGATGTGGGAA | 3UTR | 1 | 0.690384 | 0.43861 |
| Ttc3 | NM_009441.1-3612s1c1 | NA | TEST | CGAGTTAAACTACCACTAAAT | CDS | 1 | 1.413328 | 0.502301 |
| Cldn14 | NM_019500.3-1230s1c1 | NA | TEST | ACAGGCTGAATGACTACGTGT | CDS | 1 | 0.614018 | 0.50669 |
| Mx2 | NM_013606.1-2168s1c1 | NA | TEST | CCAGCCTTTATGCTCTGATAA | 3UTR | 1 | 1.209092 | 0.508494 |
| Wrb | NM_207301.1-374s1c1 | NA | TEST | GTTGCTTTCTACATACTACAA | CDS | 1 | 0.73874 | 0.512005 |
| Dscam | NM_031174.2-4698s1c1 | NA | TEST | CCTGCAATACTCCGAGGATAA | CDS | 1 | 0.615097 | 0.515247 |
| Cbr3 | NM_173047.3-404s21c1 | NA | TEST | CCAACACCCTTCGACATTCAA | CDS | 1 | 0.853094 | 0.518373 |
| Dyrk1a | NM_007890.1-933s1c1 | NA | TEST | CGGAGTGCAATCAAGATTGTT | CDS | 1 | 1.044202 | 0.519929 |
| Wrb | NM_207301.1-371s1c1 | NA | TEST | AGTGTTGCTTTCTACATACTA | CDS | 1 | 1.329557 | 0.525817 |
| Pcp4 | NM_008791.1-82s1c1 | NA | TEST | CAGAAGAAAGTCCAAGAAGAA | CDS | 1 | 1.700277 | 0.555452 |
| Fam3b | NM_020622.1-589s1c1 | NA | TEST | GCTCAAGCAAAGGATGCCATA | CDS | 1 | 0.638145 | 0.576912 |
| Ttc3 | NM_009441.2-2781s21c1 | NA | TEST | AGAGTAAAGACACGGATATTT | CDS | 1 | 1.125443 | 0.585033 |
| Sim2 | NM_011377.1-1452s1c1 | NA | TEST | CCTAAAGATCAGACAGTACAT | CDS | 1 | 1.023242 | 0.60602 |
| Pigp | NM_019543.2-491s1c1 | NA | TEST | CCTCCTTATCACAGTTGTAAT | CDS | 1 | 1.22691 | 0.626382 |
| Pcp4 | NM_008791.1-100s1c1 | NA | TEST | GAATTTGATATCGACATGGAT | CDS | 1 | 2.220634 | 0.636148 |
| Ripply3 | NM_133229.1-1090s1c1 | NA | TEST | CCAGAGATCCTGAGTTCAATT | 3UTR | 1 | 0.595448 | 0.640482 |
| Ripply3 | NM_133229.1-334s1c1 | NA | TEST | CCGTTTCAAAGCGTCAAGAAT | CDS | 1 | 0.710238 | 0.644686 |
| Cbr1 | NM_007620.1-158s1c1 | NA | TEST | GACCGGTGCTAACAAAGGAAT | CDS | 1 | 0.821093 | 0.665043 |
| Ets2 | NM_011809.2-3074s21c1 | NA | TEST | CATTGATAAAGAGCCGTTATA | 3UTR | 1 | 1.337104 | 0.689839 |
| Psmg1 | NM_019537.1-707s1c1 | NA | TEST | CGGTTCTGTATCTGTGCTACA | CDS | 1 | 0.925217 | 0.746869 |
| Dopey2 | NM_026700.2-436s1c1 | NA | TEST | CCTAGAAACCTACGAGATCAT | CDS | 1 | 0.910334 | 0.750453 |
| Chaf1b | NM_028083.1-370s1c1 | NA | TEST | CGTCATTCTGTTGTGGAAGAT | CDS | 1 | 0.85193 | 0.768556 |
| Brwd1 | NM_145125.3-1598s21c1 | NA | TEST | GCAGCATATTTATATGGGATA | CDS | 1 | 1.236461 | 0.774918 |
| Itgb2l | NM_008405.1-694s1c1 | NA | TEST | GCTGTGGTTCAAGTTGCCATA | CDS | 1 | 1.04169 | 0.790009 |
| Wrb | NM_207301.1-244s1c1 | NA | TEST | CGTCAACATGATGGACGAGTT | CDS | 1 | 1.472302 | 0.792522 |
| Mx1 | NM_010846.1-2088s1c1 | NA | TEST | GCTTGCCAAATTCTCCGATTA | CDS | 1 | 0.562346 | 0.883028 |
| Igsf5 | NM_028078.2-303s21c1 | NA | TEST | CGCTTCACCTATGCCAGTTAC | CDS | 1 | 0.690036 | 0.910138 |
| Mx1 | NM_010846.1-1024s1c1 | NA | TEST | GATCACTCATACTTCAGCATT | CDS | 1 | 0.618863 | 0.921351 |
| Ttc3 | NM_009441.1-6951s1c1 | NA | TEST | CACTCCTTATTCTGAGACATT | 3UTR | 1 | 0.866533 | 0.928361 |
| Pigp | NM_019543.3-486s21c1 | NA | TEST | CCCATTAGTGAAGTAAACAAA | CDS | 1 | 1.667867 | 0.935228 |
| Sh3bgr | NM_015825.1-135s1c1 | NA | TEST | CAGAAAGTGGATGAGAGAGAA | CDS | 1 | 0.737922 | 1.00398 |
| Erg | NM_133659.1-782s1c1 | NA | TEST | CCGATGACGTTGATAAGGCTT | CDS | 1 | 0.563659 | 1.017303 |
| Lca5l | NM_001001492.2-2607s21c1 | NA | TEST | TGGGTGGACTGTGGGTAATTT | 3UTR | 1 | 1.212686 | 1.036888 |
| Sim2 | NM_011377.1-2919s1c1 | NA | TEST | GCGAACTGTATATGCACGATA | CDS | 1 | 0.841429 | 1.044894 |
| Mx2 | NM_013606.1-1336s1c1 | NA | TEST | CCTGGAGTAAGGAGATCGAAA | CDS | 1 | 1.303446 | 1.142799 |
| Morc3 | NM_001045529.3-545s21c1 | NA | TEST | ACACCGTCAGATGATTAATTT | CDS | 1 | 1.161566 | 1.160833 |
| Pigp | NM_019543.2-566s1c1 | NA | TEST | CATTCATACGATCACAGATAA | CDS | 1 | 1.102988 | 1.197092 |
| Dscr3 | NM_007834.1-380s1c1 | NA | TEST | CGGCGTGTTTGTCAACATTCA | CDS | 1 | 0.672708 | 1.223353 |
| Brwd1 | NM_145125.3-7514s21c1 | NA | TEST | AGACTGTCATTAATGTCTTAT | 3UTR | 1 | 0.904536 | 1.256228 |
| Erg | NM_133659.1-1410s1c1 | NA | TEST | CCTGCCATACATGGGCTCCTA | CDS | 1 | 1.592418 | 1.333153 |
| B3galt5 | NM_033149.2-339s1c1 | NA | TEST | CACGGGAAGTTCCTTCAGATT | CDS | 1 | 1.099297 | 1.395305 |
| Ets2 | NM_011809.2-644s21c1 | NA | TEST | ATCTAGAGCAGATGATCAAAG | CDS | 1 | 1.126559 | 1.403791 |
| Bace2 | NM_019517.2-333s1c1 | NA | TEST | CCGCAGAAGGTACAGATTCTT | CDS | 1 | 1.343831 | 1.4183 |
| Cbr1 | NM_007620.2-683s21c1 | NA | TEST | CGGAAGAAGGTTGGCCTAATA | CDS | 1 | 2.665387 | 1.431895 |
| Setd4 | NM_145482.1-1165s1c1 | NA | TEST | CCAGGTGCTATGAGATTAGAA | CDS | 1 | 0.935685 | 1.568282 |
| Itgb2l | NM_008405.1-2124s1c1 | NA | TEST | GCTGGTTTACTGTATGGTTTA | CDS | 1 | 0.786507 | 1.634061 |
| Erg | NM_133659.1-216s1c1 | NA | TEST | GTCACTATTTGAGTGTGCCTA | CDS | 1 | 1.003252 | 1.670309 |
| Ripply3 | NM_133229.1-311s1c1 | NA | TEST | GCATCCTGTCAGACTTTACTT | CDS | 1 | 1.060587 | 1.750872 |
| Hics | NM_139145.2-2215s1c1 | NA | TEST | GCATCTATTGTGGGCCTTGAT | CDS | 1 | 1.162177 | 1.780441 |
| Bace2 | NM_019517.2-1266s1c1 | NA | TEST | GAAGGCTTCTACGTGGTCTTT | CDS | 1 | 1.084699 | 1.781672 |
| Bace2 | NM_019517.2-689s1c1 | NA | TEST | CCAAGCAAAGATTCCAGACAT | CDS | 1 | 1.042177 | 1.803735 |
| Cbr1 | NM_007620.1-164s1c1 | NA | TEST | TGCTAACAAAGGAATCGGATT | CDS | 1 | 0.451442 | 1.874891 |
| Setd4 | NM_145482.1-1505s1c1 | NA | TEST | CGAAGTCATCTCCGATACAAA | CDS | 1 | 1.079801 | 1.950667 |
| Kcnj15 | NM_019664.3-1191s1c1 | NA | TEST | GTGGCTGATTTCAGTCAATTT | CDS | 1 | 0.535004 | 1.957336 |
| Erg | NM_133659.1-721s1c1 | NA | TEST | GCCGACATTCTTCTCTCACAT | CDS | 1 | 1.041478 | 2.025188 |
| Setd4 | NM_145482.1-492s1c1 | NA | TEST | GAGAGCTACAGATCAGAATTT | CDS | 1 | 0.682807 | 2.067491 |
| Bace2 | NM_019517.2-2680s1c1 | NA | TEST | GCCCAAGTGTAGCAATCCAAA | 3UTR | 1 | 1.323638 | 2.129183 |
| Pigp | NM_019543.2-413s1c1 | NA | TEST | CGTTCCCGAATCTTGGTTAAA | CDS | 1 | 1.232785 | 2.129253 |
| Ripply3 | NM_133229.1-757s1c1 | NA | TEST | GCCAGGAACTCCACTTTCTTT | 3UTR | 1 | 1.126719 | 2.206198 |
| Sim2 | NM_011377.1-1359s1c1 | NA | TEST | GCGGTCTTTCTTTCTTCGAAT | CDS | 1 | 1.816283 | 2.360479 |
| Dscam | NM_031174.2-2379s1c1 | NA | TEST | CCTCGGAGTAACCATTGACAA | CDS | 1 | 0.903466 | 2.528164 |
| Brwd1 | NM_145125.3-3829s21c1 | NA | TEST | CATAATGCAAGAACGTTTAAT | CDS | 1 | 0.95516 | 2.53426 |
| Cldn14 | NM_019500.3-950s1c1 | NA | TEST | ACGAATGACGTGGTGCAGAAT | CDS | 1 | 0.713436 | 2.780841 |
| Mx2 | NM_013606.1-1609s1c1 | NA | TEST | CCAAACTTGAAGACATCAGAT | CDS | 1 | 1.304919 | 2.893474 |
| Sh3bgr | NM_015825.1-421s1c1 | NA | TEST | GCACAGAAAGAGGACAGTGAA | CDS | 1 | 0.927064 | 3.275411 |
| Hics | NM_139145.2-655s1c2 | NA | TEST | GCGCCCAATATCTTGCTGTAT | CDS | 1 | 0.914589 | 3.397188 |
| Itgb2l | NM_008405.1-2010s1c1 | NA | TEST | CCAGAGTGACATCAATTCCAT | CDS | 1 | 1.514644 | 3.49855 |
| Ets2 | NM_011809.2-390s21c1 | NA | TEST | AGTGATGAGCCAAGCCTTAAA | CDS | 1 | 3.571751 | 3.604444 |
| Dscam | NM_031174.2-3478s1c1 | NA | TEST | GCTCCCAAGAAACACTTACAA | CDS | 1 | 1.317159 | 3.849103 |
| Sh3bgr | NM_015825.1-465s1c1 | NA | TEST | CCAAGAGAAGAAGGAAGAAGA | CDS | 1 | 0.953016 | 3.912236 |
| Itgb2l | NM_008405.1-758s1c1 | NA | TEST | TGCTTGTTACTGACAACGATT | CDS | 1 | 1.433739 | 4.014413 |
| Kcnj6 | NM_010606.1-1723s1c1 | NA | TEST | GACGTGGCAAACCTAGAGAAT | CDS | 1 | 0.981347 | 4.020144 |
| Igsf5 | NM_028078.2-230s21c1 | NA | TEST | GCTTCTCATGTGGACTCTTAA | CDS | 1 | 0.707363 | 4.823075 |
| B3galt5 | NM_033149.2-794s1c1 | NA | TEST | GCAGAAGTTCAACAAGTGGTT | CDS | 1 | 0.819568 | 5.883202 |
| Bace2 | NM_019517.2-795s1c1 | NA | TEST | CCAAGTTTGTATAAAGGAGAT | CDS | 1 | 1.40239 | 5.945337 |
| Lca5l | NM_001001492.2-1319s21c1 | NA | TEST | ACATCTATACGAATCGAATAC | CDS | 1 | 1.446607 | 6.788565 |
| Dyrk1a | NM_007890.1-1232s1c1 | NA | TEST | CGATGGCACTTGGAGCTTAAA | CDS | 1 | 0.774649 | 6.989936 |
| Pcp4 | NM_008791.1-97s1c1 | NA | TEST | GAAGAATTTGATATCGACATG | CDS | 1 | 0.97128 | 7.259136 |
| Ets2 | NM_011809.2-1183s21c1 | NA | TEST | GAGCAAGGCAAACCAGTTATT | CDS | 1 | 1.821932 | 7.367064 |
| Sim2 | NM_011377.1-3699s1c1 | NA | TEST | CGCTTATATTTGCTTGCGATT | 3UTR | 1 | 1.170393 | 7.523814 |
| Fam3b | NM_020622.2-370s21c1 | NA | TEST | TTGAGGATGAAGTGCTAATAG | CDS | 1 | 0.838338 | 7.721862 |
| Igsf5 | NM_028078.2-126s21c1 | NA | TEST | ACAGCTTCCGGATCCAGTTAT | CDS | 1 | 0.946111 | 7.93552 |
| Kcnj6 | NM_010606.1-1201s1c1 | NA | TEST | GCCAAGTTGATCAAGTCCAAA | CDS | 1 | 0.68659 | 8.324798 |
| Erg | NM_133659.1-880s1c1 | NA | TEST | CCCGAAGCTACGCAAAGAATT | CDS | 1 | 1.091177 | 10.25676 |

Controls

| Symbol | TRC.Clone.Name | Annotation | CON/TEST | Target.Seq | Region | T/W1 | T/W2 | T/W3 |
|---|---|---|---|---|---|---|---|---|
| GFP | clonetechGfp_197s1c1 | NA | NEG | CCTACGGCGTGCAGTGCTTCA | 3UTR | 1 | 0.711768 | 0.019541 |
| | | | CONTROL | | | | | |
| GFP | clonetechGfp_587s1c1 | NA | NEG | TGCCCGACAACCACTACCTGA | 3UTR | 1 | 0.56886 | 0.04338 |
| | | | CONTROL | | | | | |
| RFP | rfp_401s1c1 | NA | NEG | CCGTAATGCAGAAGAAGACCA | 3UTR | 1 | 1.118535 | 0.107563 |
| | | | CONTROL | | | | | |
| LUCIFERASE | promegaLuc_229s1c1 | NA | NEG | AGAATCGTCGTATGCAGTGAA | 3UTR | 1 | 0.710737 | 0.187347 |
| | | | CONTROL | | | | | |
| lacZ | lacZ.1935s1c1 | NA | NEG | CCGTCATAGCGATAACGAGTT | 3UTR | 1 | 1.522221 | 0.220807 |
| | | | CONTROL | | | | | |
| lacZ | lacZ_305s1c1 | NA | NEG | CCAACGTCACCTATCCCATTA | 3UTR | 1 | 1.05715 | 0.355639 |
| | | | CONTROL | | | | | |
| GFP | clonetechGfp_128s1c1 | NA | NEG | TGACCCTGAAGTTCATCTGCA | 3UTR | 1 | 0.58813 | 0.558256 |
| | | | CONTROL | | | | | |
| GFP | clonetechGfp_437s1c1 | NA | NEG | ACAACAGCCACAACGTCTATA | 3UTR | 1 | 1.076918 | 0.648104 |
| | | | CONTROL | | | | | |
| lacZ | lacZ_1758s1c1 | NA | NEG | GTCGGCTTACGGCGGTGATTT | 3UTR | 1 | 0.8855 | 0.728841 |
| | | | CONTROL | | | | | |
| LUCIFERASE | promegaLuc_154s1c1 | NA | NEG | ACTTACGCTGAGTACTTCGAA | 3UTR | 1 | 0.760506 | 1.437221 |
| | | | CONTROL | | | | | |
| RFP | rfp_188s1c1 | NA | NEG | CTCAGTTCCAGTACGGCTCCA | 3UTR | 1 | 0.850796 | 1.466796 |
| | | | CONTROL | | | | | |
| GFP | clonetechGfp_231s1c1 | NA | NEG | CCACATGAAGCAGCACGACTT | 3UTR | 1 | 2.181053 | 1.817256 |
| | | | CONTROL | | | | | |
| RFP | rfp_269s1c1 | NA | NEG | GCTTCAAGTGGGAGCGCGTGA | 3UTR | 1 | 2.119001 | 5.407613 |
| | | | CONTROL | | | | | |
| Psmd2 | NM_134101.1-331s1c1 | NA | POS | GCGTCCACACTATGGCAAATT | | 1 | 1.095814 | 0.001416 |
| | | | CONTROL | | | | | |
| Eif5b | NM_198303.1-250s1c1 | NA | POS | GCAGACACTAAATGCTATCAA | | 1 | 0.687082 | 0.036215 |
| | | | CONTROL | | | | | |
| Rpl10 | NM_052835.1-87s1c1 | NA | POS | GCCATACCCAAAGTCTCGTTT | | 1 | 0.608579 | 0.101431 |
| | | | CONTROL | | | | | |
| Rps4x | NM_009094.1-204s1c1 | NA | POS | GCAGCGATTCATTAAGATTGA | | 1 | 1.639652 | 0.232242 |
| | | | CONTROL | | | | | |
| Pgk1 | NM_008828.1-146s1c1 | NA | POS | GCTGCTGTTCCAAGCATCAAA | | 1 | 0.837868 | 0.282111 |
| | | | CONTROL | | | | | |
| Rp54x | NM_009094.1-170s1c1 | NA | POS | CCCTGACTGGAGATGAAGTAA | | 1 | 2.229778 | 0.374283 |
| | | | CONTROL | | | | | |
| Eif5b | NM_198303.1-977s1c1 | NA | POS | GTGGAGCTGAAGAAAGTATTT | | 1 | 1.038583 | 0.63333 |
| | | | CONTROL | | | | | |
| Rpl10 | NM_052835.1-456s1c1 | NA | POS | CCGAACCAAGTTGCAGAACAA | | 1 | 1.9577 | 2.890988 |
| | | | CONTROL | | | | | |
| Rp15 | NM_016980.1-508s1c1 | NA | POS | CGAACTACAACTGGCAATAAA | | 1 | 0.892507 | 11.54739 |
| | | | CONTROL | | | | | |
| Rp15 | NM_016980.1-679s1c1 | NA | POS | CGCTACCTAATGGAGGAAGAT | | 1 | 2.23561 | 11.62646 |
| | | | CONTROL | | | | | |

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of determining whether a subject afflicted with a cancer or at risk for developing a cancer would benefit from modulating histone H3K27me3 levels, the method comprising:
a) obtaining a biological sample from the subject;
b) determining the copy number, level of expression, or level of activity of one or more biomarkers listed in Tables 1-5 or a fragment thereof in a subject sample;
c) determining the copy number, level of expression, or level of activity of the one or more biomarkers in a control; and
d) comparing the copy number, level of expression, or level of activity of said one or more biomarkers detected in steps b) and c);
wherein a significant modulation in the copy number, level of expression, or level of activity of the one or more biomarkers in the subject sample relative to the control copy number, level of expression, or level of activity of the one or more biomarkers indicates that the subject afflicted with the cancer or at risk for developing the cancer would benefit from modulating histone H3K27me3 levels.

2. The method of claim 1, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

3. A method for monitoring the progression of a cancer in a subject, the method comprising:
a) detecting in a subject sample at a first point in time the copy number, level of expression, or level of activity of one or more biomarkers listed in Tables 1-5 or a fragment thereof,
b) repeating step a) at a subsequent point in time; and
c) comparing the copy number, level of expression, or level of activity of said one or more biomarkers detected in steps a) and b) to monitor the progression of the cancer.

4. The method of claim 3, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

5-6. (canceled)

7. A method for stratifying subjects afflicted with a cancer according to predicted clinical outcome of treatment with one or more modulators of histone H3K27me3 levels, the method comprising:
a) determining the copy number, level of expression, or level of activity of one or more biomarkers listed in Tables 1-5 or a fragment thereof in a subject sample;
b) determining the copy number, level of expression, or level of activity of the one or more biomarkers in a control sample; and
c) comparing the copy number, level of expression, or level of activity of said one or more biomarkers detected in steps a) and b);
wherein a significant modulation in the copy number, level of expression, or level of activity of the one or more biomarkers in the subject sample relative to the normal copy number, level of expression, or level of activity of the one or more biomarkers in the control sample predicts the clinical outcome of the patient to treatment with one or more modulators of histone H3K27me3 levels.

8. The method of claim 7, wherein the predicted clinical outcome is (a) cellular growth, (b) cellular proliferation, or (c) survival time resulting from treatment with one or more modulators of histone H3K27me3 levels.

9. The method of claim 7, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

10-12. (canceled)

13. A method of determining the efficacy of a test compound for inhibiting a cancer in a subject, the method comprising:
a) determining the copy number, level of expression, or level of activity of one or more biomarkers listed in Tables 1-5 or a fragment thereof in a first sample obtained from the subject and exposed to the test compound;
b) determining the copy number, level of expression, or level of activity of the one or more biomarkers in a second sample obtained from the subject, wherein the second sample is not exposed to the test compound, and
c) comparing the copy number, level of expression, or level of activity of the one or more biomarkers in the first and second samples,
wherein a significantly modulated copy number, level of expression, or level of activity of the biomarker, relative to the second sample, is an indication that the test compound is efficacious for inhibiting the cancer in the subject.

14. The method of claim 13, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

15. (canceled)

16. A method of determining the efficacy of a therapy for inhibiting a cancer in a subject, the method comprising:
a) determining the copy number, level of expression, or level of activity of one or more biomarkers listed in Tables 1-5 or a fragment thereof in a first sample obtained from the subject prior to providing at least a portion of the therapy to the subject;
b) determining the copy number, level of expression, or level of activity of the one or more biomarkers in a second sample obtained from the subject following provision of the portion of the therapy; and
c) comparing the copy number, level of expression, or level of activity of the one or more biomarkers in the first and second samples,
wherein a significantly modulated copy number, level of expression, or level of activity of the one or more biomarkers in the second sample, relative to the first sample, is an indication that the therapy is efficacious for inhibiting the cancer in the subject.

17. (canceled)

18. A method for identifying a compound which inhibits a cancer, the method comprising:
a) contacting one or more biomarkers listed in Tables 1-5 or a fragment thereof with a test compound; and
b) determining the effect of the test compound on the copy number, level of expression, or level of activity of the one or more biomarkers to thereby identify a compound which inhibits the cancer.

19. The method of claim 18, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

20-22. (canceled)

23. A method for inhibiting a cancer, the method comprising contacting a cell with an agent that modulates the copy number, level of expression, or level of activity of one or more biomarkers listed in Tables 1-5 or a fragment thereof to thereby inhibit the cancer.

24. The method of claim 23, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

25-27. (canceled)

28. A method for treating a subject afflicted with a cancer, the method comprising administering an agent that modulates the copy number, level of expression, or level of activity of one or more biomarkers listed in Tables 1-5 or a fragment thereof such that the cancer is treated.

29. The method of claim 28, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

30-32. (canceled)

33. A composition selected from the group consisting of
a pharmaceutical composition comprising a polynucleotide encoding one or more biomarkers listed in Tables 1-5 or a fragment thereof useful for treating cancer in a pharmaceutically acceptable carrier;
a kit comprising an agent which selectively binds to one or more biomarkers listed in Tables 1-5 or a fragment thereof and instructions for use;
a kit comprising an agent which selectively hybridizes to a polynucleotide encoding one or more biomarkers listed in Tables 1-5 or fragment thereof and instructions for use; and
a biochip comprising a solid substrate, said substrate comprising a plurality of probes capable of detecting one or more biomarkers listed in Tables 1-5 or a fragment thereof wherein each probe is attached to the substrate at a spatially defined address.

34-39. (canceled)

40. The composition of claim 33, wherein the one or more biomarkers are selected from the group consisting of the set of a) “top 150 UP” biomarkers shown in Table 1, b) “the 50 UP core” biomarkers shown in Table 1, c) “top 150 DOWN” biomarkers shown in Table 1, d), “the 50 DOWN core” biomarkers shown in Table 1, e) the “triplicated gene” biomarkers shown in Table 1, f) the “chr21q22 overlap” biomarkers shown in Table 2, g) the “PRC2 cluster” biomarkers shown in Table 3, h) the “overlap” biomarkers shown in Table 4, i) the “SUZ12 target,” “Mikkelsen MEF,” and/or “Mikkelsen NPC” biomarkers shown in Table 5, j) KDM6A, k) KDM6B, l) EZH2, m) HMGN1, and subsets and/or combinations thereof.

41-60. (canceled)

61. A method of increasing the number of lymphoid progenitor cells from an initial population of lymphoid progenitor cells comprising contacting the lymphoid progenitor cells with an agent that inhibits polycomb repressor complex 2 (PRC2) activity or reduces H3K27me3 levels to thereby increase the number of lymphoid progenitor cells.

62. The method of claim 61, wherein the agent inhibits the activity of the EZH2 histone H3K27 methyltransferase subunit of PRC2.

63-66. (canceled)
